(19) United States
(12) Patent Application Publication
(10) Pub. No.: US 2025/0228131 A1
(43) Pub. Date: Jul. 10, 2025

(54) COMPOUND FOR ORGANIC OPTOELECTRONIC DEVICE, ORGANIC OPTOELECTRONIC DEVICE, AND DISPLAY DEVICE

(71) Applicant: Samsung SDI Co., Ltd., Yongin-si (KR)

(72) Inventors: Sunwoong SHIN, Suwon-si (KR); Youngmook LIM, Suwon-si (KR); Jongwoo WON, Suwon-si (KR); Junghoon KIM, Suwon-si (KR); Gibeom PARK, Suwon-si (KR); Kyoungrok KIM, Suwon-si (KR); Mijin LEE, Suwon-si (KR); Youngkyoung JO, Suwon-si (KR); Ho Kuk JUNG, Suwon-si (KR); Dalho HUH, Suwon-si (KR); Hyung Sun KIM, Suwon-si (KR); Sunha PARK, Suwon-si (KR)

(21) Appl. No.: 18/979,831

(22) Filed: Dec. 13, 2024

(30) Foreign Application Priority Data
Jan. 5, 2024 (KR) 10-2024-0002490

(51) Int. Cl. H10K 85/60; C07D 307/91; C07D 333/76; H10K 50/15

(52) U.S. Cl. CPC H10K 85/636; C07D 307/91; C07D 333/76; H10K 85/633; H10K 50/156; H10K 85/615; H10K 85/626; H10K 85/6574; H10K 85/6576

(57) ABSTRACT

A compound for an organic optoelectronic device, an organic optoelectronic device including the compound for an organic optoelectronic device, and a display device including the organic optoelectronic device, the compound for an organic optoelectronic device being represented by a combination of Chemical Formula 1 and Chemical Formula 2:

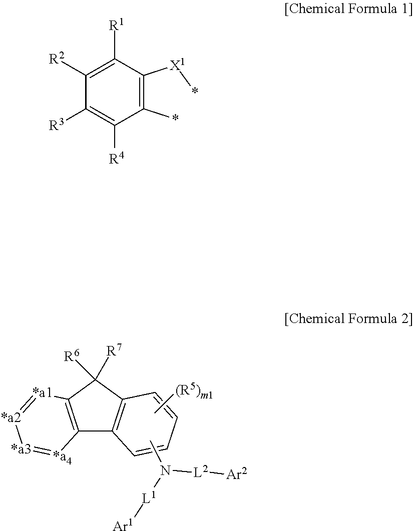

### CROSS-REFERENCE TO RELATED APPLICATION

This application claims priority to and the benefit of Korean Patent Application No. 10-2024-0002490 filed in the Korean Intellectual Property Office on Jan. 5, 2024, the entire contents of which are incorporated herein by reference.

### BACKGROUND

### 1. Field

Embodiments relate to a compound for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light by applying current to an organic light emitting material and performance of an organic light emitting diode may be affected by organic materials disposed between electrodes.

### SUMMARY

The embodiments may be realized by providing a compound for an organic optoelectronic device, the compound being represented by a combination of Chemical Formula 1 and Chemical Formula 2:

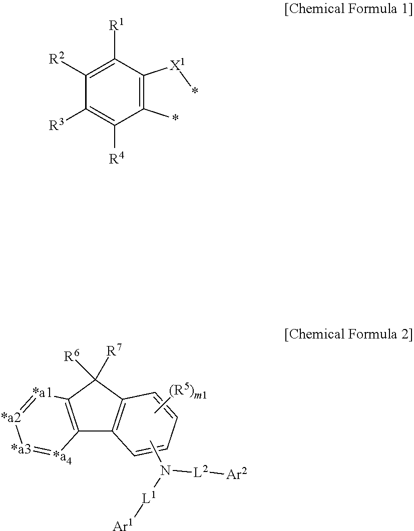

wherein, in Chemical Formula 1 and Chemical Formula 2, two adjacent ones of a1* to a4* of Chemical Formula 2 are linking carbons linked at * of Chemical Formula 1, the remaining two of a1* to a4* of Chemical Formula 2, not linked at * of Chemical Formula 1, are each independently CR^{a}, X¹ is O or S, R^{a} and R¹ to R⁵ are each independently hydrogen, deuterium, a cyano group, a substituted or unsubstituted C1 to C20 alkyl group, a substituted or unsubstituted C6 to C20 aryl group, or a combination thereof, R⁶ and R⁷ are each independently a substituted or unsubstituted C1 to C20 alkyl group or a substituted or unsubstituted C6 to C20 aryl group, L¹ and L² are each independently a single bond, a substituted or unsubstituted C6 to C20 arylene group, a substituted or unsubstituted C2 to C30 heterocyclic group, or a combination thereof, Ar¹ and Ar² are each independently a substituted or unsubstituted C6 to C30 aryl group or a substituted or unsubstituted C2 to C30 heterocyclic group, m1 is an integer of 1 to 3, provided that when m1 is 2 or 3, each R⁵ is the same or different from each other, and at least one of R¹ to R⁴ is a substituted or unsubstituted tert-butyl group.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view showing an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not necessarily an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example, the “substituted” refers to replacement of at least one hydrogen of a substituent or a compound by deuterium, a C1 to C20 alkyl group, a C6 to C30 aryl group, or a cyano group. In a specific example, the “substituted” refers to replacement of at least one hydrogen of a substituent or a compound by deuterium, a C1 to C5 alkyl group, a C6 to C18 aryl group, or a cyano group. In a specific example, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to some example embodiments is described.

A compound for an organic optoelectronic device according to some example embodiments may be represented by a combination of Chemical Formula 1 and Chemical Formula 2.

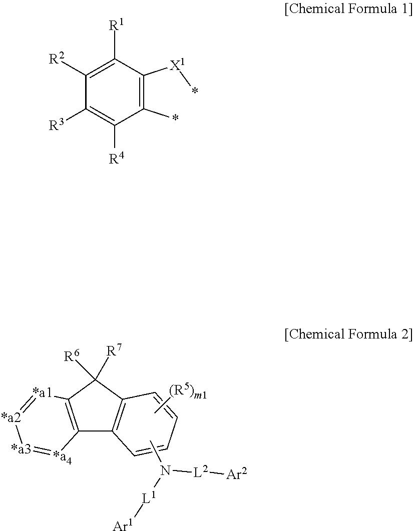

In Chemical Formula 1 and Chemical Formula 2, two adjacent ones of a1* to a4* of Chemical Formula 2 may be linking carbons linked at * of Chemical Formula 1, the remaining two of a1* to a4* of Chemical Formula 2, not linked at * of Chemical Formula 1, may each independently be CR^{a}. As used herein, the term “linking carbon” refers to a shared carbon at which fused rings are linked.

X¹ may be, e.g., O or S.

R^{a} and R¹ to R⁵ may each independently be or include, e.g., hydrogen, deuterium, a cyano group, a substituted or unsubstituted C1 to C20 alkyl group, a substituted or unsubstituted C6 to C20 aryl group, or a combination thereof.

R⁶ and R⁷ may each independently be or include, e.g., a substituted or unsubstituted C1 to C20 alkyl group or a substituted or unsubstituted C6 to C20 aryl group.

L¹ and L² may each independently be or include, e.g., a single bond, a substituted or unsubstituted C6 to C20 arylene group, a substituted or unsubstituted C2 to C30 heterocyclic group, or a combination thereof.

Ar¹ and Ar² may each independently be or include, e.g., a substituted or unsubstituted C6 to C30 aryl group or a substituted or unsubstituted C2 to C30 heterocyclic group.

m1 may be, e.g., an integer of 1 to 3. In an implementation, m1 may be 2 or 3, and each R⁵ may be the same or different from each other.

In an implementation, at least one of R¹ to R⁴ may be or may include, e.g., a substituted or unsubstituted tert-butyl group.

The compound represented by the combination of Chemical Formula 1 and Chemical Formula 2 may have a structure in which fluorene is fused with benzofuran or benzothiophene in the core, and an amine group may be substituted in or on the fluorene moiety. As the hole mobility becomes faster, the charge balance may be increased, and thus the driving voltage of the organic light emitting diode using or including the compound may be lowered and the life-span can be realized.

In an implementation, the tert-butyl group may be substituted on the benzofuran (or benzothiophene) side, and the efficiency of the organic light emitting diode can be increased as a low refractive index material.

In an implementation, the combination of Chemical Formula 1 and Chemical Formula 2 may be represented by, e.g., one of Chemical Formula 1A to Chemical Formula 1F.

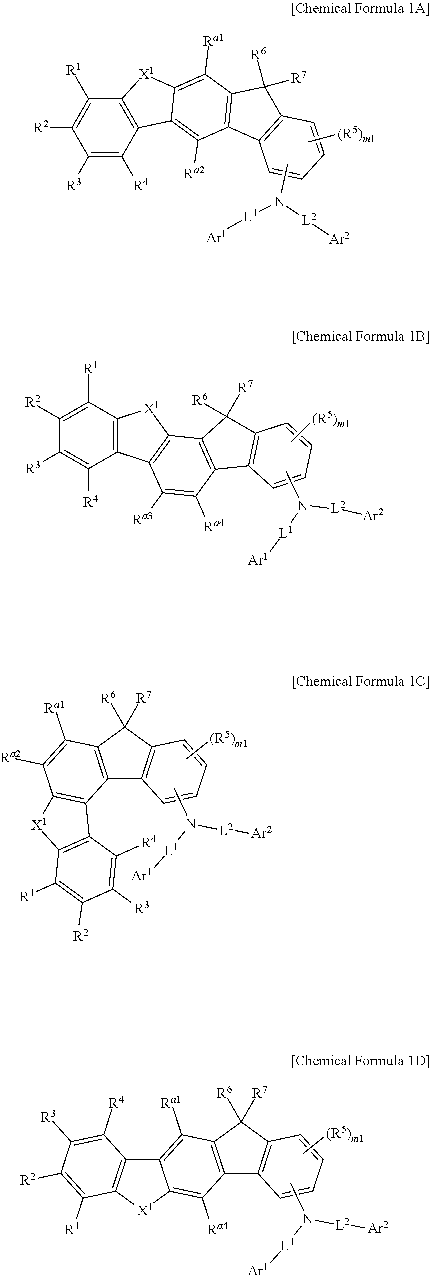

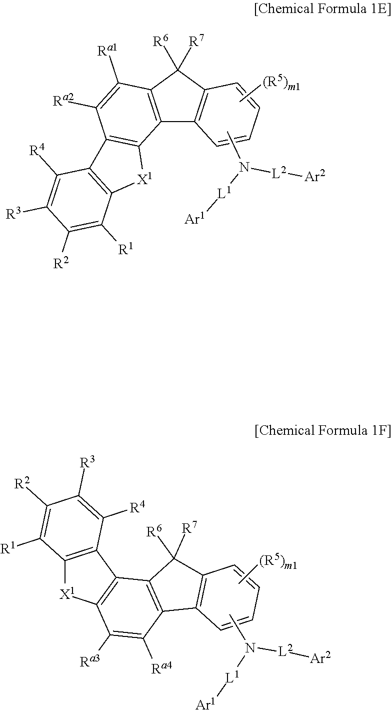

In Chemical Formula 1A to Chemical Formula 1F, X¹, R¹ to R⁷, L¹, L², Ar¹, Ar², and m1 may be defined the same as those of Chemical Formula 1 and Chemical Formula 2.

R^{a1} to R^{a4} may each independently be defined the same as R^{a} of Chemical Formula 2.

In an implementation, Chemical Formula 1A may be represented by, e.g., one of Chemical Formula 1A-1 to Chemical Formula 1A-4.

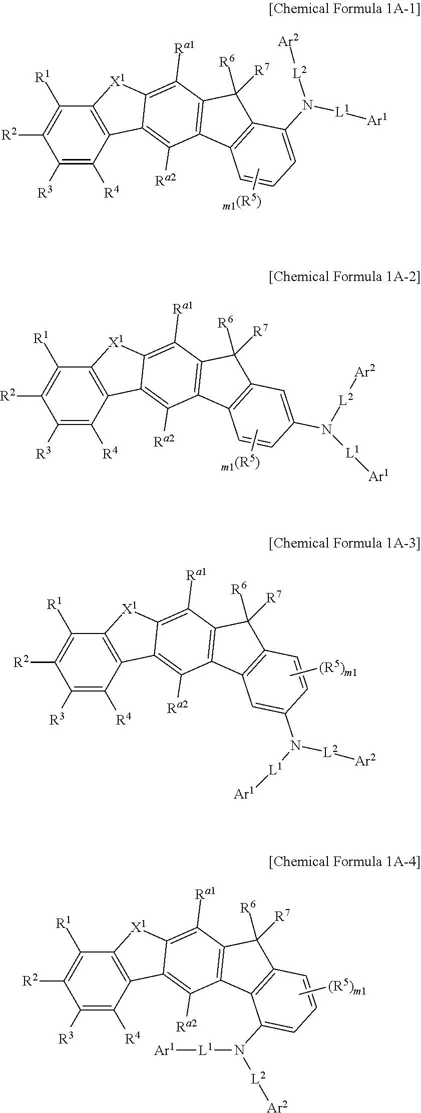

In Chemical Formula 1A-1 to Chemical Formula 1A-4, X¹, R¹ to R⁷, R^{a1}, R^{a2}, L¹, L², Ar¹, Ar², and m1 may be defined the same as those described above.

In an implementation, Chemical Formula 1B may be represented by, e.g., one of Chemical Formula 1B-1 to Chemical Formula 1B-4.

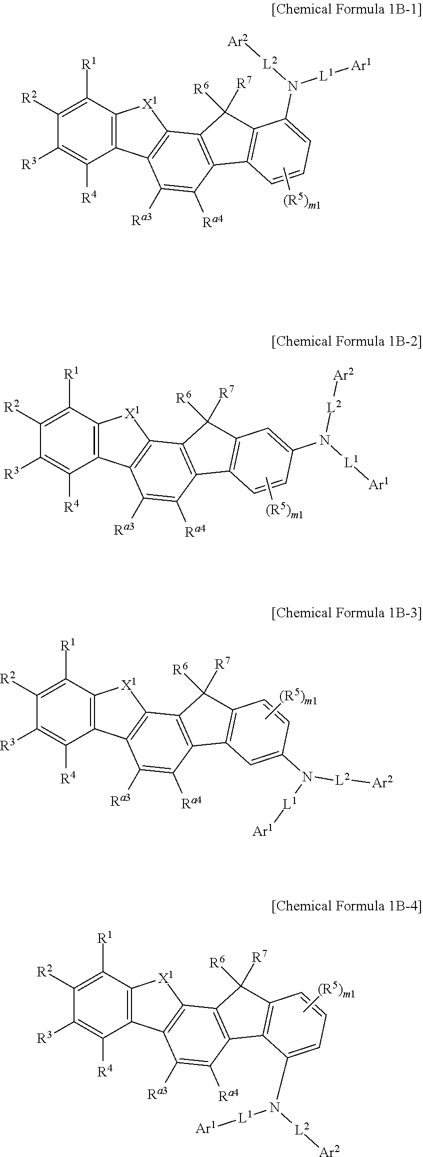

In Chemical Formula 1B-1 to Chemical Formula 1B-4, X¹, R¹ to R⁷, R^{a3}, R^{a4}, L¹, L², Ar¹, Ar², and m1 may be defined the same as those described above.

In an implementation, Chemical Formula 1C may be represented by, e.g., one of Chemical Formula 1C-1 to Chemical Formula 1C-4.

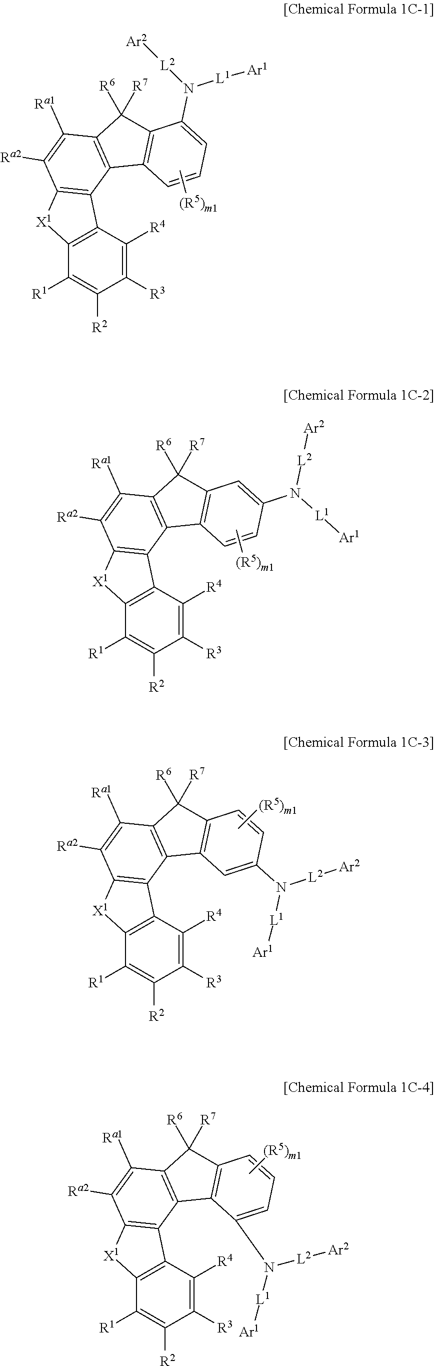

In Chemical Formula 1C-1 to Chemical Formula 1C-4, X¹, R¹ to R⁷, R^{a1}, R^{a2}, L¹, L², Ar¹, Ar², and m1 may be defined the same as those described above.

In an implementation, Chemical Formula 1D may be represented by, e.g., one of Chemical Formula 1D-1 to Chemical Formula 1D-4.

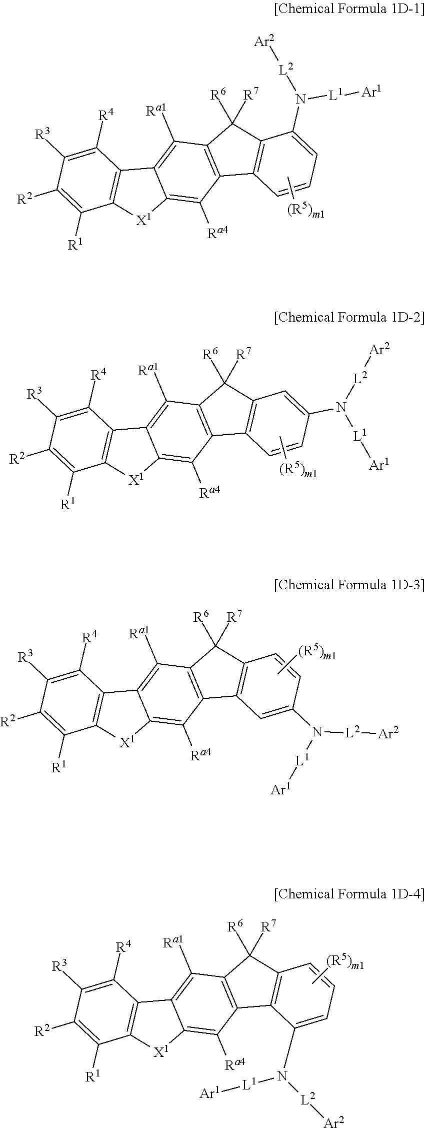

In Chemical Formula 1D-1 to Chemical Formula 1D-4, X¹, R¹ to R⁷, R^{a1}, R^{a4}, L¹, L², Ar¹, Ar², and m1 may be defined the same as those described above.

In an implementation, Chemical Formula 1E may be represented by, e.g., one of Chemical Formula 1E-1 to Chemical Formula 1E-4.

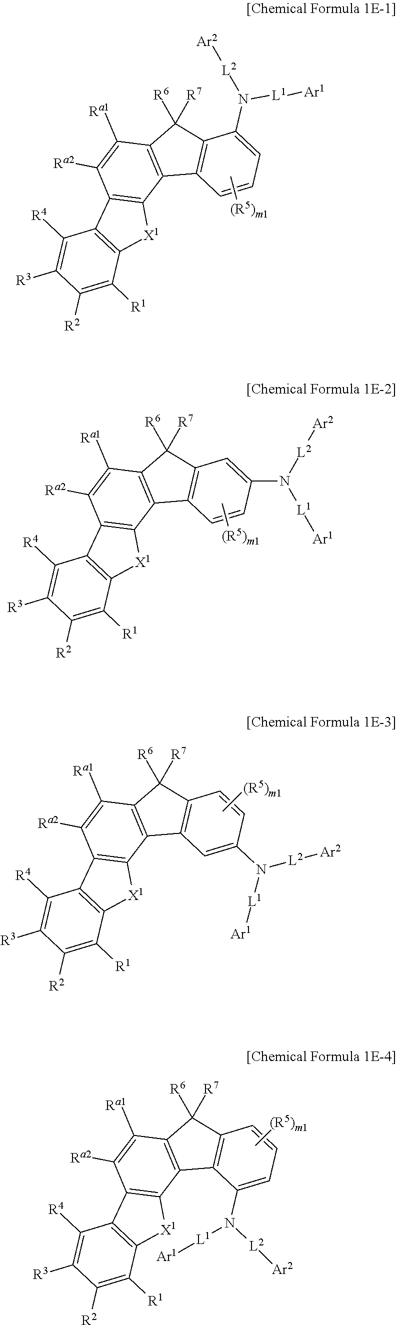

In Chemical Formula 1E-1 to Chemical Formula 1E-4, X¹, R¹ to R⁷, R^{a1}, R^{a2}, L¹, L², Ar¹, Ar², and m1 may be defined the same as those described above.

In an implementation, Chemical Formula 1F may be represented by, e.g., one of Chemical Formula 1F-1 to Chemical Formula 1F-4.

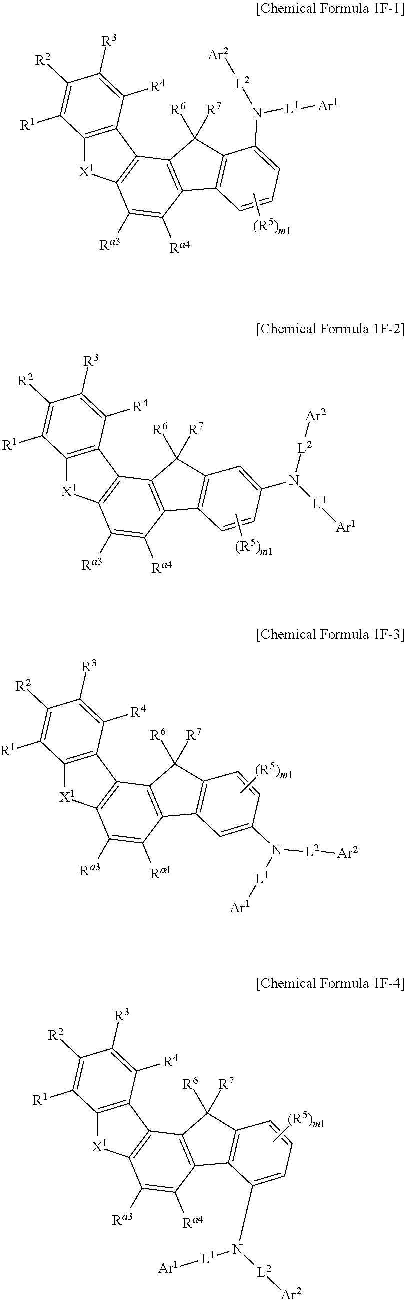

In Chemical Formula 1F-1 to Chemical Formula 1F-4, X¹, R¹ to R⁷, R^{a3}, R^{a4}, L¹, L², Ar¹, Ar², and m1 may be defined the same as those described above.

In an implementation, the combination of Chemical Formula 1 and Chemical Formula 2 may be represented by, e.g., Chemical Formula 1A-2, Chemical Formula 1A-4, Chemical Formula 1B-2, Chemical Formula 1B-4, Chemical Formula 1C-2, Chemical Formula 1C-4, Chemical Formula 1D-2, Chemical Formula 1D-4, Chemical Formula 1E-2, Chemical Formula 1E-4, Chemical Formula 1F-2, or Chemical Formula 1F-4.

In an implementation, the combination of Chemical Formula 1 and Chemical Formula 2 may be represented by, e.g., Chemical Formula 1A-4, Chemical Formula 1C-4, Chemical Formula 1E-2, Chemical Formula 1E-4, or Chemical Formula 1F-4.

In an implementation, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

In an implementation, Ar¹ and Ar² may each independently be, e.g., a substituted or unsubstituted phenyl group, a substituted or unsubstituted biphenyl group, a substituted or unsubstituted naphthyl group, a substituted or unsubstituted fluorenyl group, a substituted or unsubstituted dibenzofuranyl group, or a substituted or unsubstituted dibenzothiophenyl group.

In an implementation, L¹ and L² may each independently be, e.g., a single bond or a substituted or unsubstituted phenylene group.

In an implementation, moieties *-L¹-Ar¹ and *-L²-Ar² may each independently be, e.g., a moiety of Group I.

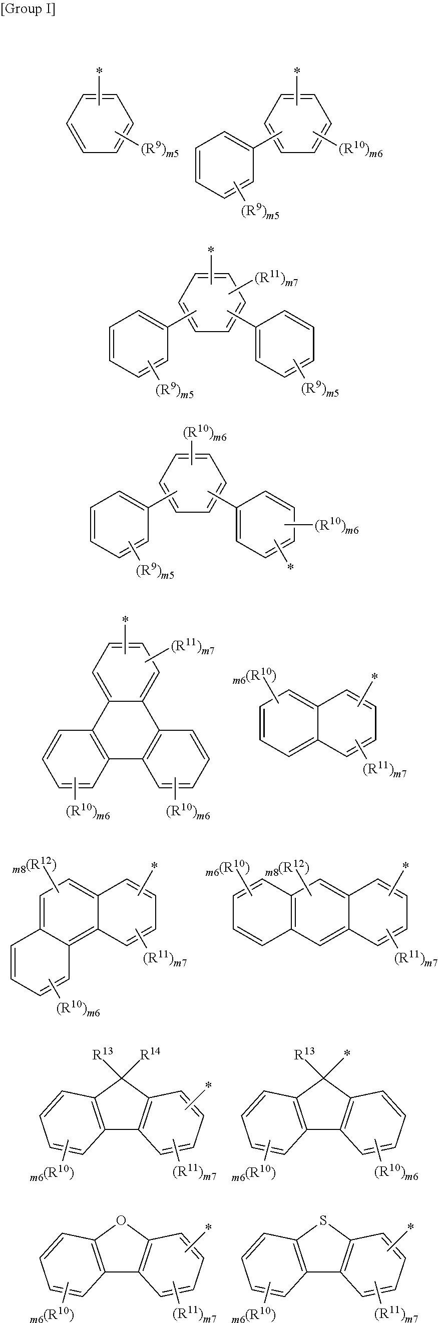

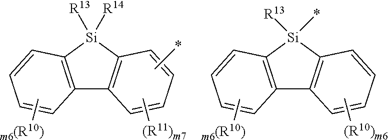

In Group I, R⁹ to R¹² may each independently be, e.g., hydrogen, deuterium, a cyano group, a substituted or unsubstituted C1 to C10 alkyl group, or a substituted or unsubstituted C6 to C12 aryl group.

R¹³ and R¹⁴ may each independently be, e.g., a substituted or unsubstituted C1 to C10 alkyl group or a substituted or unsubstituted C6 to C12 aryl group.

m5 may be, e.g., an integer of 1 to 5.

m6 may be, e.g., an integer of 1 to 4.

m7 may be, e.g., an integer of 1 to 3.

m8 may be, e.g., 1 or 2.

* is a linking point.

In an implementation, m5 may be 2 to 5, and each R⁹ may be the same or different from each other.

In an implementation, m6 may be 2 to 4, and each R¹⁰ may be the same or different from each other.

In an implementation, m7 may be 2 or 3, and each R¹¹ may be the same or different from each other.

In an implementation, m8 may be 2, each R¹² may be the same or different from each other.

In an implementation, R^{a}, R^{a1} to R^{a4}, and R¹ to R⁴ may each independently be, e.g., hydrogen, deuterium, a substituted or unsubstituted C1 to C10 alkyl group, a substituted or unsubstituted C6 to C12 aryl group, or a combination thereof. In an implementation, at least one of R¹ to R⁴ may be, e.g., a substituted or unsubstituted tert-butyl group.

In an implementation, R^{a}, R^{a1} to R^{a4}, and R¹ to R⁴ may each independently be, e.g., hydrogen, deuterium, or a substituted or unsubstituted C1 to C10 alkyl group, and at least one of R¹ to R⁴ may be a tert-butyl group.

In an implementation, R¹ may be a substituted or unsubstituted tert-butyl group.

In an implementation, R² may be a substituted or unsubstituted tert-butyl group.

In an implementation, R³ may be a substituted or unsubstituted tert-butyl group.

In an implementation, R⁴ may be a substituted or unsubstituted tert-butyl group.

In an implementation, at least two of R¹ to R⁴ may be a substituted or unsubstituted tert-butyl group.

In an implementation, R¹ and R² may be a substituted or unsubstituted tert-butyl group.

In an implementation, R¹ and R³ may be a substituted or unsubstituted tert-butyl group.

In an implementation, R¹ and R⁴ may be a substituted or unsubstituted tert-butyl group.

In an implementation, R² and R³ may be a substituted or unsubstituted tert-butyl group.

In an implementation, R² and R⁴ may be a substituted or unsubstituted tert-butyl group.

In an implementation, R³ and R⁴ may be a substituted or unsubstituted tert-butyl group.

In an implementation, R¹, R², and R³ may be a substituted or unsubstituted tert-butyl group.

In an implementation, R¹, R², and R⁴ may be a substituted or unsubstituted tert-butyl group.

In an implementation, R¹, R³, and R⁴ may be a substituted or unsubstituted tert-butyl group.

In an implementation, R², R³, and R⁴ may be a substituted or unsubstituted tert-butyl group.

In an implementation, R¹, R², R³ and R⁴ may each be a substituted or unsubstituted tert-butyl group.

In an implementation, R⁶ and R⁷ may each independently be, e.g., a substituted or unsubstituted C1 to C10 alkyl group or a substituted or unsubstituted C6 to C12 aryl group.

In an implementation, R⁶ and R⁷ may each independently be, e.g., a substituted or unsubstituted methyl group or a substituted or unsubstituted phenyl group.

In an implementation, the compound for an organic optoelectronic device represented by a combination of Chemical Formula 1 and Chemical Formula 2 may be, e.g., a compound of Group 1.

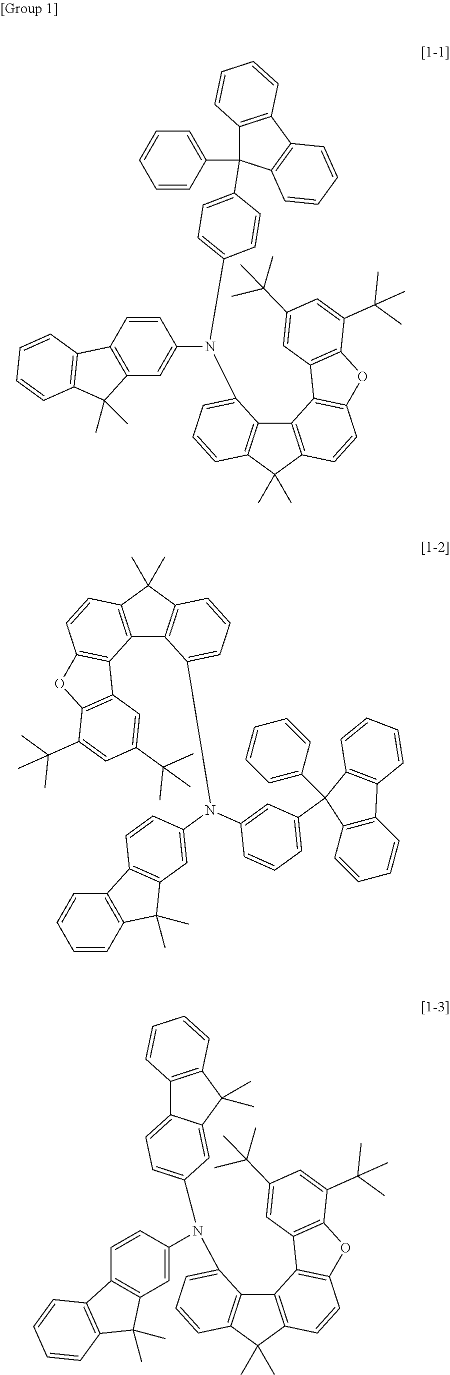

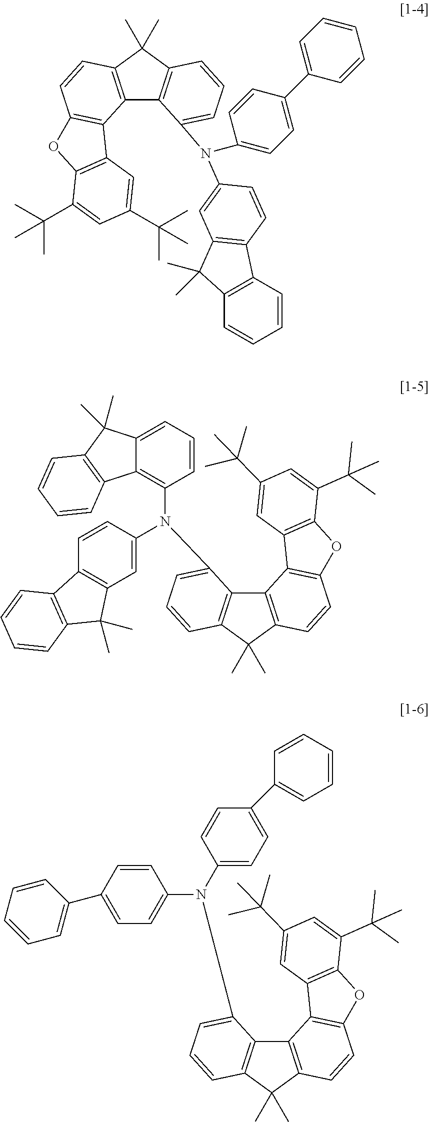 [SMILES: CC(C)(C)c1cc(C(C)(C)C)c2oc3ccc4c(c3c2c1)-c1c(N(c2ccc(-c3ccccc3)cc2)c2ccc3c(c2)C(C)(C)c2ccccc2-3)cccc1C4(C)C ; CC(C)(C)c1cc(C(C)(C)C)c2oc3ccc4c(c3c2c1)-c1c(N(c2ccc3c(c2)C(C)(C)c2ccccc2-3)c2cccc3c2-c2ccccc2C3(C)C)cccc1C4(C)C ; CC(C)(C)c1cc(C(C)(C)C)c2oc3ccc4c(c3c2c1)-c1c(N(c2ccc(-c3ccccc3)cc2)c2ccc(-c3ccccc3)cc2)cccc1C4(C)C]

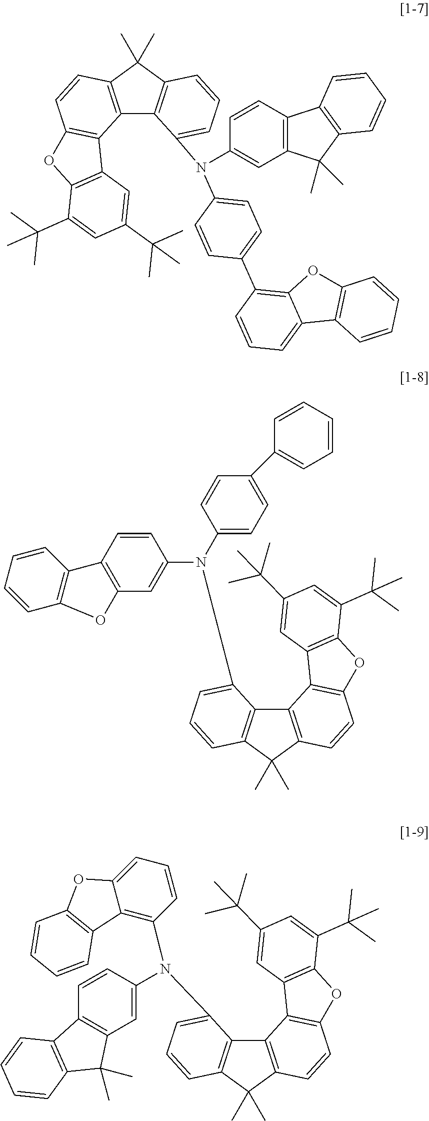 [SMILES: CC(C)(C)c1cc(C(C)(C)C)c2oc3ccc4c(c3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1cc(C(C)(C)C)c2oc3ccc4c(c3c2c1)-c1c(N(c2ccc(-c3ccccc3)cc2)c2ccc3c(c2)oc2ccccc23)cccc1C4(C)C ; CC(C)(C)c1cc(C(C)(C)C)c2oc3ccc4c(c3c2c1)-c1c(N(c2ccc3c(c2)C(C)(C)c2ccccc2-3)c2cccc3oc5ccccc5c23)cccc1C4(C)C]

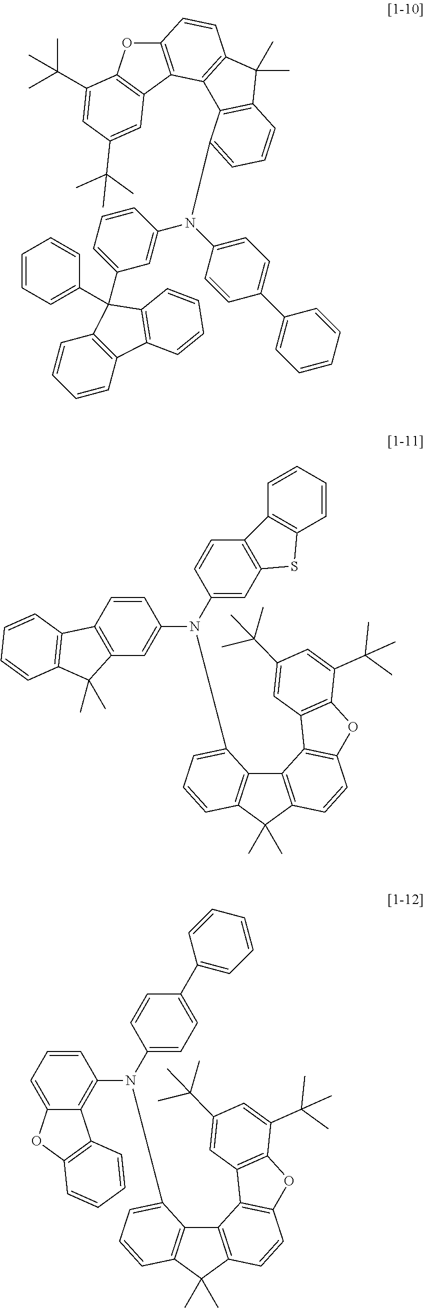 [SMILES: CC(C)(C)c1cc(C(C)(C)C)c2oc3ccc4c(c3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1cc(C(C)(C)C)c2oc3ccc4c(c3c2c1)-c1c(N(c2ccc3c(c2)C(C)(C)c2ccccc2-3)c2ccc3c(c2)sc2ccccc23)cccc1C4(C)C ; CC(C)(C)c1cc(C(C)(C)C)c2oc3ccc4c(c3c2c1)-c1c(N(c2ccc(-c3ccccc3)cc2)c2cccc3oc5ccccc5c23)cccc1C4(C)C]

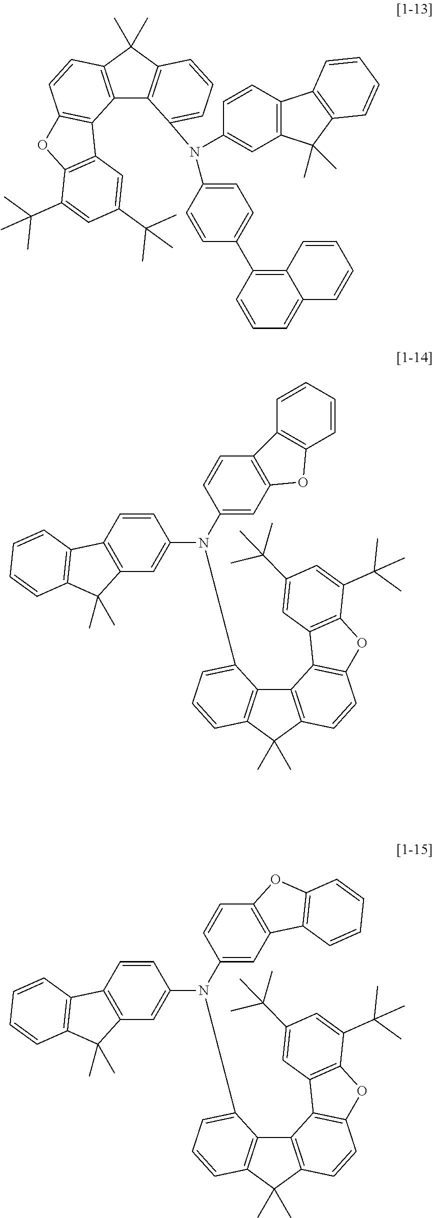 [SMILES: CC(C)(C)c1cc(C(C)(C)C)c2oc3ccc4c(c3c2c1)-c1c(N(c2ccc(-c3cccc5ccccc35)cc2)c2ccc3c(c2)C(C)(C)c2ccccc2-3)cccc1C4(C)C ; CC(C)(C)c1cc(C(C)(C)C)c2oc3ccc4c(c3c2c1)-c1c(N(c2ccc3c(c2)C(C)(C)c2ccccc2-3)c2ccc3c(c2)oc2ccccc23)cccc1C4(C)C ; CC(C)(C)c1cc(C(C)(C)C)c2oc3ccc4c(c3c2c1)-c1c(N(c2ccc3c(c2)C(C)(C)c2ccccc2-3)c2ccc3oc5ccccc5c3c2)cccc1C4(C)C]

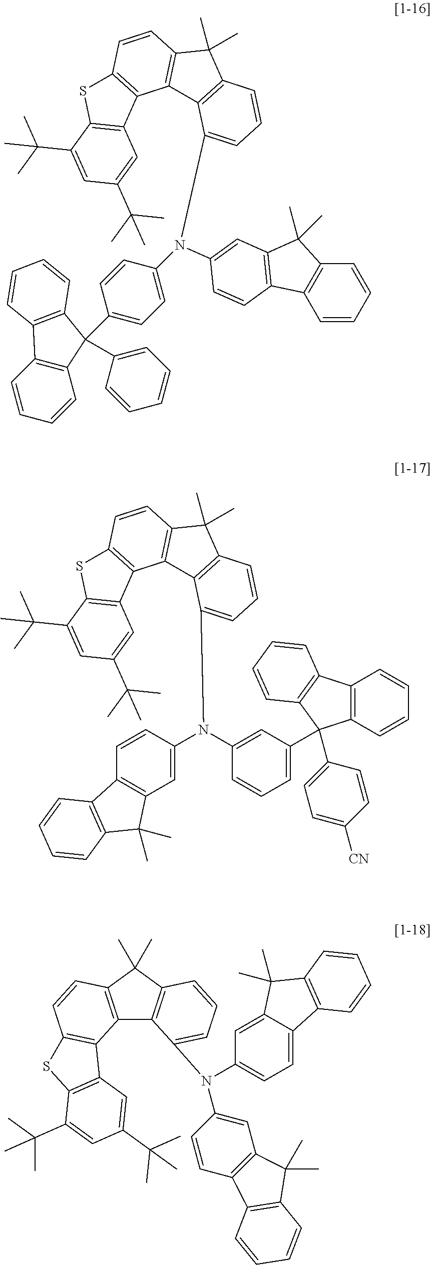 [SMILES: CC(C)(C)c1cc(C(C)(C)C)c2sc3ccc4c(c3c2c1)-c1c(N(c2ccc(C3(c5ccccc5)c5ccccc5-c5ccccc53)cc2)c2ccc3c(c2)C(C)(C)c2ccccc2-3)cccc1C4(C)C ; CC(C)(C)c1cc(C(C)(C)C)c2sc3ccc4c(c3c2c1)-c1c(N(c2cccc(C3(c5ccc(C#N)cc5)c5ccccc5-c5ccccc53)c2)c2ccc3c(c2)C(C)(C)c2ccccc2-3)cccc1C4(C)C ; CC(C)(C)c1cc(C(C)(C)C)c2sc3ccc4c(c3c2c1)-c1c(N(c2ccc3c(c2)C(C)(C)c2ccccc2-3)c2ccc3c(c2)C(C)(C)c2ccccc2-3)cccc1C4(C)C]

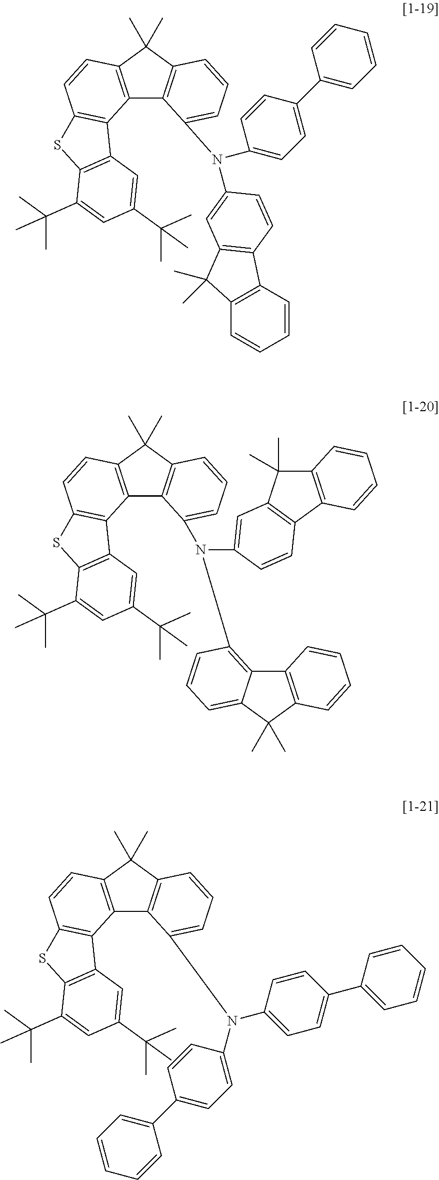 [SMILES: CC(C)(C)c1cc(C(C)(C)C)c2sc3ccc4c(c3c2c1)-c1c(N(c2ccc(-c3ccccc3)cc2)c2ccc3c(c2)C(C)(C)c2ccccc2-3)cccc1C4(C)C ; CC(C)(C)c1cc(C(C)(C)C)c2sc3ccc4c(c3c2c1)-c1c(N(c2ccc3c(c2)C(C)(C)c2ccccc2-3)c2cccc3c2-c2ccccc2C3(C)C)cccc1C4(C)C ; CC(C)(C)c1cc(C(C)(C)C)c2sc3ccc4c(c3c2c1)-c1c(N(c2ccc(-c3ccccc3)cc2)c2ccc(-c3ccccc3)cc2)cccc1C4(C)C]

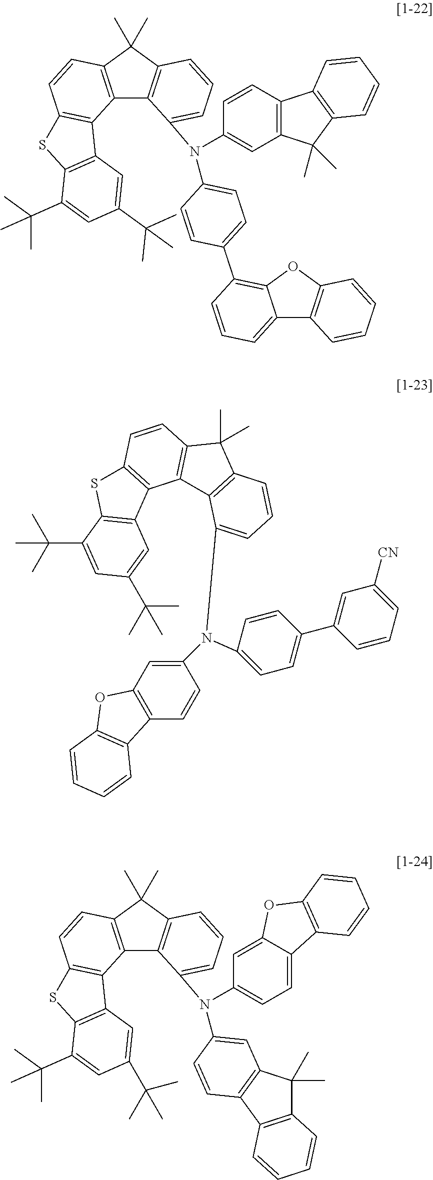 [SMILES: CC(C)(C)c1cc(C(C)(C)C)c2sc3ccc4c(c3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1cc(C(C)(C)C)c2sc3ccc4c(c3c2c1)-c1c(N(c2ccc(-c3cccc(C#N)c3)cc2)c2ccc3c(c2)oc2ccccc23)cccc1C4(C)C ; CC(C)(C)c1cc(C(C)(C)C)c2sc3ccc4c(c3c2c1)-c1c(N(c2ccc3c(c2)C(C)(C)c2ccccc2-3)c2ccc3c(c2)oc2ccccc23)cccc1C4(C)C]

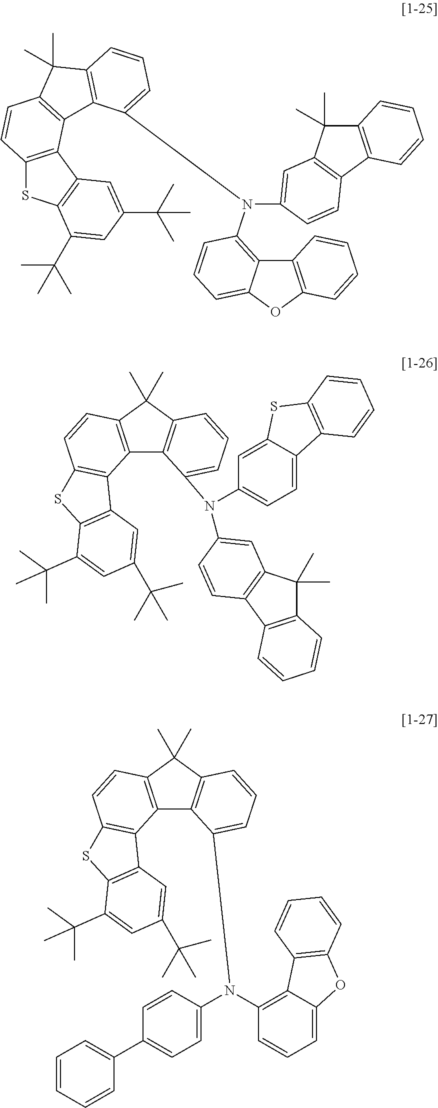 [SMILES: CC(C)(C)c1cc(C(C)(C)C)c2sc3ccc4c(c3c2c1)-c1c(N(c2ccc3c(c2)C(C)(C)c2ccccc2-3)c2cccc3oc5ccccc5c23)cccc1C4(C)C ; CC(C)(C)c1cc(C(C)(C)C)c2sc3ccc4c(c3c2c1)-c1c(N(c2ccc3c(c2)C(C)(C)c2ccccc2-3)c2ccc3c(c2)sc2ccccc23)cccc1C4(C)C ; CC(C)(C)c1cc(C(C)(C)C)c2sc3ccc4c(c3c2c1)-c1c(N(c2ccc(-c3ccccc3)cc2)c2cccc3oc5ccccc5c23)cccc1C4(C)C]

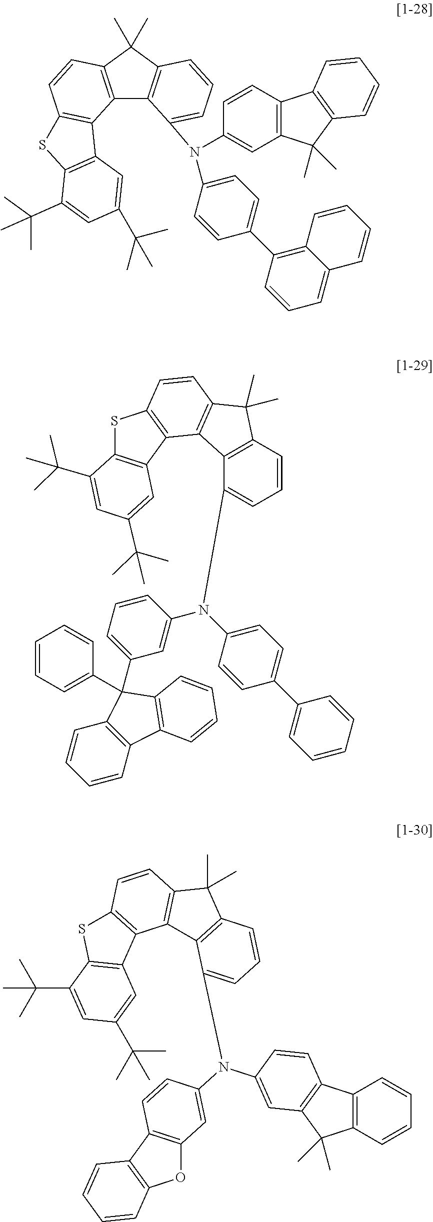 [SMILES: CC(C)(C)c1cc(C(C)(C)C)c2sc3ccc4c(c3c2c1)-c1c(N(c2ccc(-c3cccc5ccccc35)cc2)c2ccc3c(c2)C(C)(C)c2ccccc2-3)cccc1C4(C)C ; CC(C)(C)c1cc(C(C)(C)C)c2sc3ccc4c(c3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1cc(C(C)(C)C)c2sc3ccc4c(c3c2c1)-c1c(N(c2ccc3c(c2)C(C)(C)c2ccccc2-3)c2ccc3c(c2)oc2ccccc23)cccc1C4(C)C]

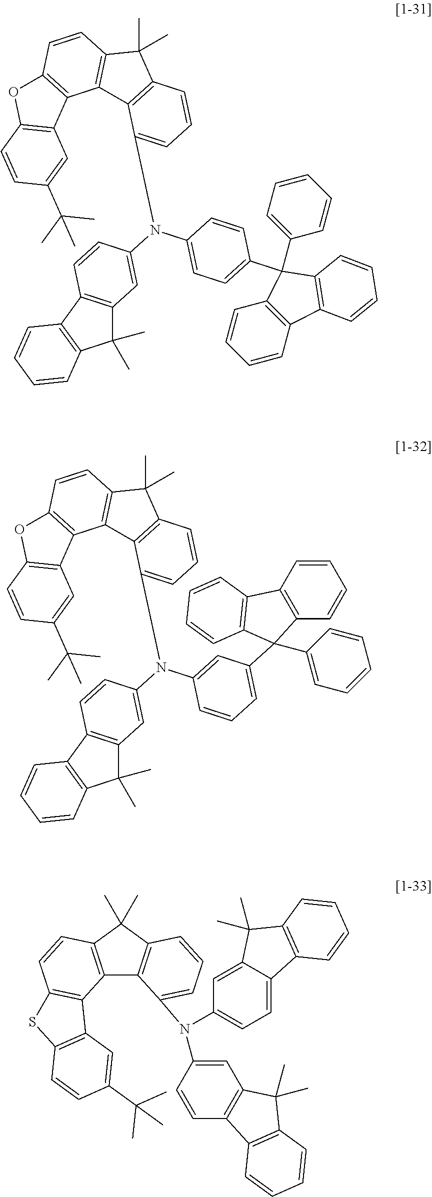 [SMILES: CC(C)(C)c1ccc2oc3ccc4c(c3c2c1)-c1c(N(c2ccc(C3(c5ccccc5)c5ccccc5-c5ccccc53)cc2)c2ccc3c(c2)C(C)(C)c2ccccc2-3)cccc1C4(C)C ; CC(C)(C)c1ccc2oc3ccc4c(c3c2c1)-c1c(N(c2cccc(C3(c5ccccc5)c5ccccc5-c5ccccc53)c2)c2ccc3c(c2)C(C)(C)c2ccccc2-3)cccc1C4(C)C ; CC(C)(C)c1ccc2sc3ccc4c(c3c2c1)-c1c(N(c2ccc3c(c2)C(C)(C)c2ccccc2-3)c2ccc3c(c2)C(C)(C)c2ccccc2-3)cccc1C4(C)C]

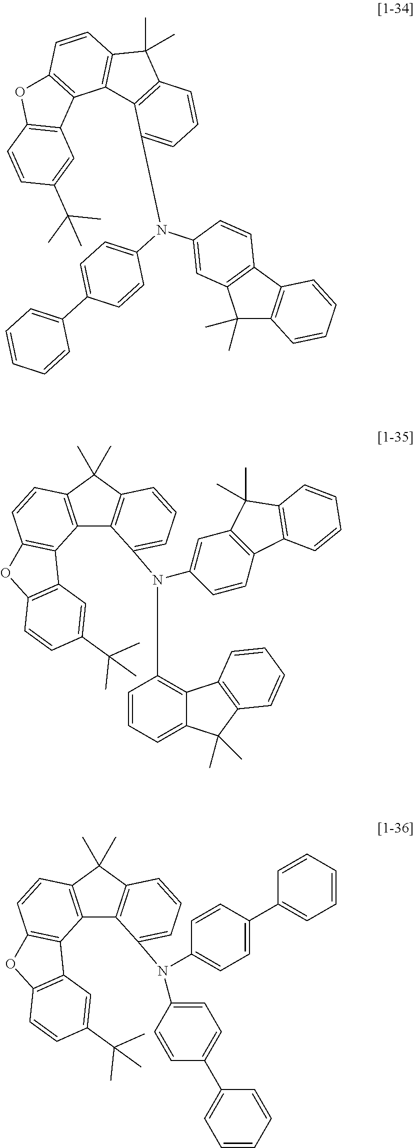 [SMILES: CC(C)(C)c1ccc2oc3ccc4c(c3c2c1)-c1c(N(c2ccc(-c3ccccc3)cc2)c2ccc3c(c2)C(C)(C)c2ccccc2-3)cccc1C4(C)C ; CC(C)(C)c1ccc2oc3ccc4c(c3c2c1)-c1c(N(c2ccc3c(c2)C(C)(C)c2ccccc2-3)c2cccc3c2-c2ccccc2C3(C)C)cccc1C4(C)C ; CC(C)(C)c1ccc2oc3ccc4c(c3c2c1)-c1c(N(c2ccc(-c3ccccc3)cc2)c2ccc(-c3ccccc3)cc2)cccc1C4(C)C]

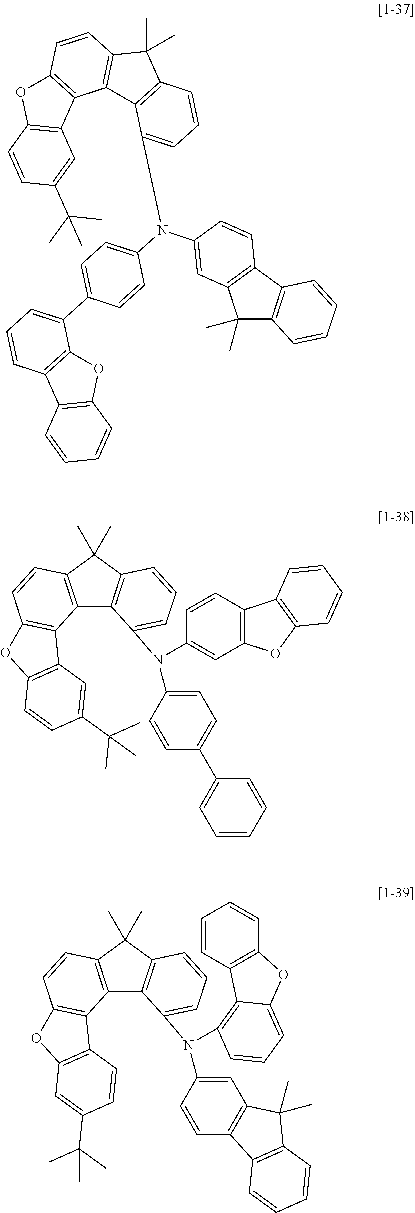 [SMILES: CC(C)(C)c1ccc2oc3ccc4c(c3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1ccc2oc3ccc4c(c3c2c1)-c1c(N(c2ccc(-c3ccccc3)cc2)c2ccc3c(c2)oc2ccccc23)cccc1C4(C)C ; CC(C)(C)c1ccc2c(c1)oc1ccc3c(c12)-c1c(N(c2ccc4c(c2)C(C)(C)c2ccccc2-4)c2cccc4oc5ccccc5c24)cccc1C3(C)C]

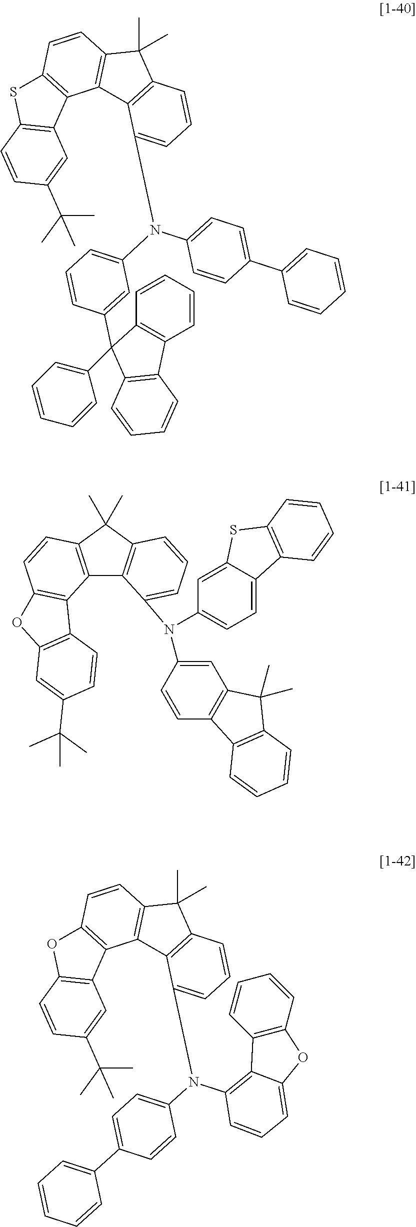 [SMILES: CC(C)(C)c1ccc2sc3ccc4c(c3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1ccc2c(c1)oc1ccc3c(c12)-c1c(N(c2ccc4c(c2)C(C)(C)c2ccccc2-4)c2ccc4c(c2)sc2ccccc24)cccc1C3(C)C ; CC(C)(C)c1ccc2oc3ccc4c(c3c2c1)-c1c(N(c2ccc(-c3ccccc3)cc2)c2cccc3oc5ccccc5c23)cccc1C4(C)C]

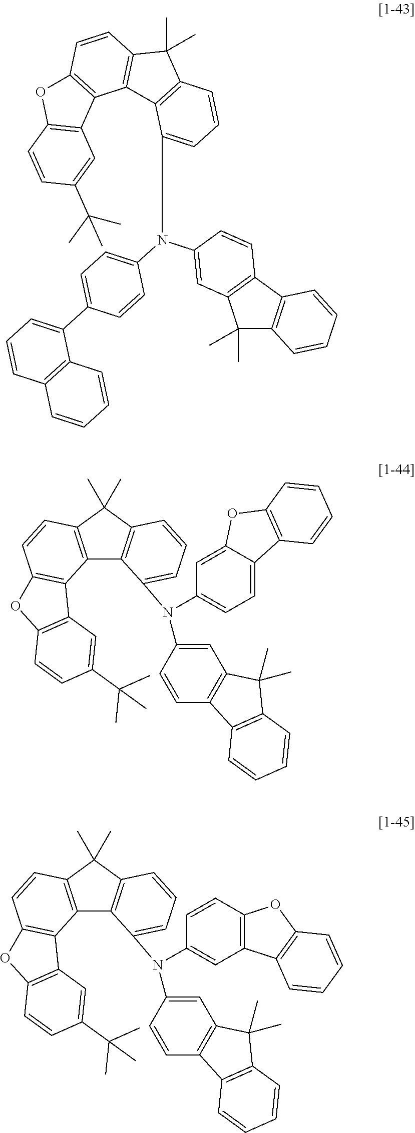 [SMILES: CC(C)(C)c1ccc2oc3ccc4c(c3c2c1)-c1c(N(c2ccc(-c3cccc5ccccc35)cc2)c2ccc3c(c2)C(C)(C)c2ccccc2-3)cccc1C4(C)C ; CC(C)(C)c1ccc2oc3ccc4c(c3c2c1)-c1c(N(c2ccc3c(c2)C(C)(C)c2ccccc2-3)c2ccc3c(c2)oc2ccccc23)cccc1C4(C)C ; CC(C)(C)c1ccc2oc3ccc4c(c3c2c1)-c1c(N(c2ccc3c(c2)C(C)(C)c2ccccc2-3)c2ccc3oc5ccccc5c3c2)cccc1C4(C)C]

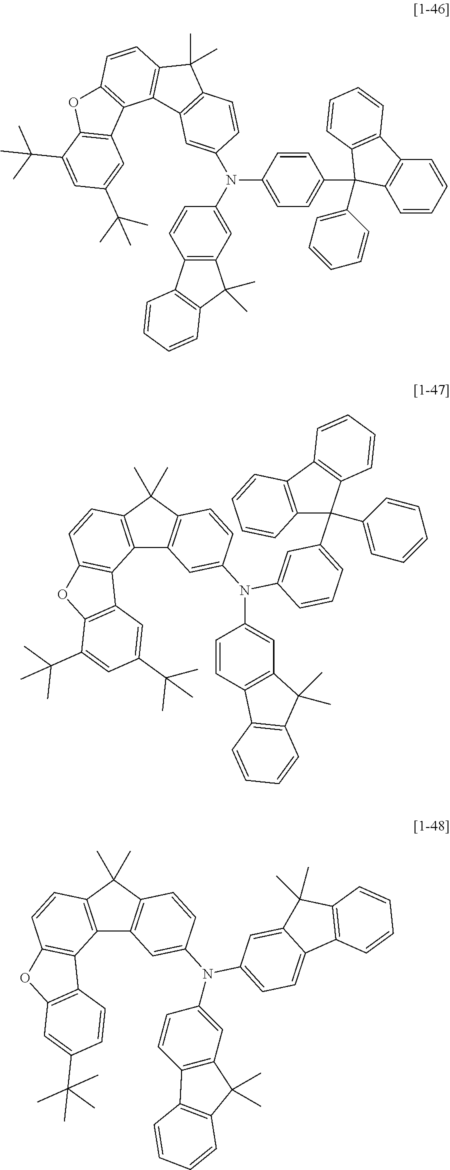

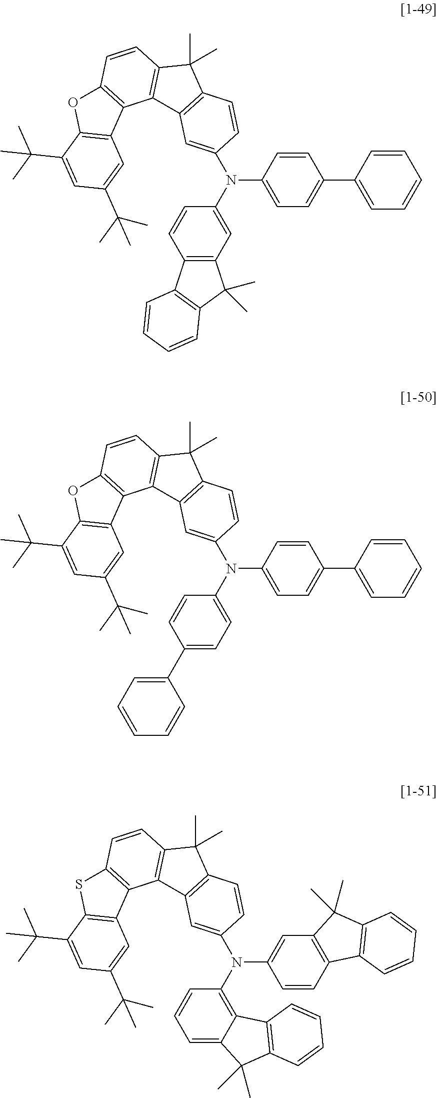 [SMILES: CC(C)(C)c1cc(C(C)(C)C)c2oc3ccc4c(c3c2c1)-c1cc(N(c2ccc(-c3ccccc3)cc2)c2ccc3c(c2)C(C)(C)c2ccccc2-3)ccc1C4(C)C ; CC(C)(C)c1cc(C(C)(C)C)c2oc3ccc4c(c3c2c1)-c1cc(N(c2ccc(-c3ccccc3)cc2)c2ccc(-c3ccccc3)cc2)ccc1C4(C)C ; CC(C)(C)c1cc(C(C)(C)C)c2sc3ccc4c(c3c2c1)-c1cc(N(c2ccc3c(c2)C(C)(C)c2ccccc2-3)c2cccc3c2-c2ccccc2C3(C)C)ccc1C4(C)C]

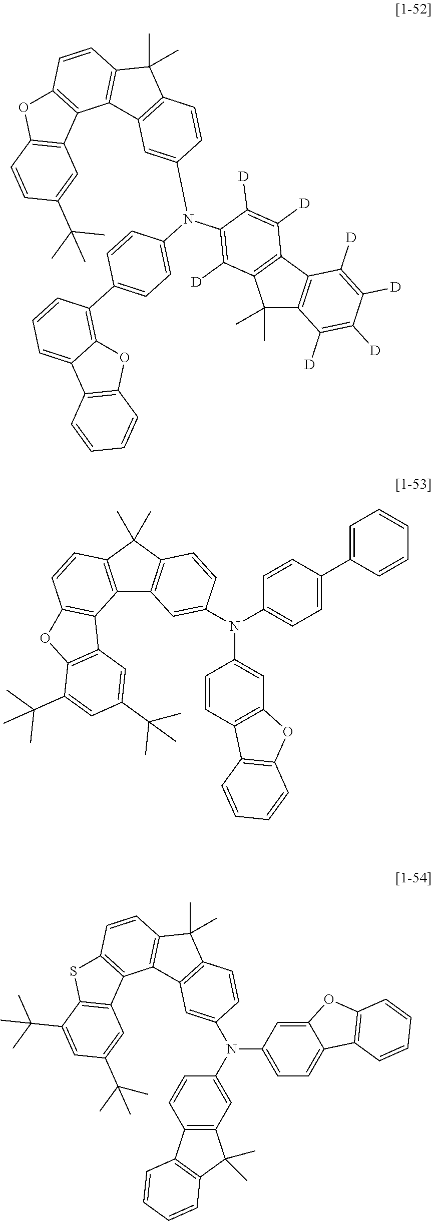 [SMILES: [2H]c1c([2H])c([2H])c2c(c1[2H])-c1c([2H])c([2H])c(N(c3ccc(-c4cccc5c4oc4ccccc45)cc3)c3ccc4c(c3)-c3c(ccc5oc6ccc(C(C)(C)C)cc6c35)C4(C)C)c([2H])c1C2(C)C ; CC(C)(C)c1cc(C(C)(C)C)c2oc3ccc4c(c3c2c1)-c1cc(N(c2ccc(-c3ccccc3)cc2)c2ccc3c(c2)oc2ccccc23)ccc1C4(C)C ; CC(C)(C)c1cc(C(C)(C)C)c2sc3ccc4c(c3c2c1)-c1cc(N(c2ccc3c(c2)C(C)(C)c2ccccc2-3)c2ccc3c(c2)oc2ccccc23)ccc1C4(C)C]

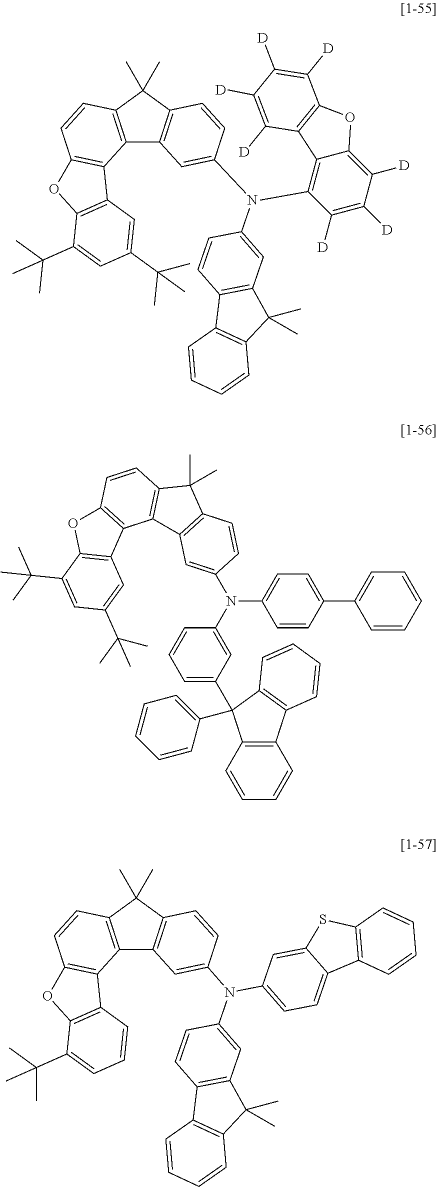 [SMILES: [2H]c1c([2H])c([2H])c2c(oc3c([2H])c([2H])c([2H])c(N(c4ccc5c(c4)-c4c(ccc6oc7c(C(C)(C)C)cc(C(C)(C)C)cc7c46)C5(C)C)c4ccc5c(c4)C(C)(C)c4ccccc4-5)c32)c1[2H] ; CC(C)(C)c1cc(C(C)(C)C)c2oc3ccc4c(c3c2c1)-c1cc(N(c2ccc(-c3ccccc3)cc2)c2cccc(C3(c5ccccc5)c5ccccc5-c5ccccc53)c2)ccc1C4(C)C ; CC(C)(C)c1cccc2c1oc1ccc3c(c12)-c1cc(N(c2ccc4c(c2)C(C)(C)c2ccccc2-4)c2ccc4c(c2)sc2ccccc24)ccc1C3(C)C]

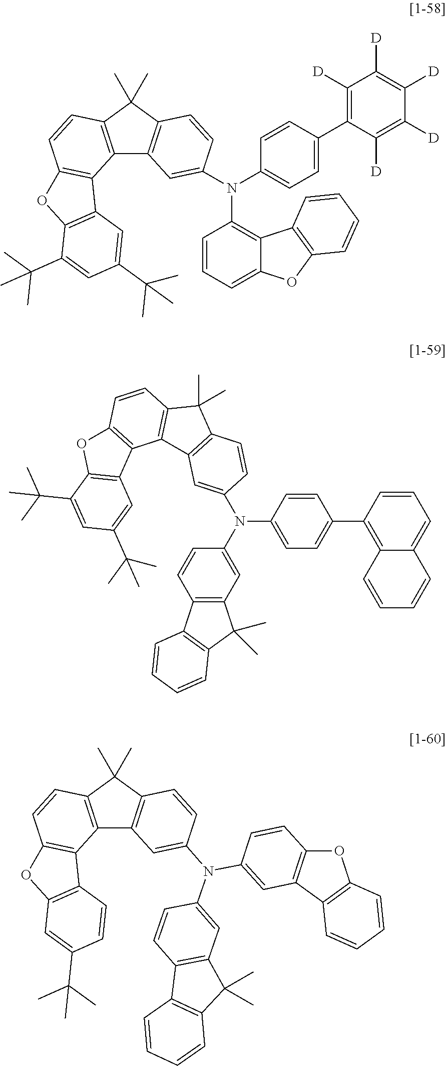 [SMILES: [2H]c1c([2H])c([2H])c(-c2ccc(N(c3ccc4c(c3)-c3c(ccc5oc6c(C(C)(C)C)cc(C(C)(C)C)cc6c35)C4(C)C)c3cccc4oc5ccccc5c34)cc2)c([2H])c1[2H] ; CC(C)(C)c1cc(C(C)(C)C)c2oc3ccc4c(c3c2c1)-c1cc(N(c2ccc(-c3cccc5ccccc35)cc2)c2ccc3c(c2)C(C)(C)c2ccccc2-3)ccc1C4(C)C ; CC(C)(C)c1ccc2c(c1)oc1ccc3c(c12)-c1cc(N(c2ccc4c(c2)C(C)(C)c2ccccc2-4)c2ccc4oc5ccccc5c4c2)ccc1C3(C)C]

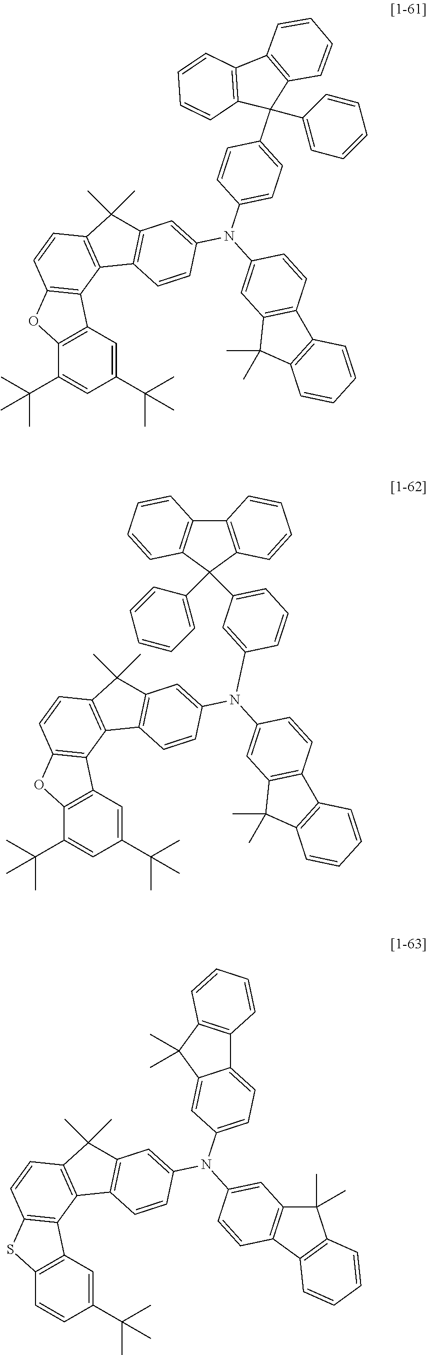 [SMILES: CC(C)(C)c1cc(C(C)(C)C)c2oc3ccc4c(c3c2c1)-c1ccc(N(c2ccc(C3(c5ccccc5)c5ccccc5-c5ccccc53)cc2)c2ccc3c(c2)C(C)(C)c2ccccc2-3)cc1C4(C)C ; CC(C)(C)c1cc(C(C)(C)C)c2oc3ccc4c(c3c2c1)-c1ccc(N(c2cccc(C3(c5ccccc5)c5ccccc5-c5ccccc53)c2)c2ccc3c(c2)C(C)(C)c2ccccc2-3)cc1C4(C)C ; CC(C)(C)c1ccc2sc3ccc4c(c3c2c1)-c1ccc(N(c2ccc3c(c2)C(C)(C)c2ccccc2-3)c2ccc3c(c2)C(C)(C)c2ccccc2-3)cc1C4(C)C]

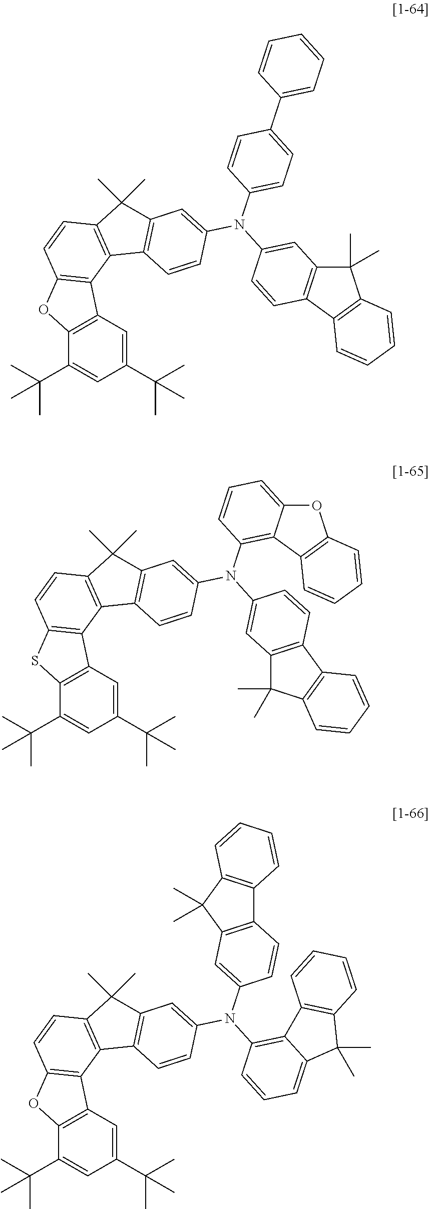 [SMILES: CC(C)(C)c1cc(C(C)(C)C)c2oc3ccc4c(c3c2c1)-c1ccc(N(c2ccc(-c3ccccc3)cc2)c2ccc3c(c2)C(C)(C)c2ccccc2-3)cc1C4(C)C ; CC(C)(C)c1cc(C(C)(C)C)c2sc3ccc4c(c3c2c1)-c1ccc(N(c2ccc3c(c2)C(C)(C)c2ccccc2-3)c2cccc3oc5ccccc5c23)cc1C4(C)C ; CC(C)(C)c1cc(C(C)(C)C)c2oc3ccc4c(c3c2c1)-c1ccc(N(c2ccc3c(c2)C(C)(C)c2ccccc2-3)c2cccc3c2-c2ccccc2C3(C)C)cc1C4(C)C]

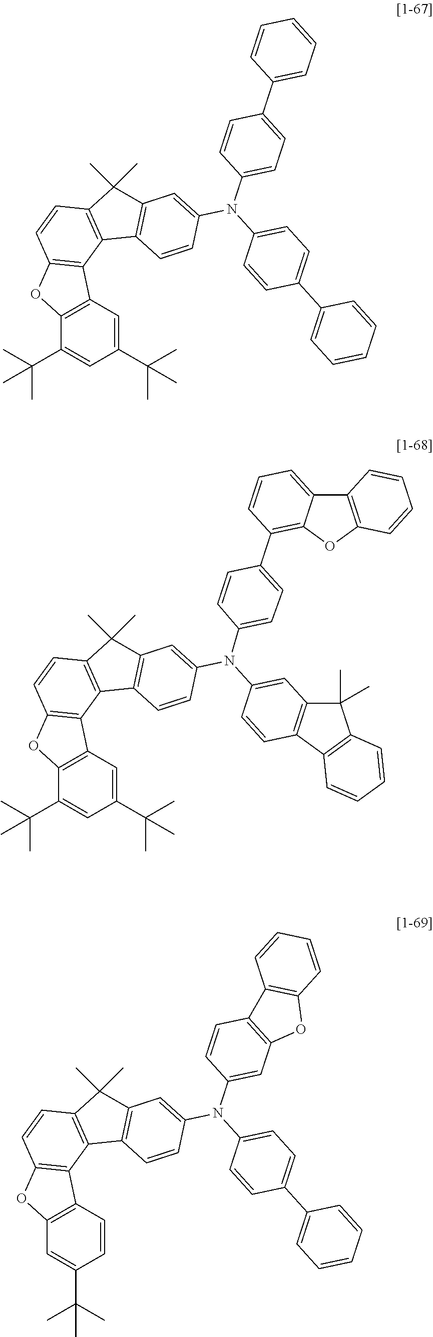 [SMILES: CC(C)(C)c1cc(C(C)(C)C)c2oc3ccc4c(c3c2c1)-c1ccc(N(c2ccc(-c3ccccc3)cc2)c2ccc(-c3ccccc3)cc2)cc1C4(C)C ; CC(C)(C)c1cc(C(C)(C)C)c2oc3ccc4c(c3c2c1)-c1ccc(N(c2ccc(-c3cccc5c3oc3ccccc35)cc2)c2ccc3c(c2)C(C)(C)c2ccccc2-3)cc1C4(C)C ; CC(C)(C)c1ccc2c(c1)oc1ccc3c(c12)-c1ccc(N(c2ccc(-c4ccccc4)cc2)c2ccc4c(c2)oc2ccccc24)cc1C3(C)C]

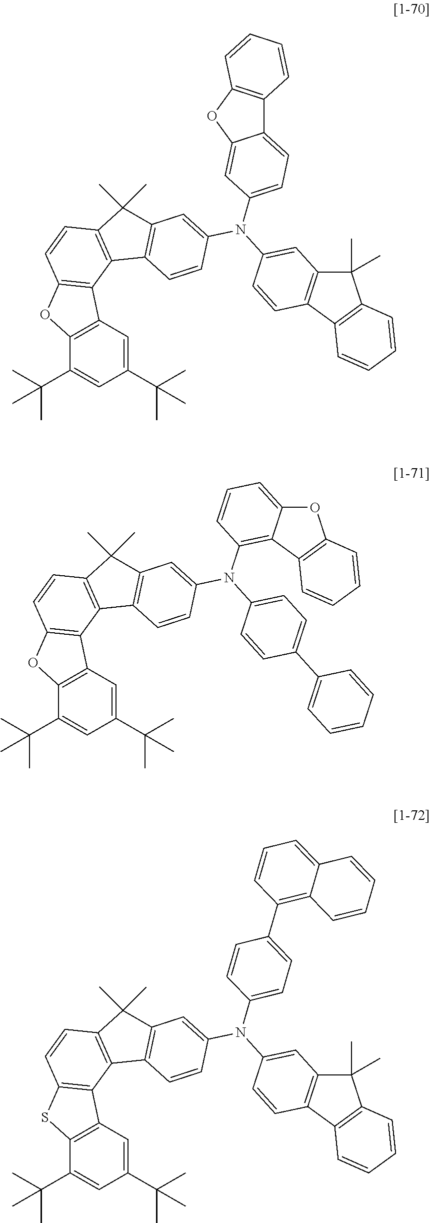 [SMILES: CC(C)(C)c1cc(C(C)(C)C)c2oc3ccc4c(c3c2c1)-c1ccc(N(c2ccc3c(c2)C(C)(C)c2ccccc2-3)c2ccc3c(c2)oc2ccccc23)cc1C4(C)C ; CC(C)(C)c1cc(C(C)(C)C)c2oc3ccc4c(c3c2c1)-c1ccc(N(c2ccc(-c3ccccc3)cc2)c2cccc3oc5ccccc5c23)cc1C4(C)C ; CC(C)(C)c1cc(C(C)(C)C)c2sc3ccc4c(c3c2c1)-c1ccc(N(c2ccc(-c3cccc5ccccc35)cc2)c2ccc3c(c2)C(C)(C)c2ccccc2-3)cc1C4(C)C]

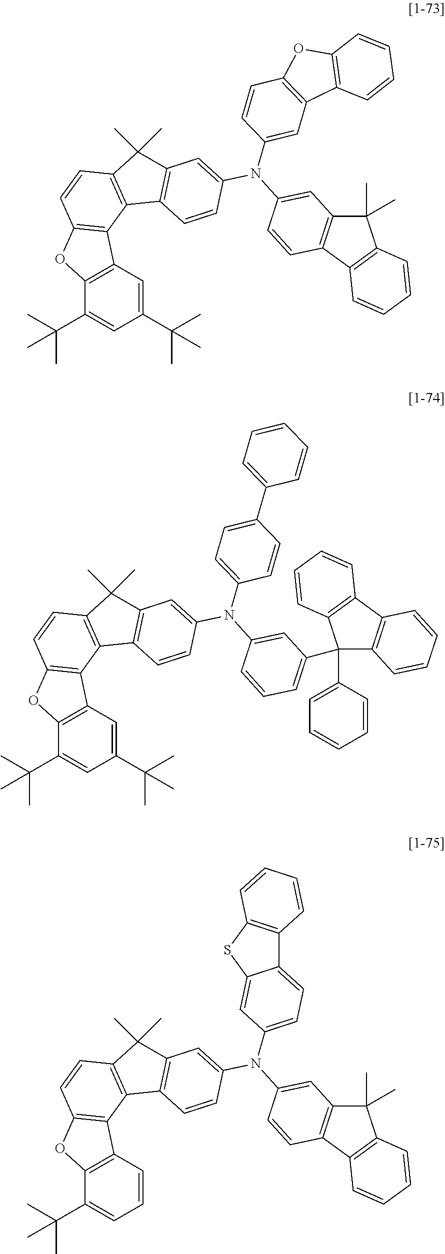 [SMILES: CC(C)(C)c1cc(C(C)(C)C)c2oc3ccc4c(c3c2c1)-c1ccc(N(c2ccc3c(c2)C(C)(C)c2ccccc2-3)c2ccc3oc5ccccc5c3c2)cc1C4(C)C ; CC(C)(C)c1cc(C(C)(C)C)c2oc3ccc4c(c3c2c1)-c1ccc(N(c2ccc(-c3ccccc3)cc2)c2cccc(C3(c5ccccc5)c5ccccc5-c5ccccc53)c2)cc1C4(C)C ; CC(C)(C)c1cccc2c1oc1ccc3c(c12)-c1ccc(N(c2ccc4c(c2)C(C)(C)c2ccccc2-4)c2ccc4c(c2)sc2ccccc24)cc1C3(C)C]

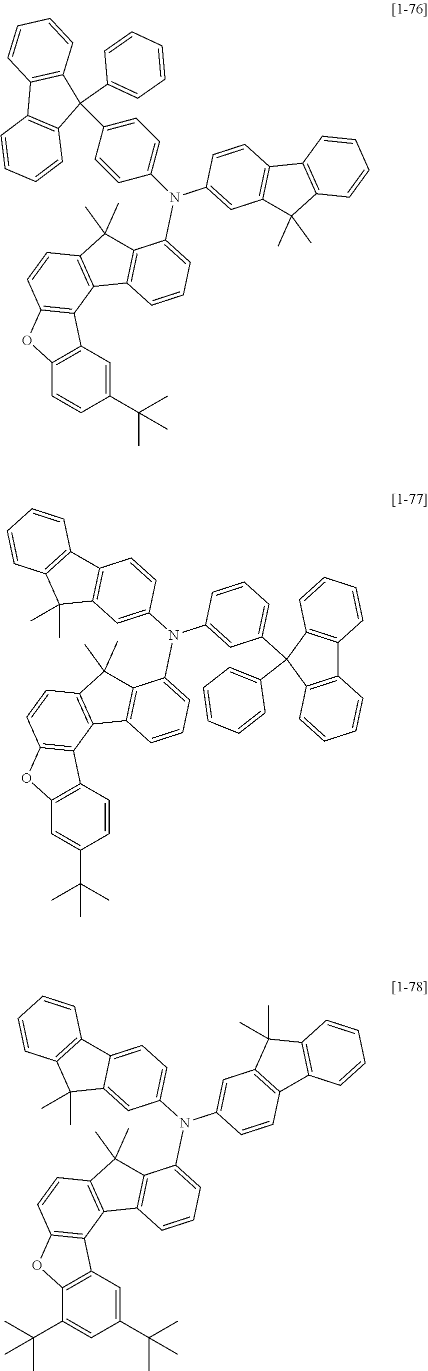 [SMILES: CC(C)(C)c1ccc2oc3ccc4c(c3c2c1)-c1cccc(N(c2ccc(C3(c5ccccc5)c5ccccc5-c5ccccc53)cc2)c2ccc3c(c2)C(C)(C)c2ccccc2-3)c1C4(C)C ; CC(C)(C)c1ccc2c(c1)oc1ccc3c(c12)-c1cccc(N(c2cccc(C4(c5ccccc5)c5ccccc5-c5ccccc54)c2)c2ccc4c(c2)C(C)(C)c2ccccc2-4)c1C3(C)C ; CC(C)(C)c1cc(C(C)(C)C)c2oc3ccc4c(c3c2c1)-c1cccc(N(c2ccc3c(c2)C(C)(C)c2ccccc2-3)c2ccc3c(c2)C(C)(C)c2ccccc2-3)c1C4(C)C]

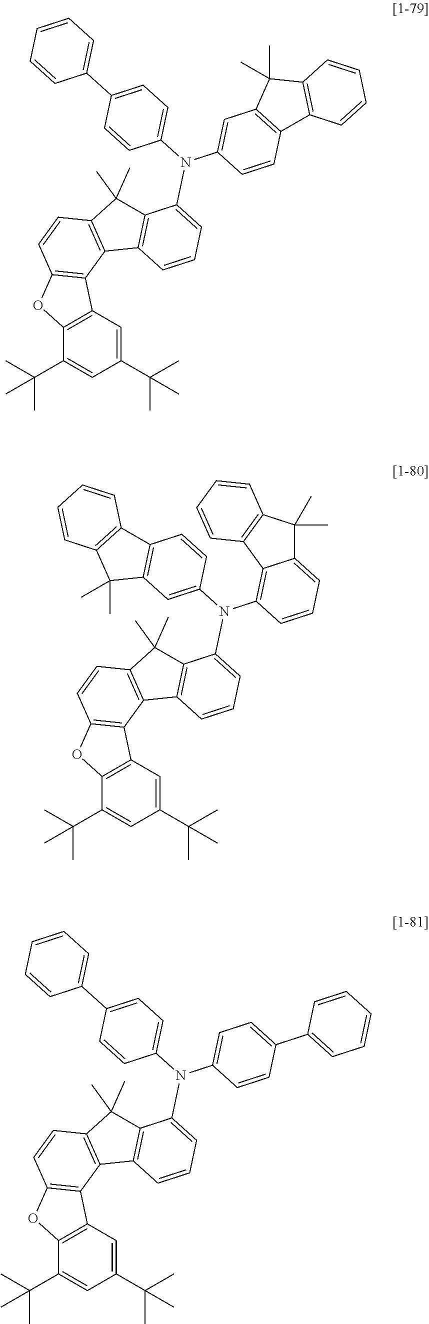

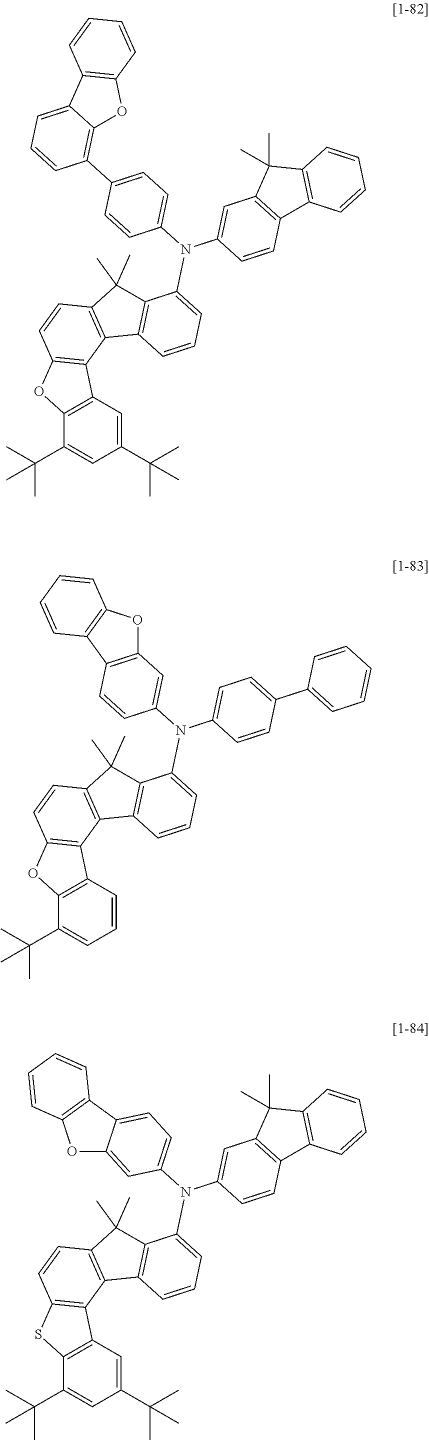 [SMILES: CC(C)(C)c1cc(C(C)(C)C)c2oc3ccc4c(c3c2c1)-c1cccc(N(c2ccc(-c3cccc5c3oc3ccccc35)cc2)c2ccc3c(c2)C(C)(C)c2ccccc2-3)c1C4(C)C ; CC(C)(C)c1cccc2c1oc1ccc3c(c12)-c1cccc(N(c2ccc(-c4ccccc4)cc2)c2ccc4c(c2)oc2ccccc24)c1C3(C)C ; CC(C)(C)c1cc(C(C)(C)C)c2sc3ccc4c(c3c2c1)-c1cccc(N(c2ccc3c(c2)C(C)(C)c2ccccc2-3)c2ccc3c(c2)oc2ccccc23)c1C4(C)C]

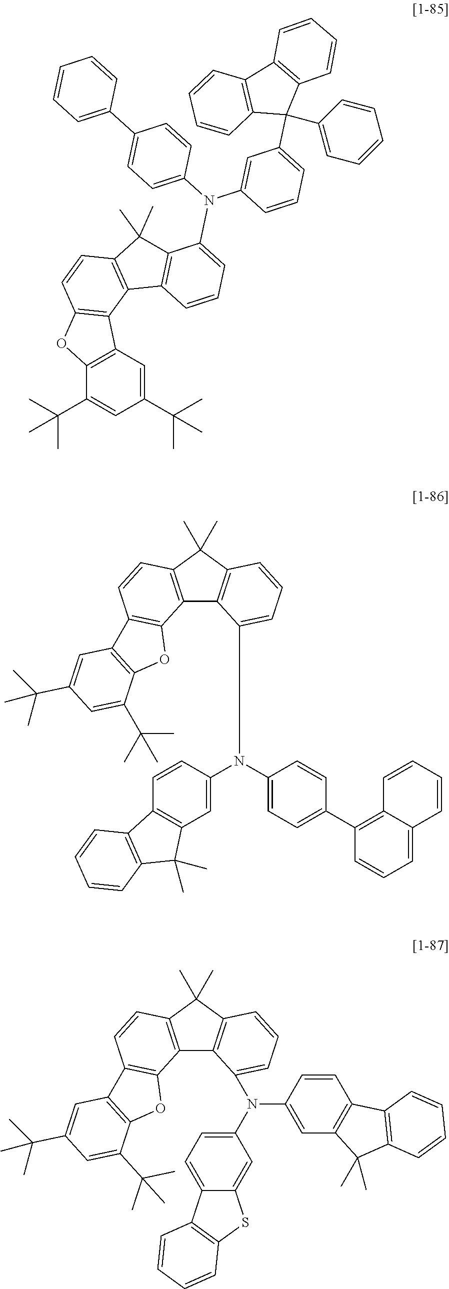 [SMILES: CC(C)(C)c1cc(C(C)(C)C)c2oc3ccc4c(c3c2c1)-c1cccc(N(c2ccc(-c3ccccc3)cc2)c2cccc(C3(c5ccccc5)c5ccccc5-c5ccccc53)c2)c1C4(C)C ; CC(C)(C)c1cc(C(C)(C)C)c2oc3c4c(ccc3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cc(C(C)(C)C)c2oc3c4c(ccc3c2c1)C(C)(C)c1cccc(N(c2ccc3c(c2)C(C)(C)c2ccccc2-3)c2ccc3c(c2)sc2ccccc23)c1-4]

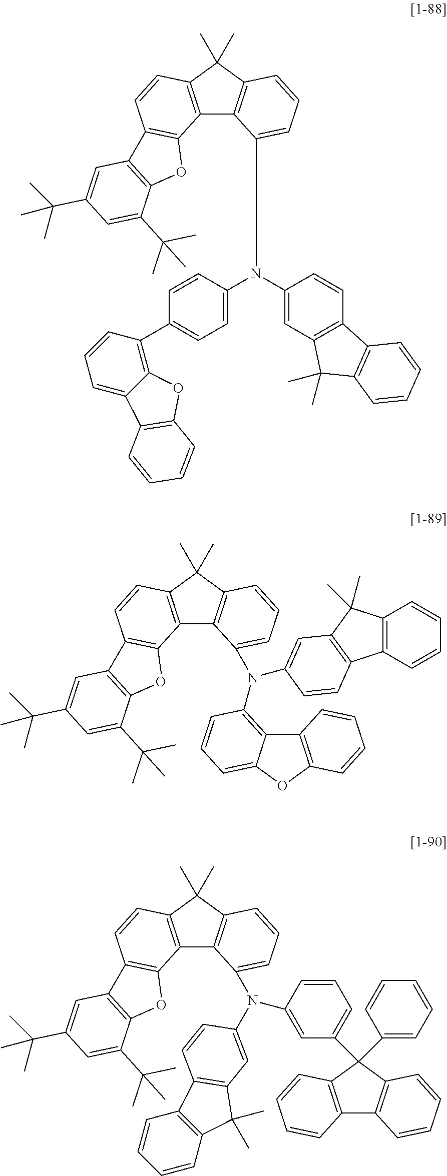 [SMILES: CC(C)(C)c1cc(C(C)(C)C)c2oc3c4c(ccc3c2c1)C(C)(C)c1cccc(N(c2ccc(-c3cccc5c3oc3ccccc35)cc2)c2ccc3c(c2)C(C)(C)c2ccccc2-3)c1-4 ; CC(C)(C)c1cc(C(C)(C)C)c2oc3c4c(ccc3c2c1)C(C)(C)c1cccc(N(c2ccc3c(c2)C(C)(C)c2ccccc2-3)c2cccc3oc5ccccc5c23)c1-4 ; CC(C)(C)c1cc(C(C)(C)C)c2oc3c4c(ccc3c2c1)C(C)(C)c1cccc(N(c2cccc(C3(c5ccccc5)c5ccccc5-c5ccccc53)c2)c2ccc3c(c2)C(C)(C)c2ccccc2-3)c1-4]

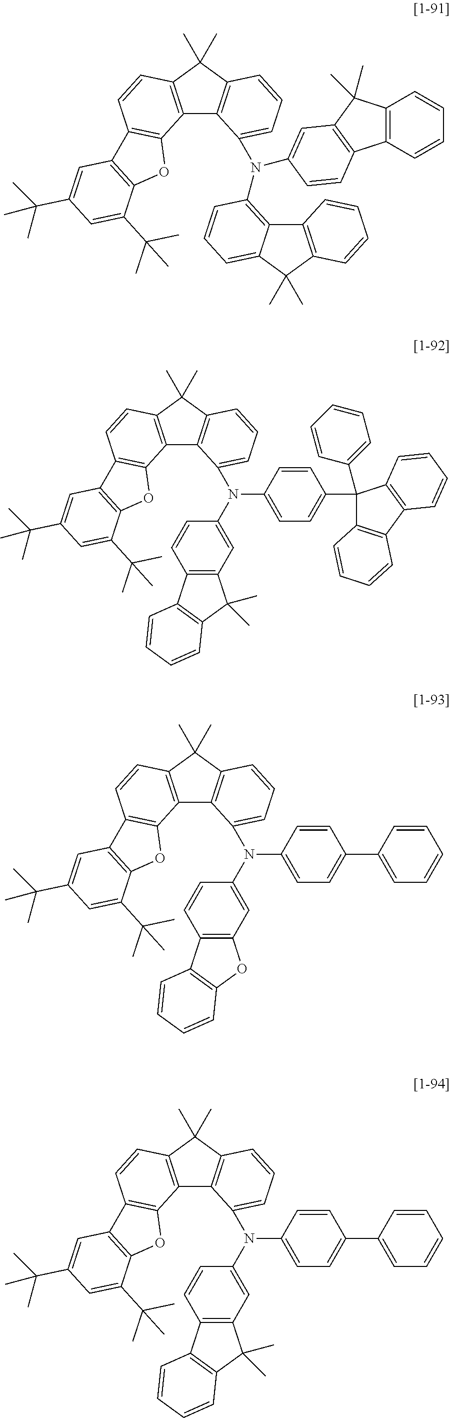 [SMILES: CC(C)(C)c1cc(C(C)(C)C)c2oc3c4c(ccc3c2c1)C(C)(C)c1cccc(N(c2ccc3c(c2)C(C)(C)c2ccccc2-3)c2cccc3c2-c2ccccc2C3(C)C)c1-4 ; CC(C)(C)c1cc(C(C)(C)C)c2oc3c4c(ccc3c2c1)C(C)(C)c1cccc(N(c2ccc(C3(c5ccccc5)c5ccccc5-c5ccccc53)cc2)c2ccc3c(c2)C(C)(C)c2ccccc2-3)c1-4 ; CC(C)(C)c1cc(C(C)(C)C)c2oc3c4c(ccc3c2c1)C(C)(C)c1cccc(N(c2ccc(-c3ccccc3)cc2)c2ccc3c(c2)oc2ccccc23)c1-4 ; CC(C)(C)c1cc(C(C)(C)C)c2oc3c4c(ccc3c2c1)C(C)(C)c1cccc(N(c2ccc(-c3ccccc3)cc2)c2ccc3c(c2)C(C)(C)c2ccccc2-3)c1-4]

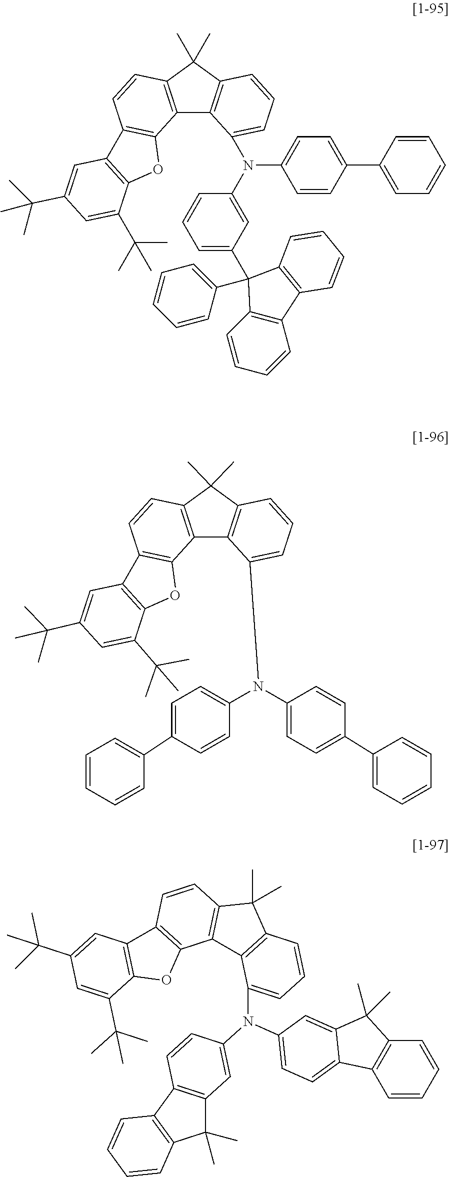 [SMILES: CC(C)(C)c1cc(C(C)(C)C)c2oc3c4c(ccc3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2oc3c4c(ccc3c2c1)C(C)(C)c1cccc(N(c2ccc(-c3ccccc3)cc2)c2ccc(-c3ccccc3)cc2)c1-4 ; CC(C)(C)c1cc(C(C)(C)C)c2oc3c4c(ccc3c2c1)C(C)(C)c1cccc(N(c2ccc3c(c2)C(C)(C)c2ccccc2-3)c2ccc3c(c2)C(C)(C)c2ccccc2-3)c1-4]

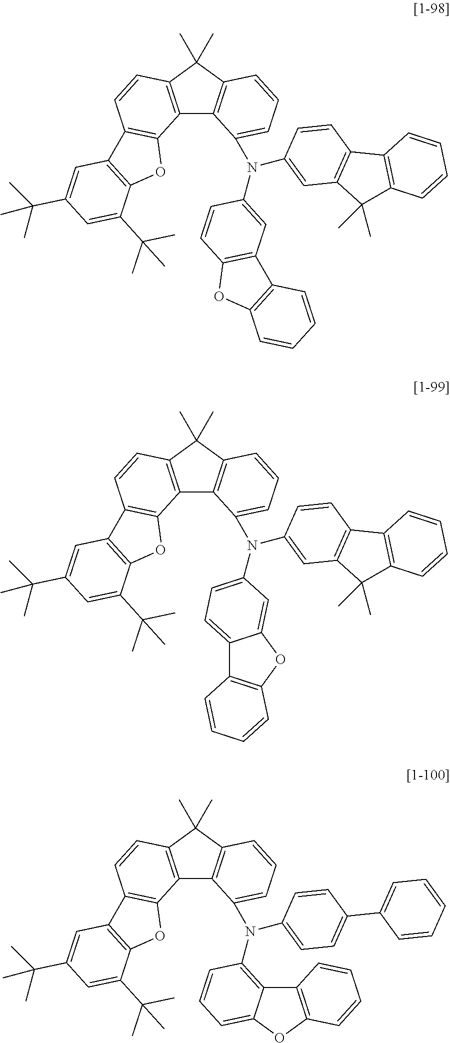 [SMILES: CC(C)(C)c1cc(C(C)(C)C)c2oc3c4c(ccc3c2c1)C(C)(C)c1cccc(N(c2ccc3c(c2)C(C)(C)c2ccccc2-3)c2ccc3oc5ccccc5c3c2)c1-4 ; CC(C)(C)c1cc(C(C)(C)C)c2oc3c4c(ccc3c2c1)C(C)(C)c1cccc(N(c2ccc3c(c2)C(C)(C)c2ccccc2-3)c2ccc3c(c2)oc2ccccc23)c1-4 ; CC(C)(C)c1cc(C(C)(C)C)c2oc3c4c(ccc3c2c1)C(C)(C)c1cccc(N(c2ccc(-c3ccccc3)cc2)c2cccc3oc5ccccc5c23)c1-4]

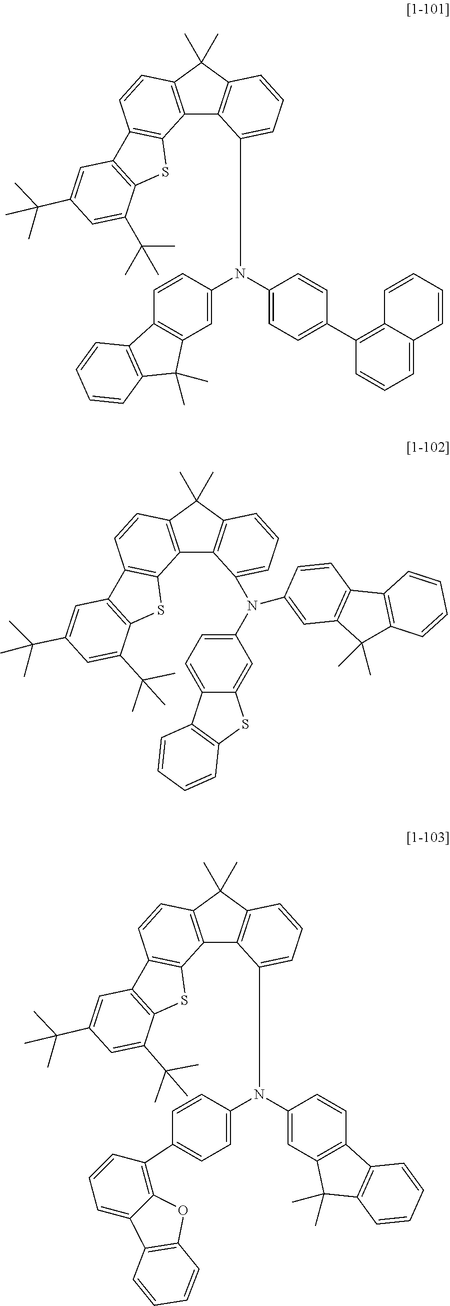 [SMILES: CC(C)(C)c1cc(C(C)(C)C)c2sc3c4c(ccc3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cc(C(C)(C)C)c2sc3c4c(ccc3c2c1)C(C)(C)c1cccc(N(c2ccc3c(c2)C(C)(C)c2ccccc2-3)c2ccc3c(c2)sc2ccccc23)c1-4 ; CC(C)(C)c1cc(C(C)(C)C)c2sc3c4c(ccc3c2c1)C(C)(C)c1cccc(N(c2ccc(-c3cccc5c3oc3ccccc35)cc2)c2ccc3c(c2)C(C)(C)c2ccccc2-3)c1-4]

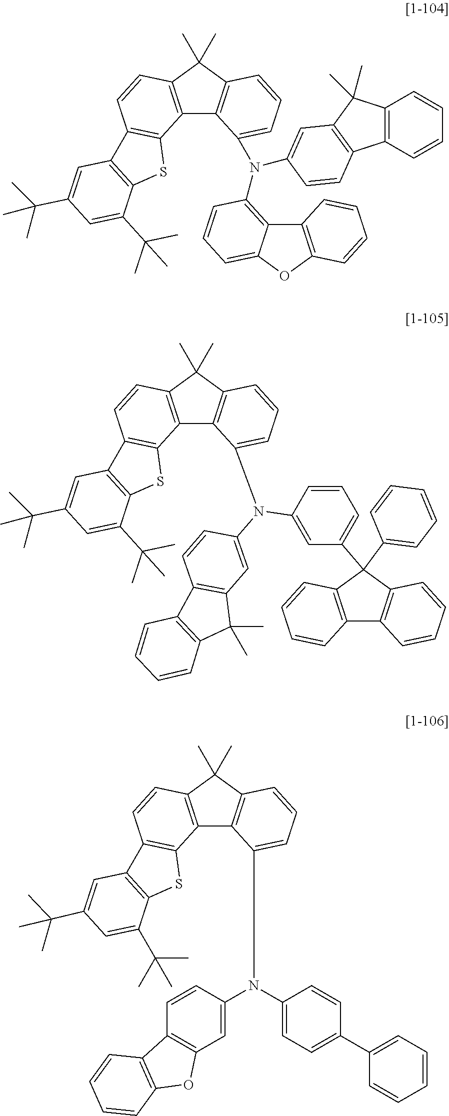 [SMILES: CC(C)(C)c1cc(C(C)(C)C)c2sc3c4c(ccc3c2c1)C(C)(C)c1cccc(N(c2ccc3c(c2)C(C)(C)c2ccccc2-3)c2cccc3oc5ccccc5c23)c1-4 ; CC(C)(C)c1cc(C(C)(C)C)c2sc3c4c(ccc3c2c1)C(C)(C)c1cccc(N(c2cccc(C3(c5ccccc5)c5ccccc5-c5ccccc53)c2)c2ccc3c(c2)C(C)(C)c2ccccc2-3)c1-4 ; CC(C)(C)c1cc(C(C)(C)C)c2sc3c4c(ccc3c2c1)C(C)(C)c1cccc(N(c2ccc(-c3ccccc3)cc2)c2ccc3c(c2)oc2ccccc23)c1-4]

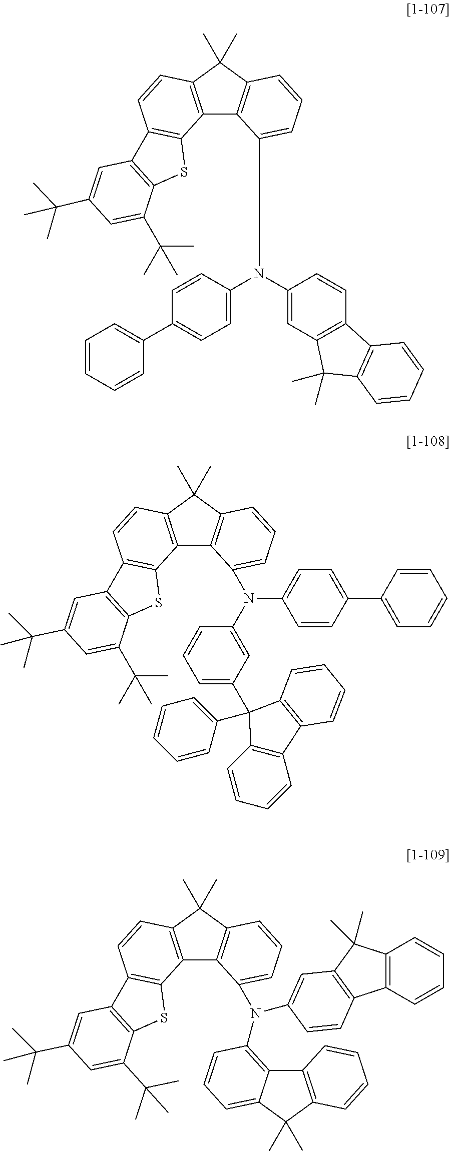 [SMILES: CC(C)(C)c1cc(C(C)(C)C)c2sc3c4c(ccc3c2c1)C(C)(C)c1cccc(N(c2ccc(-c3ccccc3)cc2)c2ccc3c(c2)C(C)(C)c2ccccc2-3)c1-4 ; CC(C)(C)c1cc(C(C)(C)C)c2sc3c4c(ccc3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2sc3c4c(ccc3c2c1)C(C)(C)c1cccc(N(c2ccc3c(c2)C(C)(C)c2ccccc2-3)c2cccc3c2-c2ccccc2C3(C)C)c1-4]

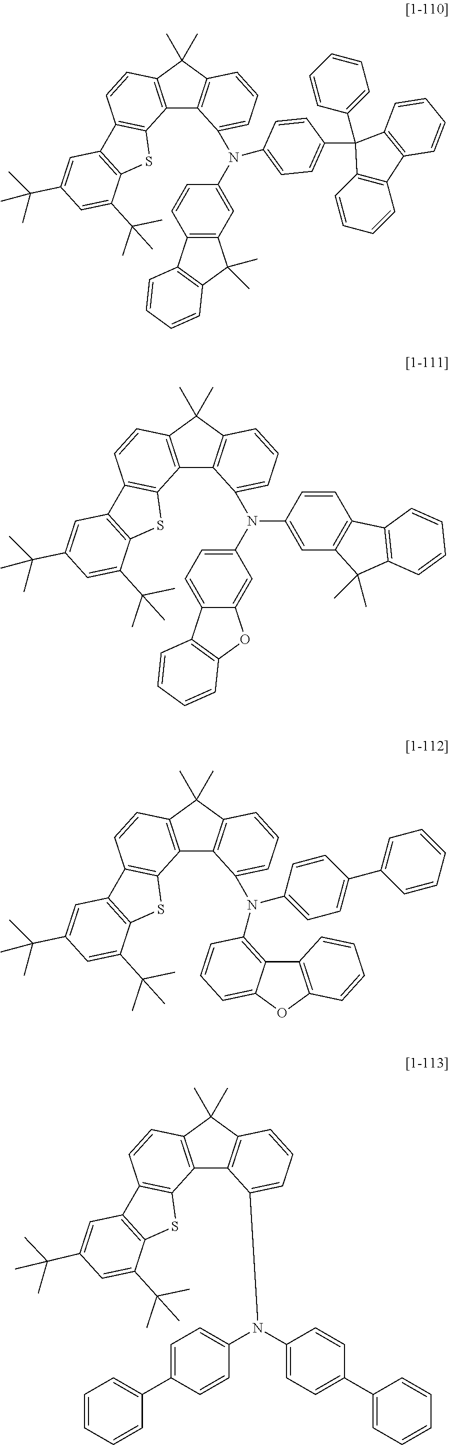 [SMILES: CC(C)(C)c1cc(C(C)(C)C)c2sc3c4c(ccc3c2c1)C(C)(C)c1cccc(N(c2ccc(C3(c5ccccc5)c5ccccc5-c5ccccc53)cc2)c2ccc3c(c2)C(C)(C)c2ccccc2-3)c1-4 ; CC(C)(C)c1cc(C(C)(C)C)c2sc3c4c(ccc3c2c1)C(C)(C)c1cccc(N(c2ccc3c(c2)C(C)(C)c2ccccc2-3)c2ccc3c(c2)oc2ccccc23)c1-4 ; CC(C)(C)c1cc(C(C)(C)C)c2sc3c4c(ccc3c2c1)C(C)(C)c1cccc(N(c2ccc(-c3ccccc3)cc2)c2cccc3oc5ccccc5c23)c1-4 ; CC(C)(C)c1cc(C(C)(C)C)c2sc3c4c(ccc3c2c1)C(C)(C)c1cccc(N(c2ccc(-c3ccccc3)cc2)c2ccc(-c3ccccc3)cc2)c1-4]

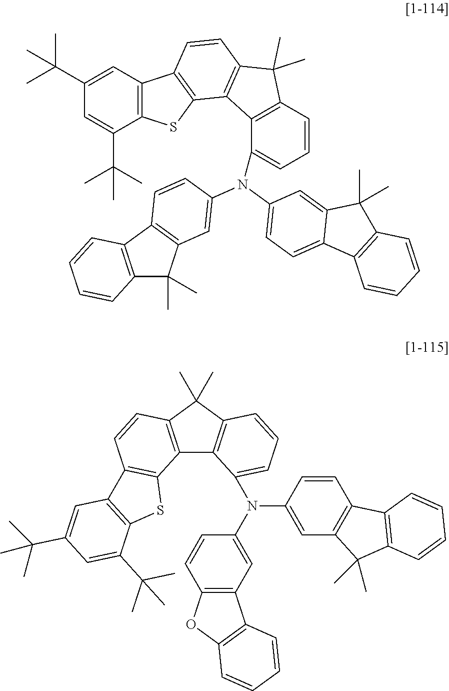

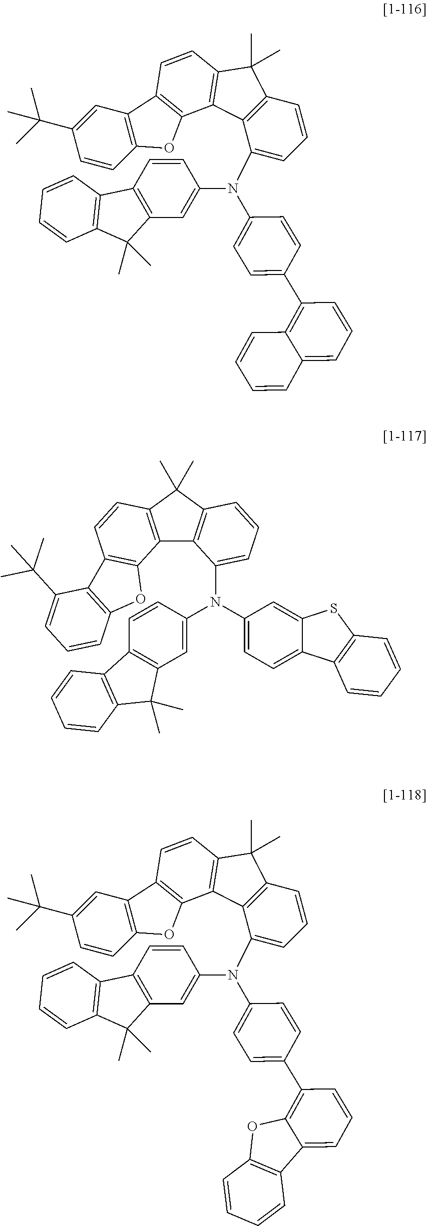 [SMILES: CC(C)(C)c1ccc2oc3c4c(ccc3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cccc2oc3c4c(ccc3c12)C(C)(C)c1cccc(N(c2ccc3c(c2)C(C)(C)c2ccccc2-3)c2ccc3c(c2)sc2ccccc23)c1-4 ; CC(C)(C)c1ccc2oc3c4c(ccc3c2c1)C(C)(C)c1cccc(N(c2ccc(-c3cccc5c3oc3ccccc35)cc2)c2ccc3c(c2)C(C)(C)c2ccccc2-3)c1-4]

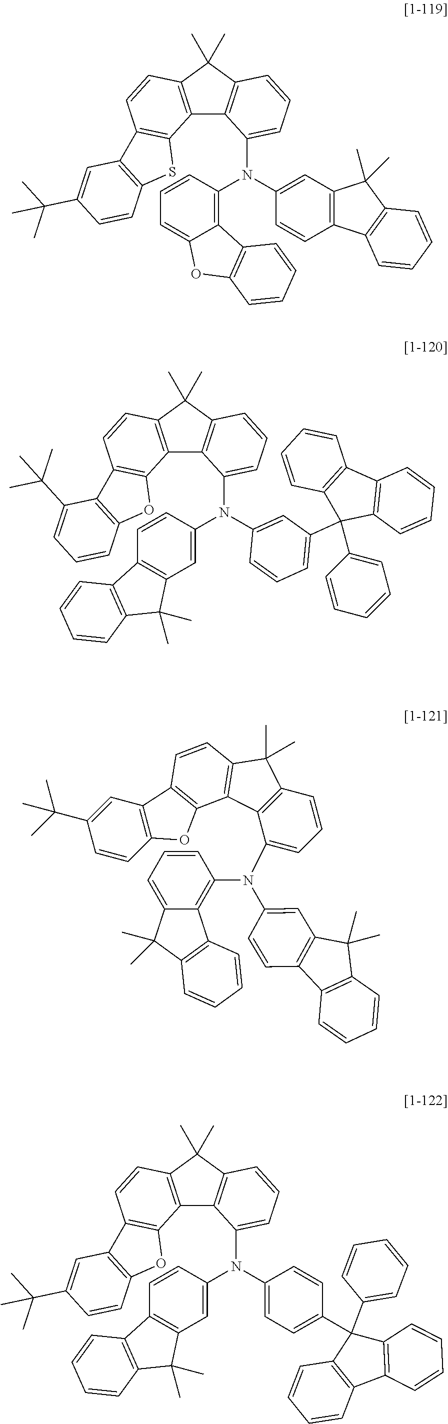

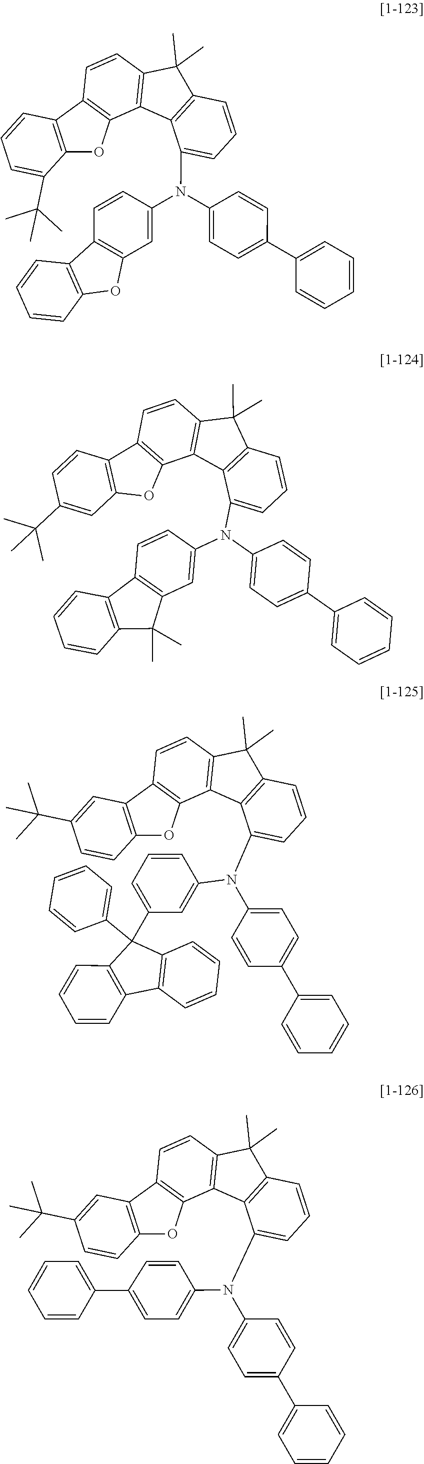 [SMILES: CC(C)(C)c1cccc2c1oc1c3c(ccc12)C(C)(C)c1cccc(N(c2ccc(-c4ccccc4)cc2)c2ccc4c(c2)oc2ccccc24)c1-3 ; CC(C)(C)c1ccc2c(c1)oc1c3c(ccc12)C(C)(C)c1cccc(N(c2ccc(-c4ccccc4)cc2)c2ccc4c(c2)C(C)(C)c2ccccc2-4)c1-3 ; CC(C)(C)c1ccc2oc3c4c(ccc3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1ccc2oc3c4c(ccc3c2c1)C(C)(C)c1cccc(N(c2ccc(-c3ccccc3)cc2)c2ccc(-c3ccccc3)cc2)c1-4]

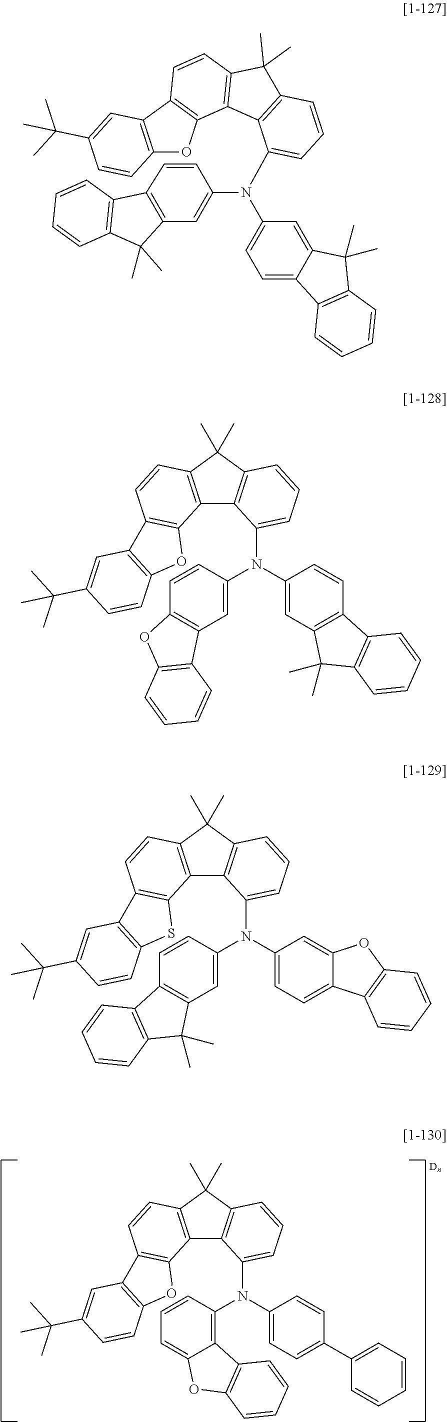

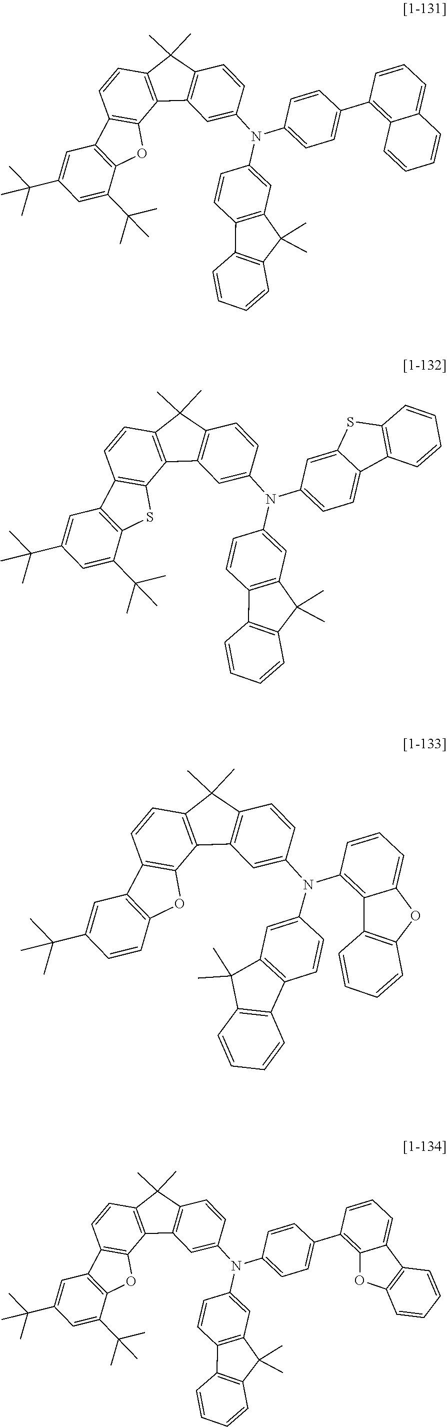 [SMILES: CC(C)(C)c1cc(C(C)(C)C)c2oc3c4c(ccc3c2c1)C(C)(C)c1ccc(N(c2ccc(-c3cccc5ccccc35)cc2)c2ccc3c(c2)C(C)(C)c2ccccc2-3)cc1-4 ; CC(C)(C)c1cc(C(C)(C)C)c2sc3c4c(ccc3c2c1)C(C)(C)c1ccc(N(c2ccc3c(c2)C(C)(C)c2ccccc2-3)c2ccc3c(c2)sc2ccccc23)cc1-4 ; CC(C)(C)c1ccc2oc3c4c(ccc3c2c1)C(C)(C)c1ccc(N(c2ccc3c(c2)C(C)(C)c2ccccc2-3)c2cccc3oc5ccccc5c23)cc1-4 ; CC(C)(C)c1cc(C(C)(C)C)c2oc3c4c(ccc3c2c1)C(C)(C)c1ccc(N(c2ccc(-c3cccc5c3oc3ccccc35)cc2)c2ccc3c(c2)C(C)(C)c2ccccc2-3)cc1-4]

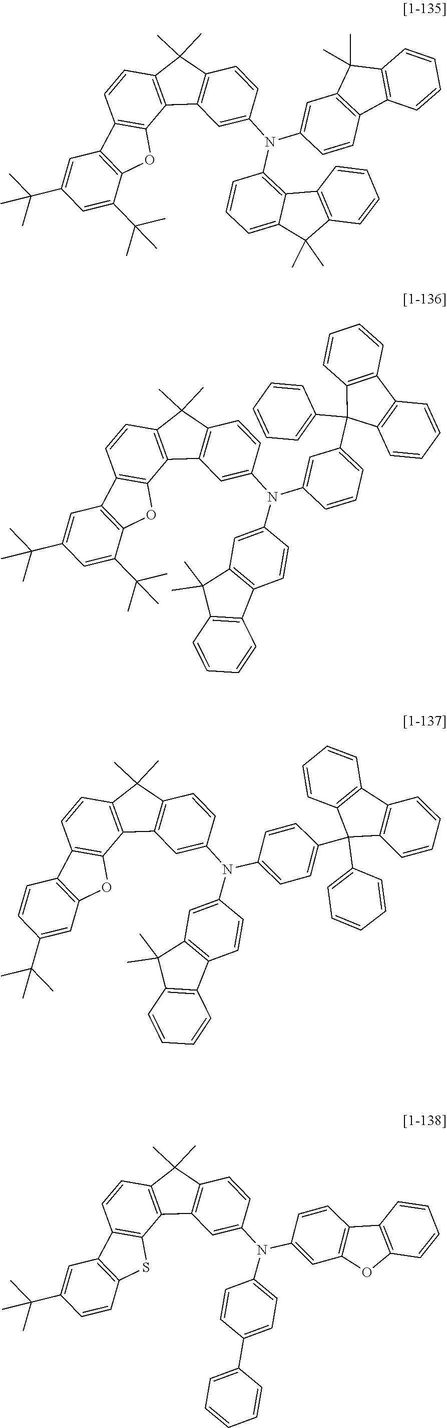 [SMILES: CC(C)(C)c1cc(C(C)(C)C)c2oc3c4c(ccc3c2c1)C(C)(C)c1ccc(N(c2ccc3c(c2)C(C)(C)c2ccccc2-3)c2cccc3c2-c2ccccc2C3(C)C)cc1-4 ; CC(C)(C)c1cc(C(C)(C)C)c2oc3c4c(ccc3c2c1)C(C)(C)c1ccc(N(c2cccc(C3(c5ccccc5)c5ccccc5-c5ccccc53)c2)c2ccc3c(c2)C(C)(C)c2ccccc2-3)cc1-4 ; CC(C)(C)c1ccc2c(c1)oc1c3c(ccc12)C(C)(C)c1ccc(N(c2ccc(C4(c5ccccc5)c5ccccc5-c5ccccc54)cc2)c2ccc4c(c2)C(C)(C)c2ccccc2-4)cc1-3 ; CC(C)(C)c1ccc2sc3c4c(ccc3c2c1)C(C)(C)c1ccc(N(c2ccc(-c3ccccc3)cc2)c2ccc3c(c2)oc2ccccc23)cc1-4]

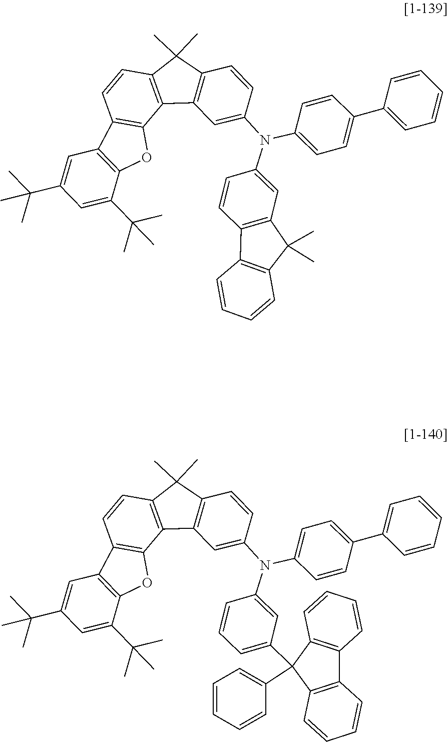 [SMILES: CC(C)(C)c1cc(C(C)(C)C)c2oc3c4c(ccc3c2c1)C(C)(C)c1ccc(N(c2ccc(-c3ccccc3)cc2)c2ccc3c(c2)C(C)(C)c2ccccc2-3)cc1-4 ; CC(C)(C)c1cc(C(C)(C)C)c2oc3c4c(ccc3c2c1)C(C)(C)c1ccc(N(c2ccc(-c3ccccc3)cc2)c2cccc(C3(c5ccccc5)c5ccccc5-c5ccccc53)c2)cc1-4]

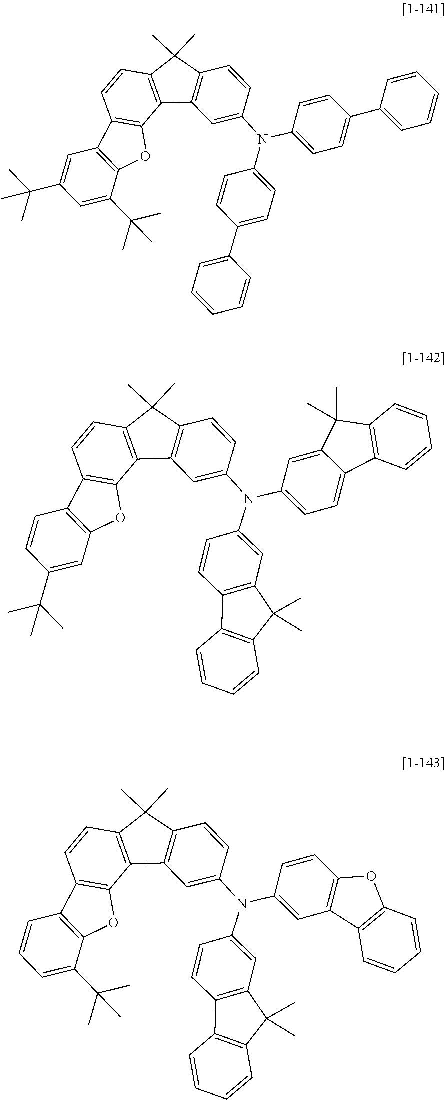 [SMILES: CC(C)(C)c1cc(C(C)(C)C)c2oc3c4c(ccc3c2c1)C(C)(C)c1ccc(N(c2ccc(-c3ccccc3)cc2)c2ccc(-c3ccccc3)cc2)cc1-4 ; CC(C)(C)c1ccc2c(c1)oc1c3c(ccc12)C(C)(C)c1ccc(N(c2ccc4c(c2)C(C)(C)c2ccccc2-4)c2ccc4c(c2)C(C)(C)c2ccccc2-4)cc1-3 ; CC(C)(C)c1cccc2c1oc1c3c(ccc12)C(C)(C)c1ccc(N(c2ccc4c(c2)C(C)(C)c2ccccc2-4)c2ccc4oc5ccccc5c4c2)cc1-3]

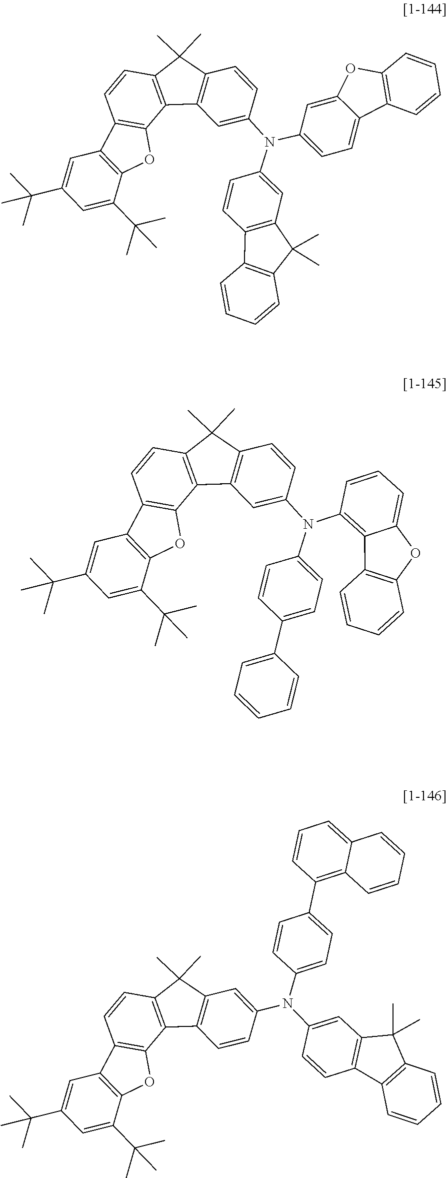 [SMILES: CC(C)(C)c1cc(C(C)(C)C)c2oc3c4c(ccc3c2c1)C(C)(C)c1ccc(N(c2ccc3c(c2)C(C)(C)c2ccccc2-3)c2ccc3c(c2)oc2ccccc23)cc1-4 ; CC(C)(C)c1cc(C(C)(C)C)c2oc3c4c(ccc3c2c1)C(C)(C)c1ccc(N(c2ccc(-c3ccccc3)cc2)c2cccc3oc5ccccc5c23)cc1-4 ; CC(C)(C)c1cc(C(C)(C)C)c2oc3c4c(ccc3c2c1)C(C)(C)c1cc(N(c2ccc(-c3cccc5ccccc35)cc2)c2ccc3c(c2)C(C)(C)c2ccccc2-3)ccc1-4]

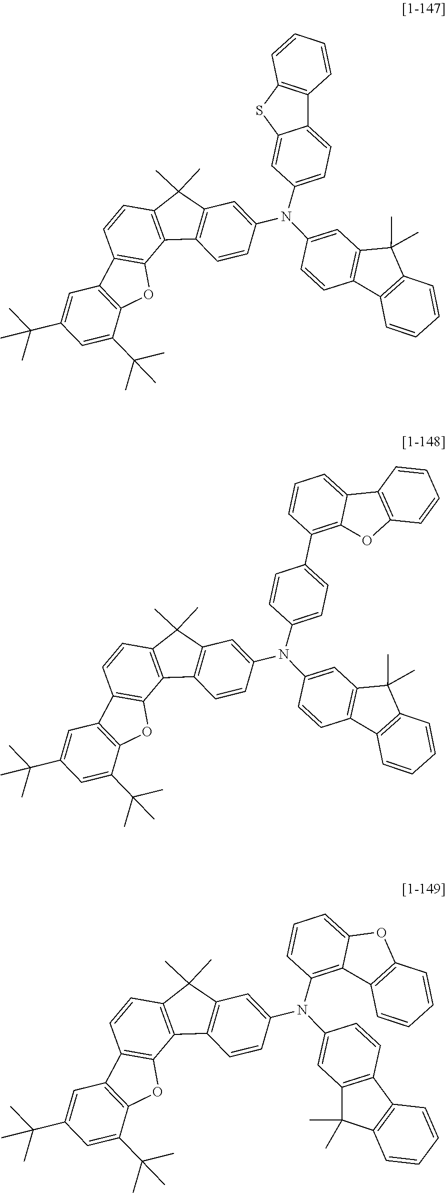 [SMILES: CC(C)(C)c1cc(C(C)(C)C)c2oc3c4c(ccc3c2c1)C(C)(C)c1cc(N(c2ccc3c(c2)C(C)(C)c2ccccc2-3)c2ccc3c(c2)sc2ccccc23)ccc1-4 ; CC(C)(C)c1cc(C(C)(C)C)c2oc3c4c(ccc3c2c1)C(C)(C)c1cc(N(c2ccc(-c3cccc5c3oc3ccccc35)cc2)c2ccc3c(c2)C(C)(C)c2ccccc2-3)ccc1-4 ; CC(C)(C)c1cc(C(C)(C)C)c2oc3c4c(ccc3c2c1)C(C)(C)c1cc(N(c2ccc3c(c2)C(C)(C)c2ccccc2-3)c2cccc3oc5ccccc5c23)ccc1-4]

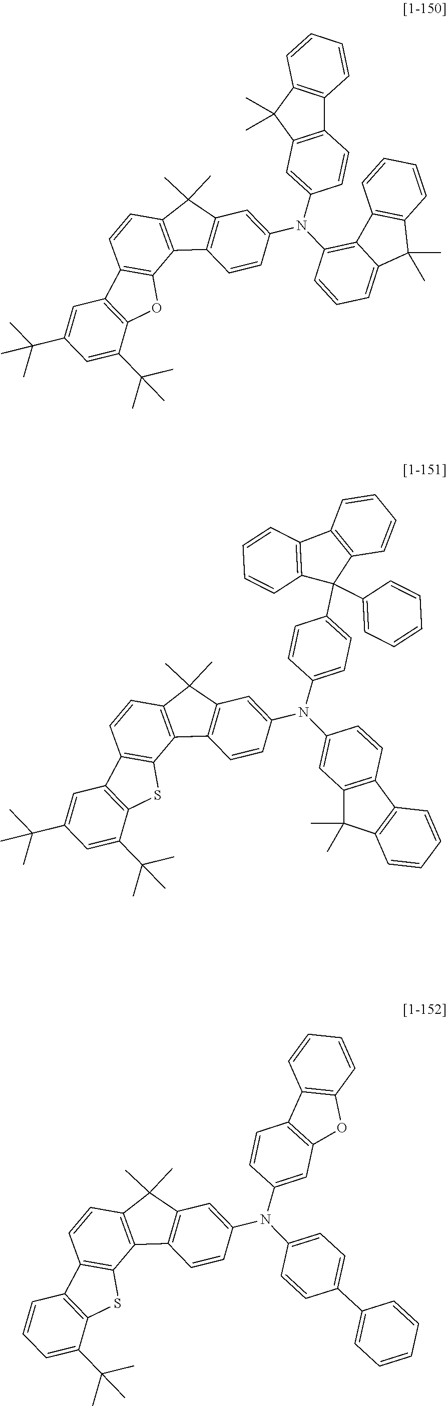

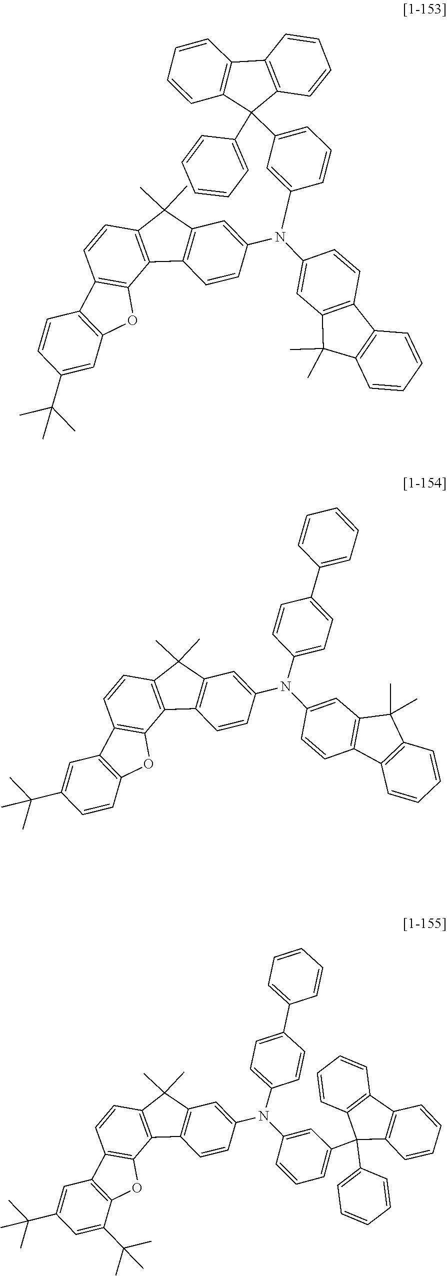

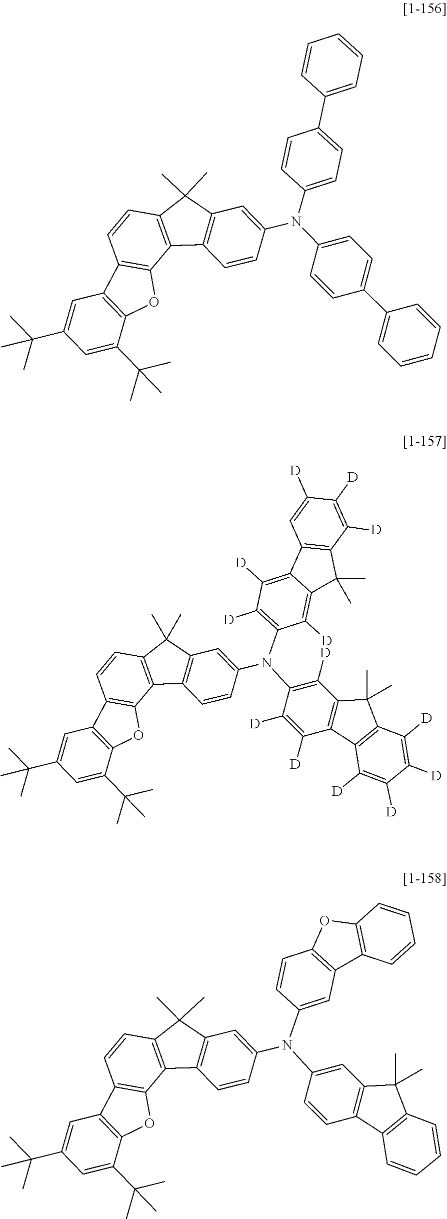

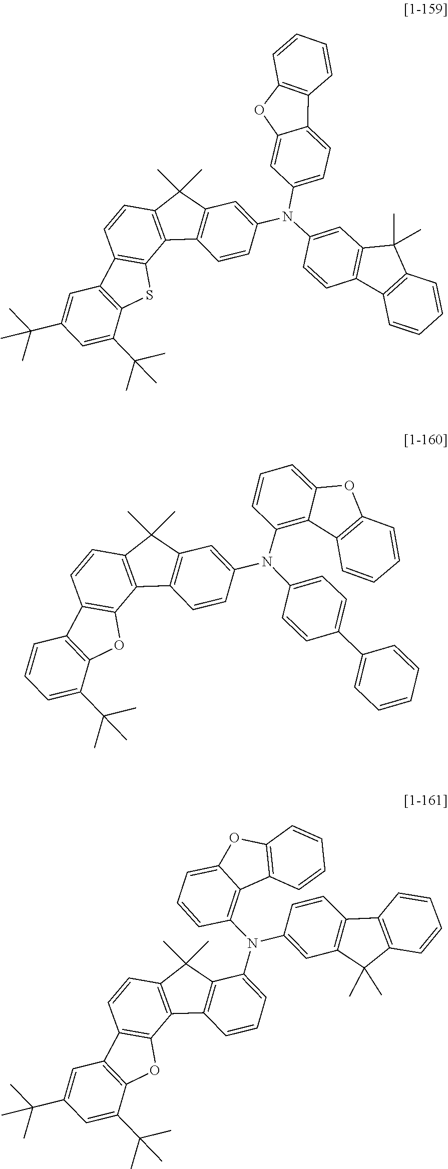 [SMILES: CC(C)(C)c1cc(C(C)(C)C)c2sc3c4c(ccc3c2c1)C(C)(C)c1cc(N(c2ccc3c(c2)C(C)(C)c2ccccc2-3)c2ccc3c(c2)oc2ccccc23)ccc1-4 ; CC(C)(C)c1cccc2c1oc1c3c(ccc12)C(C)(C)c1cc(N(c2ccc(-c4ccccc4)cc2)c2cccc4oc5ccccc5c24)ccc1-3 ; CC(C)(C)c1cc(C(C)(C)C)c2oc3c4c(ccc3c2c1)C(C)(C)c1c-4cccc1N(c1ccc2c(c1)C(C)(C)c1ccccc1-2)c1cccc2oc3ccccc3c12]

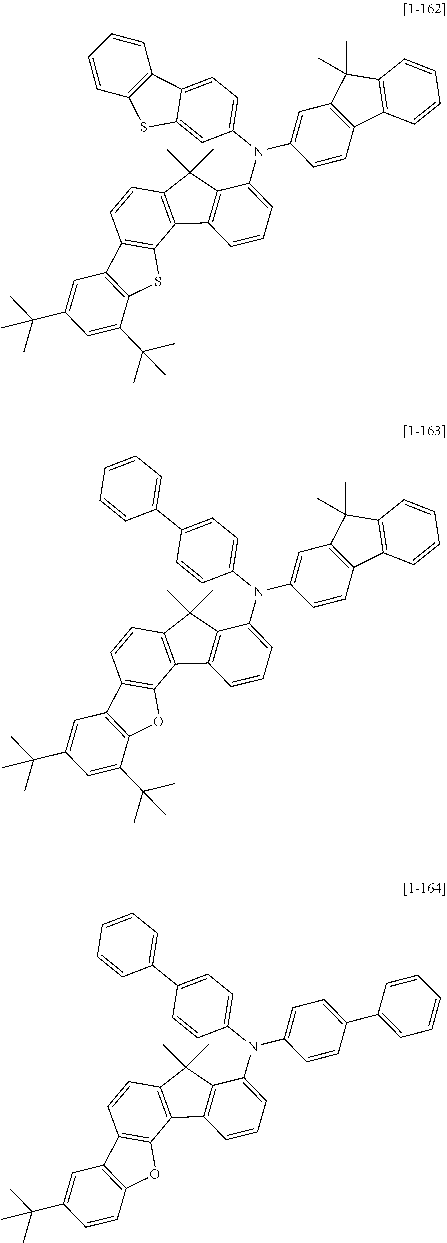 [SMILES: CC(C)(C)c1cc(C(C)(C)C)c2sc3c4c(ccc3c2c1)C(C)(C)c1c-4cccc1N(c1ccc2c(c1)C(C)(C)c1ccccc1-2)c1ccc2c(c1)sc1ccccc12 ; CC(C)(C)c1cc(C(C)(C)C)c2oc3c4c(ccc3c2c1)C(C)(C)c1c-4cccc1N(c1ccc(-c2ccccc2)cc1)c1ccc2c(c1)C(C)(C)c1ccccc1-2 ; CC(C)(C)c1ccc2oc3c4c(ccc3c2c1)C(C)(C)c1c-4cccc1N(c1ccc(-c2ccccc2)cc1)c1ccc(-c2ccccc2)cc1]

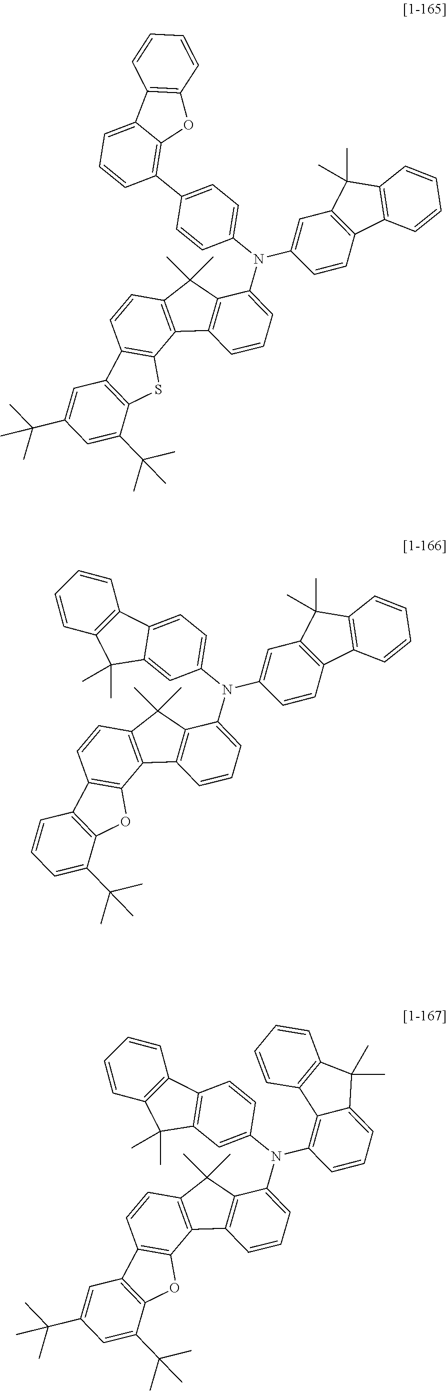 [SMILES: CC(C)(C)c1cc(C(C)(C)C)c2sc3c4c(ccc3c2c1)C(C)(C)c1c-4cccc1N(c1ccc(-c2cccc3c2oc2ccccc23)cc1)c1ccc2c(c1)C(C)(C)c1ccccc1-2 ; CC(C)(C)c1cccc2c1oc1c3c(ccc12)C(C)(C)c1c-3cccc1N(c1ccc2c(c1)C(C)(C)c1ccccc1-2)c1ccc2c(c1)C(C)(C)c1ccccc1-2 ; CC(C)(C)c1cc(C(C)(C)C)c2oc3c4c(ccc3c2c1)C(C)(C)c1c-4cccc1N(c1ccc2c(c1)C(C)(C)c1ccccc1-2)c1cccc2c1-c1ccccc1C2(C)C]

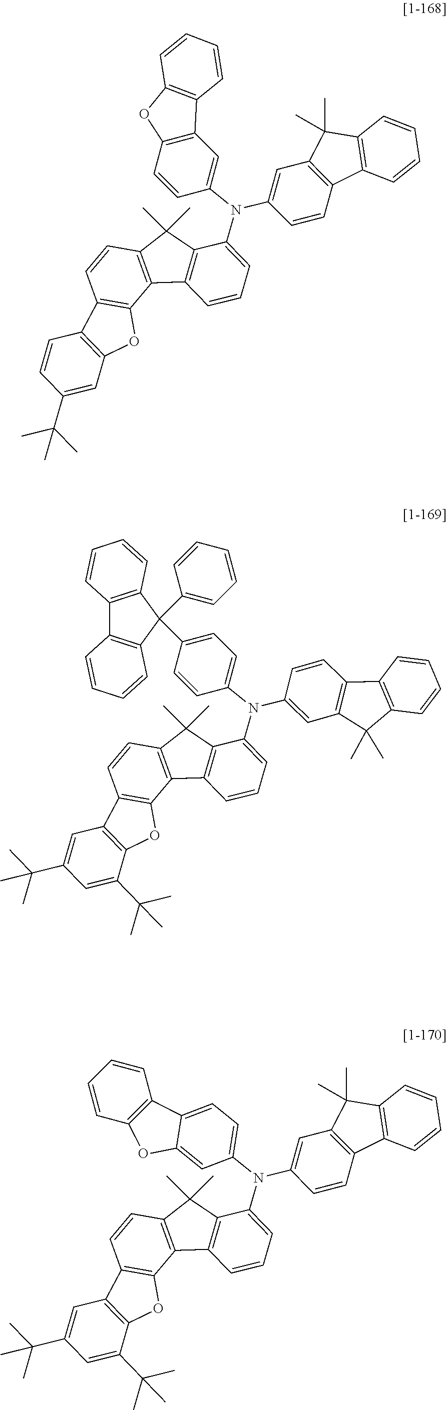 [SMILES: CC(C)(C)c1ccc2c(c1)oc1c3c(ccc12)C(C)(C)c1c-3cccc1N(c1ccc2c(c1)C(C)(C)c1ccccc1-2)c1ccc2oc3ccccc3c2c1 ; CC(C)(C)c1cc(C(C)(C)C)c2oc3c4c(ccc3c2c1)C(C)(C)c1c-4cccc1N(c1ccc(C2(c3ccccc3)c3ccccc3-c3ccccc32)cc1)c1ccc2c(c1)C(C)(C)c1ccccc1-2 ; CC(C)(C)c1cc(C(C)(C)C)c2oc3c4c(ccc3c2c1)C(C)(C)c1c-4cccc1N(c1ccc2c(c1)C(C)(C)c1ccccc1-2)c1ccc2c(c1)oc1ccccc12]

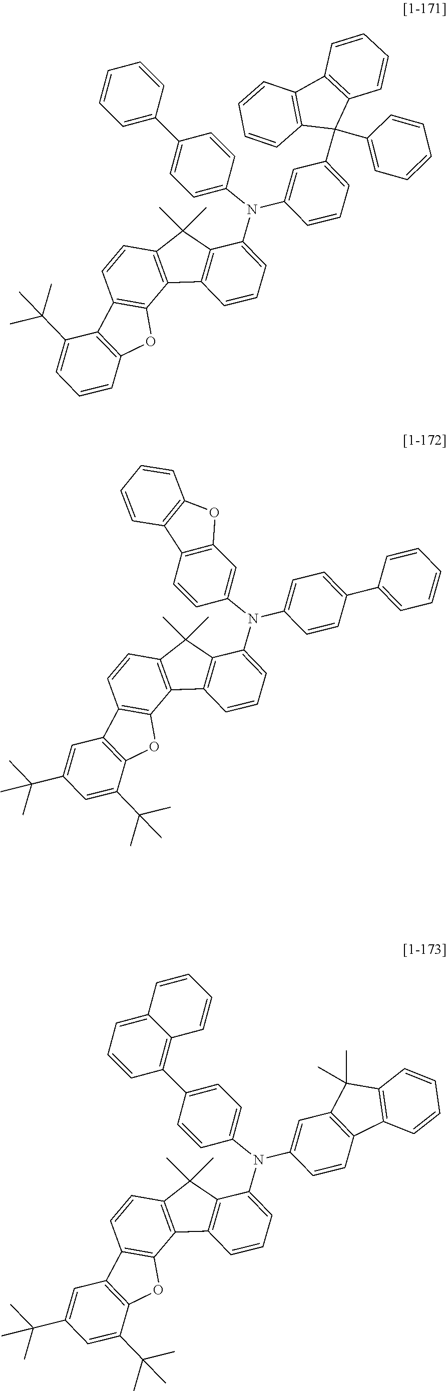 [SMILES: CC(C)(C)c1cccc2oc3c4c(ccc3c12)C(C)(C)c1c-4cccc1N(c1ccc(-c2ccccc2)cc1)c1cccc(C2(c3ccccc3)c3ccccc3-c3ccccc32)c1 ; CC(C)(C)c1cc(C(C)(C)C)c2oc3c4c(ccc3c2c1)C(C)(C)c1c-4cccc1N(c1ccc(-c2ccccc2)cc1)c1ccc2c(c1)oc1ccccc12 ; CC(C)(C)c1cc(C(C)(C)C)c2oc3c4c(ccc3c2c1)C(C)(C)c1c-4cccc1N(c1ccc(-c2cccc3ccccc23)cc1)c1ccc2c(c1)C(C)(C)c1ccccc1-2]

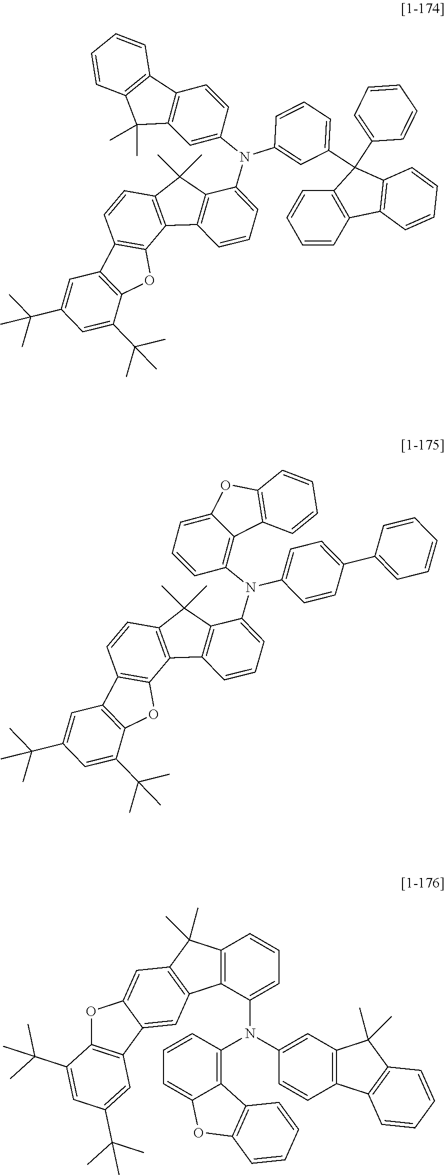 [SMILES: CC(C)(C)c1cc(C(C)(C)C)c2oc3c4c(ccc3c2c1)C(C)(C)c1c-4cccc1N(c1cccc(C2(c3ccccc3)c3ccccc3-c3ccccc32)c1)c1ccc2c(c1)C(C)(C)c1ccccc1-2 ; CC(C)(C)c1cc(C(C)(C)C)c2oc3c4c(ccc3c2c1)C(C)(C)c1c-4cccc1N(c1ccc(-c2ccccc2)cc1)c1cccc2oc3ccccc3c12 ; CC(C)(C)c1cc(C(C)(C)C)c2oc3cc4c(cc3c2c1)-c1c(N(c2ccc3c(c2)C(C)(C)c2ccccc2-3)c2cccc3oc5ccccc5c23)cccc1C4(C)C]

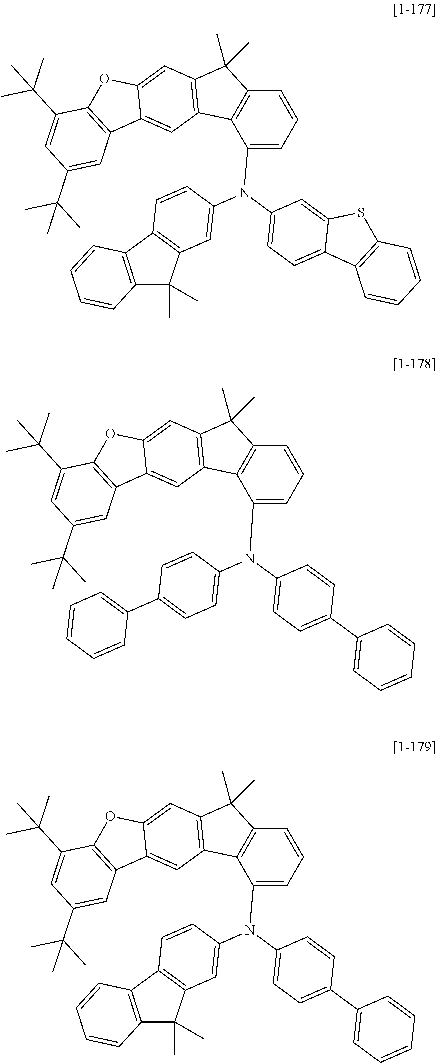 [SMILES: CC(C)(C)c1cc(C(C)(C)C)c2oc3cc4c(cc3c2c1)-c1c(N(c2ccc3c(c2)C(C)(C)c2ccccc2-3)c2ccc3c(c2)sc2ccccc23)cccc1C4(C)C ; CC(C)(C)c1cc(C(C)(C)C)c2oc3cc4c(cc3c2c1)-c1c(N(c2ccc(-c3ccccc3)cc2)c2ccc(-c3ccccc3)cc2)cccc1C4(C)C ; CC(C)(C)c1cc(C(C)(C)C)c2oc3cc4c(cc3c2c1)-c1c(N(c2ccc(-c3ccccc3)cc2)c2ccc3c(c2)C(C)(C)c2ccccc2-3)cccc1C4(C)C]

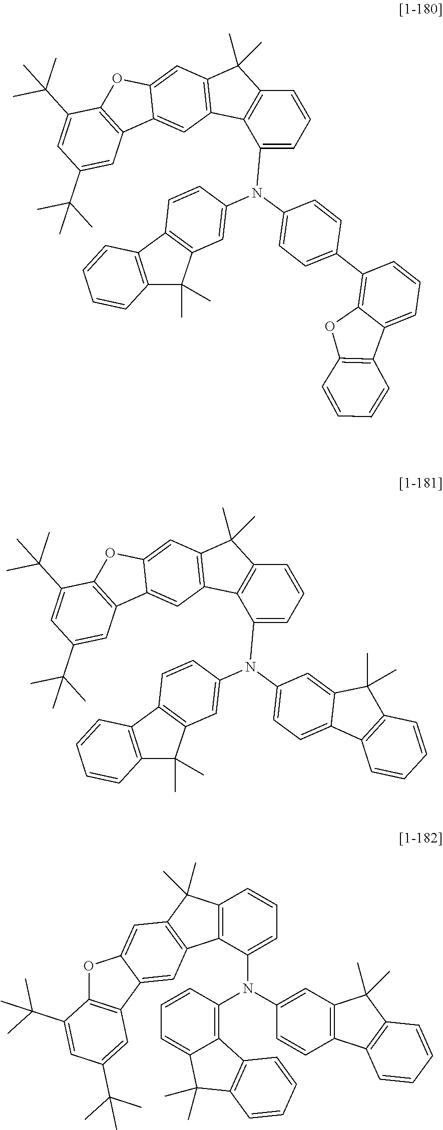 [SMILES: CC(C)(C)c1cc(C(C)(C)C)c2oc3cc4c(cc3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1cc(C(C)(C)C)c2oc3cc4c(cc3c2c1)-c1c(N(c2ccc3c(c2)C(C)(C)c2ccccc2-3)c2ccc3c(c2)C(C)(C)c2ccccc2-3)cccc1C4(C)C ; CC(C)(C)c1cc(C(C)(C)C)c2oc3cc4c(cc3c2c1)-c1c(N(c2ccc3c(c2)C(C)(C)c2ccccc2-3)c2cccc3c2-c2ccccc2C3(C)C)cccc1C4(C)C]

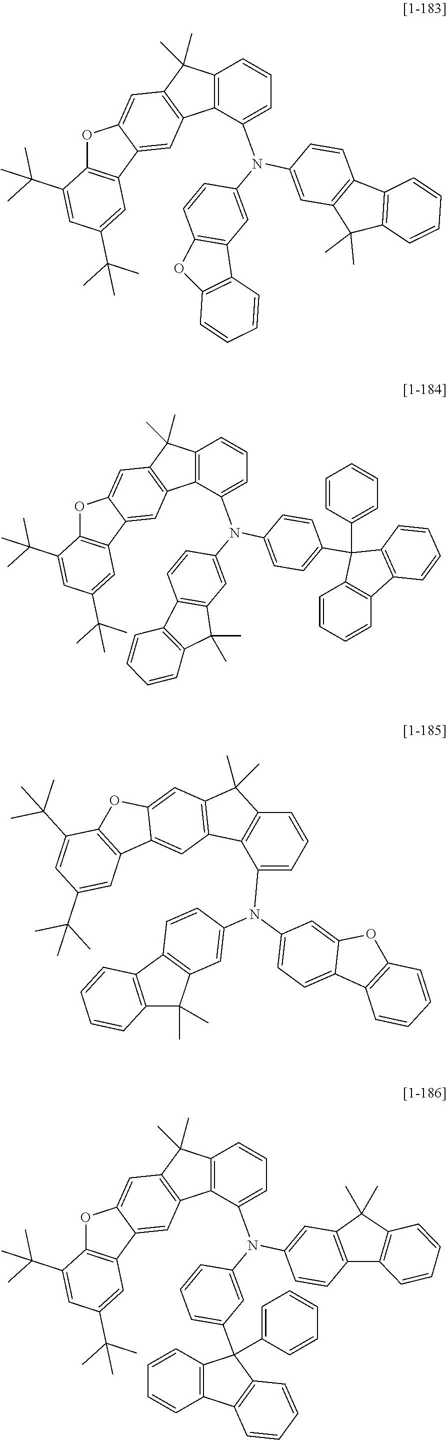 [SMILES: CC(C)(C)c1cc(C(C)(C)C)c2oc3cc4c(cc3c2c1)-c1c(N(c2ccc3c(c2)C(C)(C)c2ccccc2-3)c2ccc3oc5ccccc5c3c2)cccc1C4(C)C ; CC(C)(C)c1cc(C(C)(C)C)c2oc3cc4c(cc3c2c1)-c1c(N(c2ccc(C3(c5ccccc5)c5ccccc5-c5ccccc53)cc2)c2ccc3c(c2)C(C)(C)c2ccccc2-3)cccc1C4(C)C ; CC(C)(C)c1cc(C(C)(C)C)c2oc3cc4c(cc3c2c1)-c1c(N(c2ccc3c(c2)C(C)(C)c2ccccc2-3)c2ccc3c(c2)oc2ccccc23)cccc1C4(C)C ; CC(C)(C)c1cc(C(C)(C)C)c2oc3cc4c(cc3c2c1)-c1c(N(c2cccc(C3(c5ccccc5)c5ccccc5-c5ccccc53)c2)c2ccc3c(c2)C(C)(C)c2ccccc2-3)cccc1C4(C)C]

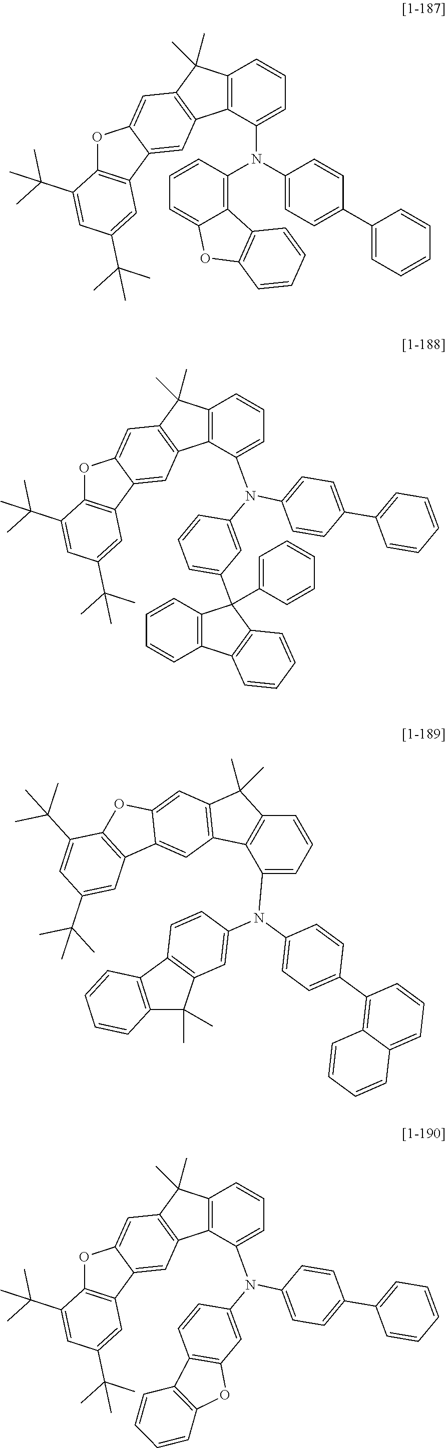 [SMILES: CC(C)(C)c1cc(C(C)(C)C)c2oc3cc4c(cc3c2c1)-c1c(N(c2ccc(-c3ccccc3)cc2)c2cccc3oc5ccccc5c23)cccc1C4(C)C ; CC(C)(C)c1cc(C(C)(C)C)c2oc3cc4c(cc3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1cc(C(C)(C)C)c2oc3cc4c(cc3c2c1)-c1c(N(c2ccc(-c3cccc5ccccc35)cc2)c2ccc3c(c2)C(C)(C)c2ccccc2-3)cccc1C4(C)C ; CC(C)(C)c1cc(C(C)(C)C)c2oc3cc4c(cc3c2c1)-c1c(N(c2ccc(-c3ccccc3)cc2)c2ccc3c(c2)oc2ccccc23)cccc1C4(C)C]

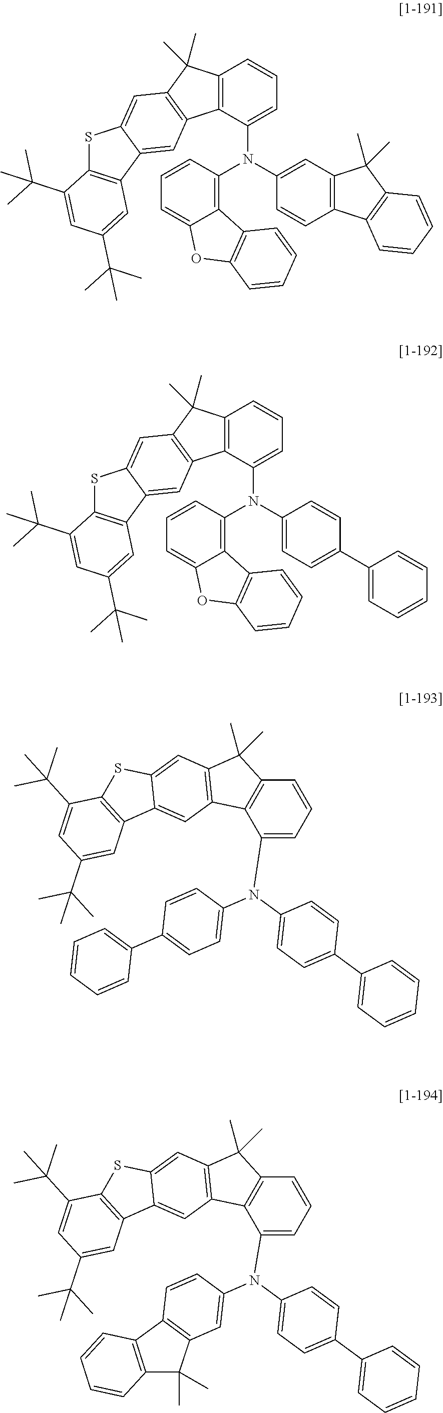 [SMILES: CC(C)(C)c1cc(C(C)(C)C)c2sc3cc4c(cc3c2c1)-c1c(N(c2ccc3c(c2)C(C)(C)c2ccccc2-3)c2cccc3oc5ccccc5c23)cccc1C4(C)C ; CC(C)(C)c1cc(C(C)(C)C)c2sc3cc4c(cc3c2c1)-c1c(N(c2ccc(-c3ccccc3)cc2)c2cccc3oc5ccccc5c23)cccc1C4(C)C ; CC(C)(C)c1cc(C(C)(C)C)c2sc3cc4c(cc3c2c1)-c1c(N(c2ccc(-c3ccccc3)cc2)c2ccc(-c3ccccc3)cc2)cccc1C4(C)C ; CC(C)(C)c1cc(C(C)(C)C)c2sc3cc4c(cc3c2c1)-c1c(N(c2ccc(-c3ccccc3)cc2)c2ccc3c(c2)C(C)(C)c2ccccc2-3)cccc1C4(C)C]

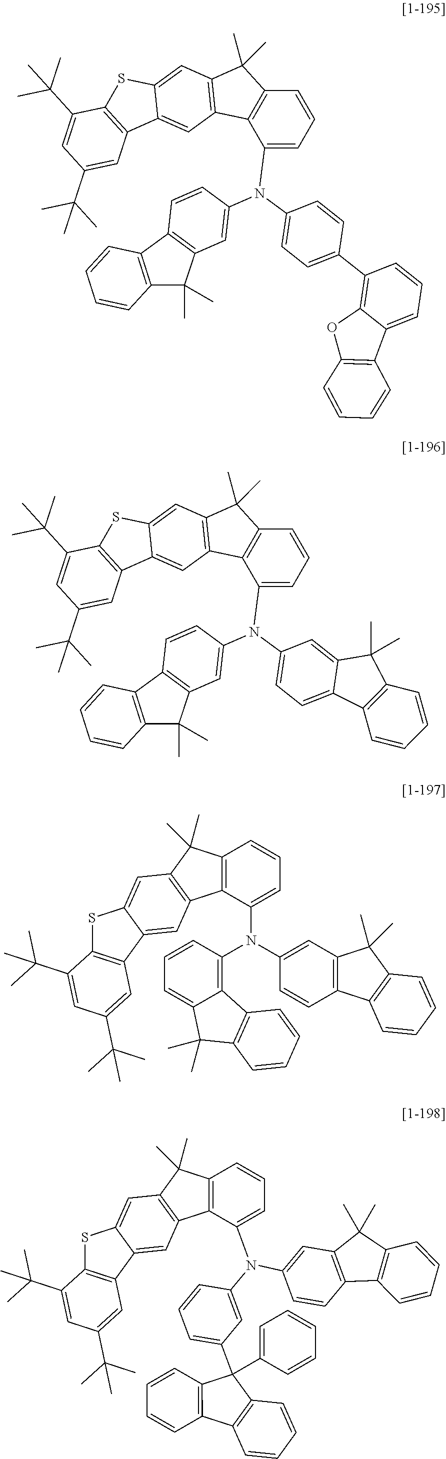 [SMILES: CC(C)(C)c1cc(C(C)(C)C)c2sc3cc4c(cc3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1cc(C(C)(C)C)c2sc3cc4c(cc3c2c1)-c1c(N(c2ccc3c(c2)C(C)(C)c2ccccc2-3)c2ccc3c(c2)C(C)(C)c2ccccc2-3)cccc1C4(C)C ; CC(C)(C)c1cc(C(C)(C)C)c2sc3cc4c(cc3c2c1)-c1c(N(c2ccc3c(c2)C(C)(C)c2ccccc2-3)c2cccc3c2-c2ccccc2C3(C)C)cccc1C4(C)C ; CC(C)(C)c1cc(C(C)(C)C)c2sc3cc4c(cc3c2c1)-c1c(N(c2cccc(C3(c5ccccc5)c5ccccc5-c5ccccc53)c2)c2ccc3c(c2)C(C)(C)c2ccccc2-3)cccc1C4(C)C]

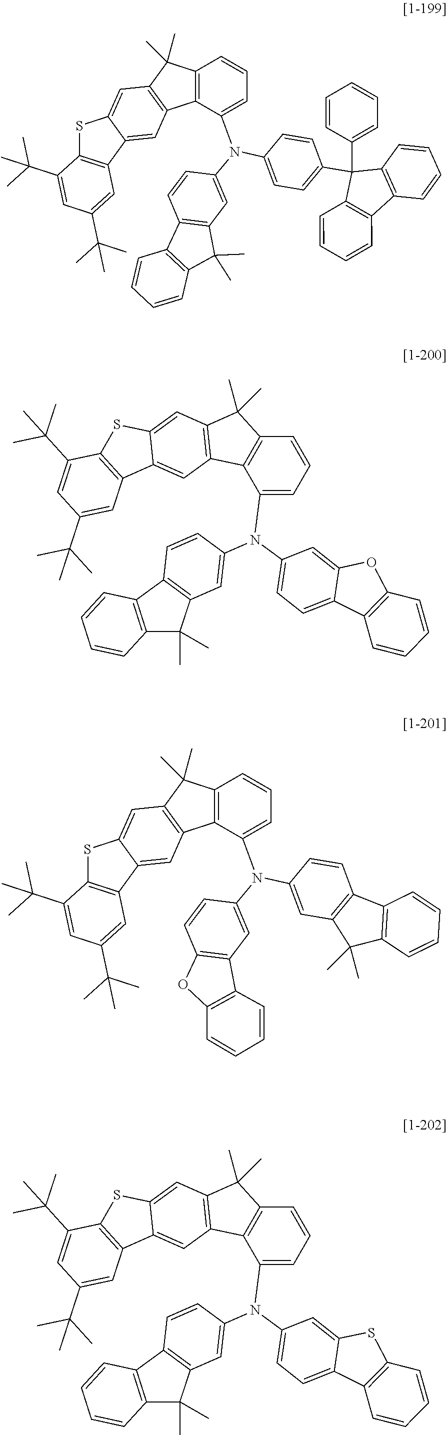 [SMILES: CC(C)(C)c1cc(C(C)(C)C)c2sc3cc4c(cc3c2c1)-c1c(N(c2ccc(C3(c5ccccc5)c5ccccc5-c5ccccc53)cc2)c2ccc3c(c2)C(C)(C)c2ccccc2-3)cccc1C4(C)C ; CC(C)(C)c1cc(C(C)(C)C)c2sc3cc4c(cc3c2c1)-c1c(N(c2ccc3c(c2)C(C)(C)c2ccccc2-3)c2ccc3c(c2)oc2ccccc23)cccc1C4(C)C ; CC(C)(C)c1cc(C(C)(C)C)c2sc3cc4c(cc3c2c1)-c1c(N(c2ccc3c(c2)C(C)(C)c2ccccc2-3)c2ccc3oc5ccccc5c3c2)cccc1C4(C)C ; CC(C)(C)c1cc(C(C)(C)C)c2sc3cc4c(cc3c2c1)-c1c(N(c2ccc3c(c2)C(C)(C)c2ccccc2-3)c2ccc3c(c2)sc2ccccc23)cccc1C4(C)C]

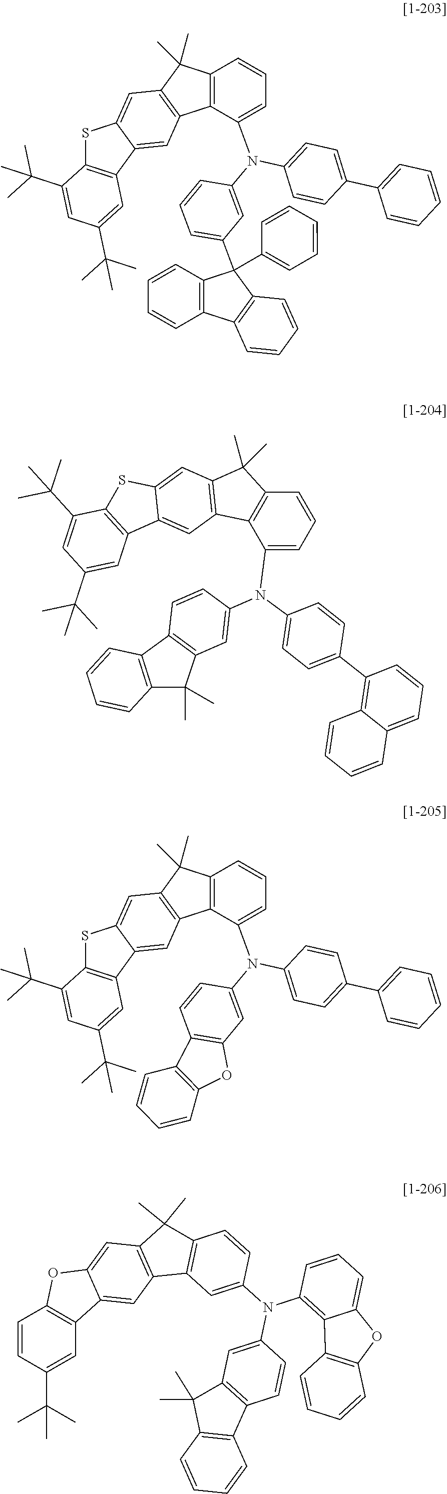 [SMILES: CC(C)(C)c1cc(C(C)(C)C)c2sc3cc4c(cc3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1cc(C(C)(C)C)c2sc3cc4c(cc3c2c1)-c1c(N(c2ccc(-c3cccc5ccccc35)cc2)c2ccc3c(c2)C(C)(C)c2ccccc2-3)cccc1C4(C)C ; CC(C)(C)c1cc(C(C)(C)C)c2sc3cc4c(cc3c2c1)-c1c(N(c2ccc(-c3ccccc3)cc2)c2ccc3c(c2)oc2ccccc23)cccc1C4(C)C ; CC(C)(C)c1ccc2oc3cc4c(cc3c2c1)-c1cc(N(c2ccc3c(c2)C(C)(C)c2ccccc2-3)c2cccc3oc5ccccc5c23)ccc1C4(C)C]

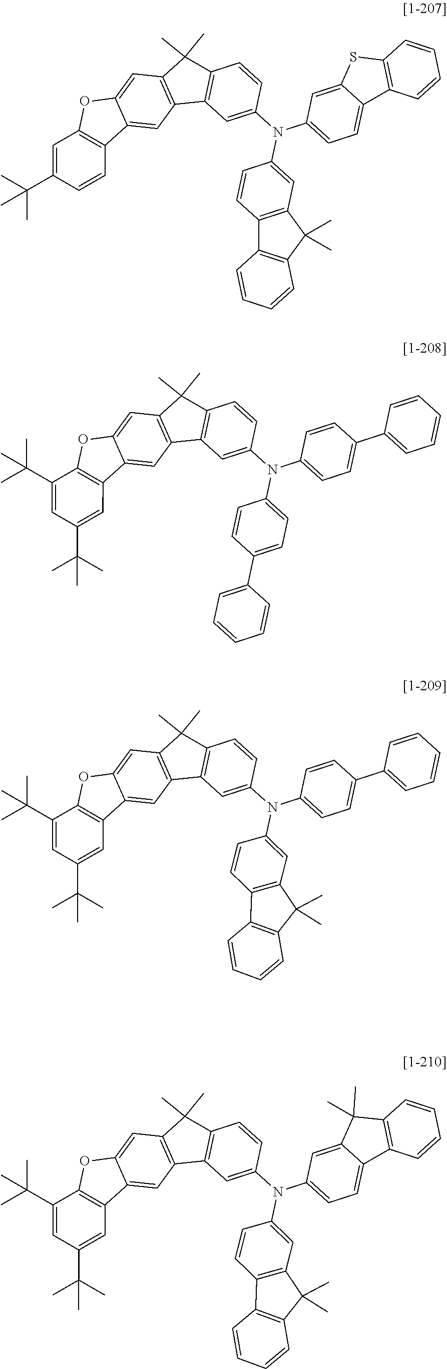 [SMILES: CC(C)(C)c1ccc2c(c1)oc1cc3c(cc12)-c1cc(N(c2ccc4c(c2)C(C)(C)c2ccccc2-4)c2ccc4c(c2)sc2ccccc24)ccc1C3(C)C ; CC(C)(C)c1cc(C(C)(C)C)c2oc3cc4c(cc3c2c1)-c1cc(N(c2ccc(-c3ccccc3)cc2)c2ccc(-c3ccccc3)cc2)ccc1C4(C)C ; CC(C)(C)c1cc(C(C)(C)C)c2oc3cc4c(cc3c2c1)-c1cc(N(c2ccc(-c3ccccc3)cc2)c2ccc3c(c2)C(C)(C)c2ccccc2-3)ccc1C4(C)C ; CC(C)(C)c1cc(C(C)(C)C)c2oc3cc4c(cc3c2c1)-c1cc(N(c2ccc3c(c2)C(C)(C)c2ccccc2-3)c2ccc3c(c2)C(C)(C)c2ccccc2-3)ccc1C4(C)C]

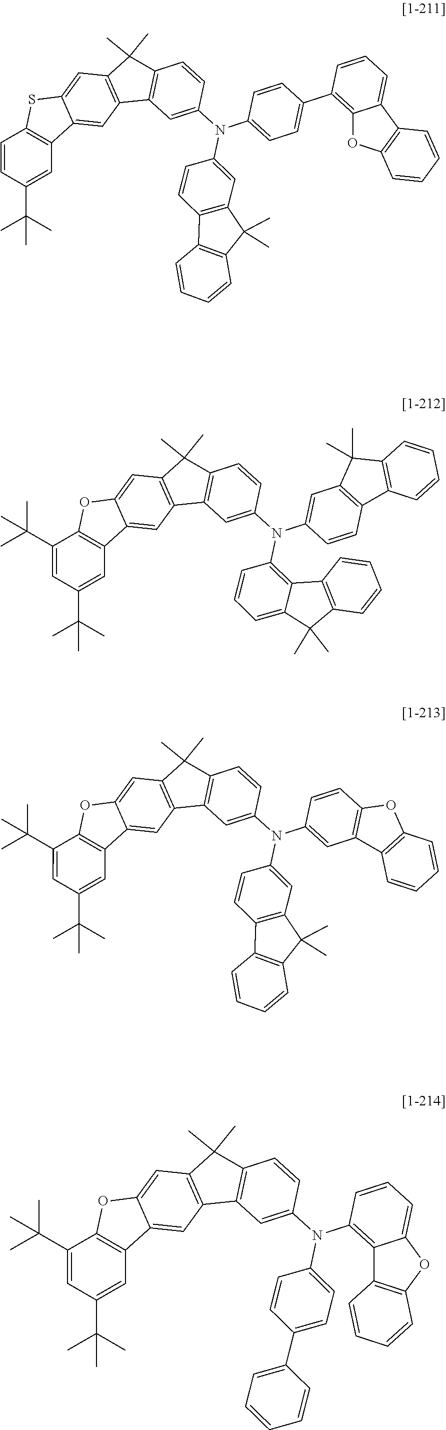 [SMILES: CC(C)(C)c1ccc2sc3cc4c(cc3c2c1)-c1cc(N(c2ccc(-c3cccc5c3oc3ccccc35)cc2)c2ccc3c(c2)C(C)(C)c2ccccc2-3)ccc1C4(C)C ; CC(C)(C)c1cc(C(C)(C)C)c2oc3cc4c(cc3c2c1)-c1cc(N(c2ccc3c(c2)C(C)(C)c2ccccc2-3)c2cccc3c2-c2ccccc2C3(C)C)ccc1C4(C)C ; CC(C)(C)c1cc(C(C)(C)C)c2oc3cc4c(cc3c2c1)-c1cc(N(c2ccc3c(c2)C(C)(C)c2ccccc2-3)c2ccc3oc5ccccc5c3c2)ccc1C4(C)C ; CC(C)(C)c1cc(C(C)(C)C)c2oc3cc4c(cc3c2c1)-c1cc(N(c2ccc(-c3ccccc3)cc2)c2cccc3oc5ccccc5c23)ccc1C4(C)C]

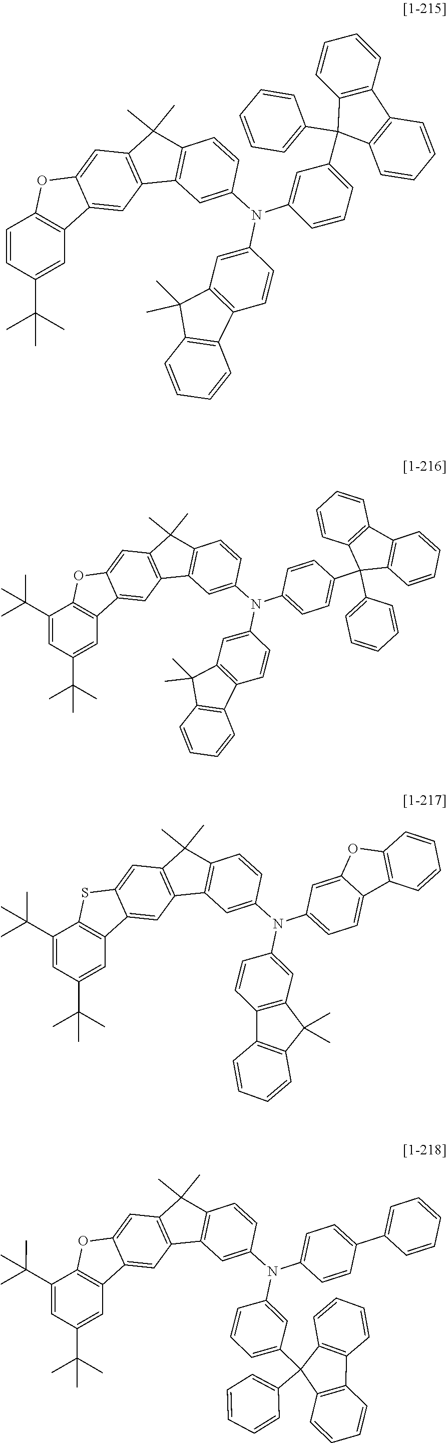 [SMILES: CC(C)(C)c1ccc2oc3cc4c(cc3c2c1)-c1cc(N(c2cccc(C3(c5ccccc5)c5ccccc5-c5ccccc53)c2)c2ccc3c(c2)C(C)(C)c2ccccc2-3)ccc1C4(C)C ; CC(C)(C)c1cc(C(C)(C)C)c2oc3cc4c(cc3c2c1)-c1cc(N(c2ccc(C3(c5ccccc5)c5ccccc5-c5ccccc53)cc2)c2ccc3c(c2)C(C)(C)c2ccccc2-3)ccc1C4(C)C ; CC(C)(C)c1cc(C(C)(C)C)c2sc3cc4c(cc3c2c1)-c1cc(N(c2ccc3c(c2)C(C)(C)c2ccccc2-3)c2ccc3c(c2)oc2ccccc23)ccc1C4(C)C ; CC(C)(C)c1cc(C(C)(C)C)c2oc3cc4c(cc3c2c1)-c1cc(N(c2ccc(-c3ccccc3)cc2)c2cccc(C3(c5ccccc5)c5ccccc5-c5ccccc53)c2)ccc1C4(C)C]

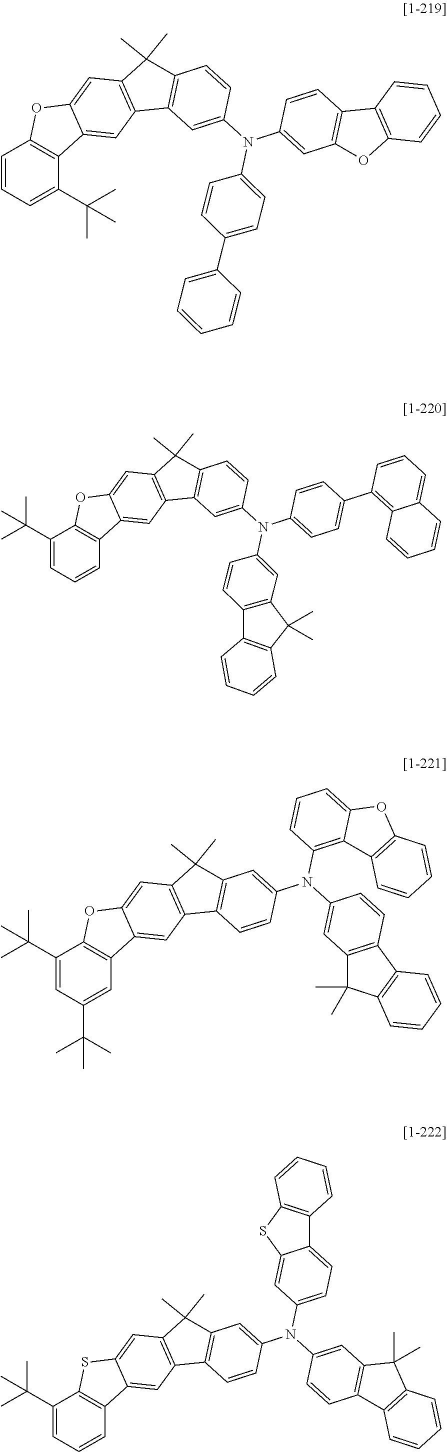 [SMILES: CC(C)(C)c1cccc2oc3cc4c(cc3c12)-c1cc(N(c2ccc(-c3ccccc3)cc2)c2ccc3c(c2)oc2ccccc23)ccc1C4(C)C ; CC(C)(C)c1cccc2c1oc1cc3c(cc12)-c1cc(N(c2ccc(-c4cccc5ccccc45)cc2)c2ccc4c(c2)C(C)(C)c2ccccc2-4)ccc1C3(C)C ; CC(C)(C)c1cc(C(C)(C)C)c2oc3cc4c(cc3c2c1)-c1ccc(N(c2ccc3c(c2)C(C)(C)c2ccccc2-3)c2cccc3oc5ccccc5c23)cc1C4(C)C ; CC(C)(C)c1cccc2c1sc1cc3c(cc12)-c1ccc(N(c2ccc4c(c2)C(C)(C)c2ccccc2-4)c2ccc4c(c2)sc2ccccc24)cc1C3(C)C]

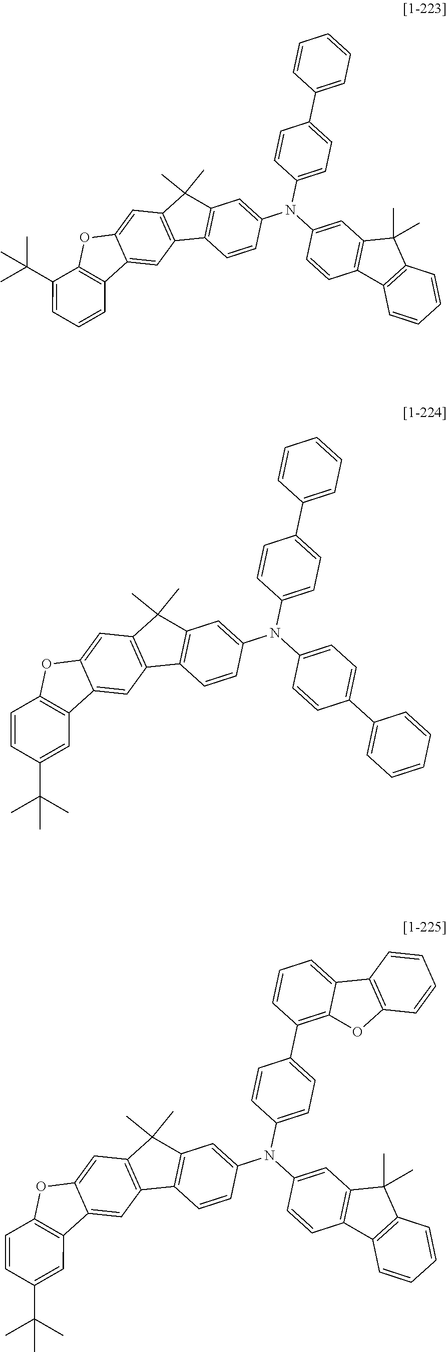 [SMILES: CC(C)(C)c1cccc2c1oc1cc3c(cc12)-c1ccc(N(c2ccc(-c4ccccc4)cc2)c2ccc4c(c2)C(C)(C)c2ccccc2-4)cc1C3(C)C ; CC(C)(C)c1ccc2oc3cc4c(cc3c2c1)-c1ccc(N(c2ccc(-c3ccccc3)cc2)c2ccc(-c3ccccc3)cc2)cc1C4(C)C ; CC(C)(C)c1ccc2oc3cc4c(cc3c2c1)-c1ccc(N(c2ccc(-c3cccc5c3oc3ccccc35)cc2)c2ccc3c(c2)C(C)(C)c2ccccc2-3)cc1C4(C)C]

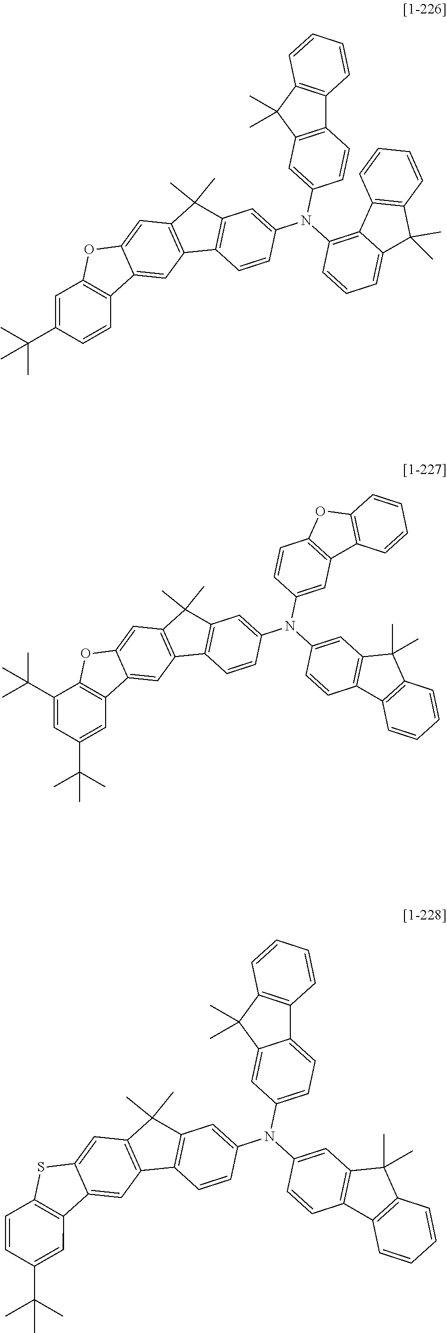

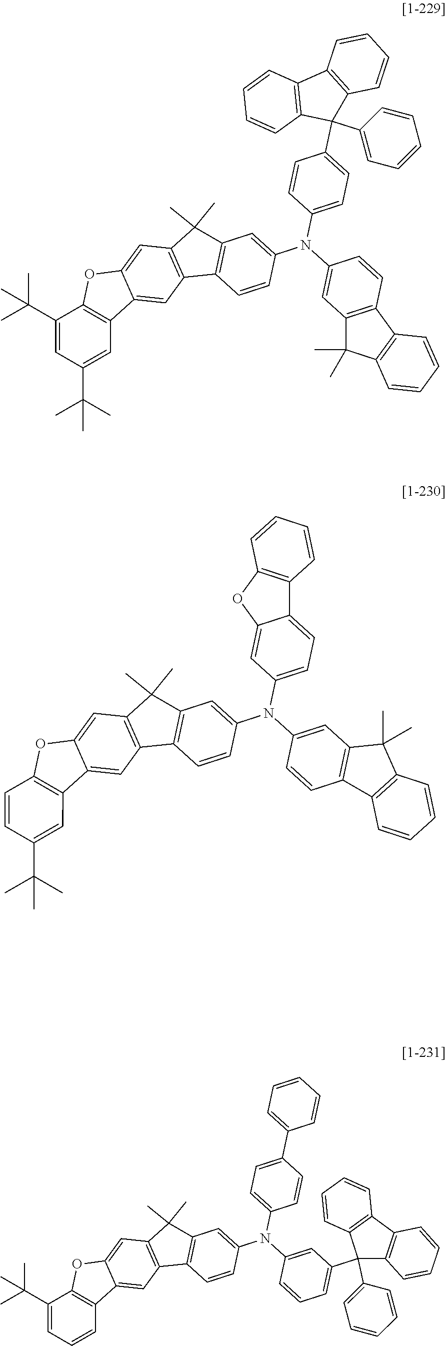 [SMILES: CC(C)(C)c1cc(C(C)(C)C)c2oc3cc4c(cc3c2c1)-c1ccc(N(c2ccc(C3(c5ccccc5)c5ccccc5-c5ccccc53)cc2)c2ccc3c(c2)C(C)(C)c2ccccc2-3)cc1C4(C)C ; CC(C)(C)c1ccc2oc3cc4c(cc3c2c1)-c1ccc(N(c2ccc3c(c2)C(C)(C)c2ccccc2-3)c2ccc3c(c2)oc2ccccc23)cc1C4(C)C ; CC(C)(C)c1cccc2c1oc1cc3c(cc12)-c1ccc(N(c2ccc(-c4ccccc4)cc2)c2cccc(C4(c5ccccc5)c5ccccc5-c5ccccc54)c2)cc1C3(C)C]

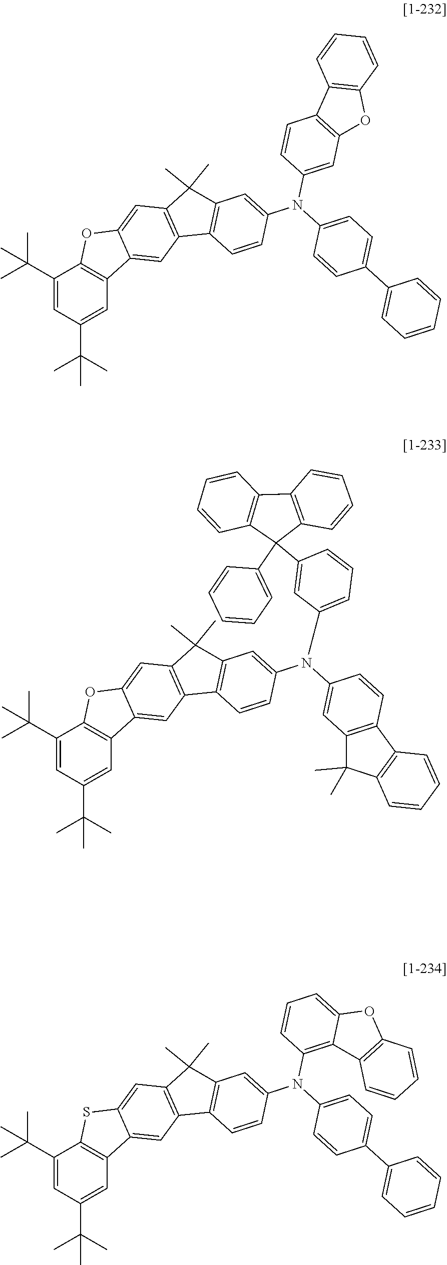 [SMILES: CC(C)(C)c1cc(C(C)(C)C)c2oc3cc4c(cc3c2c1)-c1ccc(N(c2ccc(-c3ccccc3)cc2)c2ccc3c(c2)oc2ccccc23)cc1C4(C)C ; CC(C)(C)c1cc(C(C)(C)C)c2oc3cc4c(cc3c2c1)-c1ccc(N(c2cccc(C3(c5ccccc5)c5ccccc5-c5ccccc53)c2)c2ccc3c(c2)C(C)(C)c2ccccc2-3)cc1C4(C)C ; CC(C)(C)c1cc(C(C)(C)C)c2sc3cc4c(cc3c2c1)-c1ccc(N(c2ccc(-c3ccccc3)cc2)c2cccc3oc5ccccc5c23)cc1C4(C)C]

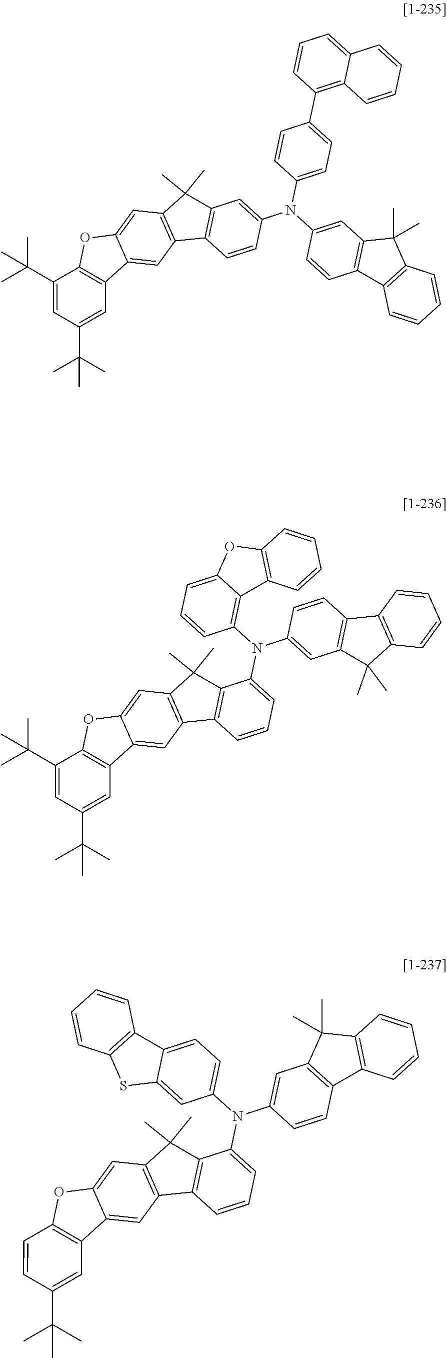 [SMILES: CC(C)(C)c1cc(C(C)(C)C)c2oc3cc4c(cc3c2c1)-c1ccc(N(c2ccc(-c3cccc5ccccc35)cc2)c2ccc3c(c2)C(C)(C)c2ccccc2-3)cc1C4(C)C ; CC(C)(C)c1cc(C(C)(C)C)c2oc3cc4c(cc3c2c1)-c1cccc(N(c2ccc3c(c2)C(C)(C)c2ccccc2-3)c2cccc3oc5ccccc5c23)c1C4(C)C ; CC(C)(C)c1ccc2oc3cc4c(cc3c2c1)-c1cccc(N(c2ccc3c(c2)C(C)(C)c2ccccc2-3)c2ccc3c(c2)sc2ccccc23)c1C4(C)C]

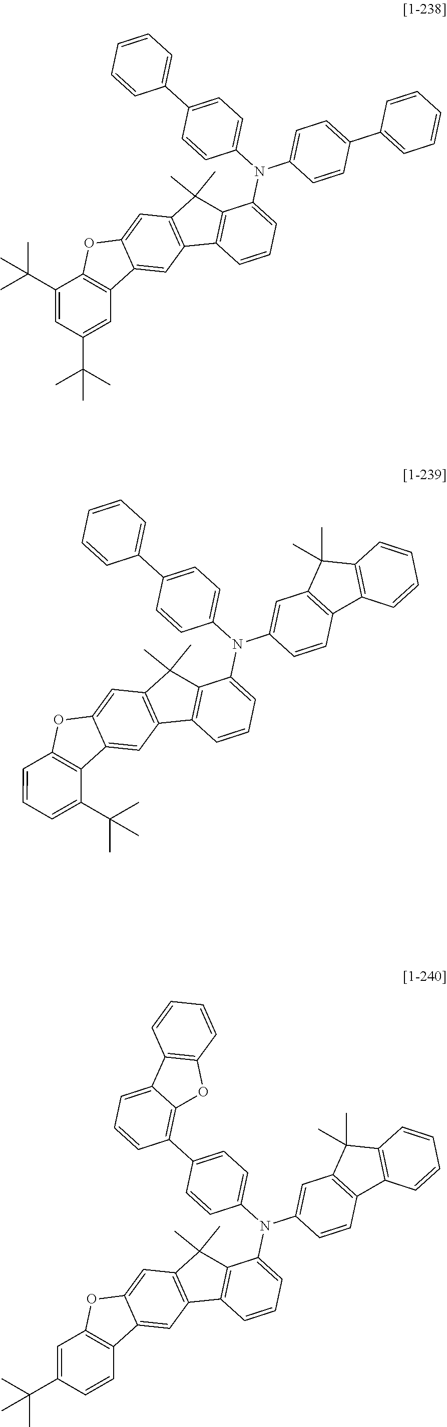 [SMILES: CC(C)(C)c1cc(C(C)(C)C)c2oc3cc4c(cc3c2c1)-c1cccc(N(c2ccc(-c3ccccc3)cc2)c2ccc(-c3ccccc3)cc2)c1C4(C)C ; CC(C)(C)c1cccc2oc3cc4c(cc3c12)-c1cccc(N(c2ccc(-c3ccccc3)cc2)c2ccc3c(c2)C(C)(C)c2ccccc2-3)c1C4(C)C ; CC(C)(C)c1ccc2c(c1)oc1cc3c(cc12)-c1cccc(N(c2ccc(-c4cccc5c4oc4ccccc45)cc2)c2ccc4c(c2)C(C)(C)c2ccccc2-4)c1C3(C)C]

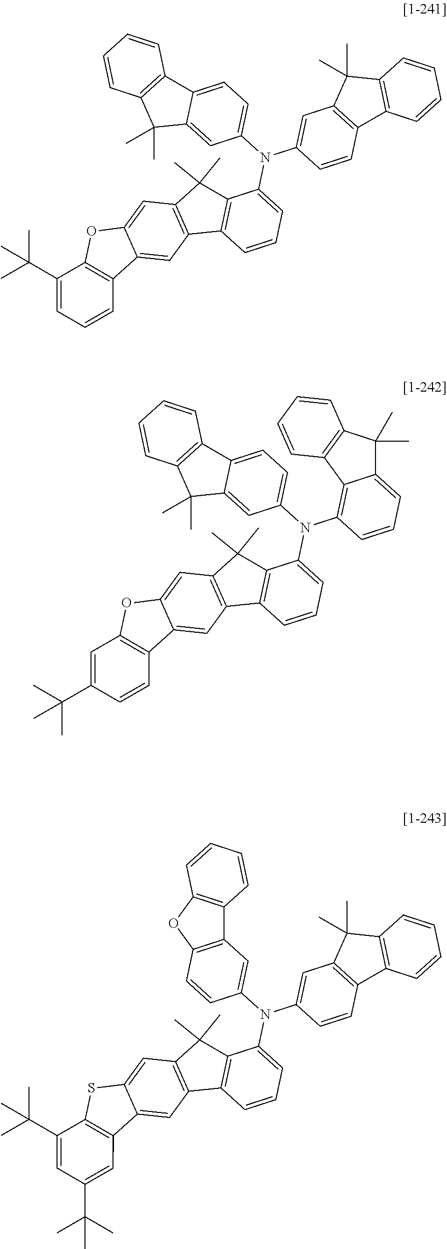

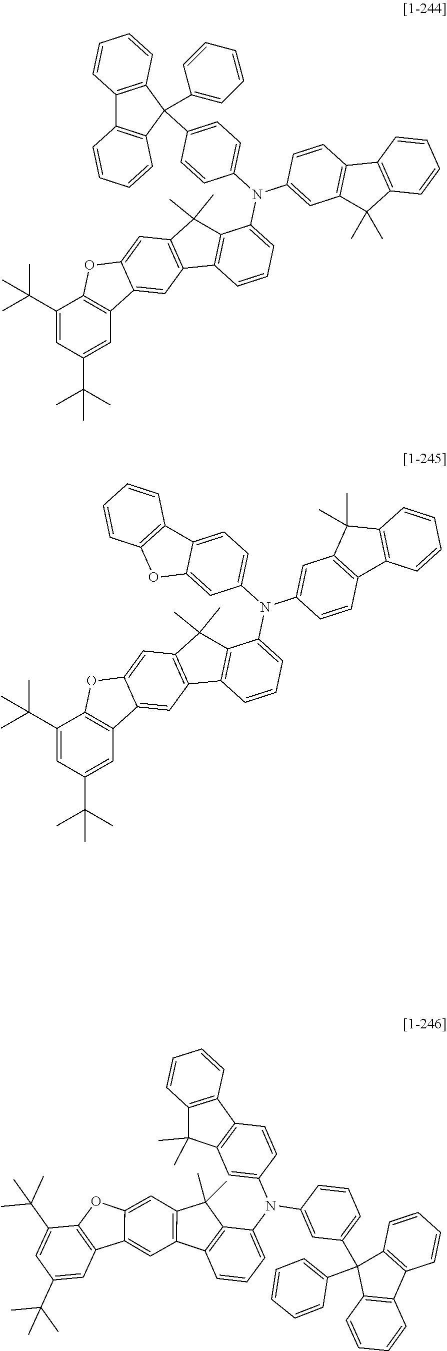 [SMILES: CC(C)(C)c1cc(C(C)(C)C)c2oc3cc4c(cc3c2c1)-c1cccc(N(c2ccc(C3(c5ccccc5)c5ccccc5-c5ccccc53)cc2)c2ccc3c(c2)C(C)(C)c2ccccc2-3)c1C4(C)C ; CC(C)(C)c1cc(C(C)(C)C)c2oc3cc4c(cc3c2c1)-c1cccc(N(c2ccc3c(c2)C(C)(C)c2ccccc2-3)c2ccc3c(c2)oc2ccccc23)c1C4(C)C ; CC(C)(C)c1cc(C(C)(C)C)c2oc3cc4c(cc3c2c1)-c1cccc(N(c2cccc(C3(c5ccccc5)c5ccccc5-c5ccccc53)c2)c2ccc3c(c2)C(C)(C)c2ccccc2-3)c1C4(C)C]

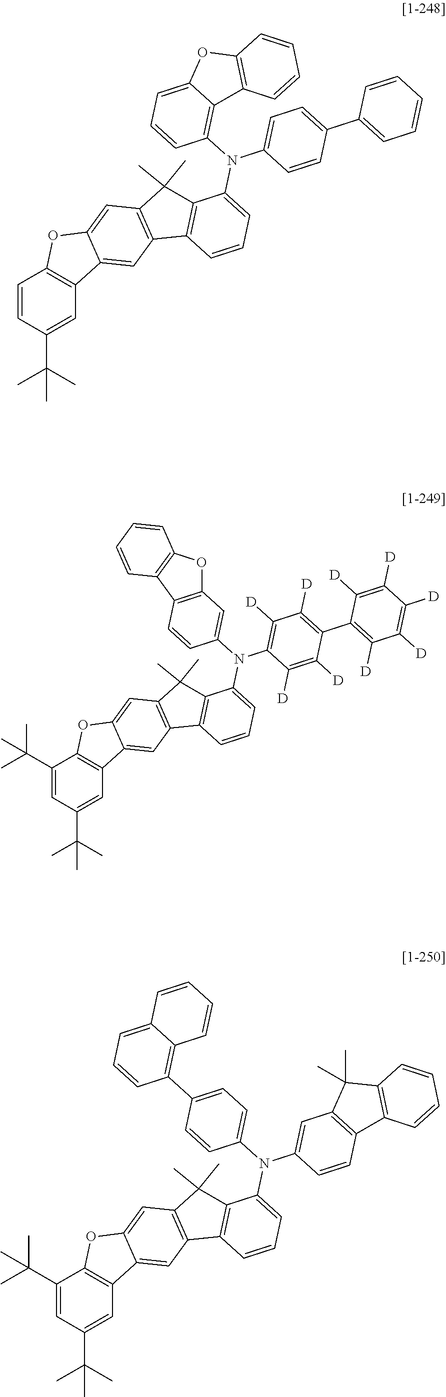 [SMILES: CC(C)(C)c1ccc2oc3cc4c(cc3c2c1)-c1cccc(N(c2ccc(-c3ccccc3)cc2)c2cccc3oc5ccccc5c23)c1C4(C)C ; [2H]c1c([2H])c([2H])c(-c2c([2H])c([2H])c(N(c3ccc4c(c3)oc3ccccc34)c3cccc4c3C(C)(C)c3cc5oc6c(C(C)(C)C)cc(C(C)(C)C)cc6c5cc3-4)c([2H])c2[2H])c([2H])c1[2H] ; CC(C)(C)c1cc(C(C)(C)C)c2oc3cc4c(cc3c2c1)-c1cccc(N(c2ccc(-c3cccc5ccccc35)cc2)c2ccc3c(c2)C(C)(C)c2ccccc2-3)c1C4(C)C]

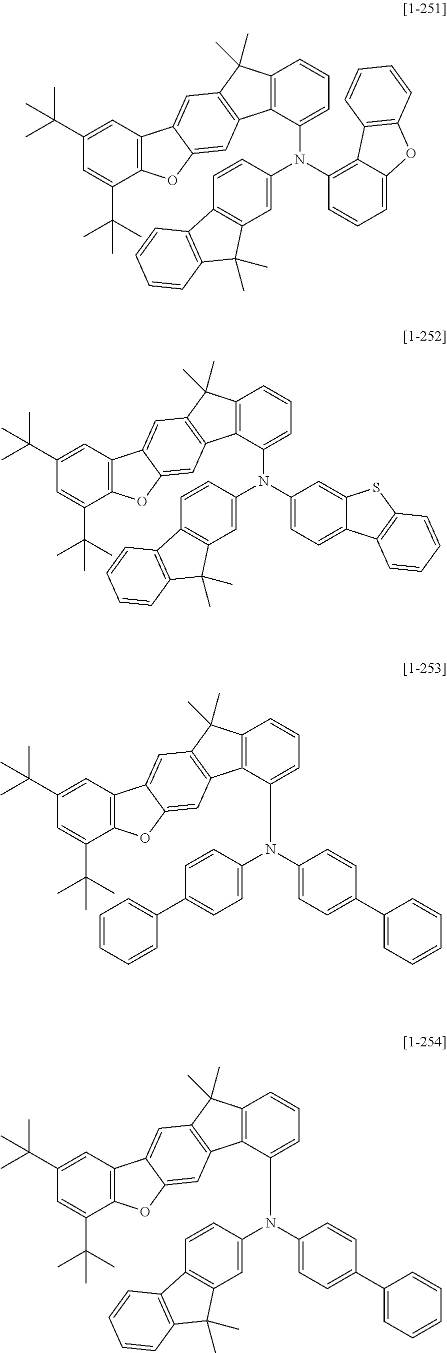 [SMILES: CC(C)(C)c1cc(C(C)(C)C)c2oc3cc4c(cc3c2c1)C(C)(C)c1cccc(N(c2ccc3c(c2)C(C)(C)c2ccccc2-3)c2cccc3oc5ccccc5c23)c1-4 ; CC(C)(C)c1cc(C(C)(C)C)c2oc3cc4c(cc3c2c1)C(C)(C)c1cccc(N(c2ccc3c(c2)C(C)(C)c2ccccc2-3)c2ccc3c(c2)sc2ccccc23)c1-4 ; CC(C)(C)c1cc(C(C)(C)C)c2oc3cc4c(cc3c2c1)C(C)(C)c1cccc(N(c2ccc(-c3ccccc3)cc2)c2ccc(-c3ccccc3)cc2)c1-4 ; CC(C)(C)c1cc(C(C)(C)C)c2oc3cc4c(cc3c2c1)C(C)(C)c1cccc(N(c2ccc(-c3ccccc3)cc2)c2ccc3c(c2)C(C)(C)c2ccccc2-3)c1-4]

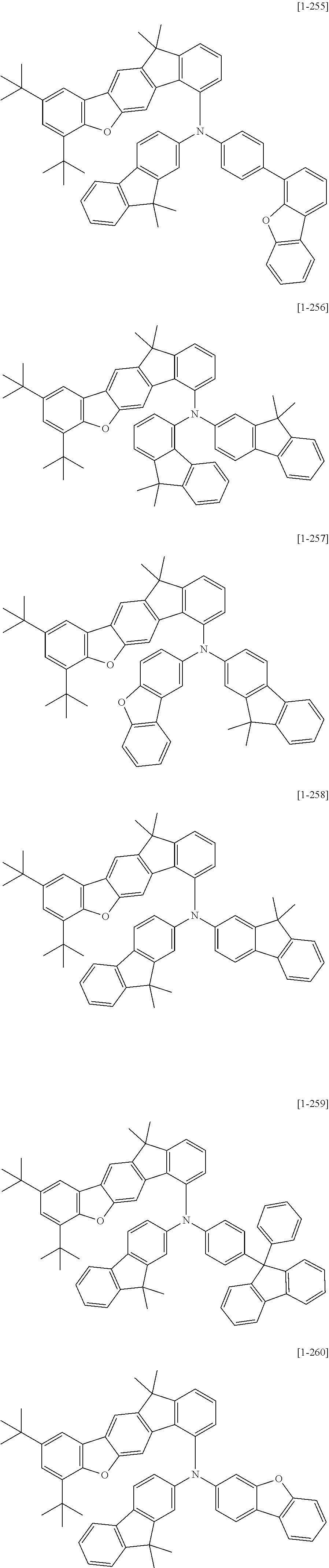

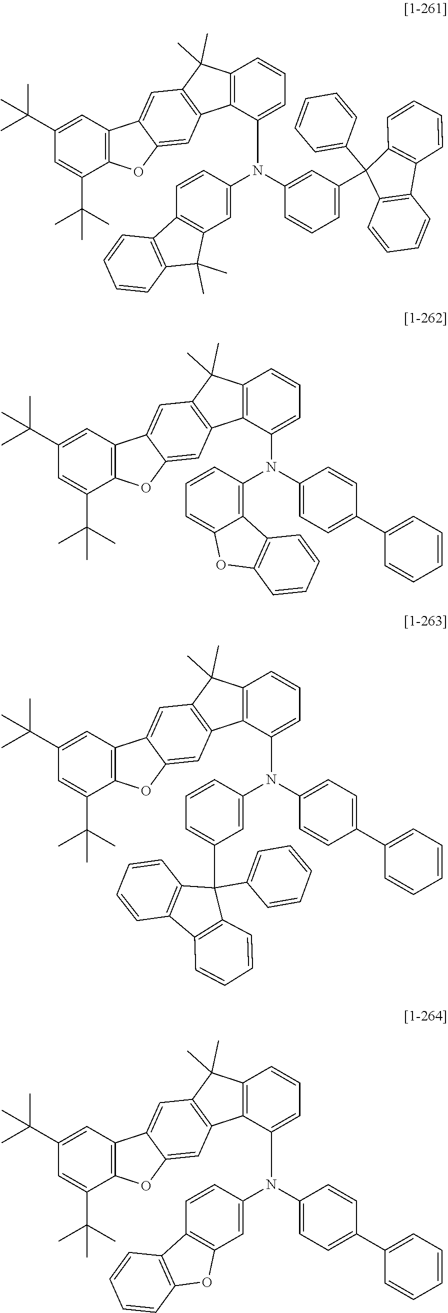 [SMILES: CC(C)(C)c1cc(C(C)(C)C)c2oc3cc4c(cc3c2c1)C(C)(C)c1cccc(N(c2cccc(C3(c5ccccc5)c5ccccc5-c5ccccc53)c2)c2ccc3c(c2)C(C)(C)c2ccccc2-3)c1-4 ; CC(C)(C)c1cc(C(C)(C)C)c2oc3cc4c(cc3c2c1)C(C)(C)c1cccc(N(c2ccc(-c3ccccc3)cc2)c2cccc3oc5ccccc5c23)c1-4 ; CC(C)(C)c1cc(C(C)(C)C)c2oc3cc4c(cc3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2oc3cc4c(cc3c2c1)C(C)(C)c1cccc(N(c2ccc(-c3ccccc3)cc2)c2ccc3c(c2)oc2ccccc23)c1-4]

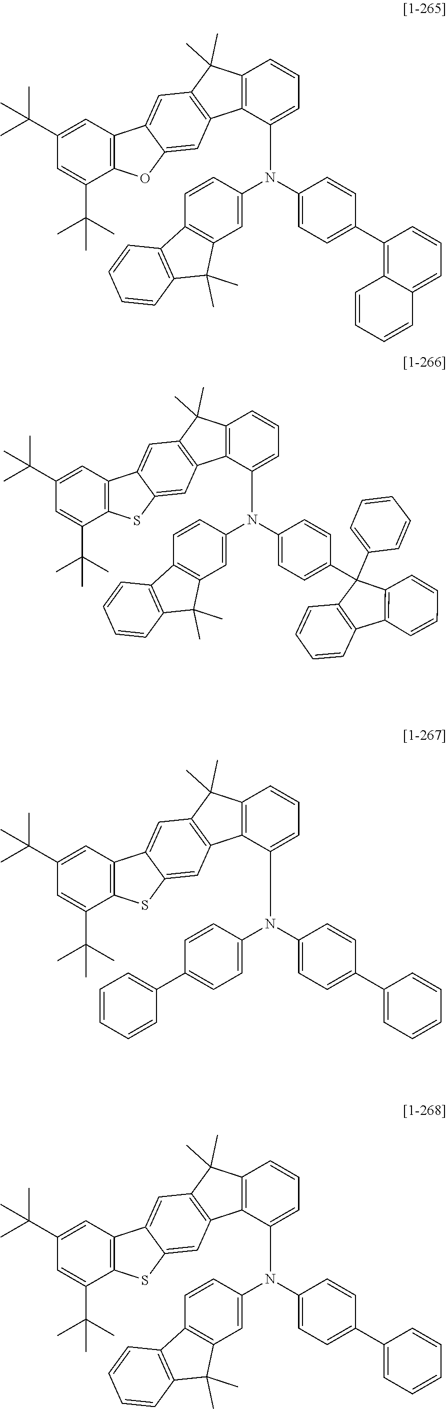 [SMILES: CC(C)(C)c1cc(C(C)(C)C)c2oc3cc4c(cc3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cc(C(C)(C)C)c2sc3cc4c(cc3c2c1)C(C)(C)c1cccc(N(c2ccc(C3(c5ccccc5)c5ccccc5-c5ccccc53)cc2)c2ccc3c(c2)C(C)(C)c2ccccc2-3)c1-4 ; CC(C)(C)c1cc(C(C)(C)C)c2sc3cc4c(cc3c2c1)C(C)(C)c1cccc(N(c2ccc(-c3ccccc3)cc2)c2ccc(-c3ccccc3)cc2)c1-4 ; CC(C)(C)c1cc(C(C)(C)C)c2sc3cc4c(cc3c2c1)C(C)(C)c1cccc(N(c2ccc(-c3ccccc3)cc2)c2ccc3c(c2)C(C)(C)c2ccccc2-3)c1-4]

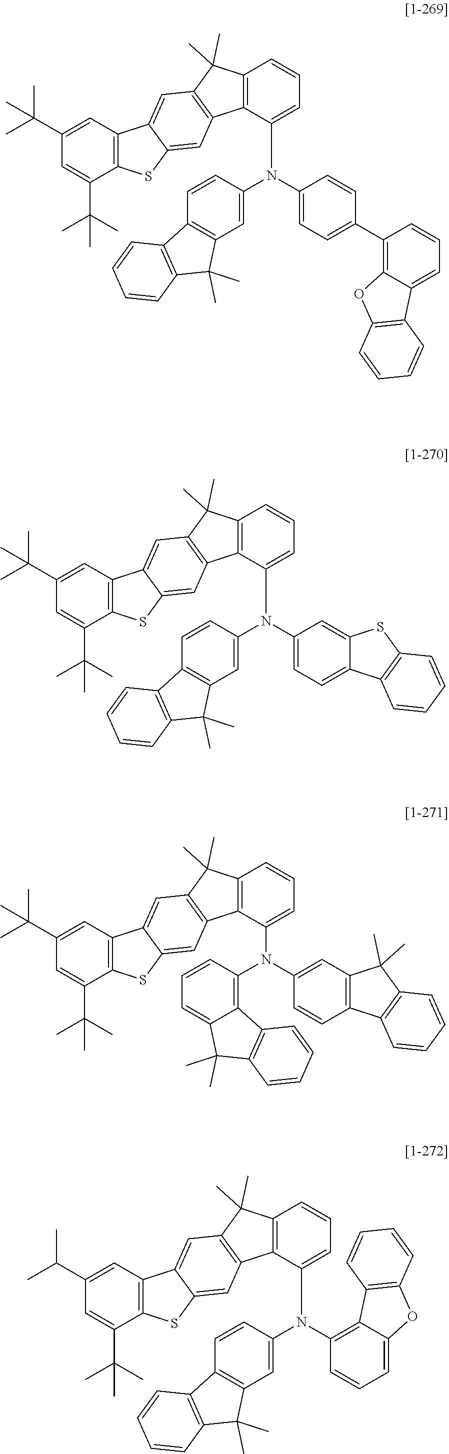 [SMILES: CC(C)(C)c1cc(C(C)(C)C)c2sc3cc4c(cc3c2c1)C(C)(C)c1cccc(N(c2ccc(-c3cccc5c3oc3ccccc35)cc2)c2ccc3c(c2)C(C)(C)c2ccccc2-3)c1-4 ; CC(C)(C)c1cc(C(C)(C)C)c2sc3cc4c(cc3c2c1)C(C)(C)c1cccc(N(c2ccc3c(c2)C(C)(C)c2ccccc2-3)c2ccc3c(c2)sc2ccccc23)c1-4 ; CC(C)(C)c1cc(C(C)(C)C)c2sc3cc4c(cc3c2c1)C(C)(C)c1cccc(N(c2ccc3c(c2)C(C)(C)c2ccccc2-3)c2cccc3c2-c2ccccc2C3(C)C)c1-4 ; CC(C)c1cc(C(C)(C)C)c2sc3cc4c(cc3c2c1)C(C)(C)c1cccc(N(c2ccc3c(c2)C(C)(C)c2ccccc2-3)c2cccc3oc5ccccc5c23)c1-4]

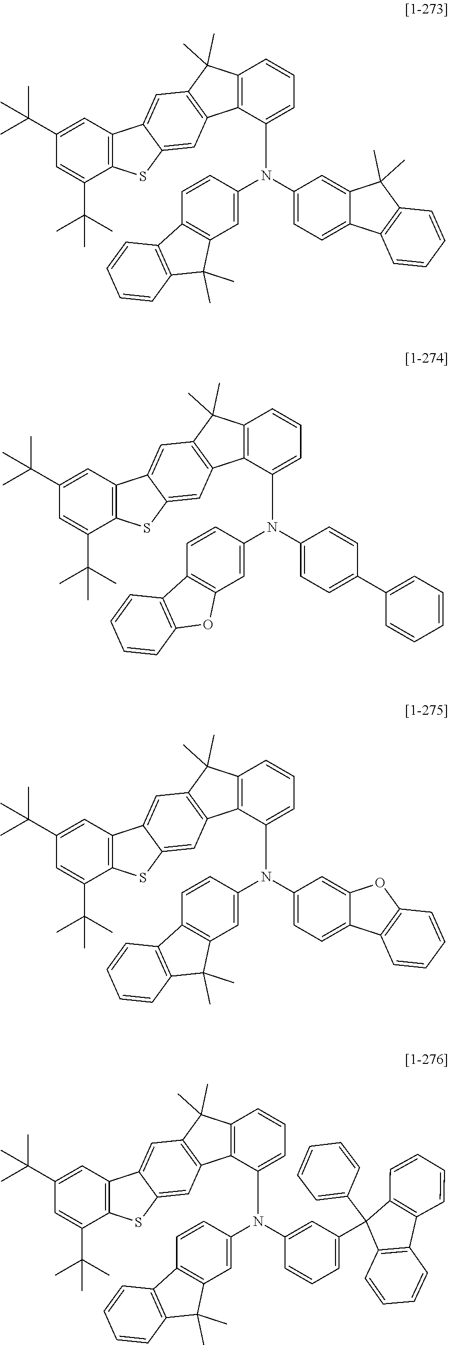 [SMILES: CC(C)(C)c1cc(C(C)(C)C)c2sc3cc4c(cc3c2c1)C(C)(C)c1cccc(N(c2ccc3c(c2)C(C)(C)c2ccccc2-3)c2ccc3c(c2)C(C)(C)c2ccccc2-3)c1-4 ; CC(C)(C)c1cc(C(C)(C)C)c2sc3cc4c(cc3c2c1)C(C)(C)c1cccc(N(c2ccc(-c3ccccc3)cc2)c2ccc3c(c2)oc2ccccc23)c1-4 ; CC(C)(C)c1cc(C(C)(C)C)c2sc3cc4c(cc3c2c1)C(C)(C)c1cccc(N(c2ccc3c(c2)C(C)(C)c2ccccc2-3)c2ccc3c(c2)oc2ccccc23)c1-4 ; CC(C)(C)c1cc(C(C)(C)C)c2sc3cc4c(cc3c2c1)C(C)(C)c1cccc(N(c2cccc(C3(c5ccccc5)c5ccccc5-c5ccccc53)c2)c2ccc3c(c2)C(C)(C)c2ccccc2-3)c1-4]

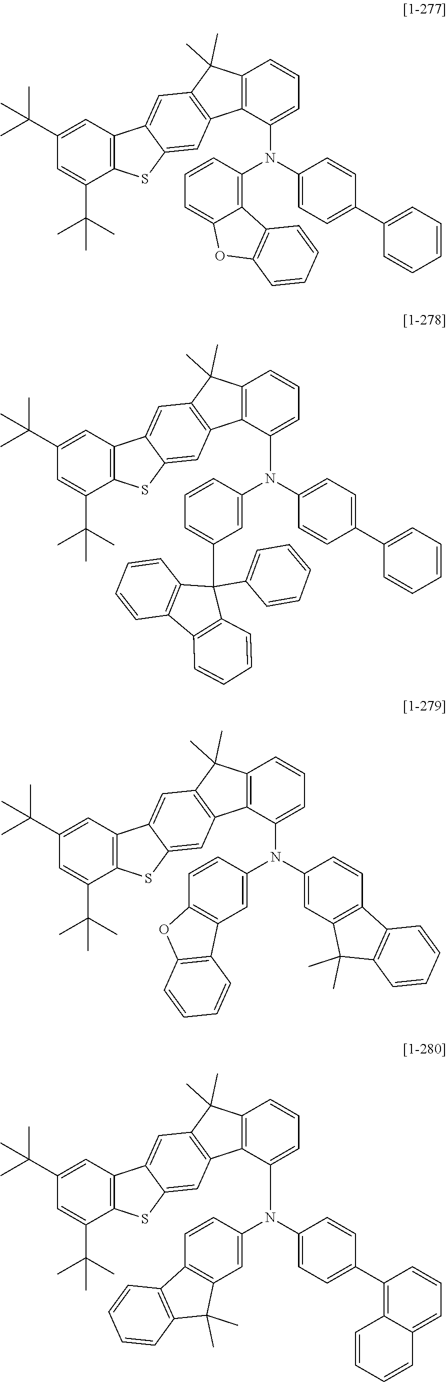 [SMILES: CC(C)(C)c1cc(C(C)(C)C)c2sc3cc4c(cc3c2c1)C(C)(C)c1cccc(N(c2ccc(-c3ccccc3)cc2)c2cccc3oc5ccccc5c23)c1-4 ; CC(C)(C)c1cc(C(C)(C)C)c2sc3cc4c(cc3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2sc3cc4c(cc3c2c1)C(C)(C)c1cccc(N(c2ccc3c(c2)C(C)(C)c2ccccc2-3)c2ccc3oc5ccccc5c3c2)c1-4 ; CC(C)(C)c1cc(C(C)(C)C)c2sc3cc4c(cc3c2c1)C(C)(C)c1cccc(N(c2ccc(-c3cccc5ccccc35)cc2)c2ccc3c(c2)C(C)(C)c2ccccc2-3)c1-4]

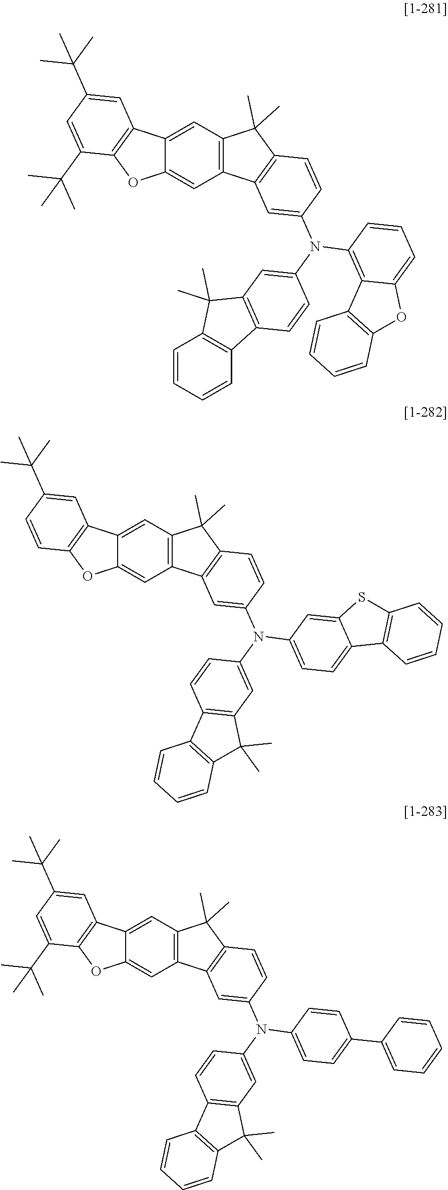 [SMILES: CC(C)(C)c1cc(C(C)(C)C)c2oc3cc4c(cc3c2c1)C(C)(C)c1ccc(N(c2ccc3c(c2)C(C)(C)c2ccccc2-3)c2cccc3oc5ccccc5c23)cc1-4 ; CC(C)(C)c1ccc2oc3cc4c(cc3c2c1)C(C)(C)c1ccc(N(c2ccc3c(c2)C(C)(C)c2ccccc2-3)c2ccc3c(c2)sc2ccccc23)cc1-4 ; CC(C)(C)c1cc(C(C)(C)C)c2oc3cc4c(cc3c2c1)C(C)(C)c1ccc(N(c2ccc(-c3ccccc3)cc2)c2ccc3c(c2)C(C)(C)c2ccccc2-3)cc1-4]

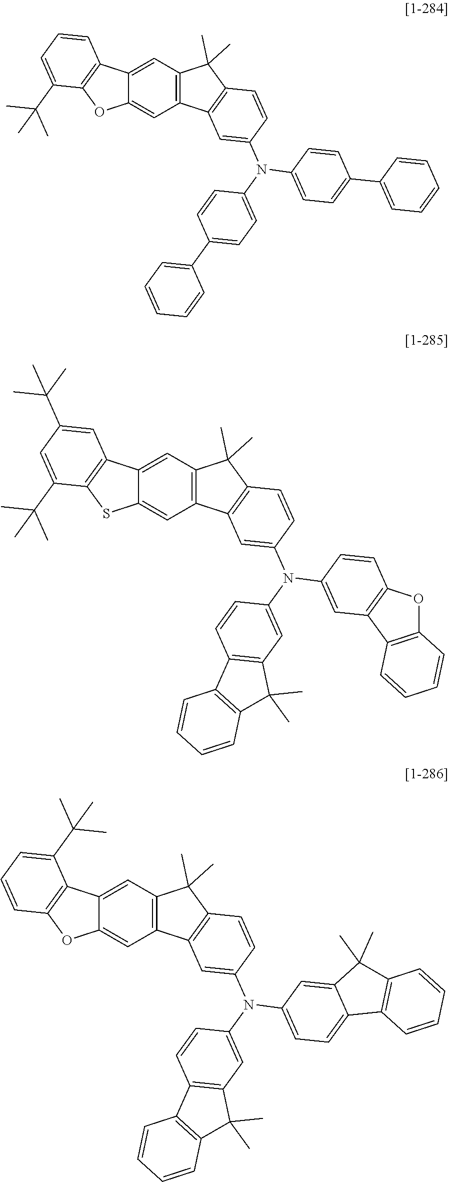 [SMILES: CC(C)(C)c1cccc2c1oc1cc3c(cc12)C(C)(C)c1ccc(N(c2ccc(-c4ccccc4)cc2)c2ccc(-c4ccccc4)cc2)cc1-3 ; CC(C)(C)c1cc(C(C)(C)C)c2sc3cc4c(cc3c2c1)C(C)(C)c1ccc(N(c2ccc3c(c2)C(C)(C)c2ccccc2-3)c2ccc3oc5ccccc5c3c2)cc1-4 ; CC(C)(C)c1cccc2oc3cc4c(cc3c12)C(C)(C)c1ccc(N(c2ccc3c(c2)C(C)(C)c2ccccc2-3)c2ccc3c(c2)C(C)(C)c2ccccc2-3)cc1-4]

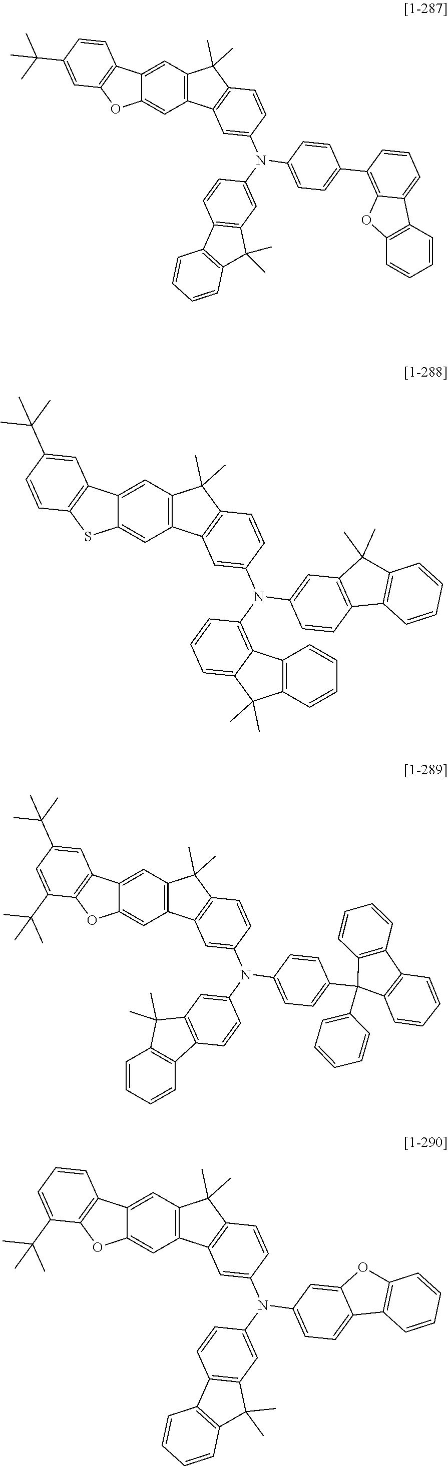 [SMILES: CC(C)(C)c1ccc2c(c1)oc1cc3c(cc12)C(C)(C)c1ccc(N(c2ccc(-c4cccc5c4oc4ccccc45)cc2)c2ccc4c(c2)C(C)(C)c2ccccc2-4)cc1-3 ; CC(C)(C)c1ccc2sc3cc4c(cc3c2c1)C(C)(C)c1ccc(N(c2ccc3c(c2)C(C)(C)c2ccccc2-3)c2cccc3c2-c2ccccc2C3(C)C)cc1-4 ; CC(C)(C)c1cc(C(C)(C)C)c2oc3cc4c(cc3c2c1)C(C)(C)c1ccc(N(c2ccc(C3(c5ccccc5)c5ccccc5-c5ccccc53)cc2)c2ccc3c(c2)C(C)(C)c2ccccc2-3)cc1-4 ; CC(C)(C)c1cccc2c1oc1cc3c(cc12)C(C)(C)c1ccc(N(c2ccc4c(c2)C(C)(C)c2ccccc2-4)c2ccc4c(c2)oc2ccccc24)cc1-3]

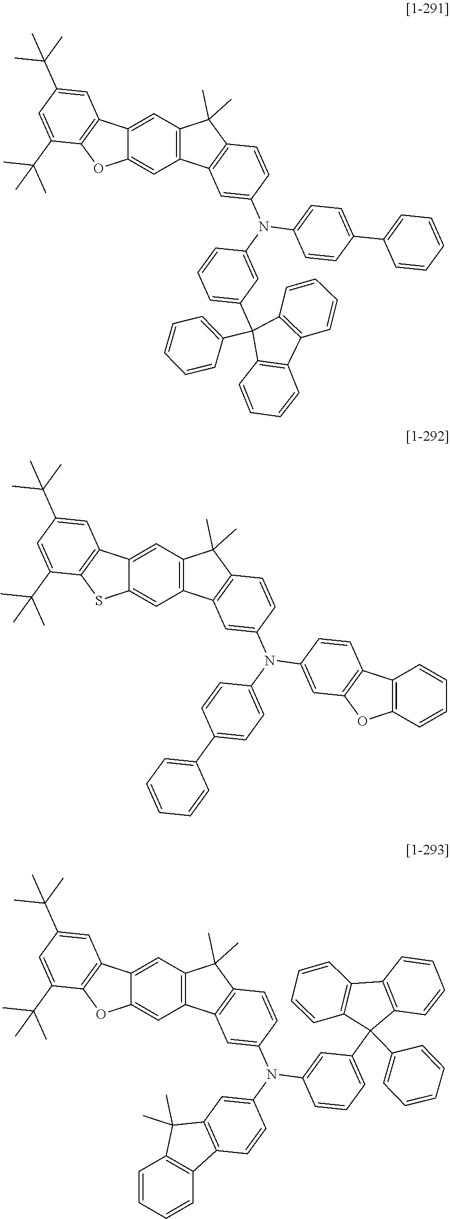 [SMILES: CC(C)(C)c1cc(C(C)(C)C)c2oc3cc4c(cc3c2c1)C(C)(C)c1ccc(N(c2ccc(-c3ccccc3)cc2)c2cccc(C3(c5ccccc5)c5ccccc5-c5ccccc53)c2)cc1-4 ; CC(C)(C)c1cc(C(C)(C)C)c2sc3cc4c(cc3c2c1)C(C)(C)c1ccc(N(c2ccc(-c3ccccc3)cc2)c2ccc3c(c2)oc2ccccc23)cc1-4 ; CC(C)(C)c1cc(C(C)(C)C)c2oc3cc4c(cc3c2c1)C(C)(C)c1ccc(N(c2cccc(C3(c5ccccc5)c5ccccc5-c5ccccc53)c2)c2ccc3c(c2)C(C)(C)c2ccccc2-3)cc1-4]

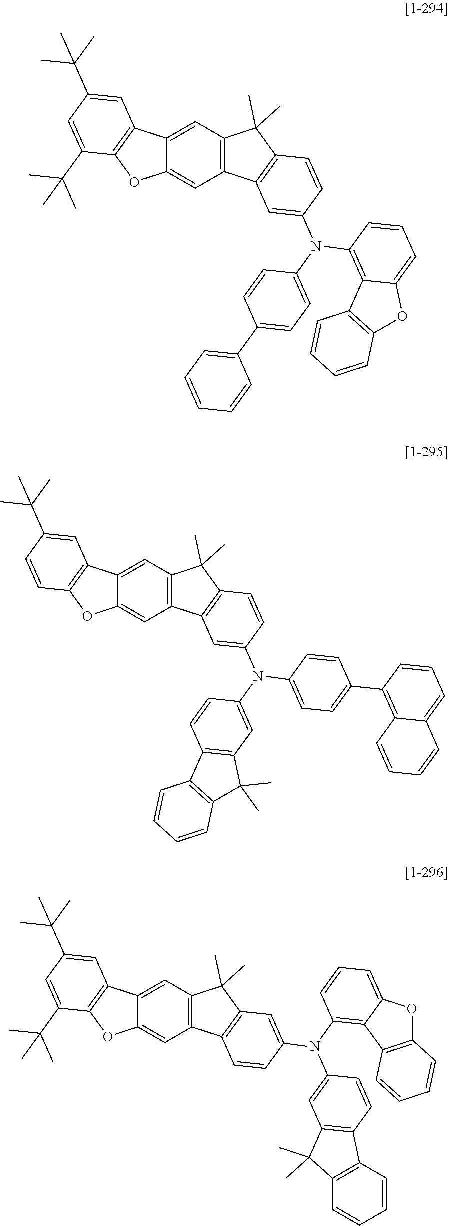 [SMILES: CC(C)(C)c1cc(C(C)(C)C)c2oc3cc4c(cc3c2c1)C(C)(C)c1ccc(N(c2ccc(-c3ccccc3)cc2)c2cccc3oc5ccccc5c23)cc1-4 ; CC(C)(C)c1ccc2oc3cc4c(cc3c2c1)C(C)(C)c1ccc(N(c2ccc(-c3cccc5ccccc35)cc2)c2ccc3c(c2)C(C)(C)c2ccccc2-3)cc1-4 ; CC(C)(C)c1cc(C(C)(C)C)c2oc3cc4c(cc3c2c1)C(C)(C)c1cc(N(c2ccc3c(c2)C(C)(C)c2ccccc2-3)c2cccc3oc5ccccc5c23)ccc1-4]

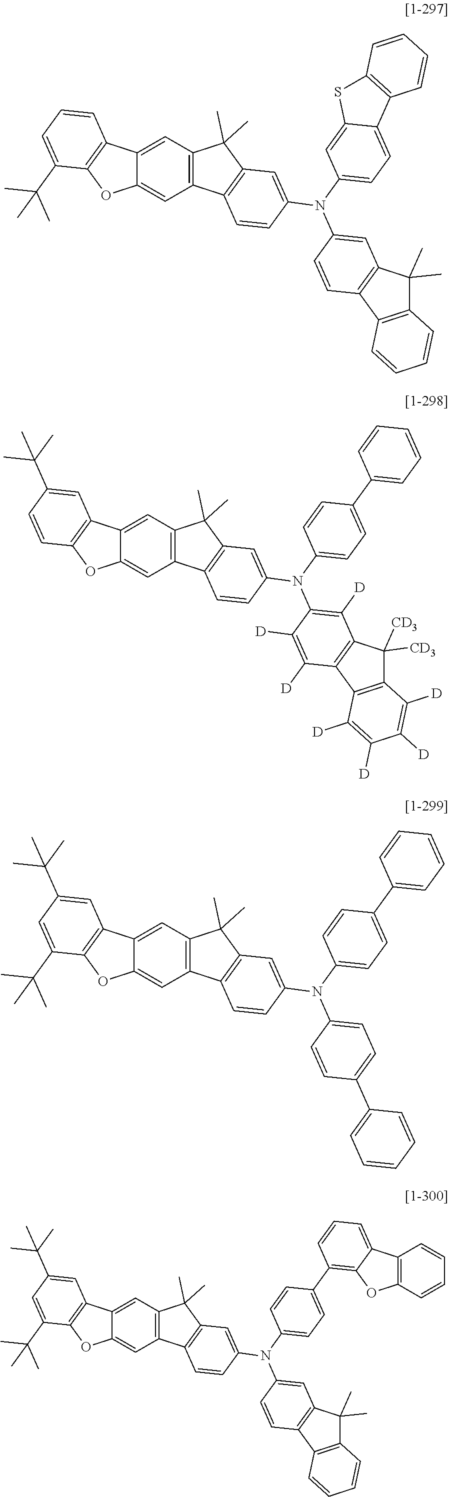

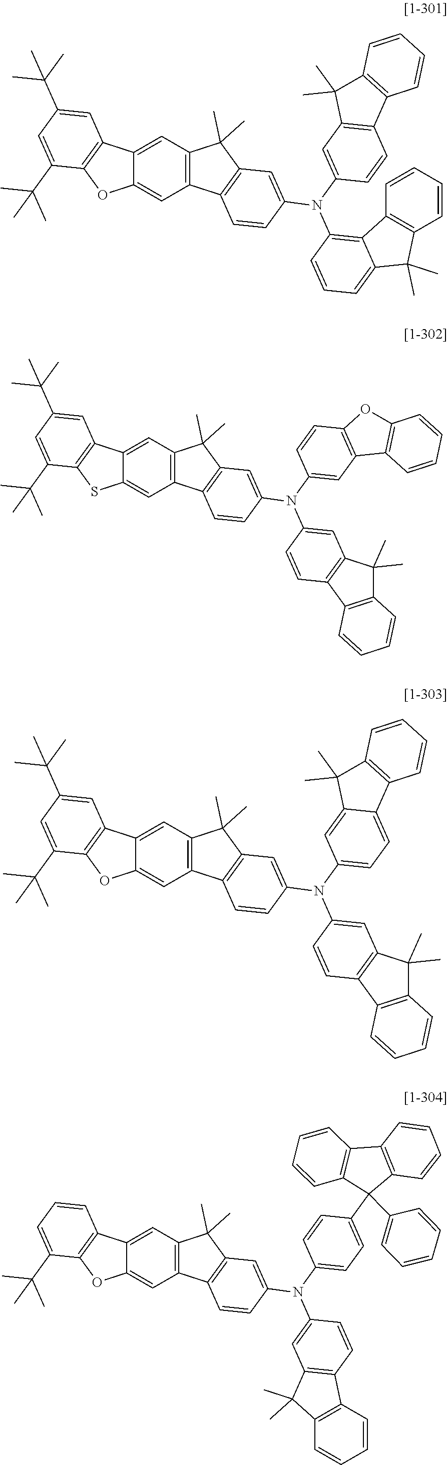

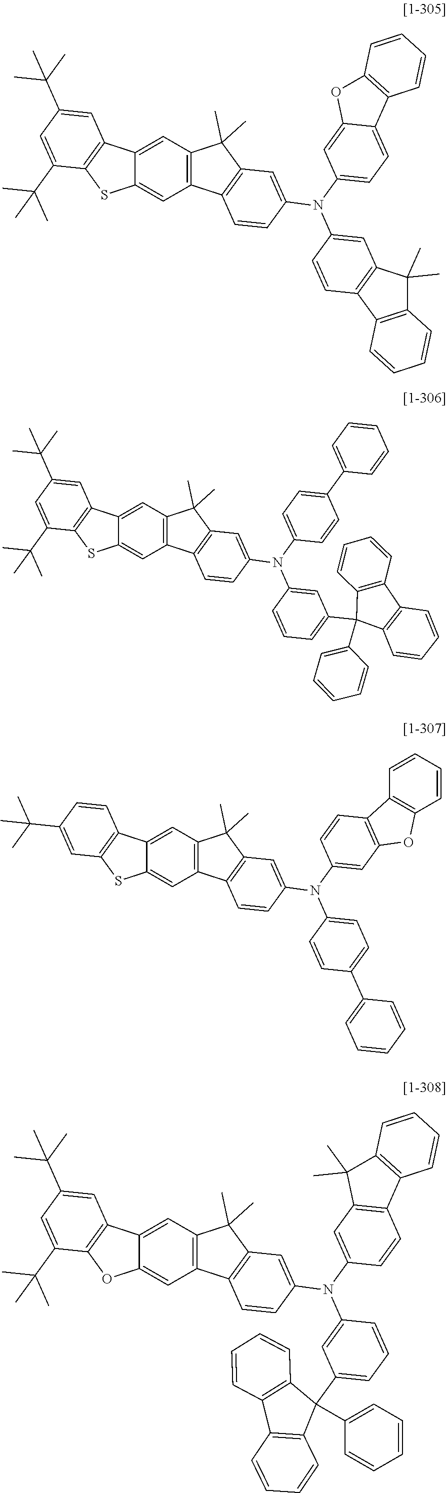

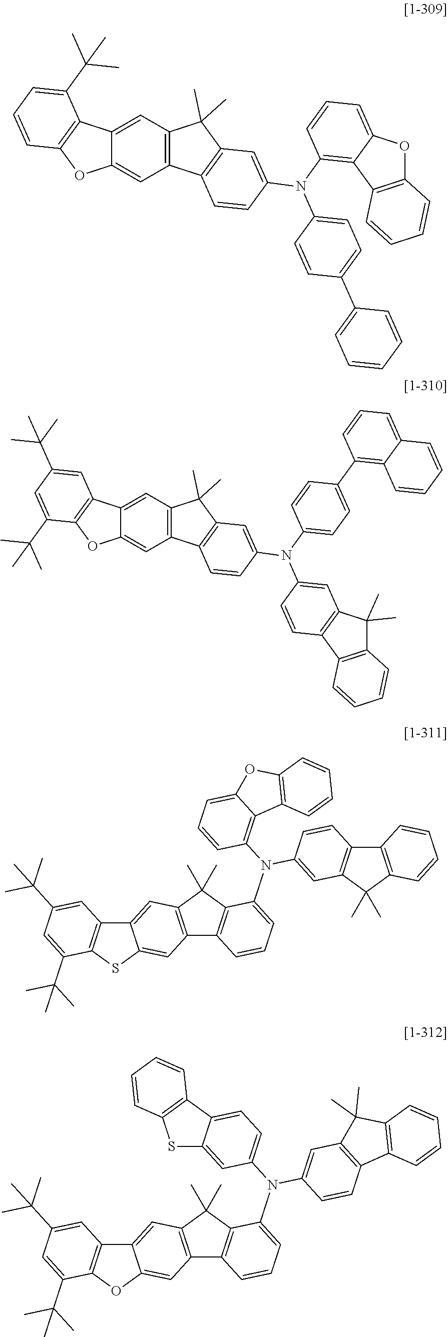 [SMILES: CC(C)(C)c1cccc2oc3cc4c(cc3c12)C(C)(C)c1cc(N(c2ccc(-c3ccccc3)cc2)c2cccc3oc5ccccc5c23)ccc1-4 ; CC(C)(C)c1cc(C(C)(C)C)c2oc3cc4c(cc3c2c1)C(C)(C)c1cc(N(c2ccc(-c3cccc5ccccc35)cc2)c2ccc3c(c2)C(C)(C)c2ccccc2-3)ccc1-4 ; CC(C)(C)c1cc(C(C)(C)C)c2sc3cc4c(cc3c2c1)C(C)(C)c1c-4cccc1N(c1ccc2c(c1)C(C)(C)c1ccccc1-2)c1cccc2oc3ccccc3c12 ; CC(C)(C)c1cc(C(C)(C)C)c2oc3cc4c(cc3c2c1)C(C)(C)c1c-4cccc1N(c1ccc2c(c1)C(C)(C)c1ccccc1-2)c1ccc2c(c1)sc1ccccc12]

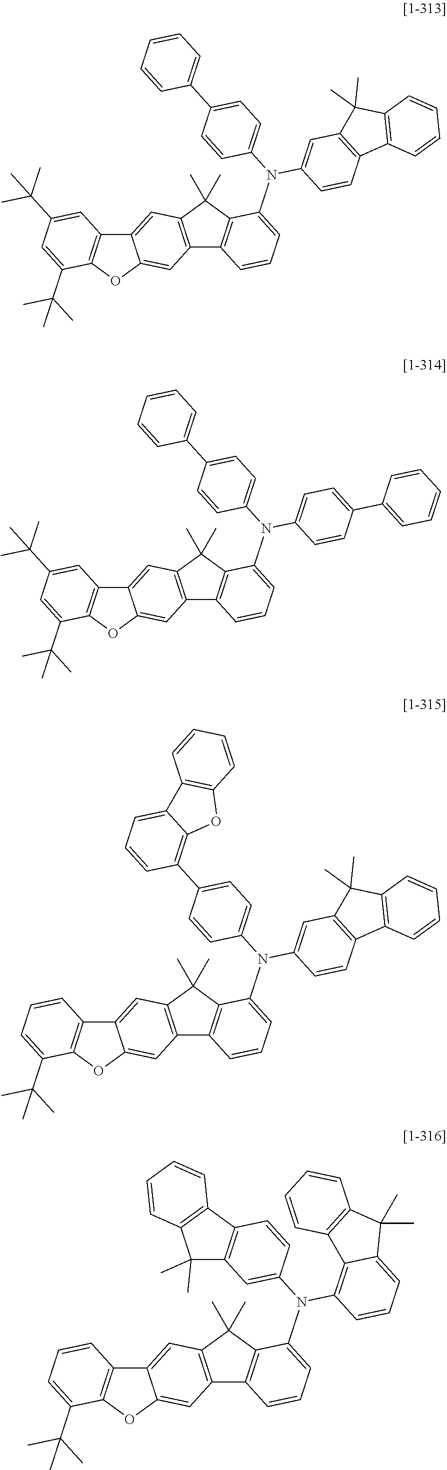 [SMILES: CC(C)(C)c1cc(C(C)(C)C)c2oc3cc4c(cc3c2c1)C(C)(C)c1c-4cccc1N(c1ccc(-c2ccccc2)cc1)c1ccc2c(c1)C(C)(C)c1ccccc1-2 ; CC(C)(C)c1cc(C(C)(C)C)c2oc3cc4c(cc3c2c1)C(C)(C)c1c-4cccc1N(c1ccc(-c2ccccc2)cc1)c1ccc(-c2ccccc2)cc1 ; CC(C)(C)c1cccc2c1oc1cc3c(cc12)C(C)(C)c1c-3cccc1N(c1ccc(-c2cccc3c2oc2ccccc23)cc1)c1ccc2c(c1)C(C)(C)c1ccccc1-2 ; CC(C)(C)c1cccc2c1oc1cc3c(cc12)C(C)(C)c1c-3cccc1N(c1ccc2c(c1)C(C)(C)c1ccccc1-2)c1cccc2c1-c1ccccc1C2(C)C]

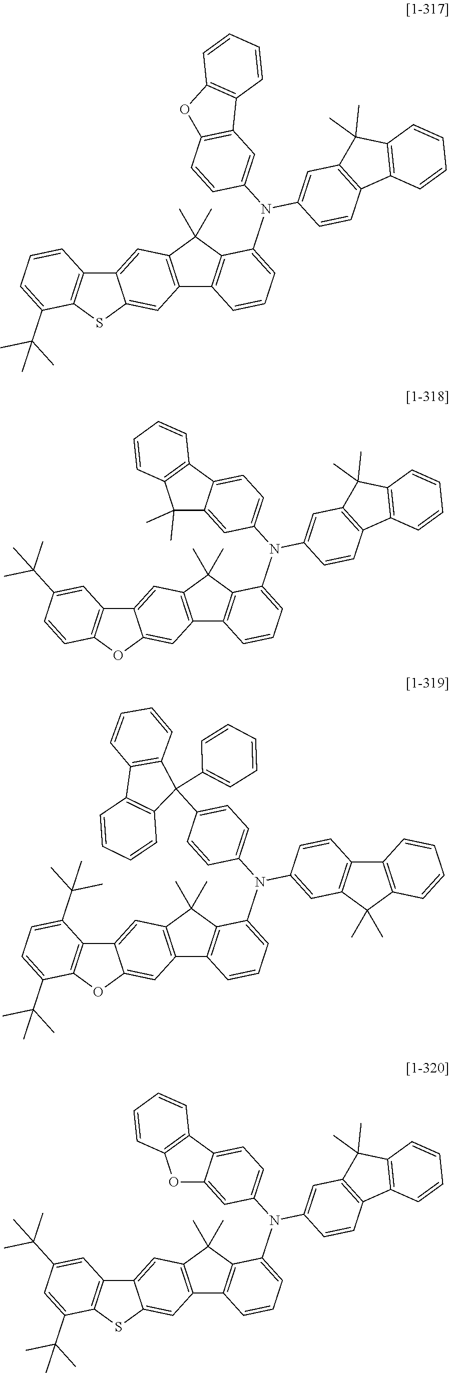 [SMILES: CC(C)(C)c1cccc2c1sc1cc3c(cc12)C(C)(C)c1c-3cccc1N(c1ccc2c(c1)C(C)(C)c1ccccc1-2)c1ccc2oc3ccccc3c2c1 ; CC(C)(C)c1ccc2oc3cc4c(cc3c2c1)C(C)(C)c1c-4cccc1N(c1ccc2c(c1)C(C)(C)c1ccccc1-2)c1ccc2c(c1)C(C)(C)c1ccccc1-2 ; CC(C)(C)c1ccc(C(C)(C)C)c2c1oc1cc3c(cc12)C(C)(C)c1c-3cccc1N(c1ccc(C2(c3ccccc3)c3ccccc3-c3ccccc32)cc1)c1ccc2c(c1)C(C)(C)c1ccccc1-2 ; CC(C)(C)c1cc(C(C)(C)C)c2sc3cc4c(cc3c2c1)C(C)(C)c1c-4cccc1N(c1ccc2c(c1)C(C)(C)c1ccccc1-2)c1ccc2c(c1)oc1ccccc12]

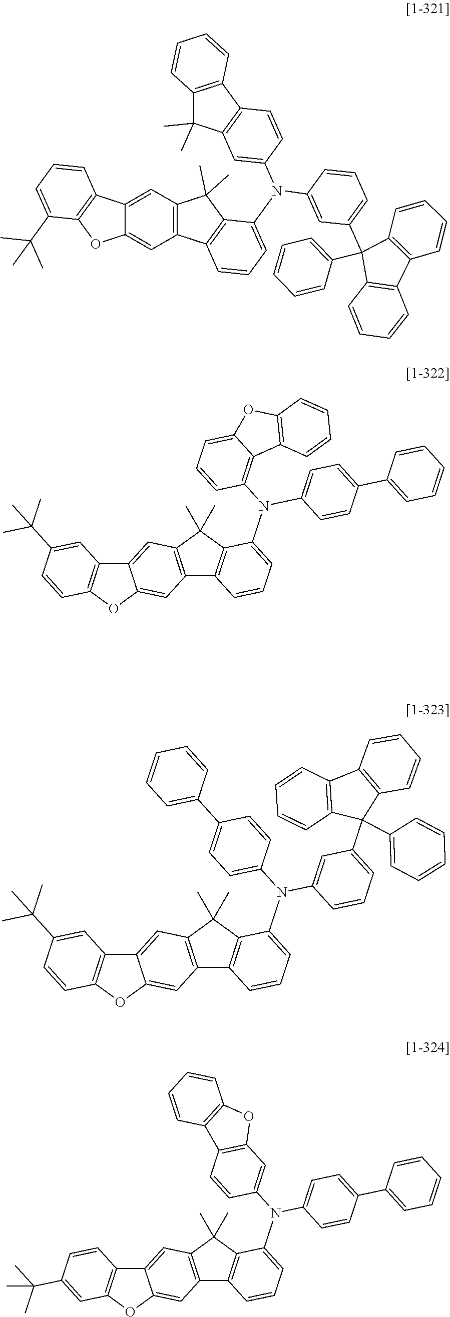 [SMILES: CC(C)(C)c1cccc2c1oc1cc3c(cc12)C(C)(C)c1c-3cccc1N(c1cccc(C2(c3ccccc3)c3ccccc3-c3ccccc32)c1)c1ccc2c(c1)C(C)(C)c1ccccc1-2 ; CC(C)(C)c1ccc2oc3cc4c(cc3c2c1)C(C)(C)c1c-4cccc1N(c1ccc(-c2ccccc2)cc1)c1cccc2oc3ccccc3c12 ; CC(C)(C)c1ccc2oc3cc4c(cc3c2c1)C(C)(C)c1c-4cccc1N(c1ccc(-c2ccccc2)cc1)c1cccc(C2(c3ccccc3)c3ccccc3-c3ccccc32)c1 ; CC(C)(C)c1ccc2c(c1)oc1cc3c(cc12)C(C)(C)c1c-3cccc1N(c1ccc(-c2ccccc2)cc1)c1ccc2c(c1)oc1ccccc12]

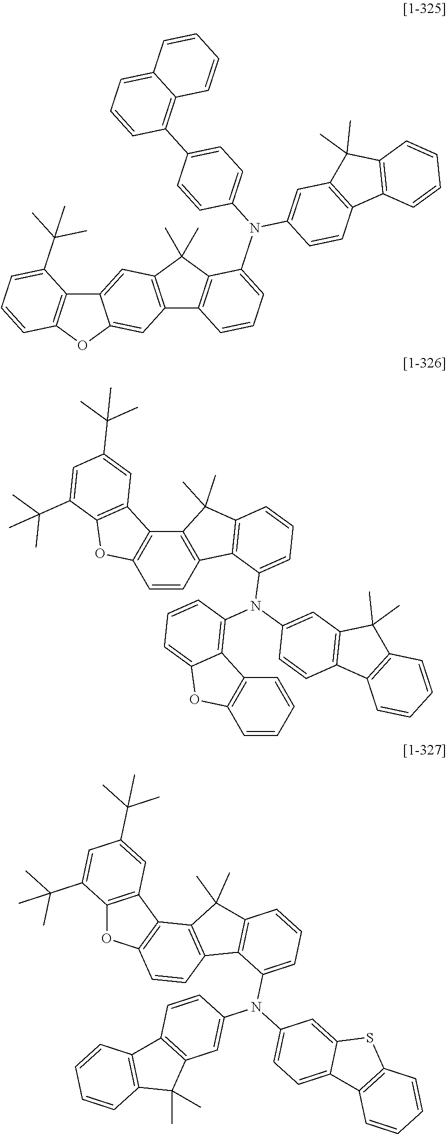 [SMILES: CC(C)(C)c1cccc2oc3cc4c(cc3c12)C(C)(C)c1c-4cccc1N(c1ccc(-c2cccc3ccccc23)cc1)c1ccc2c(c1)C(C)(C)c1ccccc1-2 ; CC(C)(C)c1cc(C(C)(C)C)c2oc3ccc4c(c3c2c1)C(C)(C)c1cccc(N(c2ccc3c(c2)C(C)(C)c2ccccc2-3)c2cccc3oc5ccccc5c23)c1-4 ; CC(C)(C)c1cc(C(C)(C)C)c2oc3ccc4c(c3c2c1)C(C)(C)c1cccc(N(c2ccc3c(c2)C(C)(C)c2ccccc2-3)c2ccc3c(c2)sc2ccccc23)c1-4]

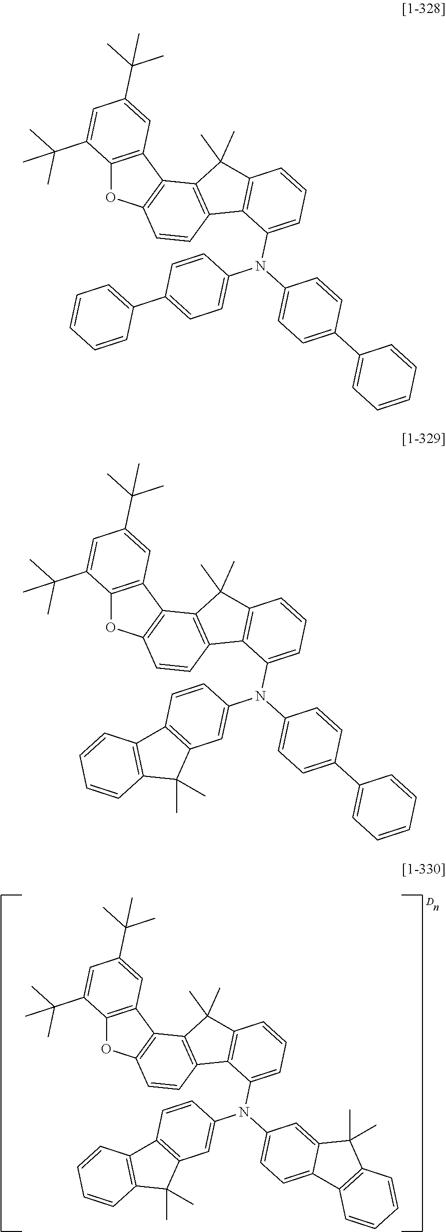

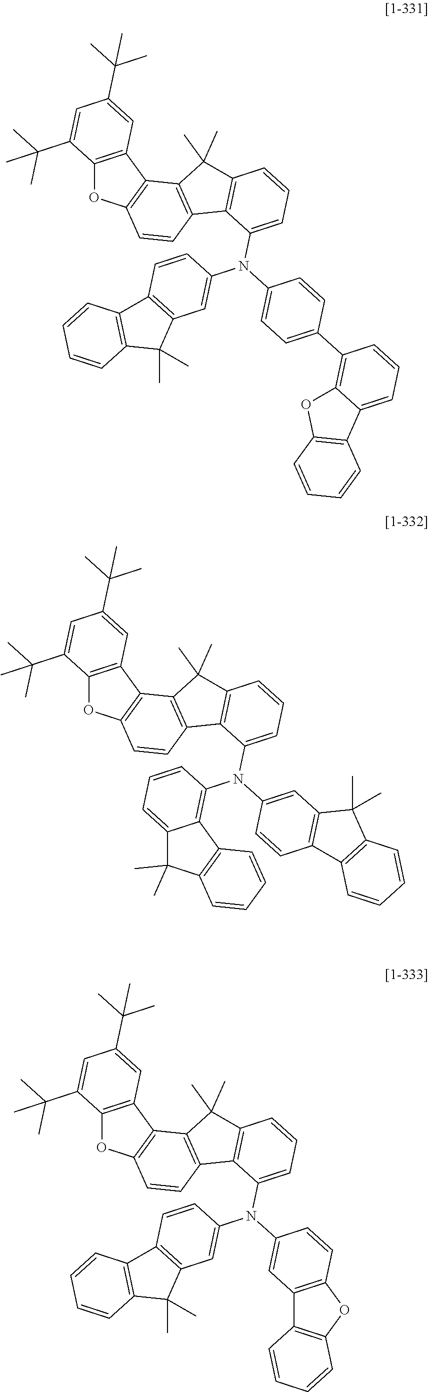 [SMILES: CC(C)(C)c1cc(C(C)(C)C)c2oc3ccc4c(c3c2c1)C(C)(C)c1cccc(N(c2ccc(-c3cccc5c3oc3ccccc35)cc2)c2ccc3c(c2)C(C)(C)c2ccccc2-3)c1-4 ; CC(C)(C)c1cc(C(C)(C)C)c2oc3ccc4c(c3c2c1)C(C)(C)c1cccc(N(c2ccc3c(c2)C(C)(C)c2ccccc2-3)c2cccc3c2-c2ccccc2C3(C)C)c1-4 ; CC(C)(C)c1cc(C(C)(C)C)c2oc3ccc4c(c3c2c1)C(C)(C)c1cccc(N(c2ccc3c(c2)C(C)(C)c2ccccc2-3)c2ccc3oc5ccccc5c3c2)c1-4]

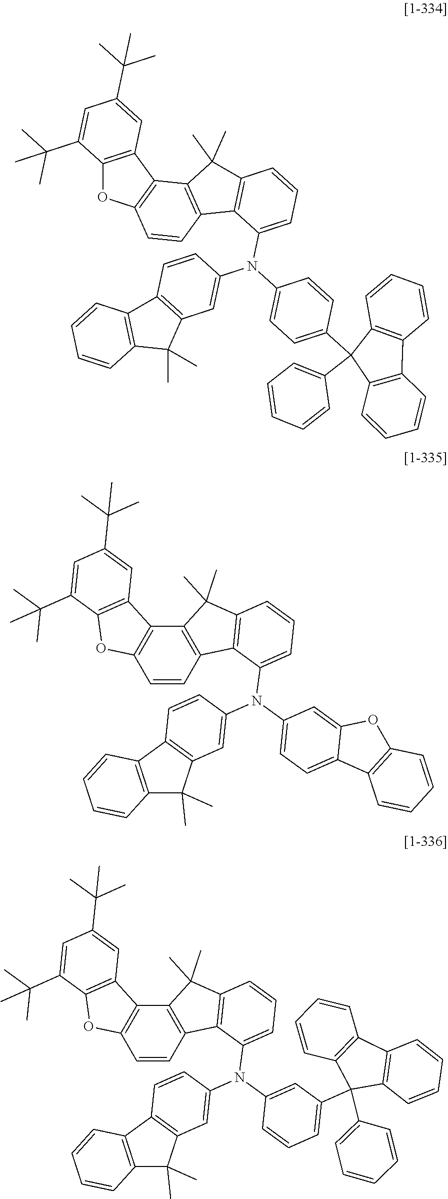 [SMILES: CC(C)(C)c1cc(C(C)(C)C)c2oc3ccc4c(c3c2c1)C(C)(C)c1cccc(N(c2ccc(C3(c5ccccc5)c5ccccc5-c5ccccc53)cc2)c2ccc3c(c2)C(C)(C)c2ccccc2-3)c1-4 ; CC(C)(C)c1cc(C(C)(C)C)c2oc3ccc4c(c3c2c1)C(C)(C)c1cccc(N(c2ccc3c(c2)C(C)(C)c2ccccc2-3)c2ccc3c(c2)oc2ccccc23)c1-4 ; CC(C)(C)c1cc(C(C)(C)C)c2oc3ccc4c(c3c2c1)C(C)(C)c1cccc(N(c2cccc(C3(c5ccccc5)c5ccccc5-c5ccccc53)c2)c2ccc3c(c2)C(C)(C)c2ccccc2-3)c1-4]

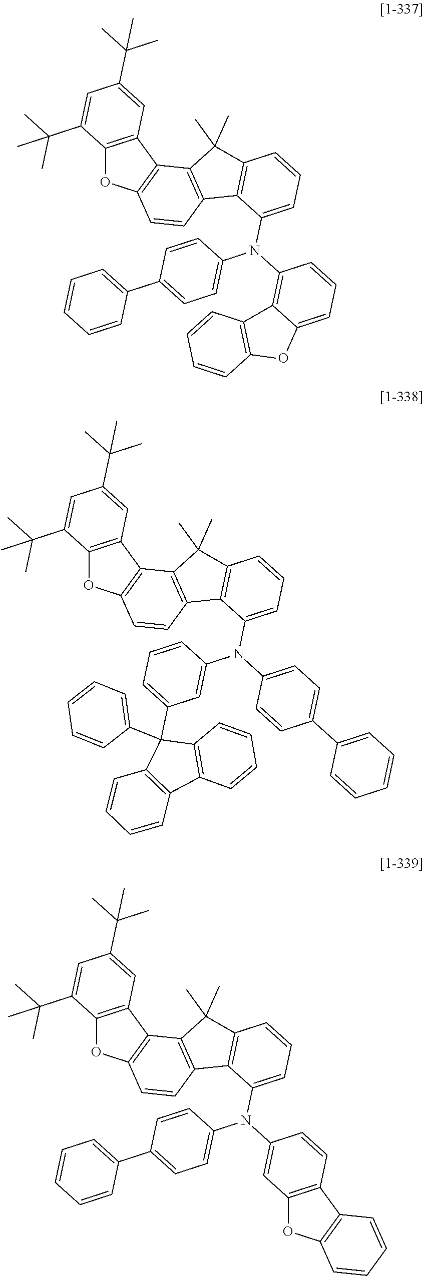 [SMILES: CC(C)(C)c1cc(C(C)(C)C)c2oc3ccc4c(c3c2c1)C(C)(C)c1cccc(N(c2ccc(-c3ccccc3)cc2)c2cccc3oc5ccccc5c23)c1-4 ; CC(C)(C)c1cc(C(C)(C)C)c2oc3ccc4c(c3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2oc3ccc4c(c3c2c1)C(C)(C)c1cccc(N(c2ccc(-c3ccccc3)cc2)c2ccc3c(c2)oc2ccccc23)c1-4]

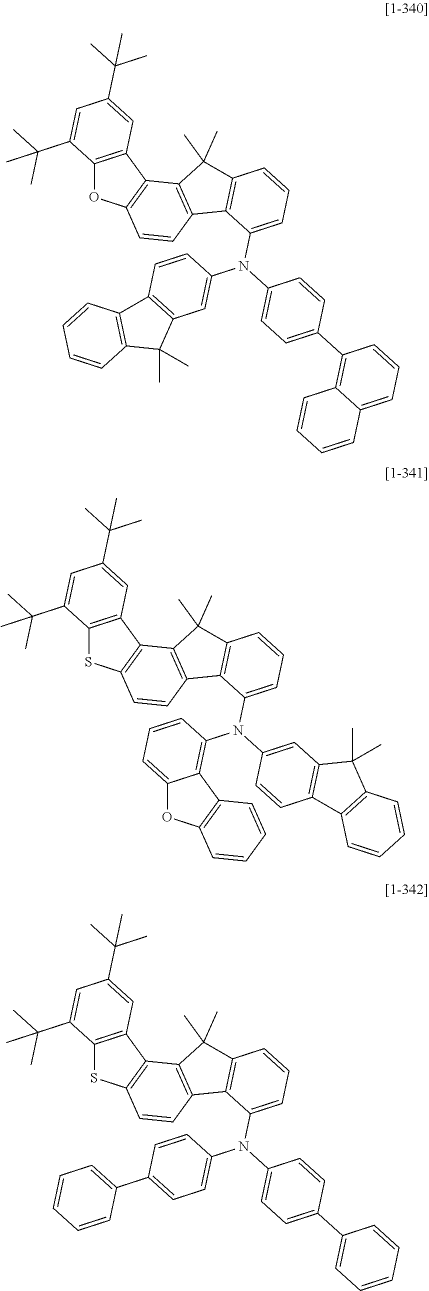 [SMILES: CC(C)(C)c1cc(C(C)(C)C)c2oc3ccc4c(c3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2cccc3oc5ccccc5c23)c1-4 ; CC(C)(C)c1cc(C(C)(C)C)c2sc3ccc4c(c3c2c1)C(C)(C)c1cccc(N(c2ccc(-c3ccccc3)cc2)c2ccc(-c3ccccc3)cc2)c1-4]

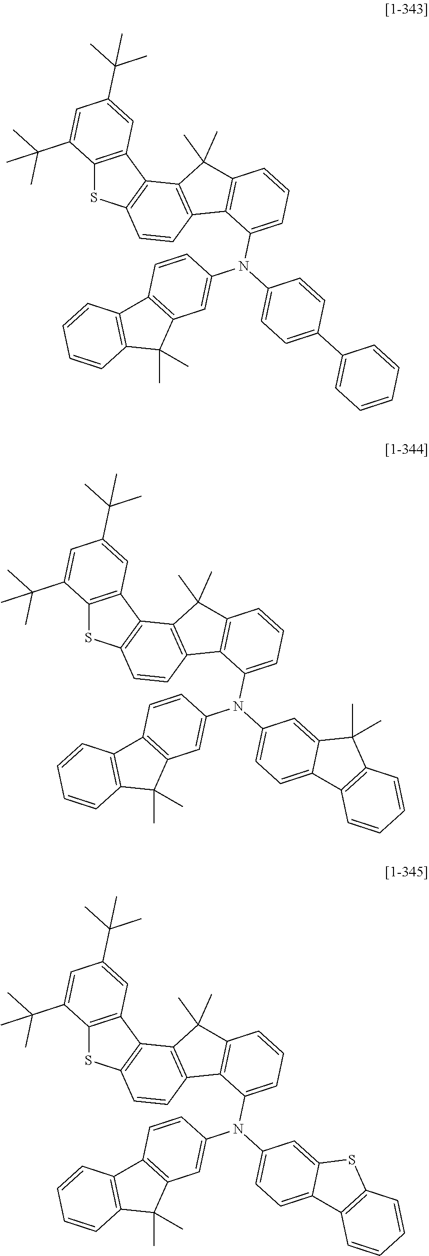 [SMILES: CC(C)(C)c1cc(C(C)(C)C)c2sc3ccc4c(c3c2c1)C(C)(C)c1cccc(N(c2ccc(-c3ccccc3)cc2)c2ccc3c(c2)C(C)(C)c2ccccc2-3)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2ccc3c(c2)C(C)(C)c2ccccc2-3)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2ccc3c(c2)sc2ccccc23)c1-4]

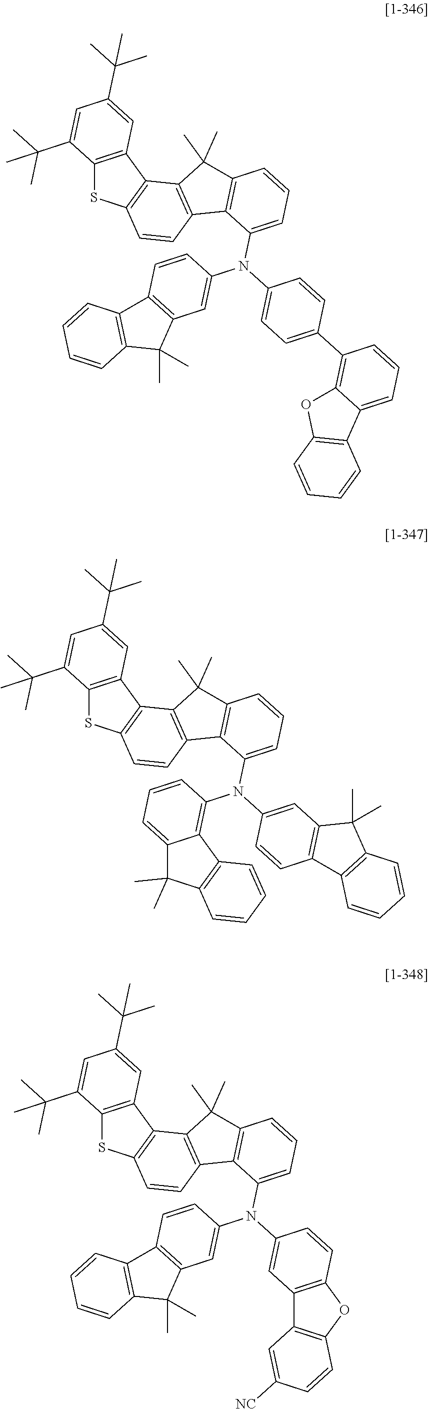 [SMILES: CC(C)(C)c1cc(C(C)(C)C)c2sc3ccc4c(c3c2c1)C(C)(C)c1cccc(N(c2ccc(-c3cccc5c3oc3ccccc35)cc2)c2ccc3c(c2)C(C)(C)c2ccccc2-3)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2cccc3c2-c2ccccc2C3(C)C)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2ccc3oc5ccc(C#N)cc5c3c2)c1-4]

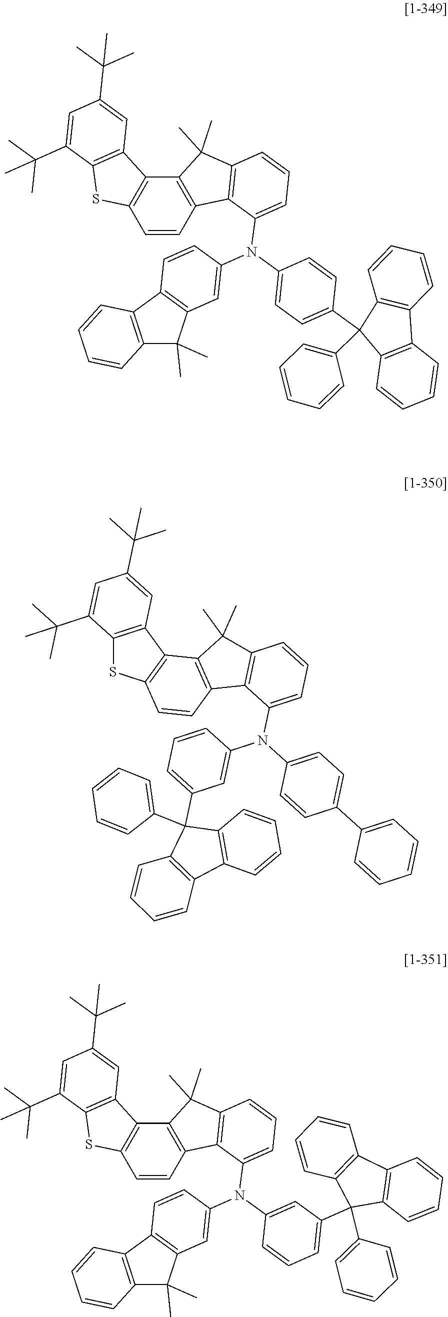 [SMILES: CC(C)(C)c1cc(C(C)(C)C)c2sc3ccc4c(c3c2c1)C(C)(C)c1cccc(N(c2ccc(C3(c5ccccc5)c5ccccc5-c5ccccc53)cc2)c2ccc3c(c2)C(C)(C)c2ccccc2-3)c1-4 ; CC(C)(C)c1cc(C(C)(C)C)c2sc3ccc4c(c3c2c1)C(C)(C)c1cccc(N(c2ccc(-c3ccccc3)cc2)c2cccc(C3(c5ccccc5)c5ccccc5-c5ccccc53)c2)c1-4 ; CC(C)(C)c1cc(C(C)(C)C)c2sc3ccc4c(c3c2c1)C(C)(C)c1cccc(N(c2cccc(C3(c5ccccc5)c5ccccc5-c5ccccc53)c2)c2ccc3c(c2)C(C)(C)c2ccccc2-3)c1-4]

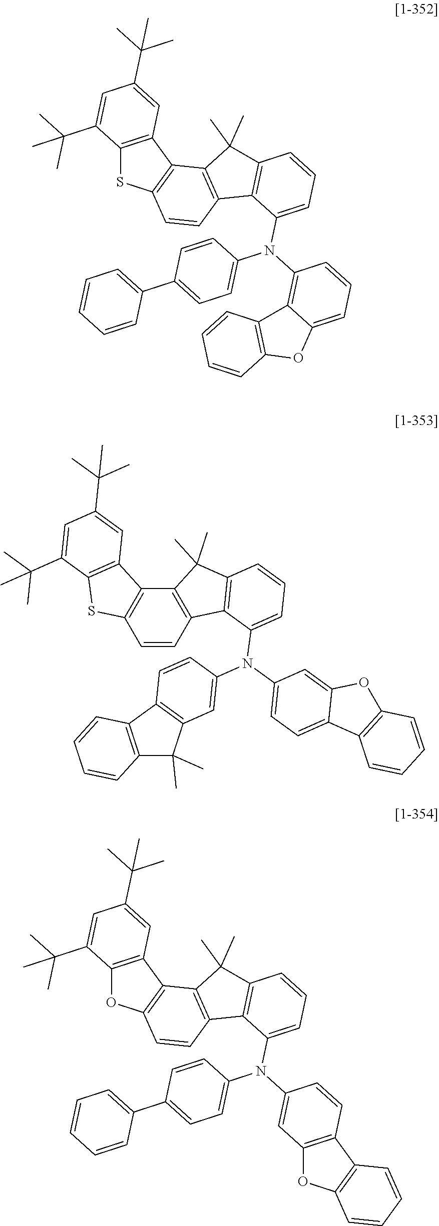 [SMILES: CC(C)(C)c1cc(C(C)(C)C)c2sc3ccc4c(c3c2c1)C(C)(C)c1cccc(N(c2ccc(-c3ccccc3)cc2)c2cccc3oc5ccccc5c23)c1-4 ; CC(C)(C)c1cc(C(C)(C)C)c2sc3ccc4c(c3c2c1)C(C)(C)c1cccc(N(c2ccc3c(c2)C(C)(C)c2ccccc2-3)c2ccc3c(c2)oc2ccccc23)c1-4 ; CC(C)(C)c1cc(C(C)(C)C)c2oc3ccc4c(c3c2c1)C(C)(C)c1cccc(N(c2ccc(-c3ccccc3)cc2)c2ccc3c(c2)oc2ccccc23)c1-4]

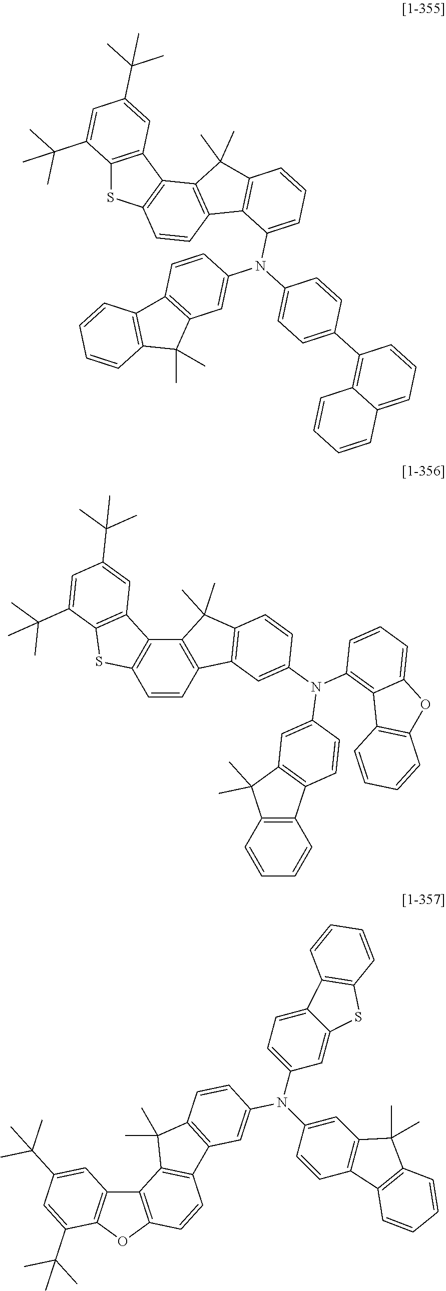 [SMILES: CC(C)(C)c1cc(C(C)(C)C)c2sc3ccc4c(c3c2c1)C(C)(C)c1cccc(N(c2ccc(-c3cccc5ccccc35)cc2)c2ccc3c(c2)C(C)(C)c2ccccc2-3)c1-4 ; CC(C)(C)c1cc(C(C)(C)C)c2sc3ccc4c(c3c2c1)C(C)(C)c1ccc(N(c2ccc3c(c2)C(C)(C)c2ccccc2-3)c2cccc3oc5ccccc5c23)cc1-4 ; CC(C)(C)c1cc(C(C)(C)C)c2oc3ccc4c(c3c2c1)C(C)(C)c1ccc(N(c2ccc3c(c2)C(C)(C)c2ccccc2-3)c2ccc3c(c2)sc2ccccc23)cc1-4]

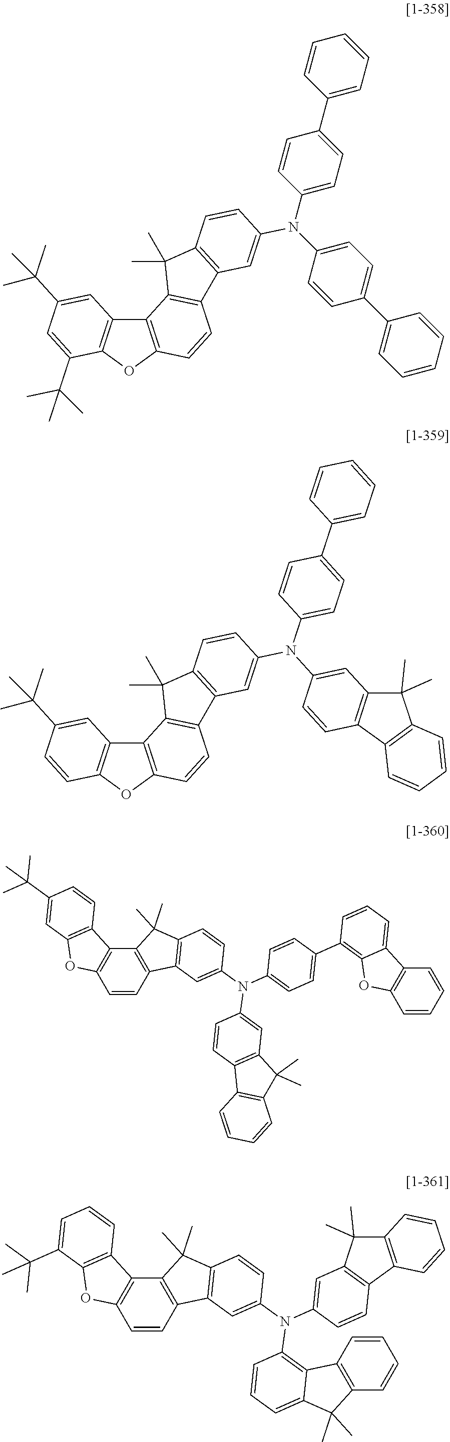 [SMILES: CC(C)(C)c1cc(C(C)(C)C)c2oc3ccc4c(c3c2c1)C(C)(C)c1ccc(N(c2ccc(-c3ccccc3)cc2)c2ccc(-c3ccccc3)cc2)cc1-4 ; CC(C)(C)c1ccc2oc3ccc4c(c3c2c1)C(C)(C)c1ccc(N(c2ccc(-c3ccccc3)cc2)c2ccc3c(c2)C(C)(C)c2ccccc2-3)cc1-4 ; CC(C)(C)c1ccc2c(c1)oc1ccc3c(c12)C(C)(C)c1ccc(N(c2ccc(-c4cccc5c4oc4ccccc45)cc2)c2ccc4c(c2)C(C)(C)c2ccccc2-4)cc1-3 ; CC(C)(C)c1cccc2c1oc1ccc3c(c12)C(C)(C)c1ccc(N(c2ccc4c(c2)C(C)(C)c2ccccc2-4)c2cccc4c2-c2ccccc2C4(C)C)cc1-3]

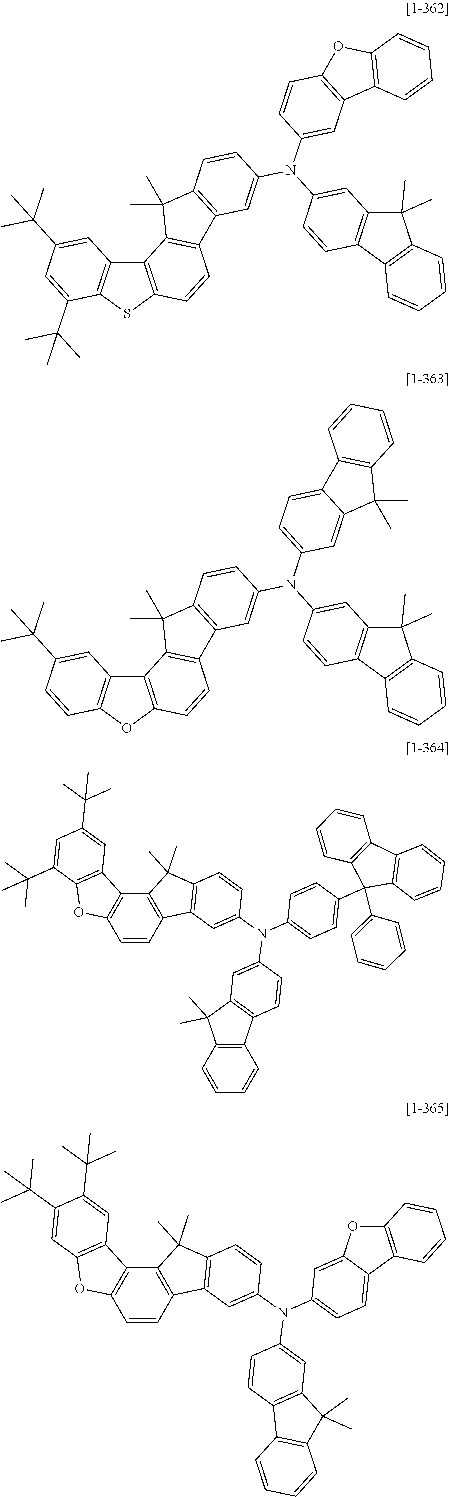 [SMILES: CC(C)(C)c1cc(C(C)(C)C)c2sc3ccc4c(c3c2c1)C(C)(C)c1ccc(N(c2ccc3c(c2)C(C)(C)c2ccccc2-3)c2ccc3oc5ccccc5c3c2)cc1-4 ; CC(C)(C)c1ccc2oc3ccc4c(c3c2c1)C(C)(C)c1ccc(N(c2ccc3c(c2)C(C)(C)c2ccccc2-3)c2ccc3c(c2)C(C)(C)c2ccccc2-3)cc1-4 ; CC(C)(C)c1cc(C(C)(C)C)c2oc3ccc4c(c3c2c1)C(C)(C)c1ccc(N(c2ccc(C3(c5ccccc5)c5ccccc5-c5ccccc53)cc2)c2ccc3c(c2)C(C)(C)c2ccccc2-3)cc1-4 ; CC(C)(C)c1cc2oc3ccc4c(c3c2cc1C(C)(C)C)C(C)(C)c1ccc(N(c2ccc3c(c2)C(C)(C)c2ccccc2-3)c2ccc3c(c2)oc2ccccc23)cc1-4]

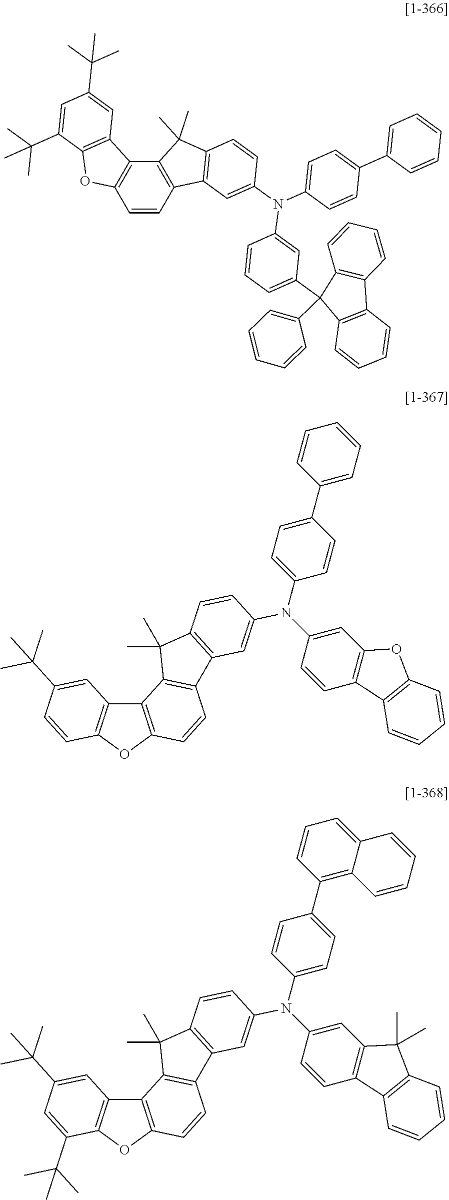 [SMILES: CC(C)(C)c1cc(C(C)(C)C)c2oc3ccc4c(c3c2c1)C(C)(C)c1ccc(N(c2ccc(-c3ccccc3)cc2)c2cccc(C3(c5ccccc5)c5ccccc5-c5ccccc53)c2)cc1-4 ; CC(C)(C)c1ccc2oc3ccc4c(c3c2c1)C(C)(C)c1ccc(N(c2ccc(-c3ccccc3)cc2)c2ccc3c(c2)oc2ccccc23)cc1-4 ; CC(C)(C)c1cc(C(C)(C)C)c2oc3ccc4c(c3c2c1)C(C)(C)c1ccc(N(c2ccc(-c3cccc5ccccc35)cc2)c2ccc3c(c2)C(C)(C)c2ccccc2-3)cc1-4]

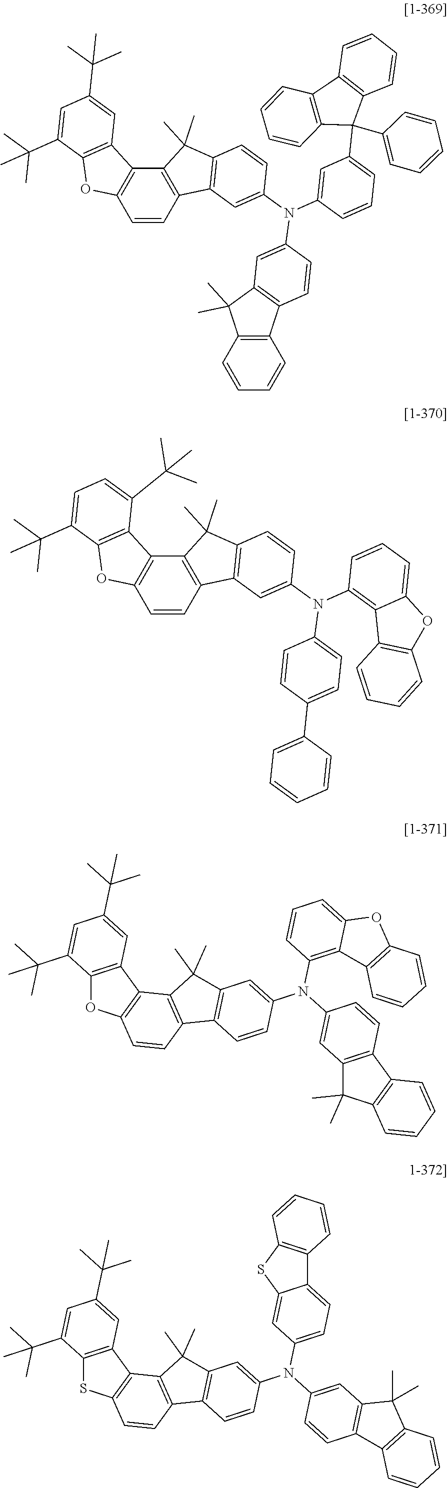 [SMILES: CC(C)(C)c1cc(C(C)(C)C)c2oc3ccc4c(c3c2c1)C(C)(C)c1ccc(N(c2cccc(C3(c5ccccc5)c5ccccc5-c5ccccc53)c2)c2ccc3c(c2)C(C)(C)c2ccccc2-3)cc1-4 ; CC(C)(C)c1ccc(C(C)(C)C)c2c1oc1ccc3c(c12)C(C)(C)c1ccc(N(c2ccc(-c4ccccc4)cc2)c2cccc4oc5ccccc5c24)cc1-3 ; CC(C)(C)c1cc(C(C)(C)C)c2oc3ccc4c(c3c2c1)C(C)(C)c1cc(N(c2ccc3c(c2)C(C)(C)c2ccccc2-3)c2cccc3oc5ccccc5c23)ccc1-4 ; CC(C)(C)c1cc(C(C)(C)C)c2sc3ccc4c(c3c2c1)C(C)(C)c1cc(N(c2ccc3c(c2)C(C)(C)c2ccccc2-3)c2ccc3c(c2)sc2ccccc23)ccc1-4]

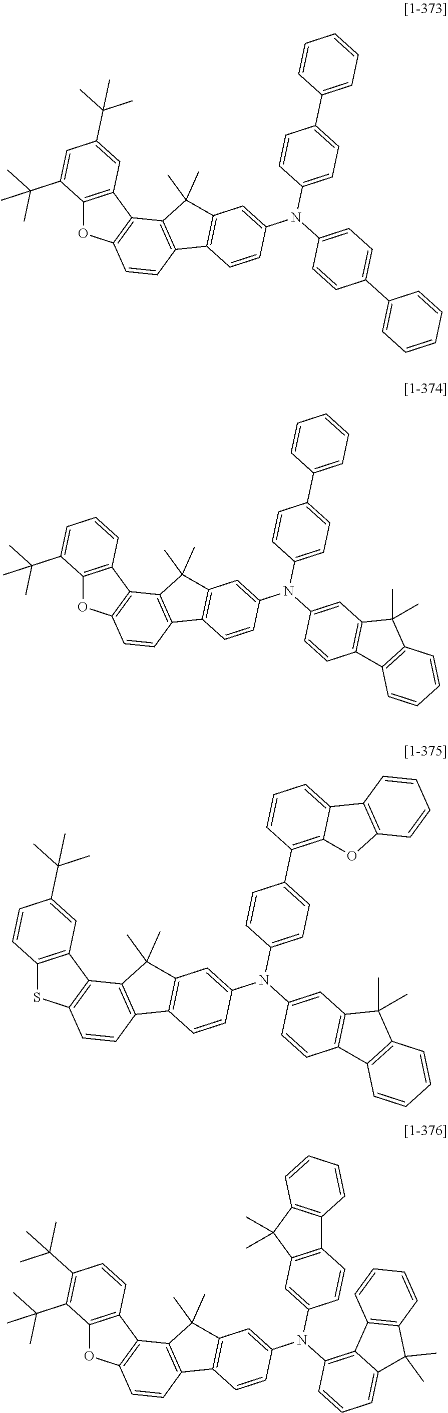 [SMILES: CC(C)(C)c1cc(C(C)(C)C)c2oc3ccc4c(c3c2c1)C(C)(C)c1cc(N(c2ccc(-c3ccccc3)cc2)c2ccc(-c3ccccc3)cc2)ccc1-4 ; CC(C)(C)c1cccc2c1oc1ccc3c(c12)C(C)(C)c1cc(N(c2ccc(-c4ccccc4)cc2)c2ccc4c(c2)C(C)(C)c2ccccc2-4)ccc1-3 ; CC(C)(C)c1ccc2sc3ccc4c(c3c2c1)C(C)(C)c1cc(N(c2ccc(-c3cccc5c3oc3ccccc35)cc2)c2ccc3c(c2)C(C)(C)c2ccccc2-3)ccc1-4 ; CC(C)(C)c1ccc2c(oc3ccc4c(c32)C(C)(C)c2cc(N(c3ccc5c(c3)C(C)(C)c3ccccc3-5)c3cccc5c3-c3ccccc3C5(C)C)ccc2-4)c1C(C)(C)C]

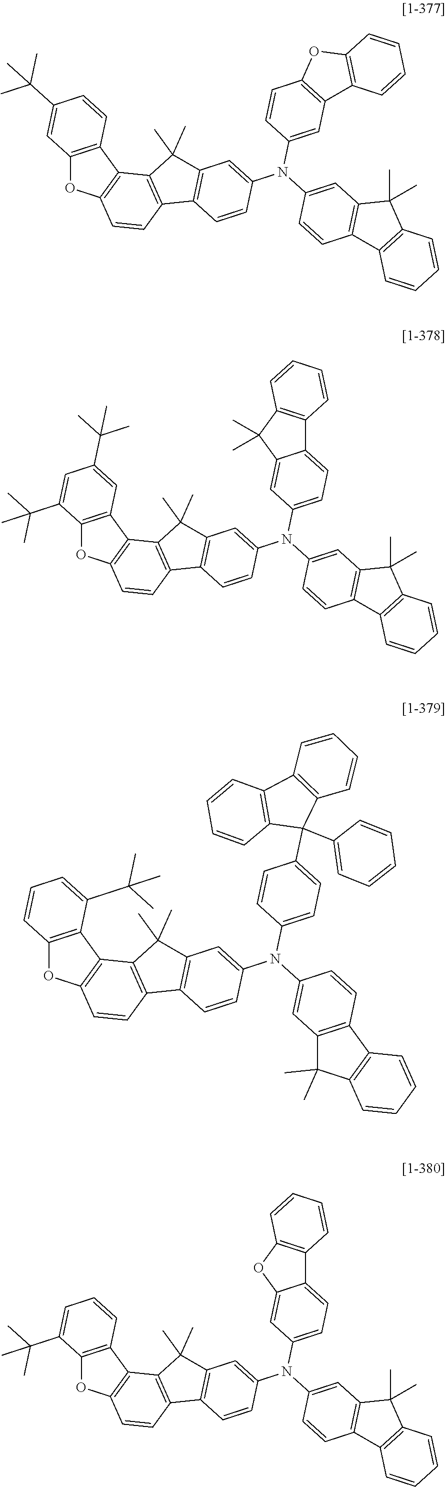 [SMILES: CC(C)(C)c1ccc2c(c1)oc1ccc3c(c12)C(C)(C)c1cc(N(c2ccc4c(c2)C(C)(C)c2ccccc2-4)c2ccc4oc5ccccc5c4c2)ccc1-3 ; CC(C)(C)c1cc(C(C)(C)C)c2oc3ccc4c(c3c2c1)C(C)(C)c1cc(N(c2ccc3c(c2)C(C)(C)c2ccccc2-3)c2ccc3c(c2)C(C)(C)c2ccccc2-3)ccc1-4 ; CC(C)(C)c1cccc2oc3ccc4c(c3c12)C(C)(C)c1cc(N(c2ccc(C3(c5ccccc5)c5ccccc5-c5ccccc53)cc2)c2ccc3c(c2)C(C)(C)c2ccccc2-3)ccc1-4 ; CC(C)(C)c1cccc2c1oc1ccc3c(c12)C(C)(C)c1cc(N(c2ccc4c(c2)C(C)(C)c2ccccc2-4)c2ccc4c(c2)oc2ccccc24)ccc1-3]

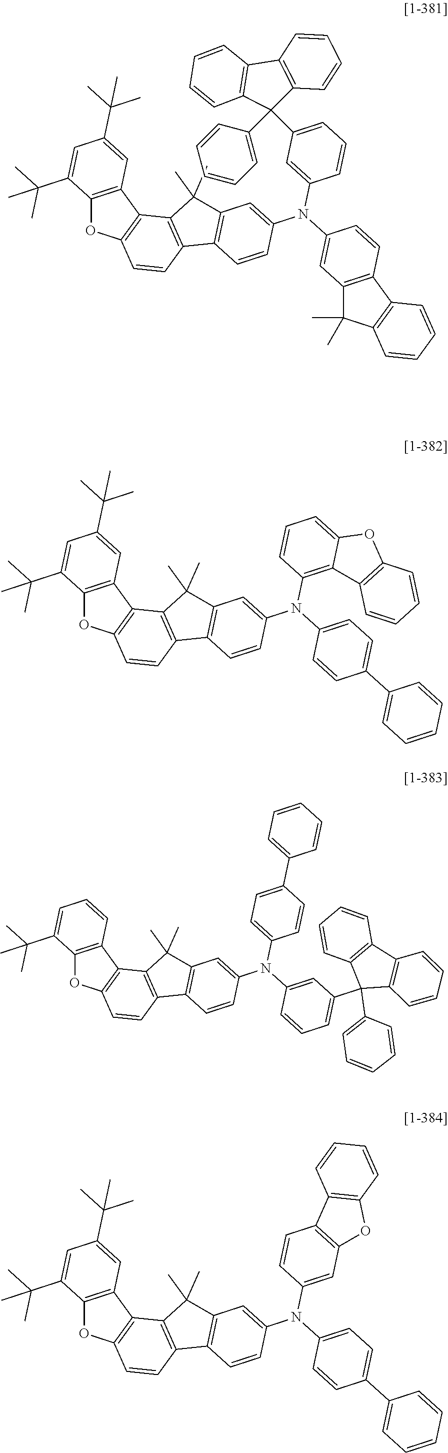

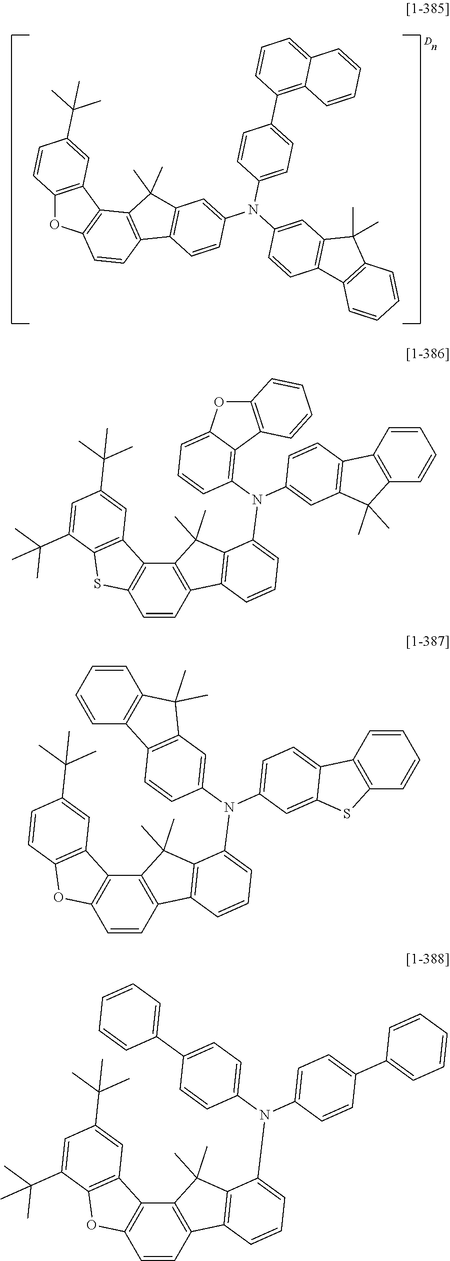

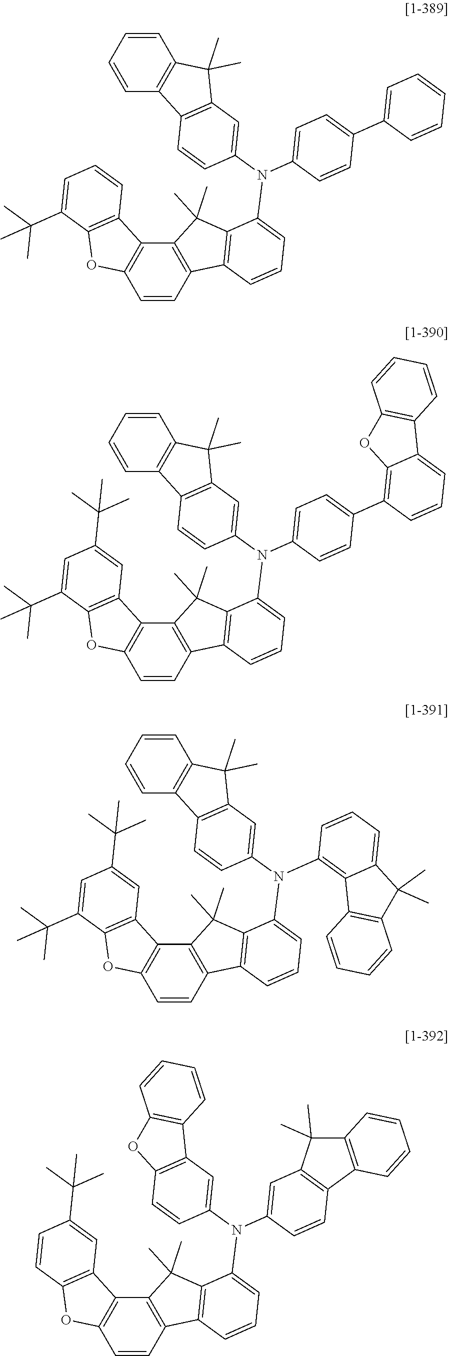 [SMILES: CC(C)(C)c1cccc2c1oc1ccc3c(c12)C(C)(C)c1c-3cccc1N(c1ccc(-c2ccccc2)cc1)c1ccc2c(c1)C(C)(C)c1ccccc1-2 ; CC(C)(C)c1cc(C(C)(C)C)c2oc3ccc4c(c3c2c1)C(C)(C)c1c-4cccc1N(c1ccc(-c2cccc3c2oc2ccccc23)cc1)c1ccc2c(c1)C(C)(C)c1ccccc1-2 ; CC(C)(C)c1cc(C(C)(C)C)c2oc3ccc4c(c3c2c1)C(C)(C)c1c-4cccc1N(c1ccc2c(c1)C(C)(C)c1ccccc1-2)c1cccc2c1-c1ccccc1C2(C)C ; CC(C)(C)c1ccc2oc3ccc4c(c3c2c1)C(C)(C)c1c-4cccc1N(c1ccc2c(c1)C(C)(C)c1ccccc1-2)c1ccc2oc3ccccc3c2c1]

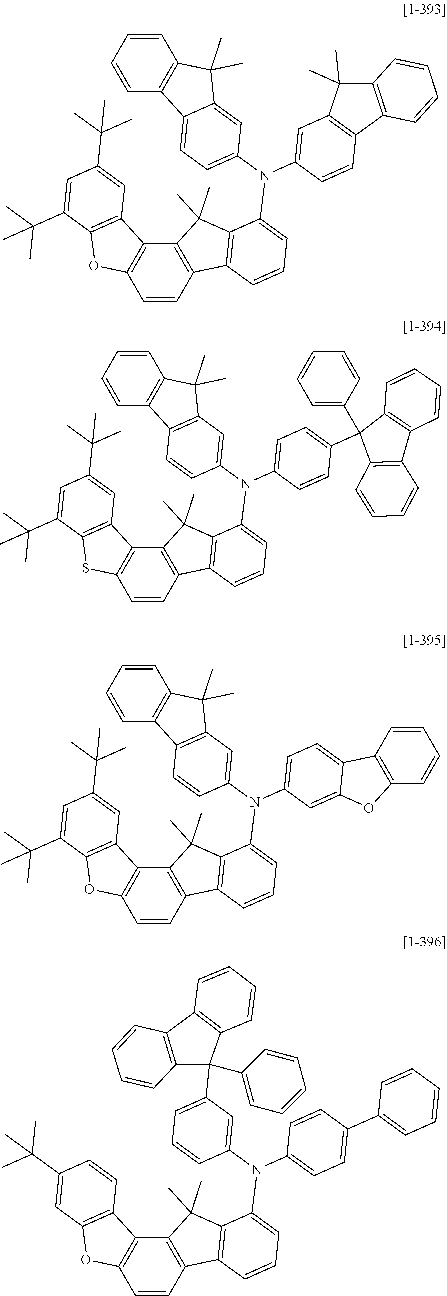 [SMILES: CC(C)(C)c1cc(C(C)(C)C)c2oc3ccc4c(c3c2c1)C(C)(C)c1c-4cccc1N(c1ccc2c(c1)C(C)(C)c1ccccc1-2)c1ccc2c(c1)C(C)(C)c1ccccc1-2 ; CC(C)(C)c1cc(C(C)(C)C)c2sc3ccc4c(c3c2c1)C(C)(C)c1c-4cccc1N(c1ccc(C2(c3ccccc3)c3ccccc3-c3ccccc32)cc1)c1ccc2c(c1)C(C)(C)c1ccccc1-2 ; CC(C)(C)c1cc(C(C)(C)C)c2oc3ccc4c(c3c2c1)C(C)(C)c1c-4cccc1N(c1ccc2c(c1)C(C)(C)c1ccccc1-2)c1ccc2c(c1)oc1ccccc12 ; CC(C)(C)c1ccc2c(c1)oc1ccc3c(c12)C(C)(C)c1c-3cccc1N(c1ccc(-c2ccccc2)cc1)c1cccc(C2(c3ccccc3)c3ccccc3-c3ccccc32)c1]

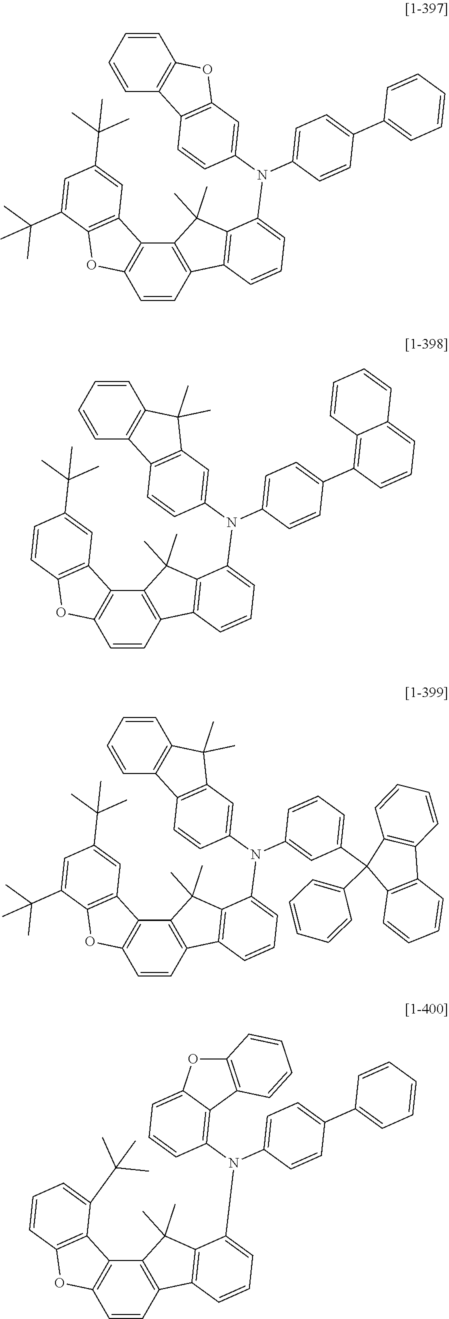 [SMILES: CC(C)(C)c1cc(C(C)(C)C)c2oc3ccc4c(c3c2c1)C(C)(C)c1c-4cccc1N(c1ccc(-c2ccccc2)cc1)c1ccc2c(c1)oc1ccccc12 ; CC(C)(C)c1ccc2oc3ccc4c(c3c2c1)C(C)(C)c1c-4cccc1N(c1ccc(-c2cccc3ccccc23)cc1)c1ccc2c(c1)C(C)(C)c1ccccc1-2 ; CC(C)(C)c1cc(C(C)(C)C)c2oc3ccc4c(c3c2c1)C(C)(C)c1c-4cccc1N(c1cccc(C2(c3ccccc3)c3ccccc3-c3ccccc32)c1)c1ccc2c(c1)C(C)(C)c1ccccc1-2 ; CC(C)(C)c1cccc2oc3ccc4c(c3c12)C(C)(C)c1c-4cccc1N(c1ccc(-c2ccccc2)cc1)c1cccc2oc3ccccc3c12]

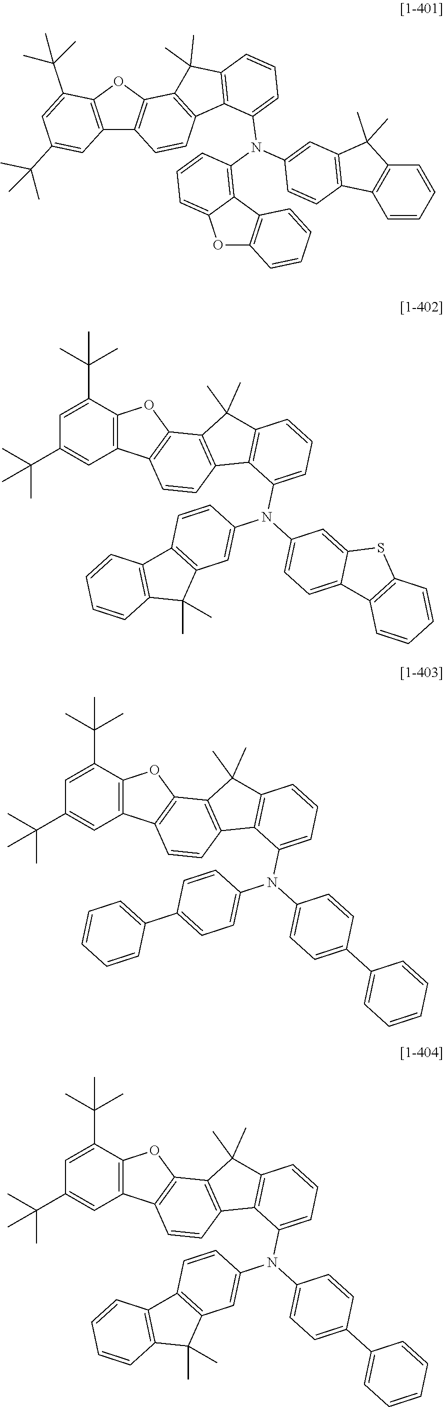 [SMILES: CC(C)(C)c1cc(C(C)(C)C)c2oc3c4c(ccc3c2c1)-c1c(N(c2ccc3c(c2)C(C)(C)c2ccccc2-3)c2cccc3oc5ccccc5c23)cccc1C4(C)C ; CC(C)(C)c1cc(C(C)(C)C)c2oc3c4c(ccc3c2c1)-c1c(N(c2ccc3c(c2)C(C)(C)c2ccccc2-3)c2ccc3c(c2)sc2ccccc23)cccc1C4(C)C ; CC(C)(C)c1cc(C(C)(C)C)c2oc3c4c(ccc3c2c1)-c1c(N(c2ccc(-c3ccccc3)cc2)c2ccc(-c3ccccc3)cc2)cccc1C4(C)C ; CC(C)(C)c1cc(C(C)(C)C)c2oc3c4c(ccc3c2c1)-c1c(N(c2ccc(-c3ccccc3)cc2)c2ccc3c(c2)C(C)(C)c2ccccc2-3)cccc1C4(C)C]

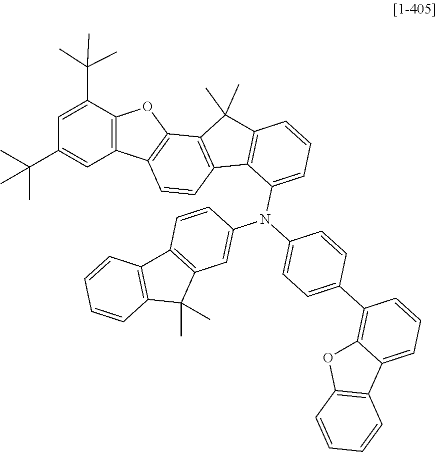 [SMILES: CC(C)(C)c1cc(C(C)(C)C)c2oc3c4c(ccc3c2c1)-c1c(N(c2ccc(-c3cccc5c3oc3ccccc35)cc2)c2ccc3c(c2)C(C)(C)c2ccccc2-3)cccc1C4(C)C]

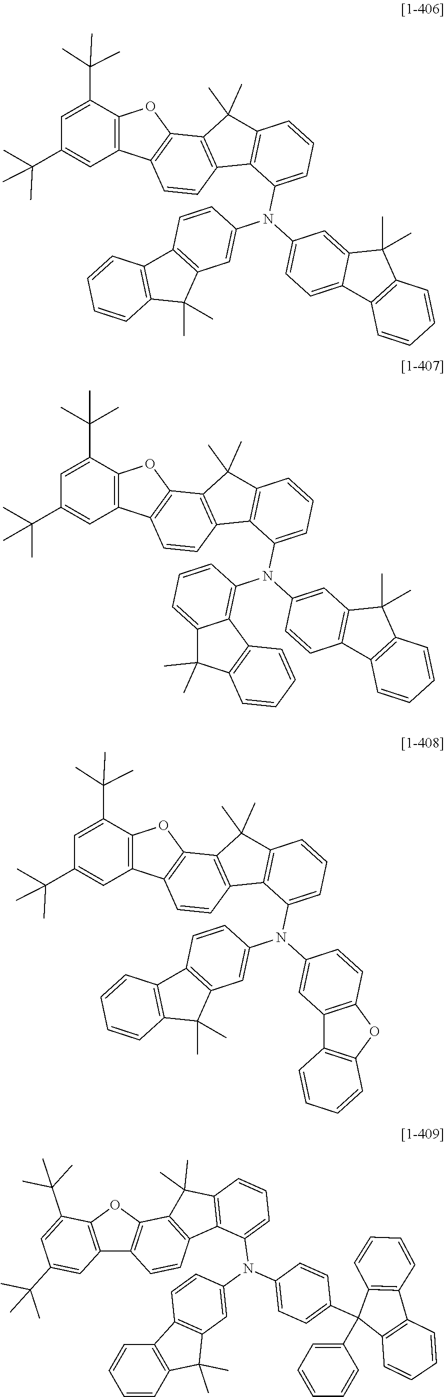 [SMILES: CC(C)(C)c1cc(C(C)(C)C)c2oc3c4c(ccc3c2c1)-c1c(N(c2ccc3c(c2)C(C)(C)c2ccccc2-3)c2ccc3c(c2)C(C)(C)c2ccccc2-3)cccc1C4(C)C ; CC(C)(C)c1cc(C(C)(C)C)c2oc3c4c(ccc3c2c1)-c1c(N(c2ccc3c(c2)C(C)(C)c2ccccc2-3)c2cccc3c2-c2ccccc2C3(C)C)cccc1C4(C)C ; CC(C)(C)c1cc(C(C)(C)C)c2oc3c4c(ccc3c2c1)-c1c(N(c2ccc3c(c2)C(C)(C)c2ccccc2-3)c2ccc3oc5ccccc5c3c2)cccc1C4(C)C ; CC(C)(C)c1cc(C(C)(C)C)c2oc3c4c(ccc3c2c1)-c1c(N(c2ccc(C3(c5ccccc5)c5ccccc5-c5ccccc53)cc2)c2ccc3c(c2)C(C)(C)c2ccccc2-3)cccc1C4(C)C]

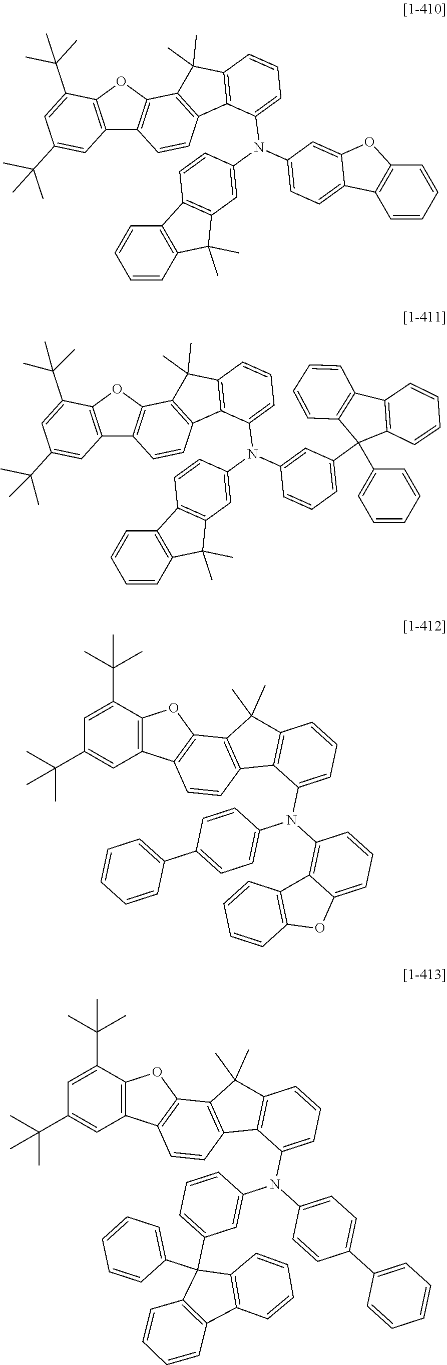 [SMILES: CC(C)(C)c1cc(C(C)(C)C)c2oc3c4c(ccc3c2c1)-c1c(N(c2ccc3c(c2)C(C)(C)c2ccccc2-3)c2ccc3c(c2)oc2ccccc23)cccc1C4(C)C ; CC(C)(C)c1cc(C(C)(C)C)c2oc3c4c(ccc3c2c1)-c1c(N(c2cccc(C3(c5ccccc5)c5ccccc5-c5ccccc53)c2)c2ccc3c(c2)C(C)(C)c2ccccc2-3)cccc1C4(C)C ; CC(C)(C)c1cc(C(C)(C)C)c2oc3c4c(ccc3c2c1)-c1c(N(c2ccc(-c3ccccc3)cc2)c2cccc3oc5ccccc5c23)cccc1C4(C)C ; CC(C)(C)c1cc(C(C)(C)C)c2oc3c4c(ccc3c2c1)-c1c(N(c2ccc(-c3ccccc3)cc2)c2cccc(C3(c5ccccc5)c5ccccc5-c5ccccc53)c2)cccc1C4(C)C]

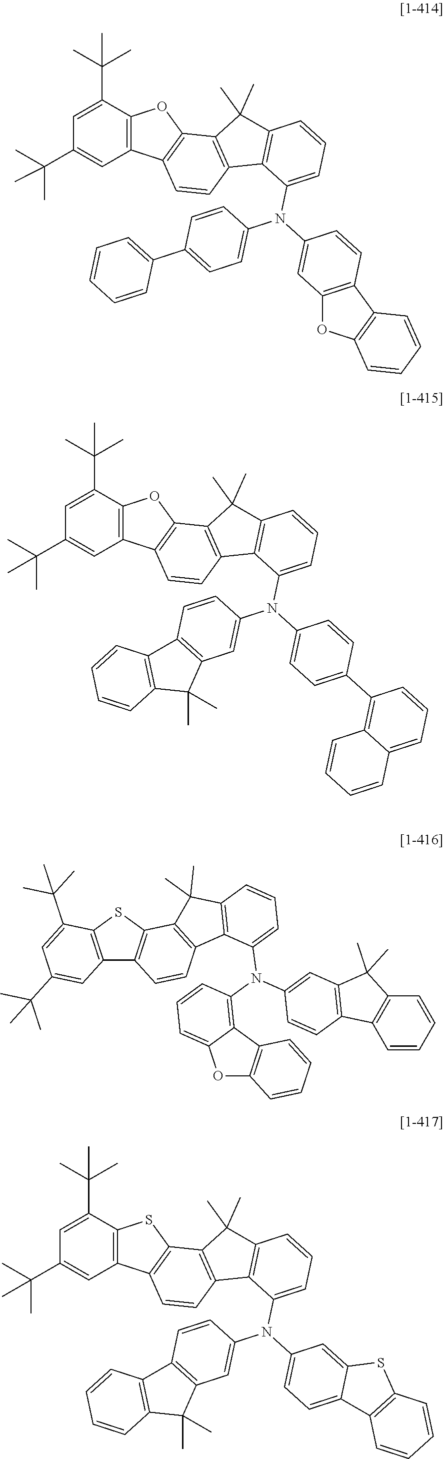 [SMILES: CC(C)(C)c1cc(C(C)(C)C)c2oc3c4c(ccc3c2c1)-c1c(N(c2ccc(-c3ccccc3)cc2)c2ccc3c(c2)oc2ccccc23)cccc1C4(C)C ; CC(C)(C)c1cc(C(C)(C)C)c2oc3c4c(ccc3c2c1)-c1c(N(c2ccc(-c3cccc5ccccc35)cc2)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc3c(c2)C(C)(C)c2ccccc2-3)c2cccc3oc5ccccc5c23)cccc1C4(C)C ; CC(C)(C)c1cc(C(C)(C)C)c2sc3c4c(ccc3c2c1)-c1c(N(c2ccc3c(c2)C(C)(C)c2ccccc2-3)c2ccc3c(c2)sc2ccccc23)cccc1C4(C)C]

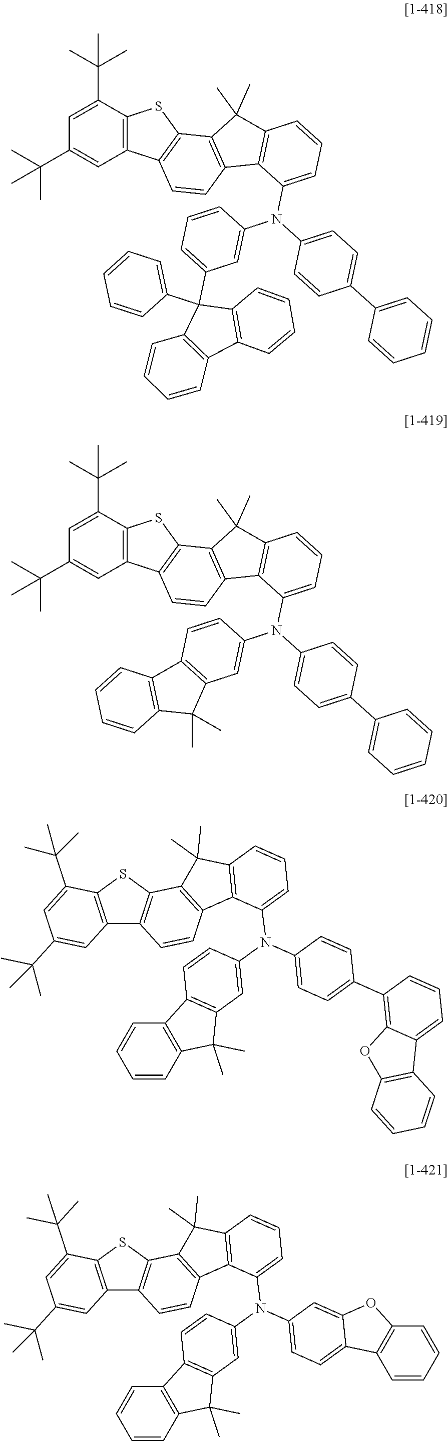 [SMILES: CC(C)(C)c1cc(C(C)(C)C)c2sc3c4c(ccc3c2c1)-c1c(N(c2ccc(-c3ccccc3)cc2)c2cccc(C3(c5ccccc5)c5ccccc5-c5ccccc53)c2)cccc1C4(C)C ; CC(C)(C)c1cc(C(C)(C)C)c2sc3c4c(ccc3c2c1)-c1c(N(c2ccc(-c3ccccc3)cc2)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc(-c3cccc5c3oc3ccccc35)cc2)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc3c(c2)C(C)(C)c2ccccc2-3)c2ccc3c(c2)oc2ccccc23)cccc1C4(C)C]

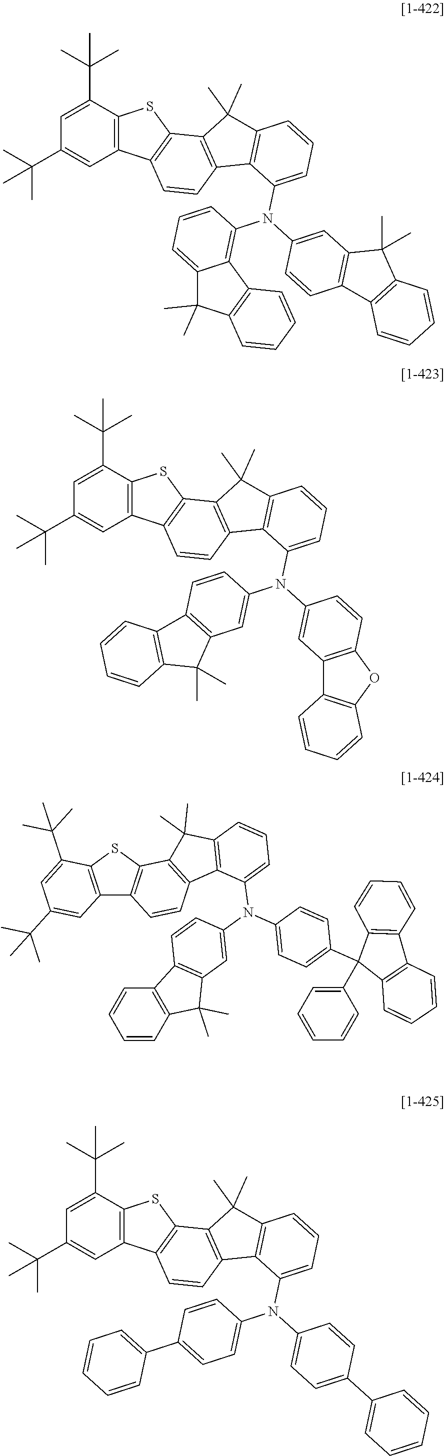 [SMILES: CC(C)(C)c1cc(C(C)(C)C)c2sc3c4c(ccc3c2c1)-c1c(N(c2ccc3c(c2)C(C)(C)c2ccccc2-3)c2cccc3c2-c2ccccc2C3(C)C)cccc1C4(C)C ; CC(C)(C)c1cc(C(C)(C)C)c2sc3c4c(ccc3c2c1)-c1c(N(c2ccc3c(c2)C(C)(C)c2ccccc2-3)c2ccc3oc5ccccc5c3c2)cccc1C4(C)C ; CC(C)(C)c1cc(C(C)(C)C)c2sc3c4c(ccc3c2c1)-c1c(N(c2ccc(C3(c5ccccc5)c5ccccc5-c5ccccc53)cc2)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc(-c3ccccc3)cc2)c2ccc(-c3ccccc3)cc2)cccc1C4(C)C]

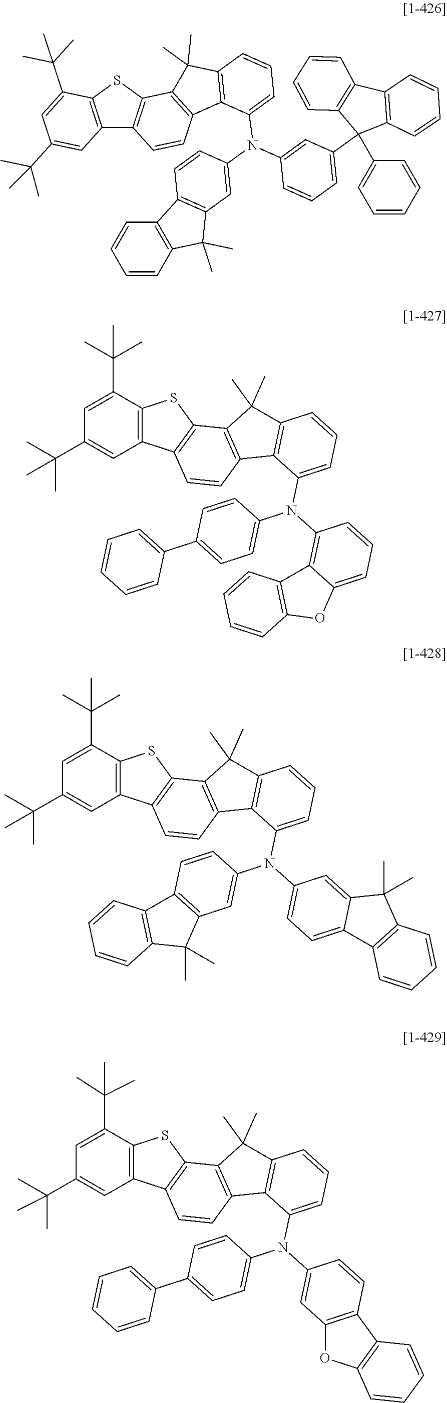 [SMILES: CC(C)(C)c1cc(C(C)(C)C)c2sc3c4c(ccc3c2c1)-c1c(N(c2cccc(C3(c5ccccc5)c5ccccc5-c5ccccc53)c2)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc(-c3ccccc3)cc2)c2cccc3oc5ccccc5c23)cccc1C4(C)C ; CC(C)(C)c1cc(C(C)(C)C)c2sc3c4c(ccc3c2c1)-c1c(N(c2ccc3c(c2)C(C)(C)c2ccccc2-3)c2ccc3c(c2)C(C)(C)c2ccccc2-3)cccc1C4(C)C ; CC(C)(C)c1cc(C(C)(C)C)c2sc3c4c(ccc3c2c1)-c1c(N(c2ccc(-c3ccccc3)cc2)c2ccc3c(c2)oc2ccccc23)cccc1C4(C)C]

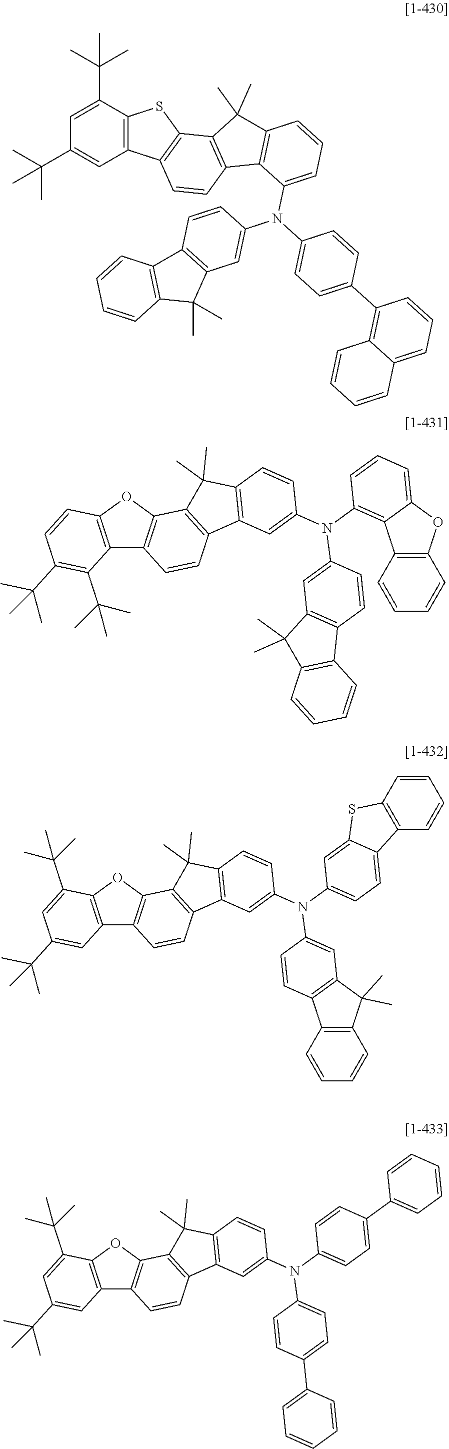 [SMILES: CC(C)(C)c1cc(C(C)(C)C)c2sc3c4c(ccc3c2c1)-c1c(N(c2ccc(-c3cccc5ccccc35)cc2)c2ccc3c(c2)C(C)(C)c2ccccc2-3)cccc1C4(C)C ; CC(C)(C)c1ccc2oc3c4c(ccc3c2c1C(C)(C)C)-c1cc(N(c2ccc3c(c2)C(C)(C)c2ccccc2-3)c2cccc3oc5ccccc5c23)ccc1C4(C)C ; CC(C)(C)c1cc(C(C)(C)C)c2oc3c4c(ccc3c2c1)-c1cc(N(c2ccc3c(c2)C(C)(C)c2ccccc2-3)c2ccc3c(c2)sc2ccccc23)ccc1C4(C)C ; CC(C)(C)c1cc(C(C)(C)C)c2oc3c4c(ccc3c2c1)-c1cc(N(c2ccc(-c3ccccc3)cc2)c2ccc(-c3ccccc3)cc2)ccc1C4(C)C]

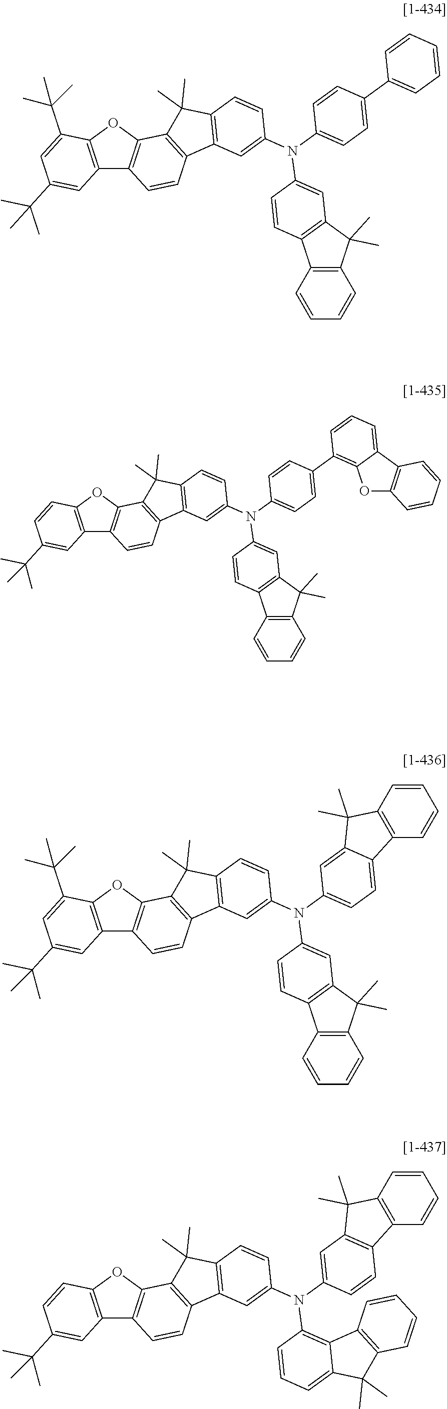 [SMILES: CC(C)(C)c1cc(C(C)(C)C)c2oc3c4c(ccc3c2c1)-c1cc(N(c2ccc(-c3ccccc3)cc2)c2ccc3c(c2)C(C)(C)c2ccccc2-3)ccc1C4(C)C ; CC(C)(C)c1ccc2oc3c4c(ccc3c2c1)-c1cc(N(c2ccc(-c3cccc5c3oc3ccccc35)cc2)c2ccc3c(c2)C(C)(C)c2ccccc2-3)ccc1C4(C)C ; CC(C)(C)c1cc(C(C)(C)C)c2oc3c4c(ccc3c2c1)-c1cc(N(c2ccc3c(c2)C(C)(C)c2ccccc2-3)c2ccc3c(c2)C(C)(C)c2ccccc2-3)ccc1C4(C)C ; CC(C)(C)c1ccc2oc3c4c(ccc3c2c1)-c1cc(N(c2ccc3c(c2)C(C)(C)c2ccccc2-3)c2cccc3c2-c2ccccc2C3(C)C)ccc1C4(C)C]

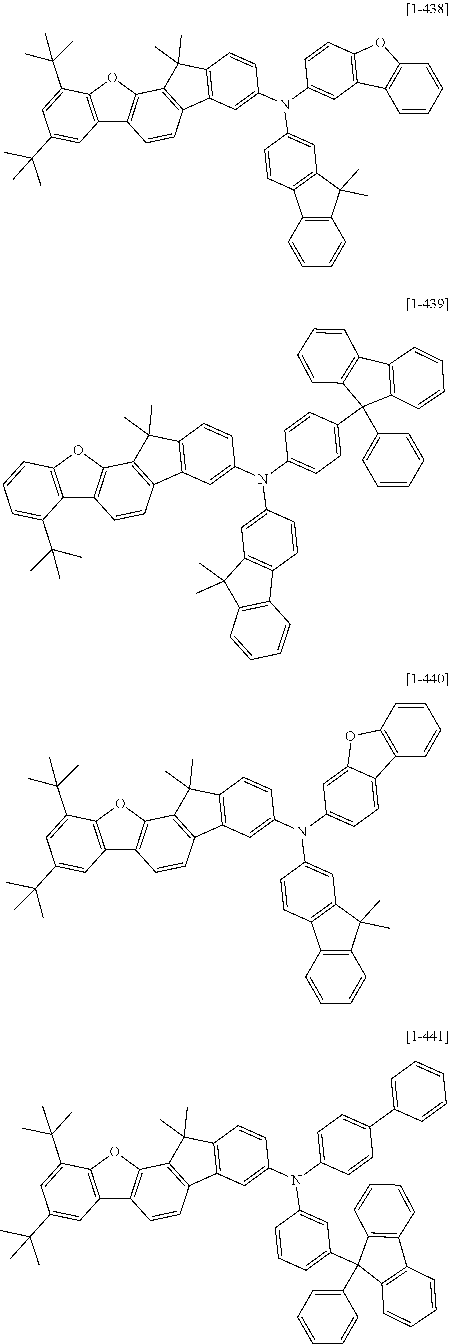 [SMILES: CC(C)(C)c1cc(C(C)(C)C)c2oc3c4c(ccc3c2c1)-c1cc(N(c2ccc3c(c2)C(C)(C)c2ccccc2-3)c2ccc3oc5ccccc5c3c2)ccc1C4(C)C ; CC(C)(C)c1cccc2oc3c4c(ccc3c12)-c1cc(N(c2ccc(C3(c5ccccc5)c5ccccc5-c5ccccc53)cc2)c2ccc3c(c2)C(C)(C)c2ccccc2-3)ccc1C4(C)C ; CC(C)(C)c1cc(C(C)(C)C)c2oc3c4c(ccc3c2c1)-c1cc(N(c2ccc3c(c2)C(C)(C)c2ccccc2-3)c2ccc3c(c2)oc2ccccc23)ccc1C4(C)C ; CC(C)(C)c1cc(C(C)(C)C)c2oc3c4c(ccc3c2c1)-c1cc(N(c2ccc(-c3ccccc3)cc2)c2cccc(C3(c5ccccc5)c5ccccc5-c5ccccc53)c2)ccc1C4(C)C]

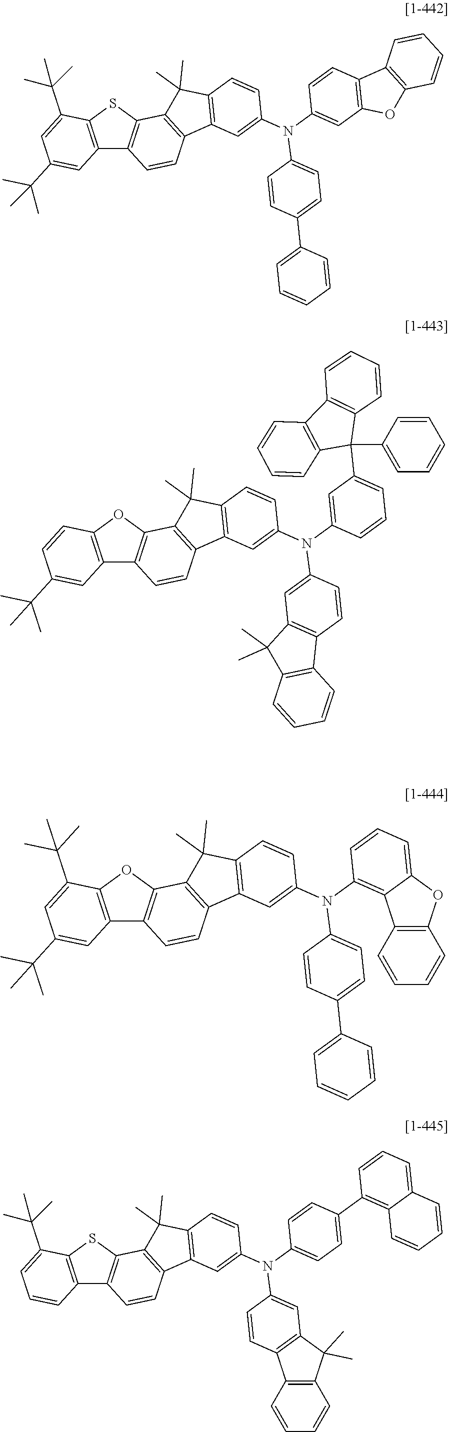 [SMILES: CC(C)(C)c1cc(C(C)(C)C)c2sc3c4c(ccc3c2c1)-c1cc(N(c2ccc(-c3ccccc3)cc2)c2ccc3c(c2)oc2ccccc23)ccc1C4(C)C ; CC(C)(C)c1ccc2oc3c4c(ccc3c2c1)-c1cc(N(c2cccc(C3(c5ccccc5)c5ccccc5-c5ccccc53)c2)c2ccc3c(c2)C(C)(C)c2ccccc2-3)ccc1C4(C)C ; CC(C)(C)c1cc(C(C)(C)C)c2oc3c4c(ccc3c2c1)-c1cc(N(c2ccc(-c3ccccc3)cc2)c2cccc3oc5ccccc5c23)ccc1C4(C)C ; CC(C)(C)c1cccc2c1sc1c3c(ccc12)-c1cc(N(c2ccc(-c4cccc5ccccc45)cc2)c2ccc4c(c2)C(C)(C)c2ccccc2-4)ccc1C3(C)C]

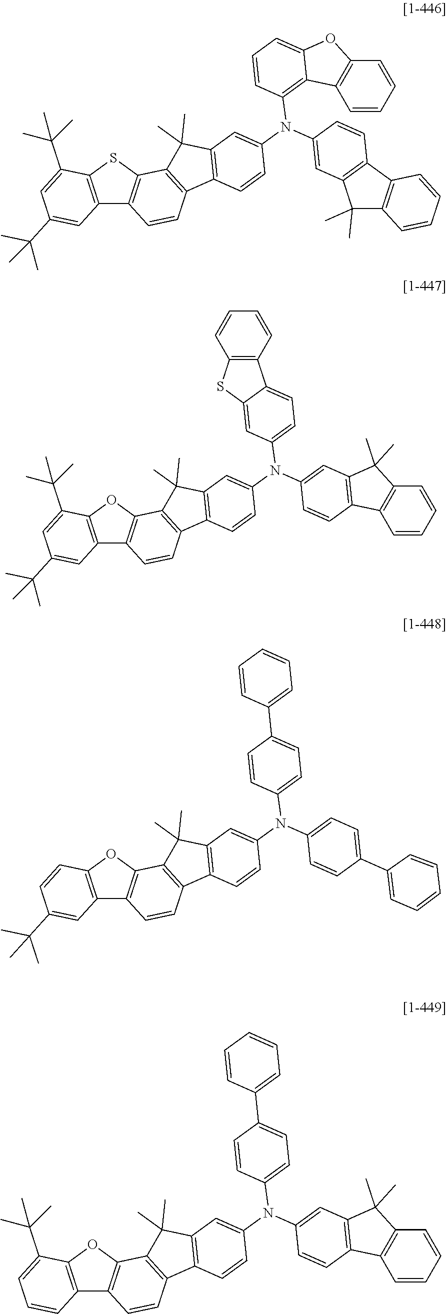 [SMILES: CC(C)(C)c1cc(C(C)(C)C)c2sc3c4c(ccc3c2c1)-c1ccc(N(c2ccc3c(c2)C(C)(C)c2ccccc2-3)c2cccc3oc5ccccc5c23)cc1C4(C)C ; CC(C)(C)c1cc(C(C)(C)C)c2oc3c4c(ccc3c2c1)-c1ccc(N(c2ccc3c(c2)C(C)(C)c2ccccc2-3)c2ccc3c(c2)sc2ccccc23)cc1C4(C)C ; CC(C)(C)c1ccc2oc3c4c(ccc3c2c1)-c1ccc(N(c2ccc(-c3ccccc3)cc2)c2ccc(-c3ccccc3)cc2)cc1C4(C)C ; CC(C)(C)c1cccc2c1oc1c3c(ccc12)-c1ccc(N(c2ccc(-c4ccccc4)cc2)c2ccc4c(c2)C(C)(C)c2ccccc2-4)cc1C3(C)C]

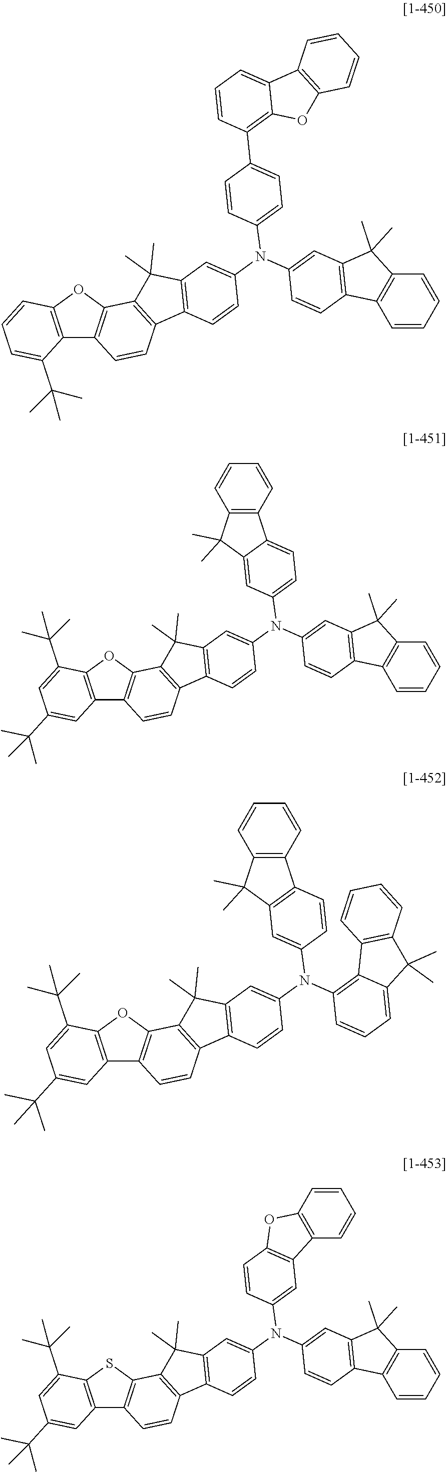

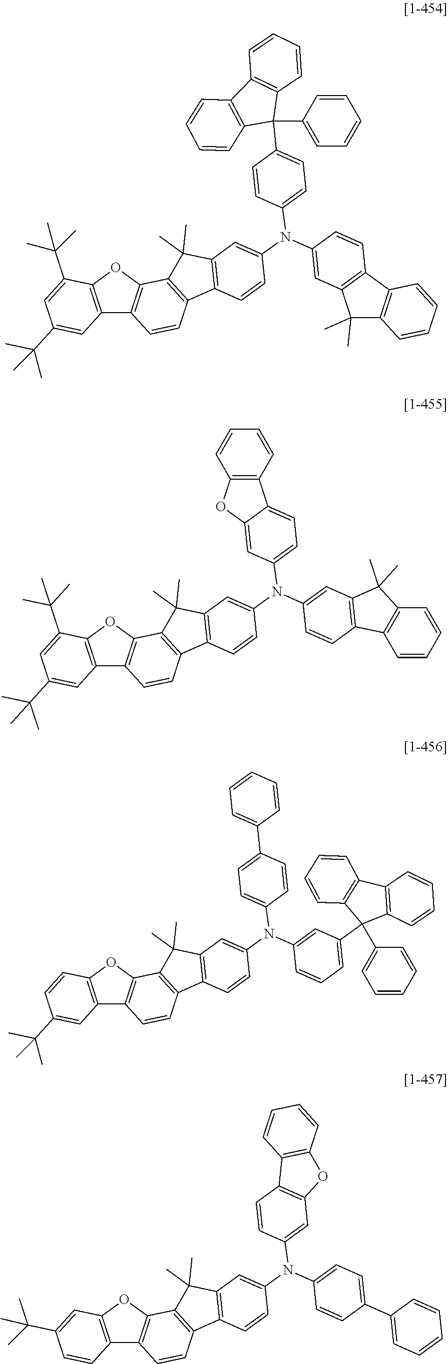 [SMILES: CC(C)(C)c1cc(C(C)(C)C)c2oc3c4c(ccc3c2c1)-c1ccc(N(c2ccc(C3(c5ccccc5)c5ccccc5-c5ccccc53)cc2)c2ccc3c(c2)C(C)(C)c2ccccc2-3)cc1C4(C)C ; CC(C)(C)c1cc(C(C)(C)C)c2oc3c4c(ccc3c2c1)-c1ccc(N(c2ccc3c(c2)C(C)(C)c2ccccc2-3)c2ccc3c(c2)oc2ccccc23)cc1C4(C)C ; CC(C)(C)c1ccc2oc3c4c(ccc3c2c1)-c1ccc(N(c2ccc(-c3ccccc3)cc2)c2cccc(C3(c5ccccc5)c5ccccc5-c5ccccc53)c2)cc1C4(C)C ; CC(C)(C)c1ccc2c(c1)oc1c3c(ccc12)-c1ccc(N(c2ccc(-c4ccccc4)cc2)c2ccc4c(c2)oc2ccccc24)cc1C3(C)C]

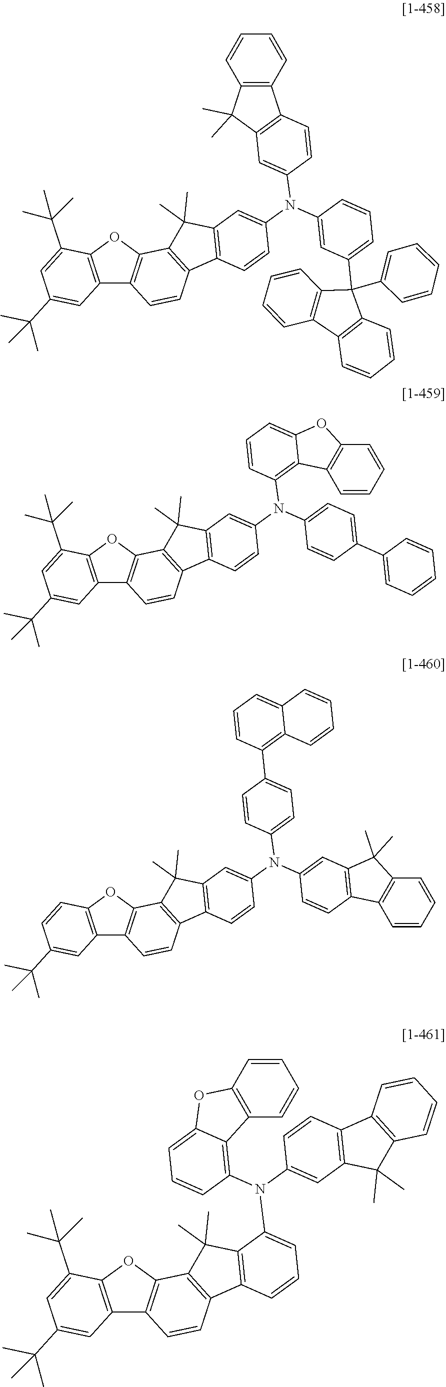 [SMILES: CC(C)(C)c1cc(C(C)(C)C)c2oc3c4c(ccc3c2c1)-c1ccc(N(c2cccc(C3(c5ccccc5)c5ccccc5-c5ccccc53)c2)c2ccc3c(c2)C(C)(C)c2ccccc2-3)cc1C4(C)C ; CC(C)(C)c1cc(C(C)(C)C)c2oc3c4c(ccc3c2c1)-c1ccc(N(c2ccc(-c3ccccc3)cc2)c2cccc3oc5ccccc5c23)cc1C4(C)C ; CC(C)(C)c1ccc2oc3c4c(ccc3c2c1)-c1ccc(N(c2ccc(-c3cccc5ccccc35)cc2)c2ccc3c(c2)C(C)(C)c2ccccc2-3)cc1C4(C)C ; CC(C)(C)c1cc(C(C)(C)C)c2oc3c4c(ccc3c2c1)-c1cccc(N(c2ccc3c(c2)C(C)(C)c2ccccc2-3)c2cccc3oc5ccccc5c23)c1C4(C)C]

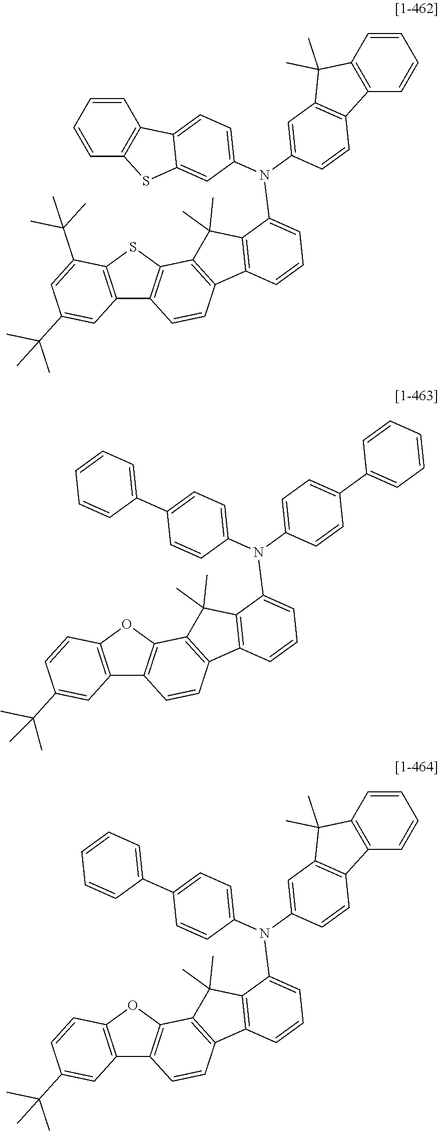 [SMILES: CC(C)(C)c1cc(C(C)(C)C)c2sc3c4c(ccc3c2c1)-c1cccc(N(c2ccc3c(c2)C(C)(C)c2ccccc2-3)c2ccc3c(c2)sc2ccccc23)c1C4(C)C ; CC(C)(C)c1ccc2oc3c4c(ccc3c2c1)-c1cccc(N(c2ccc(-c3ccccc3)cc2)c2ccc(-c3ccccc3)cc2)c1C4(C)C ; CC(C)(C)c1ccc2oc3c4c(ccc3c2c1)-c1cccc(N(c2ccc(-c3ccccc3)cc2)c2ccc3c(c2)C(C)(C)c2ccccc2-3)c1C4(C)C]

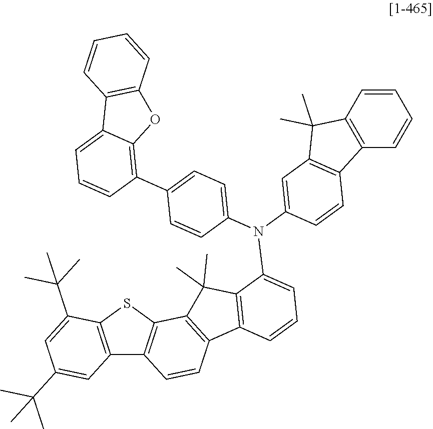 [SMILES: CC(C)(C)c1cc(C(C)(C)C)c2sc3c4c(ccc3c2c1)-c1cccc(N(c2ccc(-c3cccc5c3oc3ccccc35)cc2)c2ccc3c(c2)C(C)(C)c2ccccc2-3)c1C4(C)C]

In Group 1, Dn refers to the number of hydrogens replaced with deuterium and the number of n of Dn is 0 or more and is the maximum number that can be replaced. However, as noted above, any hydrogen in any compound may be protium, deuterium, or tritium, based on natural or artificial substitution.

In addition to the aforementioned compounds for organic optoelectronic devices described above, one or more compounds may be further included.

In an implementation, a dopant may be further included.

The dopant may be, e.g., a phosphorescent dopant, such as a red, green, or blue phosphorescent dopant, and may be, e.g., a red or green phosphorescent dopant.

The dopant may be a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L³MX² [Chemical Formula Z]

In Chemical Formula Z, M may be a metal, and L³ and X² may each independently be a ligand to form a complex compound with M.

The M may be, e.g., Ir, Pt, Os, Ti, Zr, Hf, Eu, Tb, Tm, Fe, Co, Ni, Ru, Rh, Pd, or a combination thereof, and L³ and X² may be, e.g., a bidentate ligand.

In an implementation, the ligands represented by L³ and X² may be a ligand of Group A.

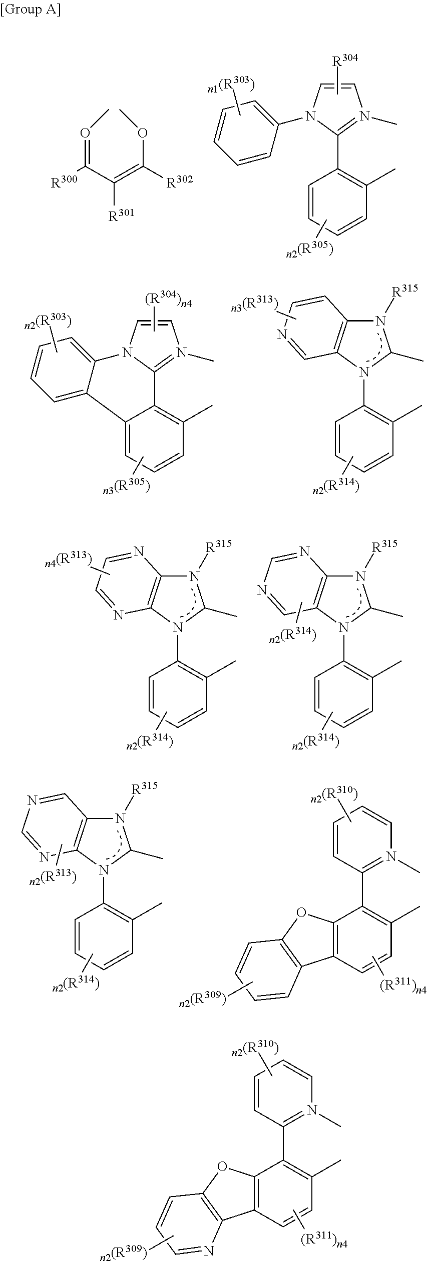

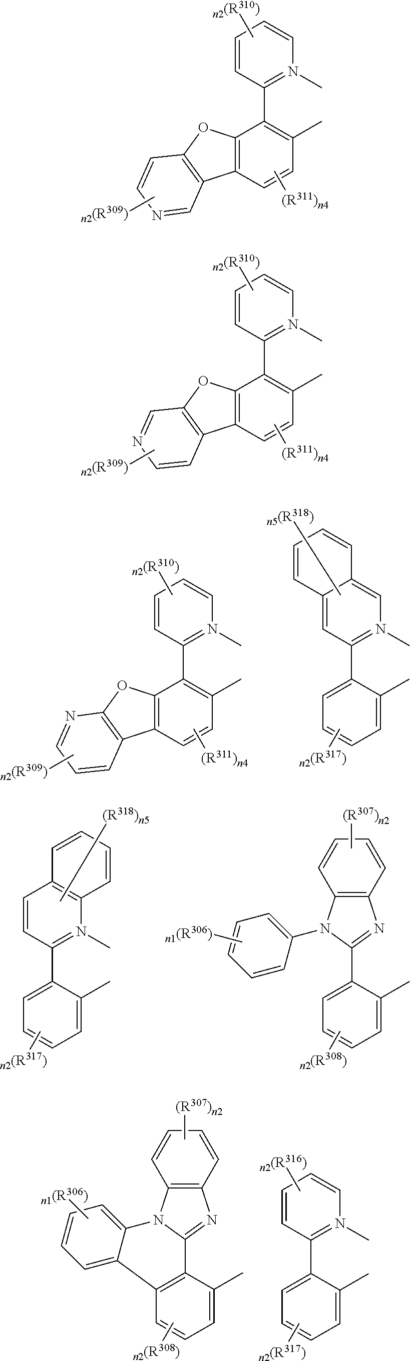

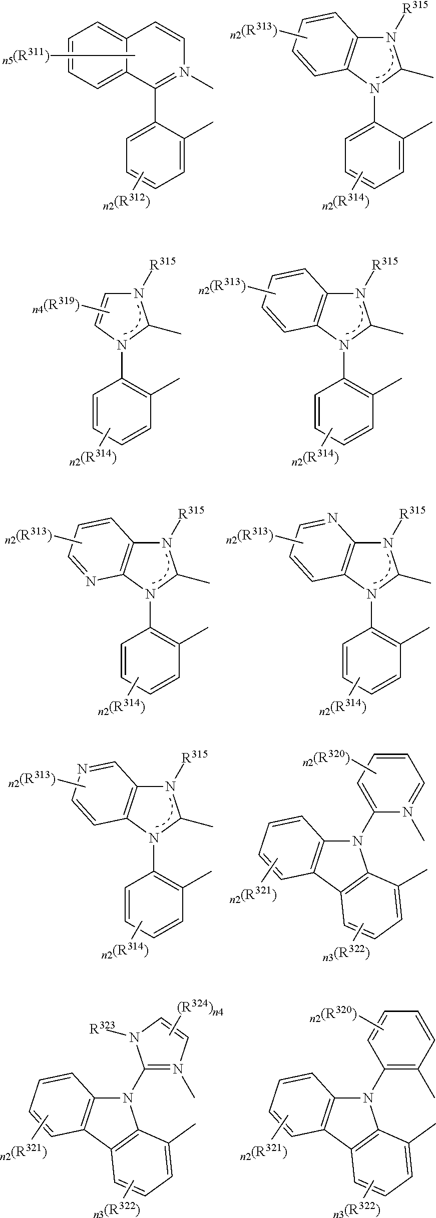

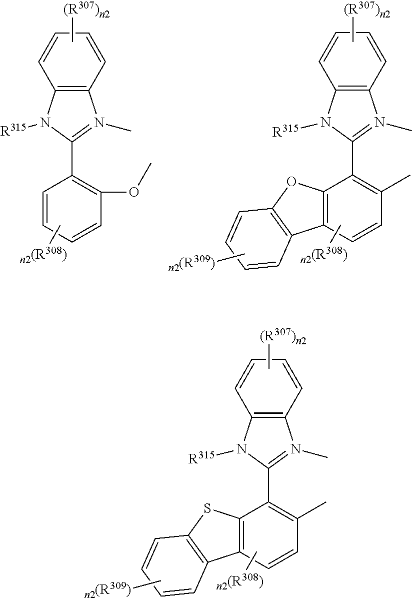

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

n1 may be, e.g., an integer of 1 to 5.

n2 may be, e.g., an integer of 1 to 4.

n3 may be, e.g., an integer of 1 to 3.

n4 may be, e.g., 1 or 2.

n5 may be, e.g., an integer of 1 to 6.

In an implementation, n1 may be 2 or more, and each substituent may be the same or different from each other.

In an implementation, n2 may be 2 or more, and each substituent may be the same or different from each other.

In an implementation, n3 may be 2 or more, and each substituent may be the same or different from each other.

In an implementation, n4 may be 2, and each substituent may be the same or different from each other.

In an implementation, n5 may be 2 or more, and each substituent may be the same or different from each other.

The dopant according to some example embodiments may be an iridium complex, and may include, e.g., a dopant represented by Chemical Formula V-1 or Chemical Formula V-2.

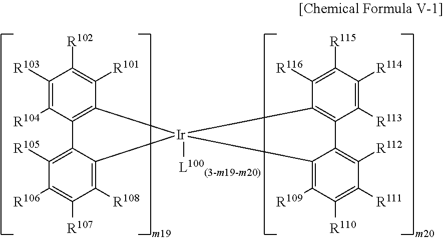

In Chemical Formula V-1, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁰¹ to R¹¹⁶ may be a functional group represented by Chemical Formula V-1-1.

L¹⁰⁰ may be, e.g., a bidentate ligand of a monovalent anion, and is a ligand that coordinates to iridium through a lone pair of carbons or heteroatoms.

m19 and m20 may each independently be, e.g., an integer of 0 to 3, and m19+m20 may be an integer of 1 to 3.

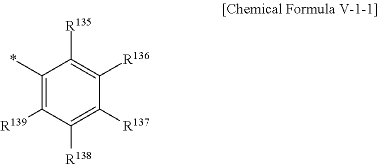

In Chemical Formula V-1-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* refers to the portion linked to the carbon atom.

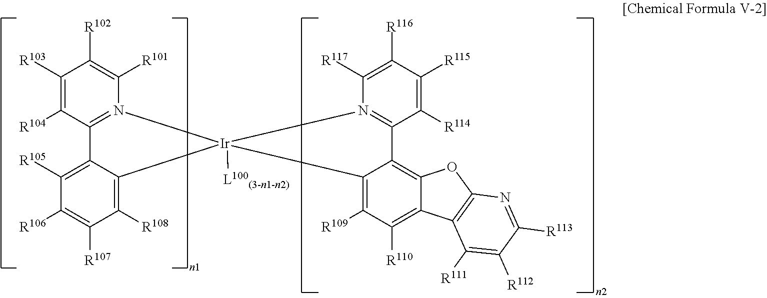

In Chemical Formula V-2, R¹⁰¹ to R¹¹⁷ may each independently be, e.g., hydrogen, deuterium, a substituted or unsubstituted C1 to C10 alkyl group, a substituted or unsubstituted C6 to C20 aryl group, or —Si R¹³³R¹³⁴R¹³⁵.

R¹³³ to R¹³⁵ may each independently be, e.g., a substituted or unsubstituted C1 to C6 alkyl group.

L¹⁰⁰ may be, e.g., a bidentate ligand of a monovalent anion, and is a ligand that coordinates to iridium through a lone pair of carbons or heteroatoms.

n1 and n2 may each independently be, e.g., an integer of 0 to 3, and n1+n2 may be an integer of 1 to 3.

In an implementation, the dopant according to some example embodiments may be a platinum complex, and may be represented, e.g., by Chemical Formula Z-1.

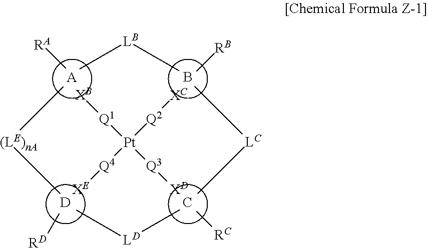

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

L^{B}, L^{C}, and LD may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be a direct bond, BR, NR, PR, O, S, Se, C═O, S═O, SO₂, CRR′, SiRR′, GeRR′, or a combination thereof. In an implementation, nA may be 0, and L^{E} does not exist;

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The platinum complex may be represented, e.g., by Chemical Formula VI-1 or Chemical Formula VI-2.

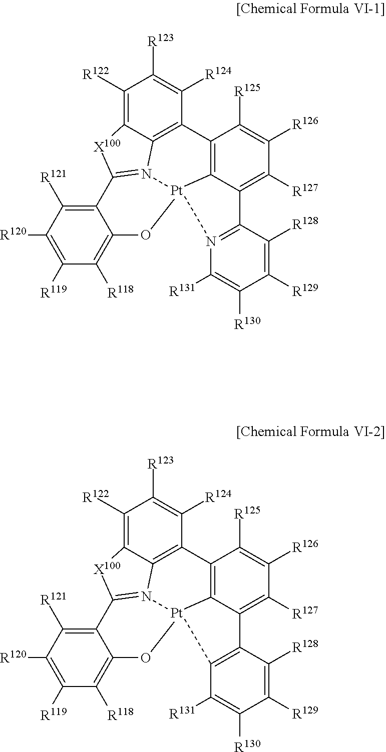

In Chemical Formula VI-1 and Chemical Formula VI-2, X¹⁰⁰ may be O, S, or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be —SiR¹³³R¹³⁴R¹³⁵ or a tert-butyl group.

R¹³³ to R¹³⁵ may each independently be, e.g., a substituted or unsubstituted C1 to C6 alkyl group.

Hereinafter, an organic optoelectronic device using the aforementioned compound for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

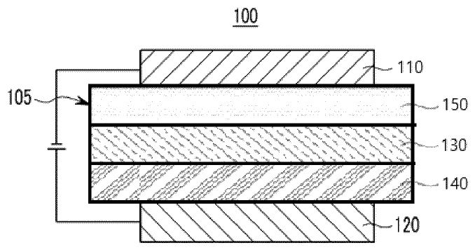

The FIG. 1s a cross-sectional view showing an organic light emitting diode according to some example embodiments.

Referring to the FIGURE, an organic light emitting diode 100 according to some example embodiments may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for an organic optoelectronic device.

The organic layer 105 may include a light emitting layer 130 and the light emitting layer 130 may include the aforementioned compound for an organic optoelectronic device.

The composition for an organic optoelectronic device further including a dopant may be, e.g., a green light emitting composition.

The light emitting layer 130 may include, e.g., the aforementioned compound for an organic optoelectronic device as a phosphorescent host.

The organic layer may further include a charge transport region in addition to the light emitting layer.

The charge transport region may be, e.g., a hole transport region 140.

The hole transport region 140 may help further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and the hole transport auxiliary layer may include the aforementioned compound for an organic optoelectronic device.

In an implementation, the light emitting layer may include a host and a dopant, and the host may be, e.g., a phosphorescent host.

The phosphorescent host may facilitate injection and transport of holes and electrons within the light emitting layer, ultimately holes and electrons should meet to form exciton well, and the formed exciton energy should be well transferred to the dopant. Examples of phosphorescent hosts may include an organic compound including carbazole, indolocarbazole, dibenzofuran, dibenzothiophene, indolodibenzofuran, indolodibenzothiophene, fluorene, indenocarbazole, triphenylene, pyrimidine, triazine, or a combination thereof.

The phosphorescent host may be a suitable material and may be, e.g., a single host or a mixed host.

In an implementation, the aforementioned compound for an organic optoelectronic device may be included in the light emitting layer, and a compound of Group C may be included in at least one layer of the hole transport layer and the hole transport auxiliary layer.

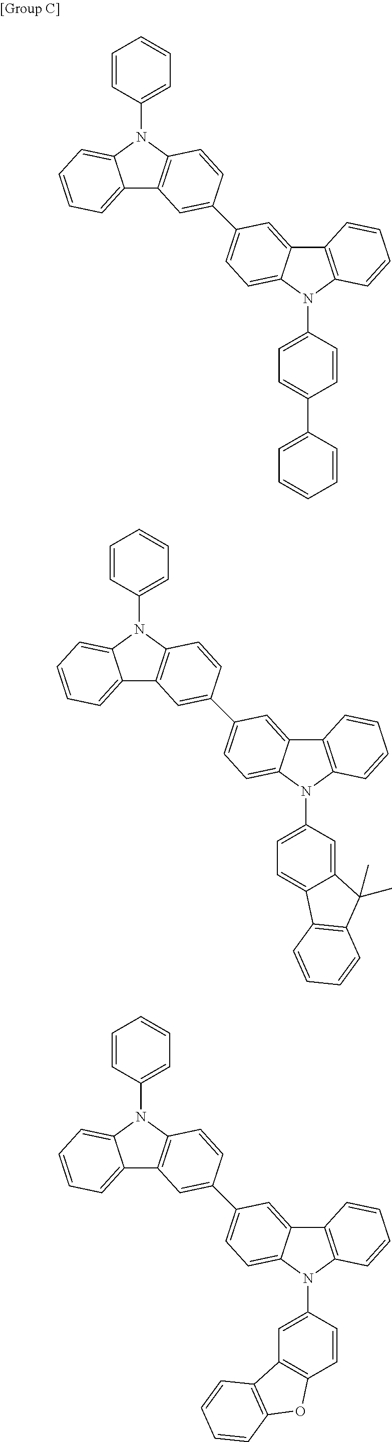 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

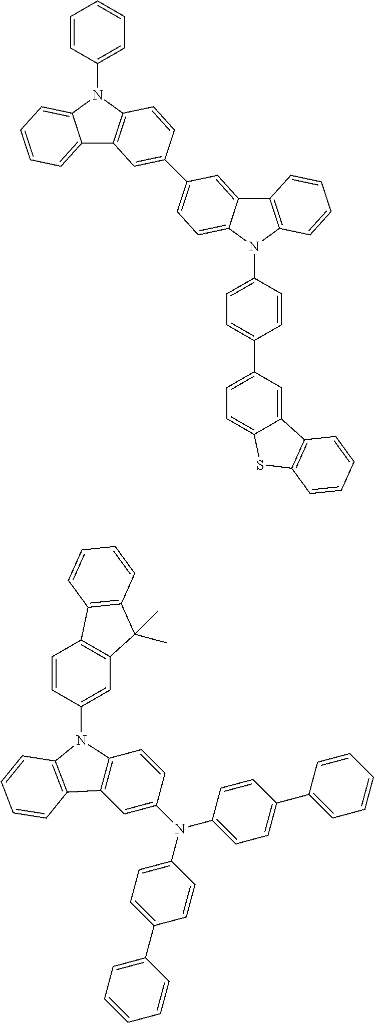 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

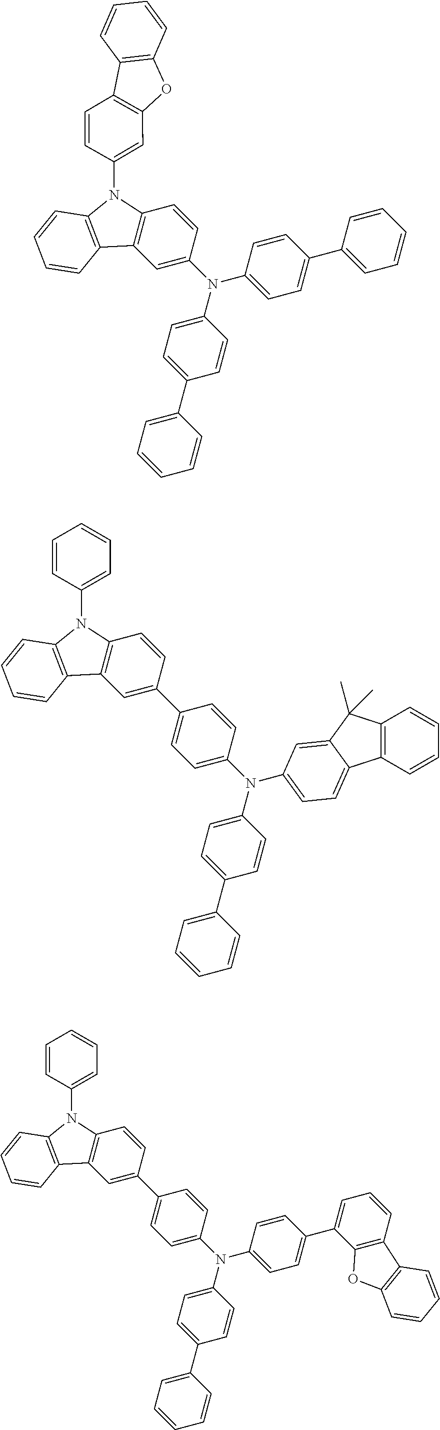 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

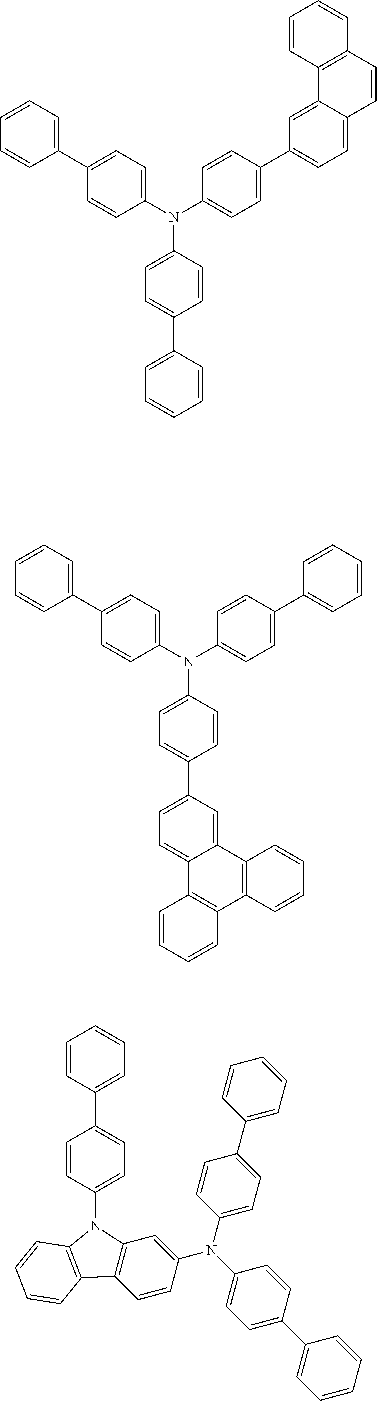 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

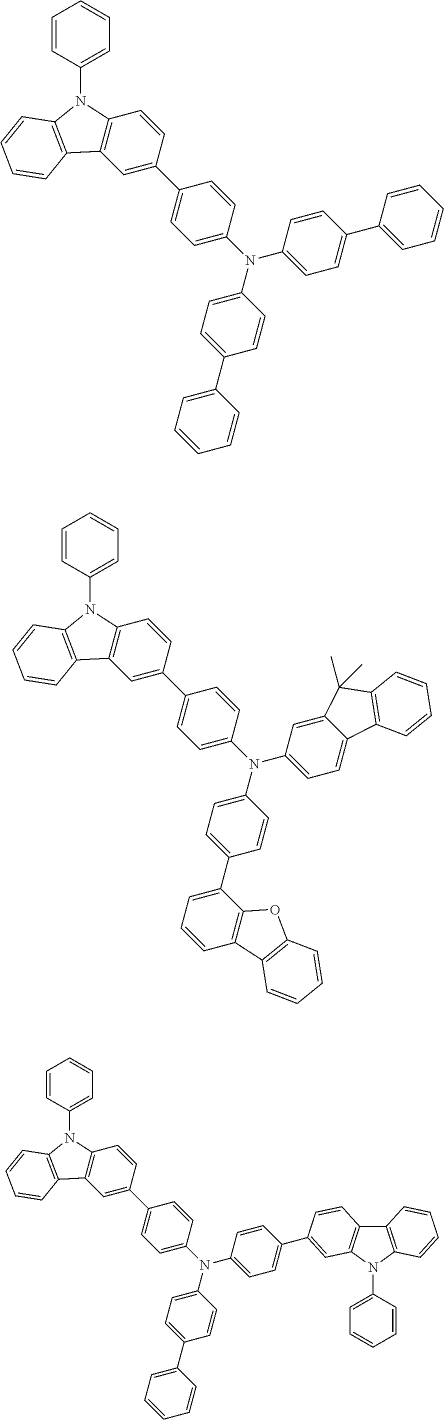 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

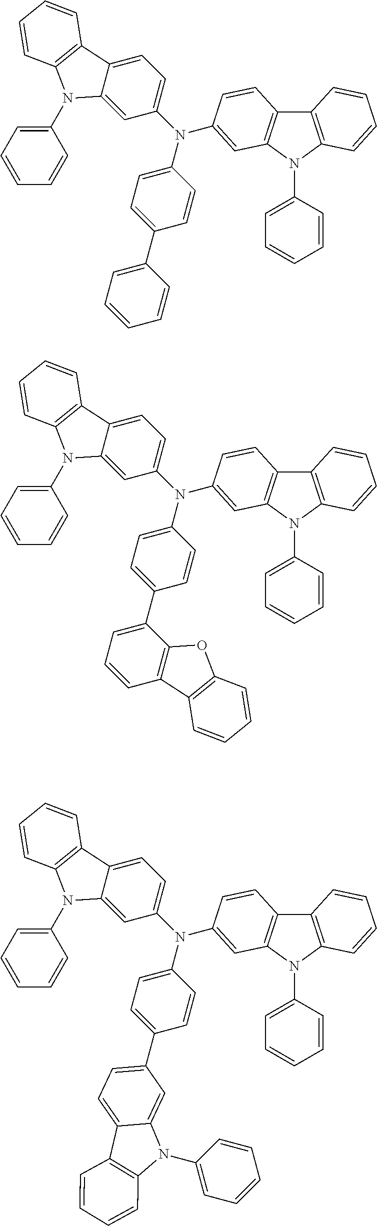 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

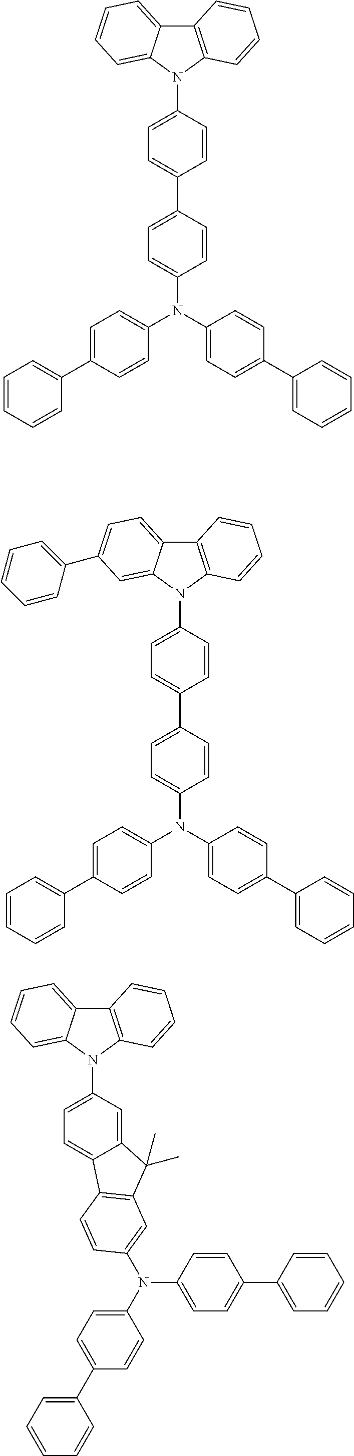

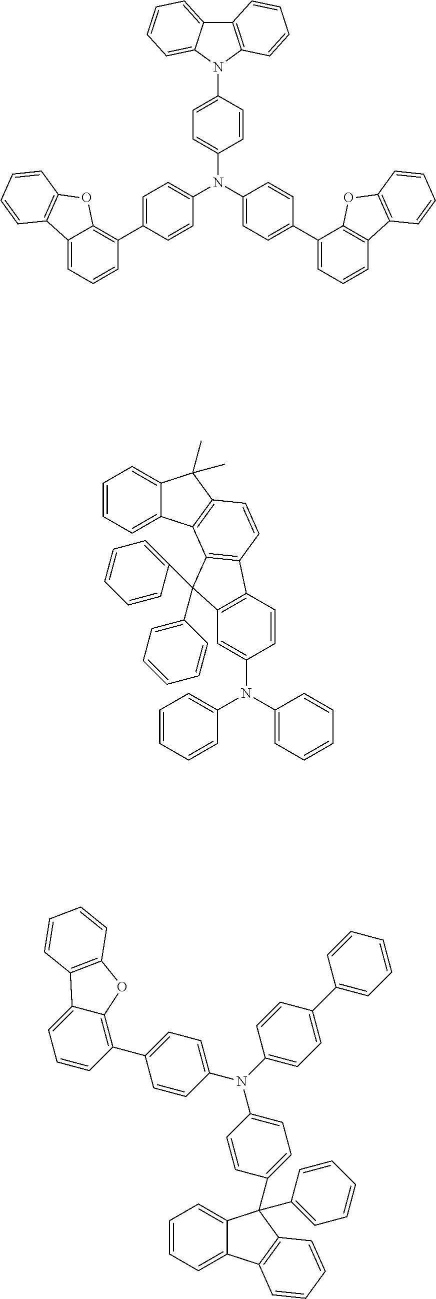 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

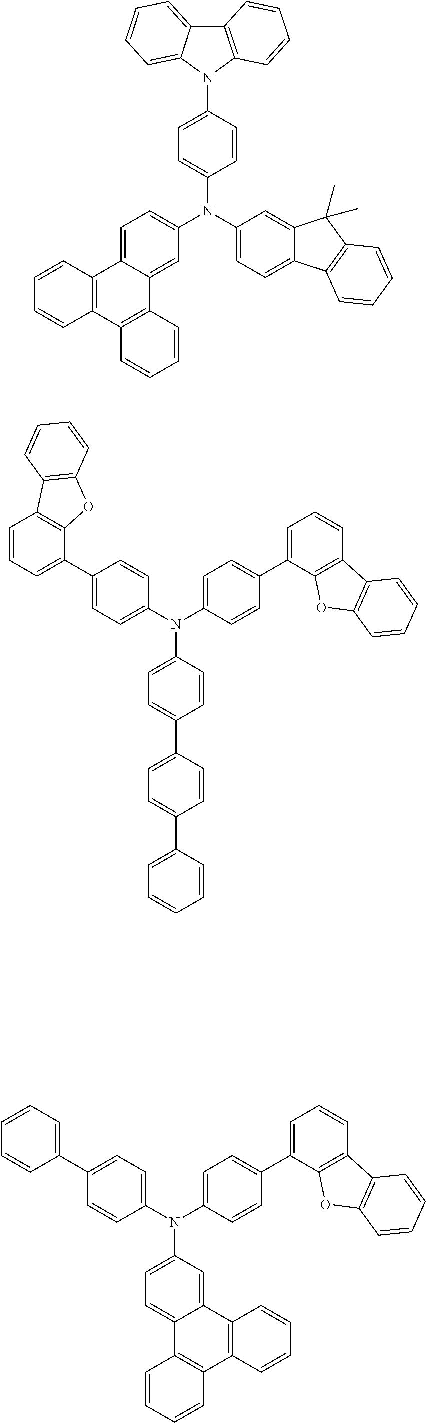 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

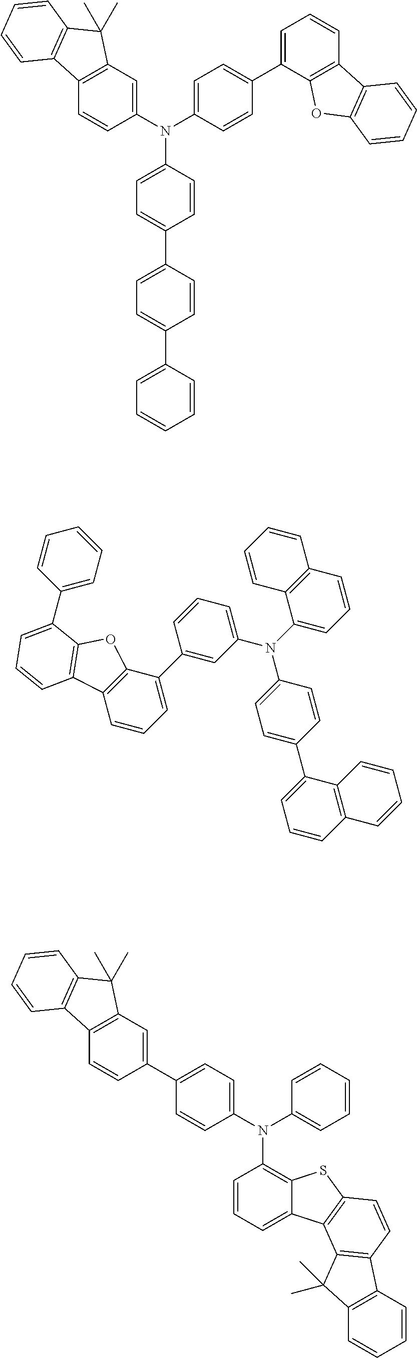 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

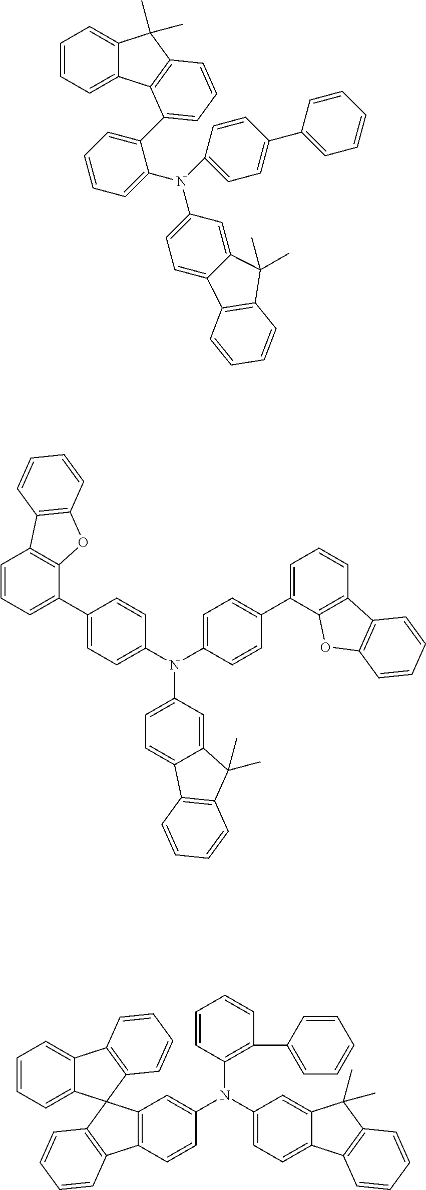 [SMILES: CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

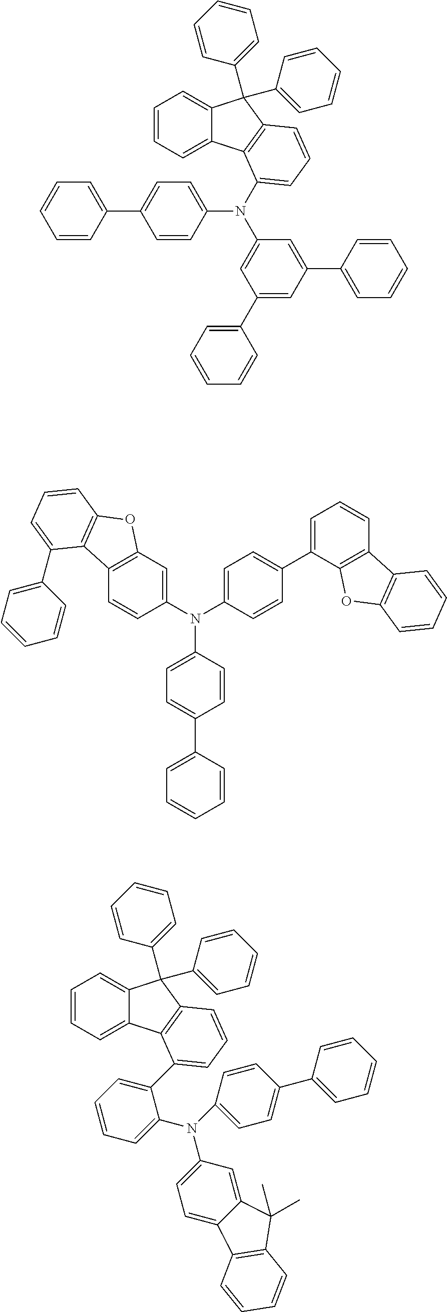 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

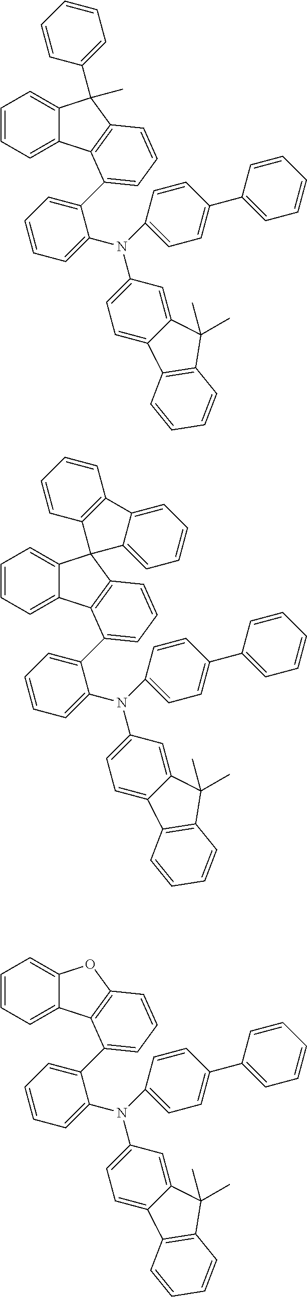 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

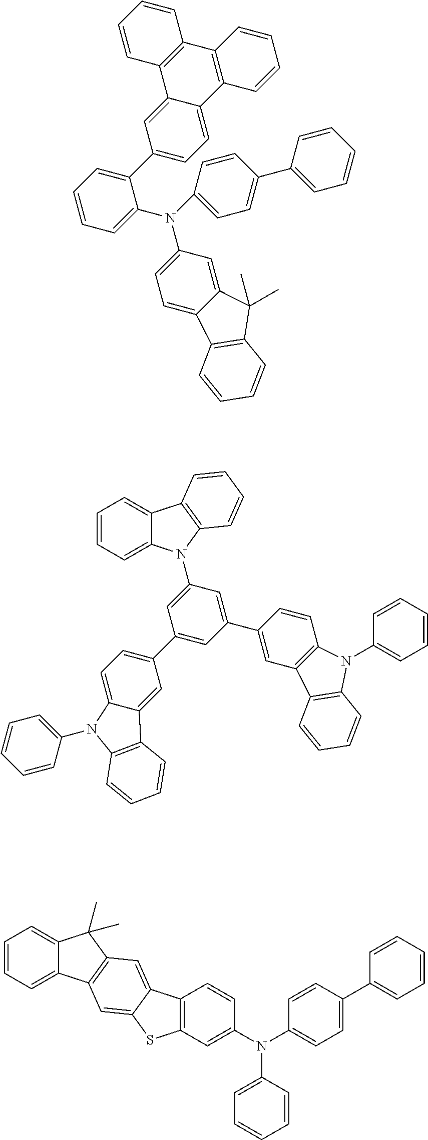 [SMILES: CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21]

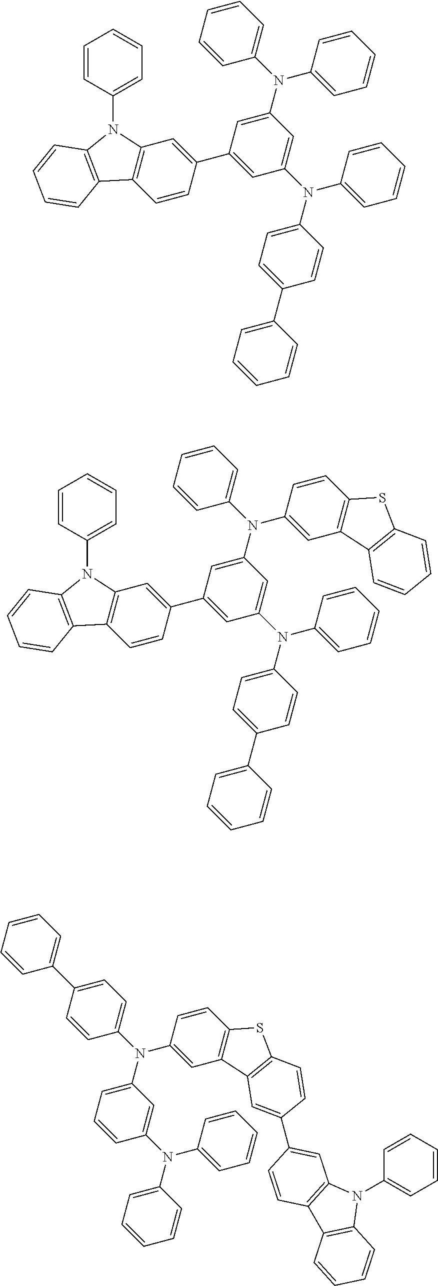 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

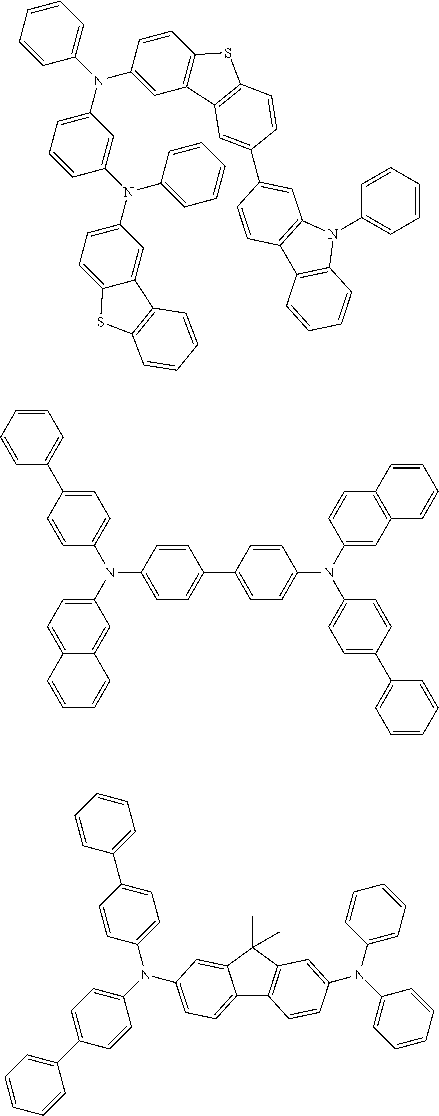 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

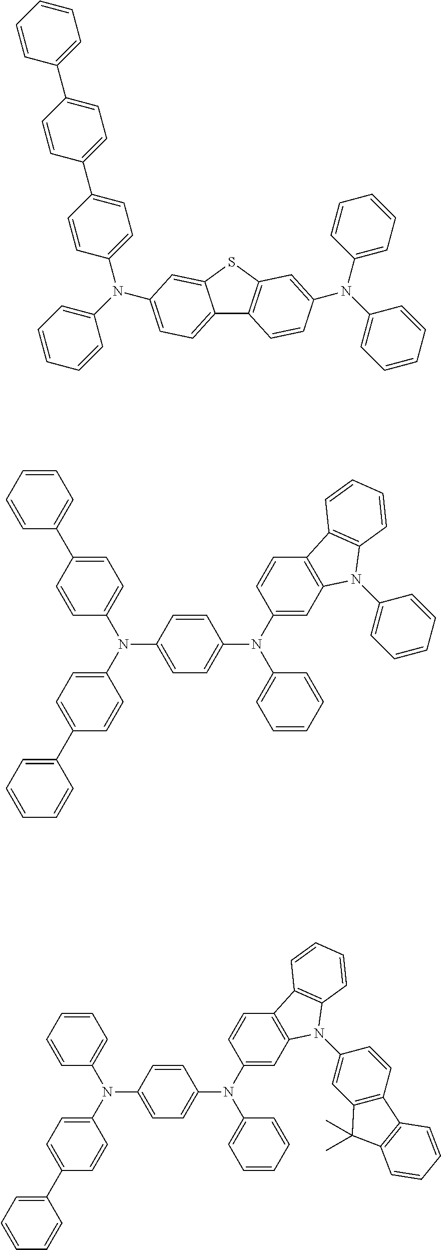 [SMILES: c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

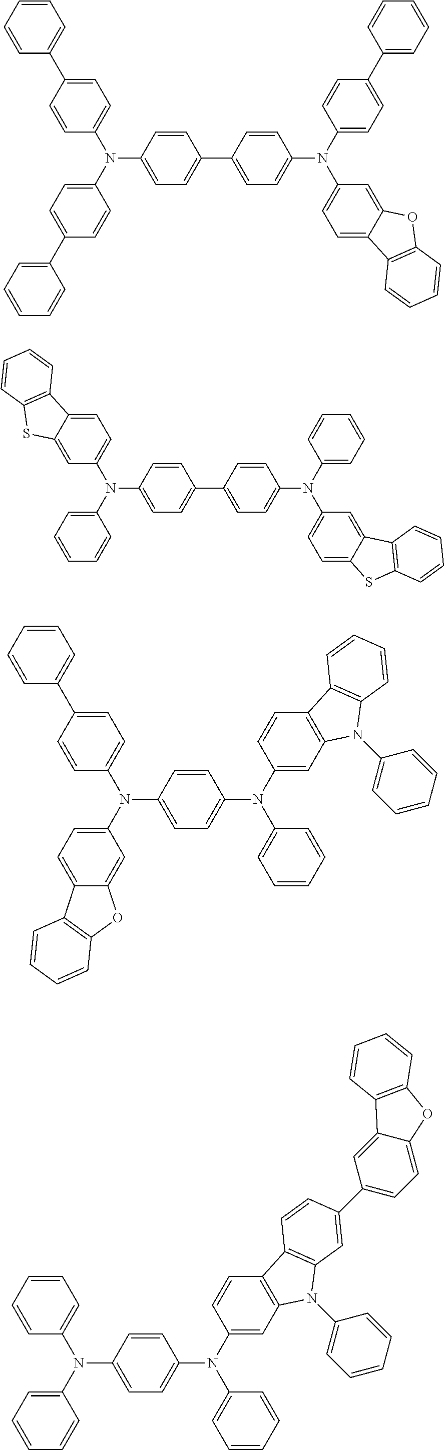

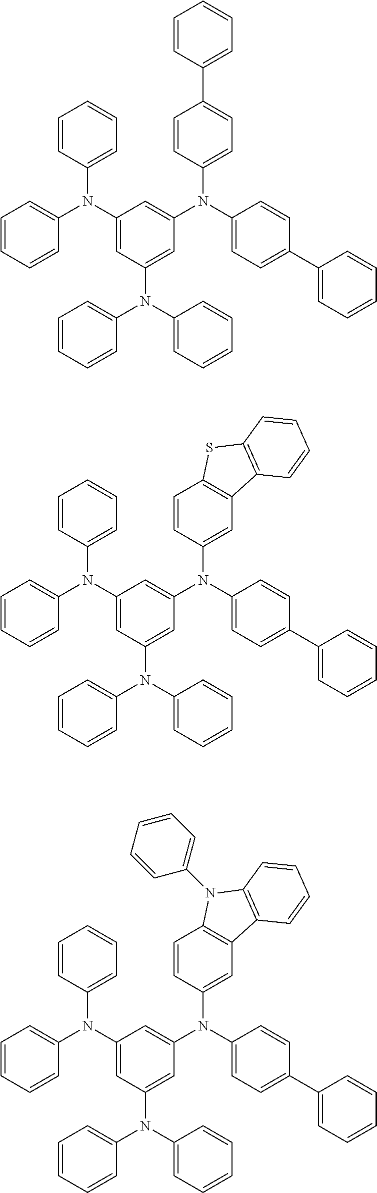 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

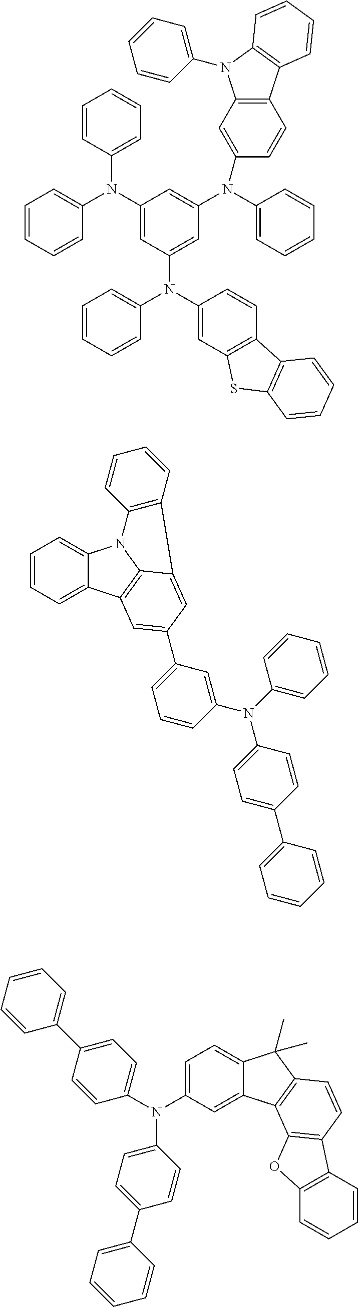 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

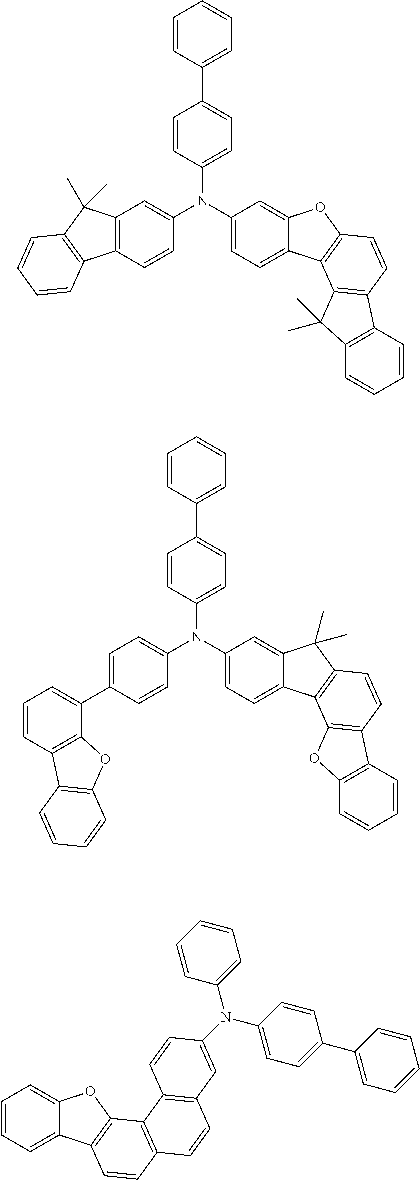 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1]

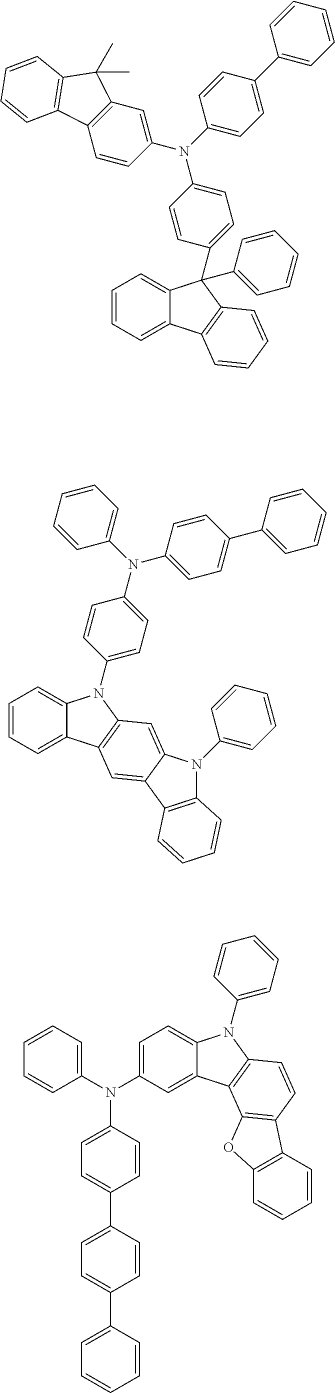 [SMILES: CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1 ; c1ccc(-c2ccc(-c3ccc(N(c4ccccc4)c4ccc5c(c4)c4c6oc7ccccc7c6ccc4n5-c4ccccc4)cc3)cc2)cc1]

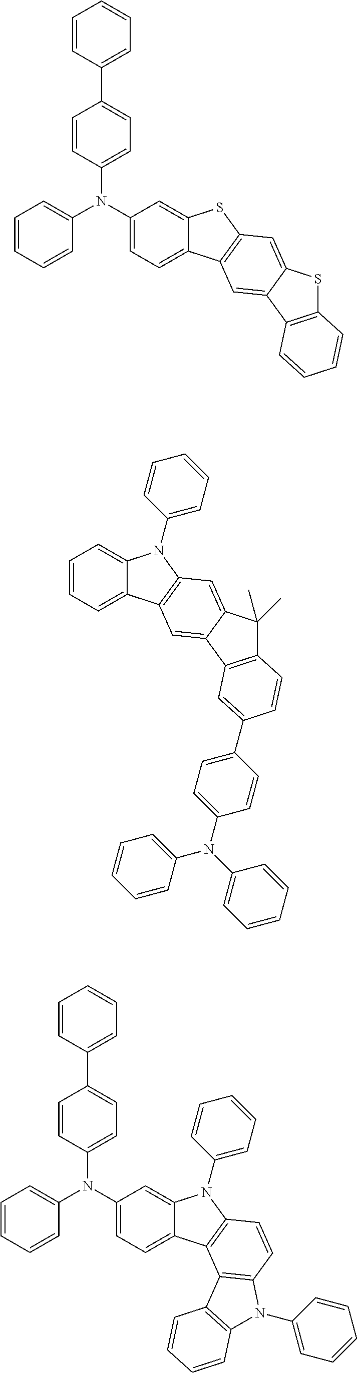 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

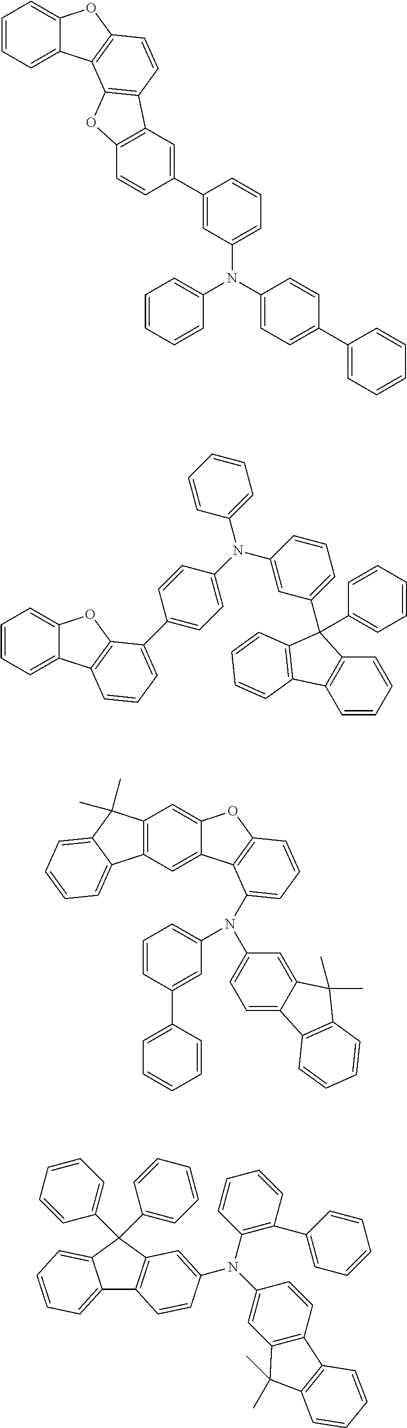 [SMILES: c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21]

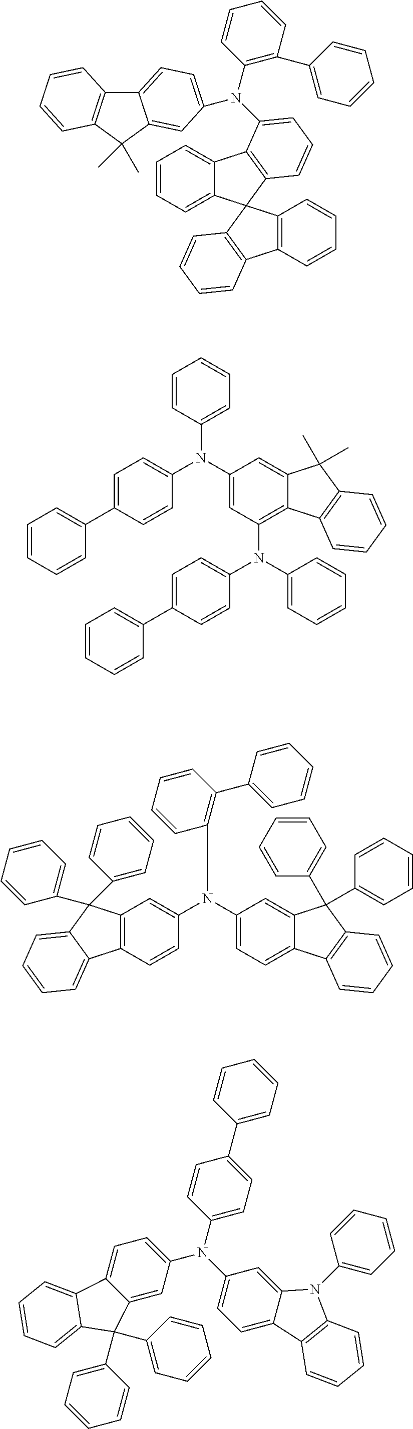 [SMILES: CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

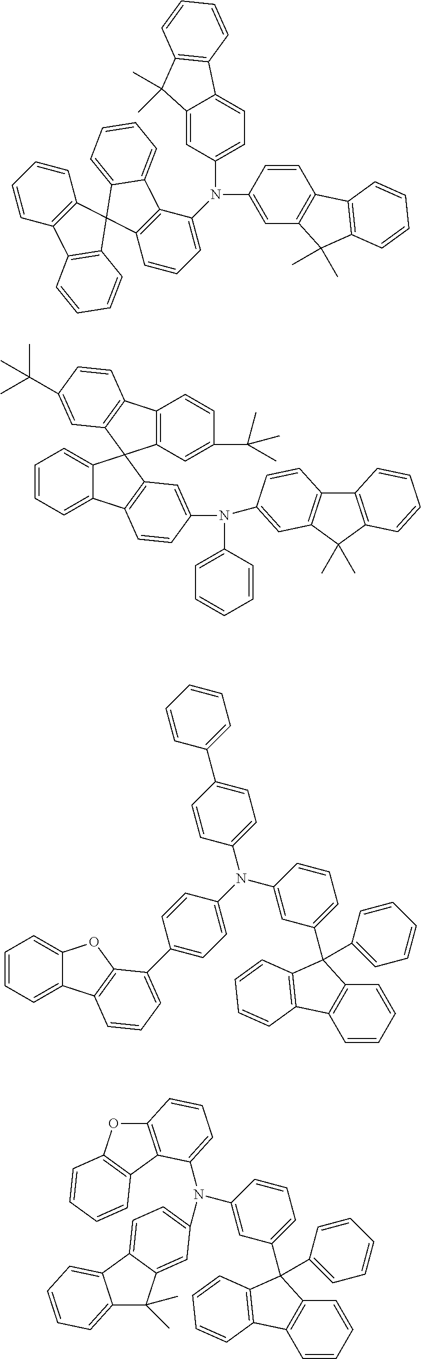 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21]

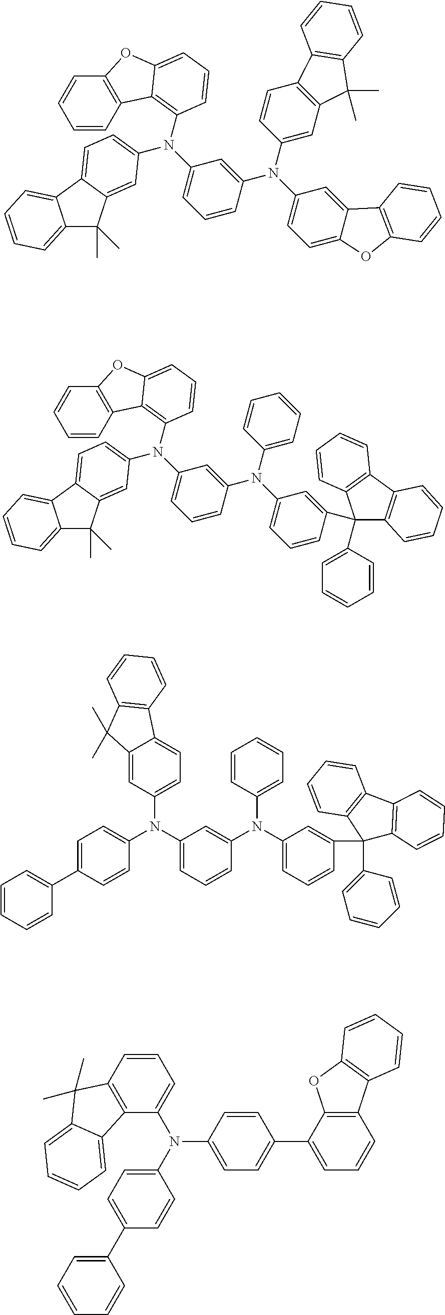 [SMILES: CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21]

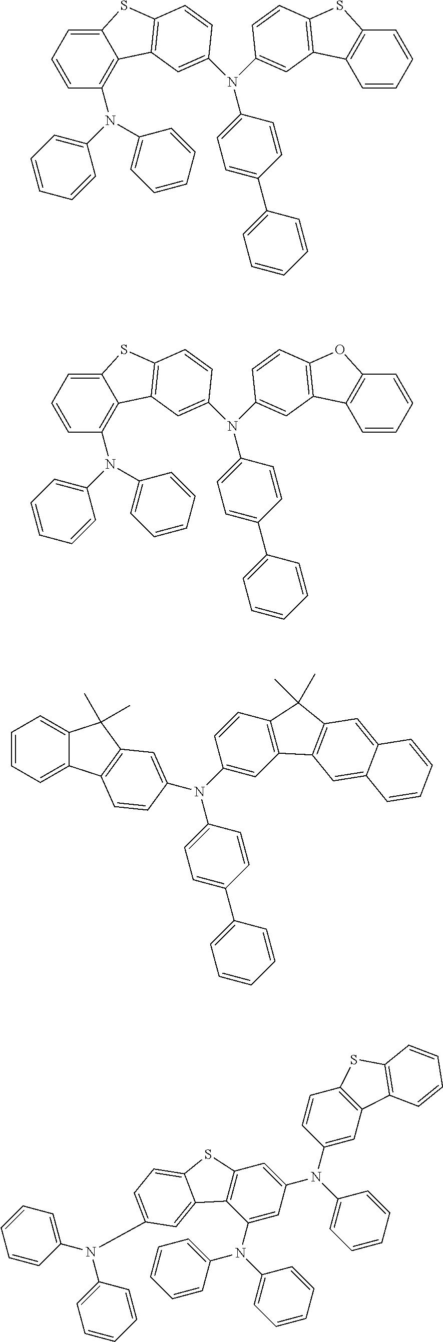 [SMILES: c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1 ; CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1]

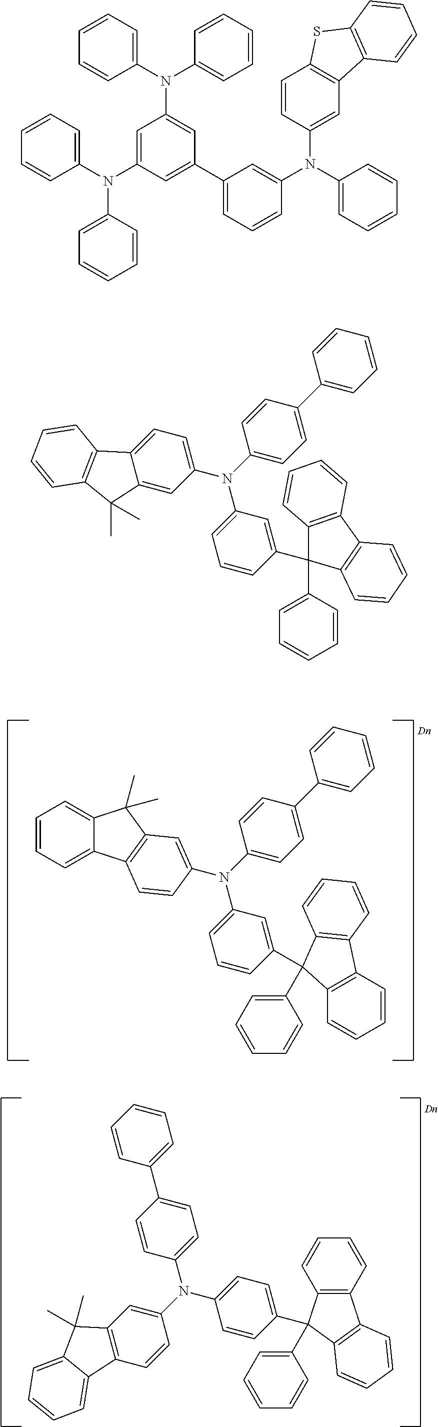

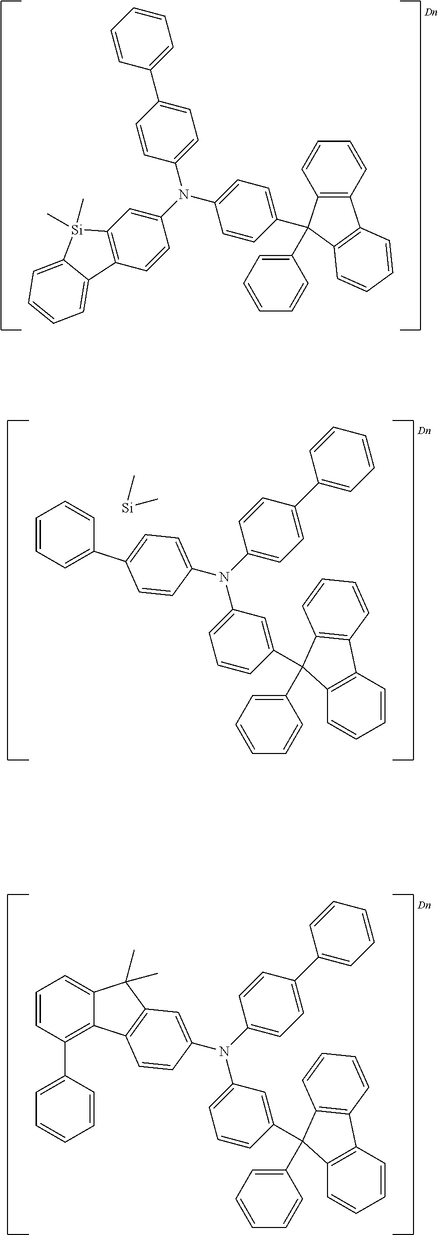

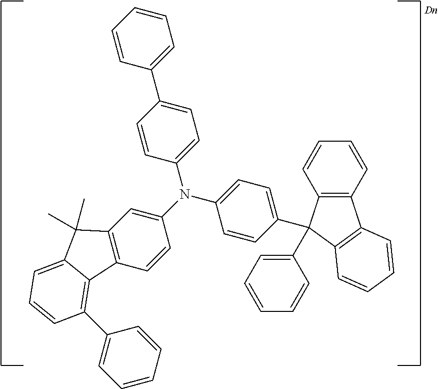

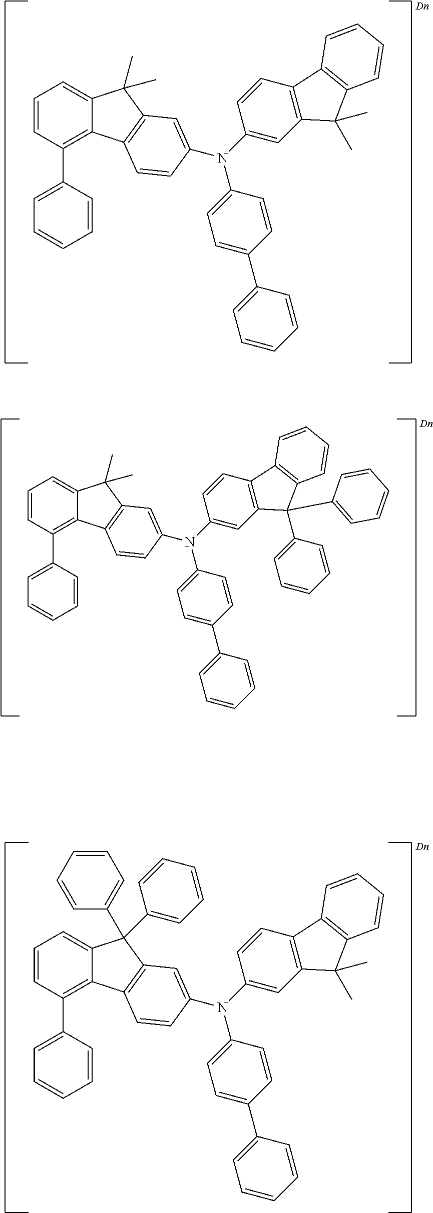

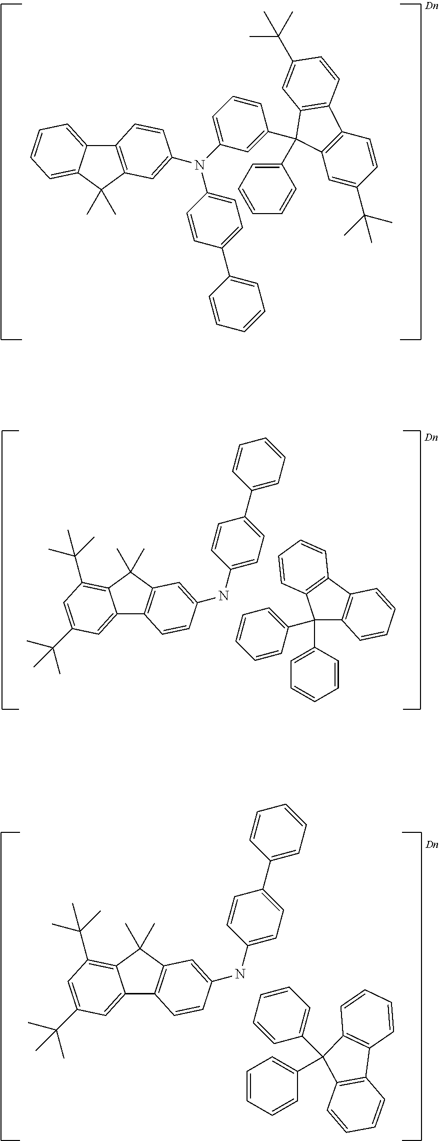

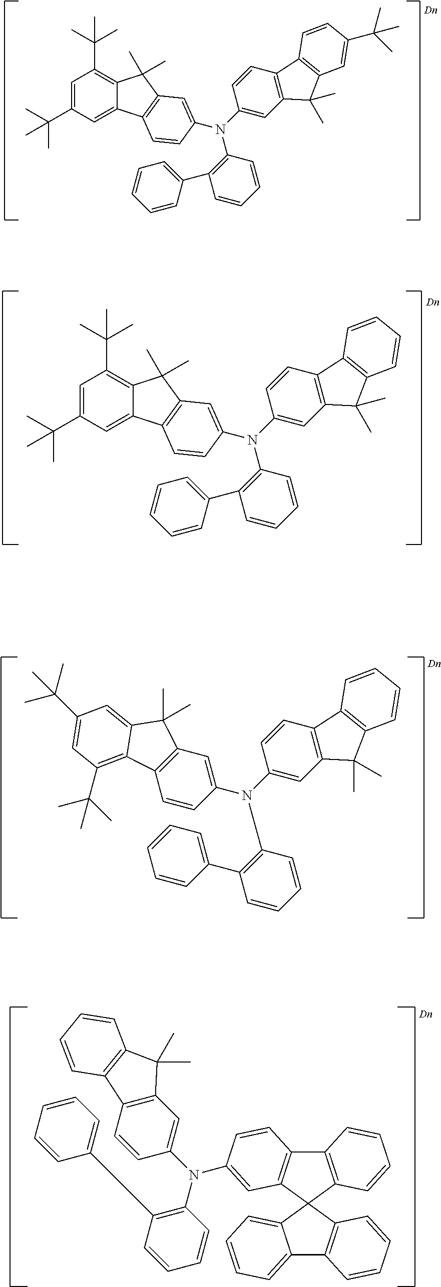

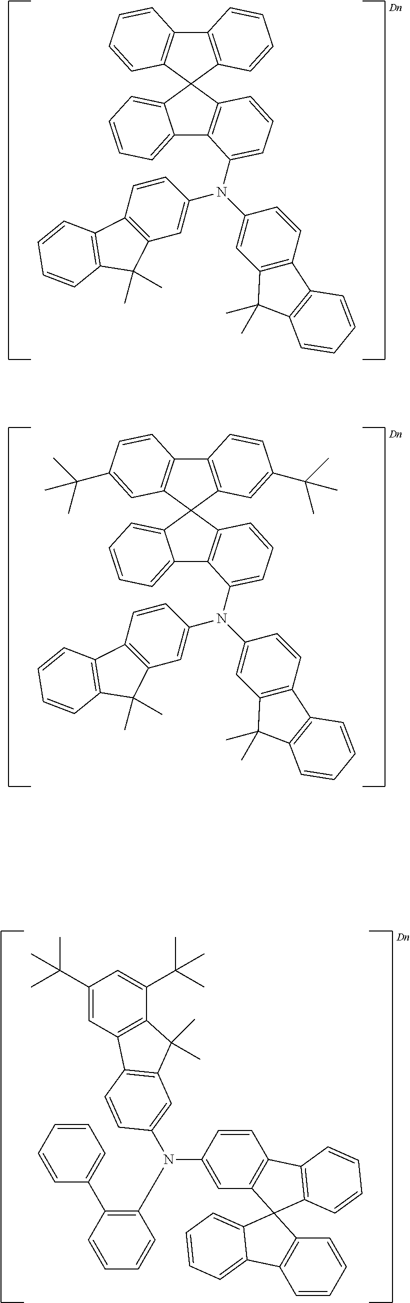

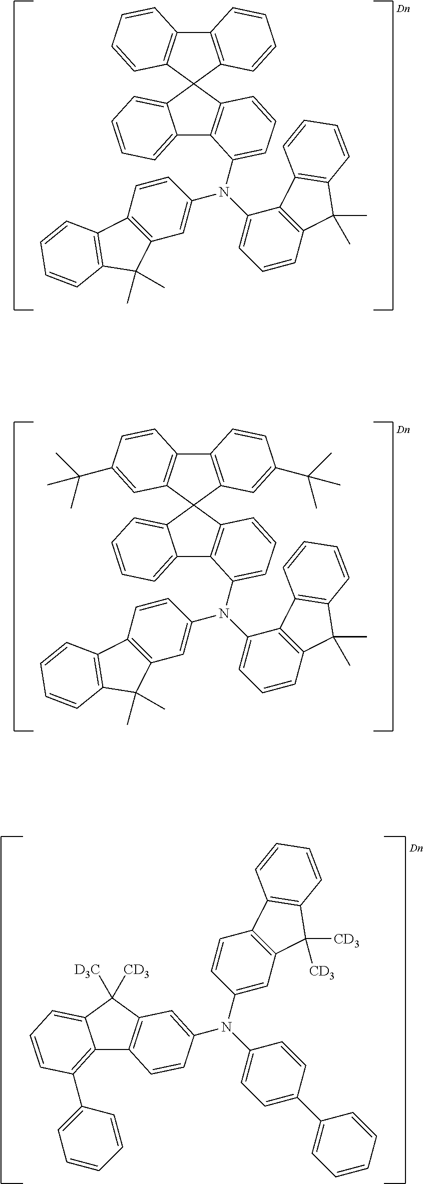

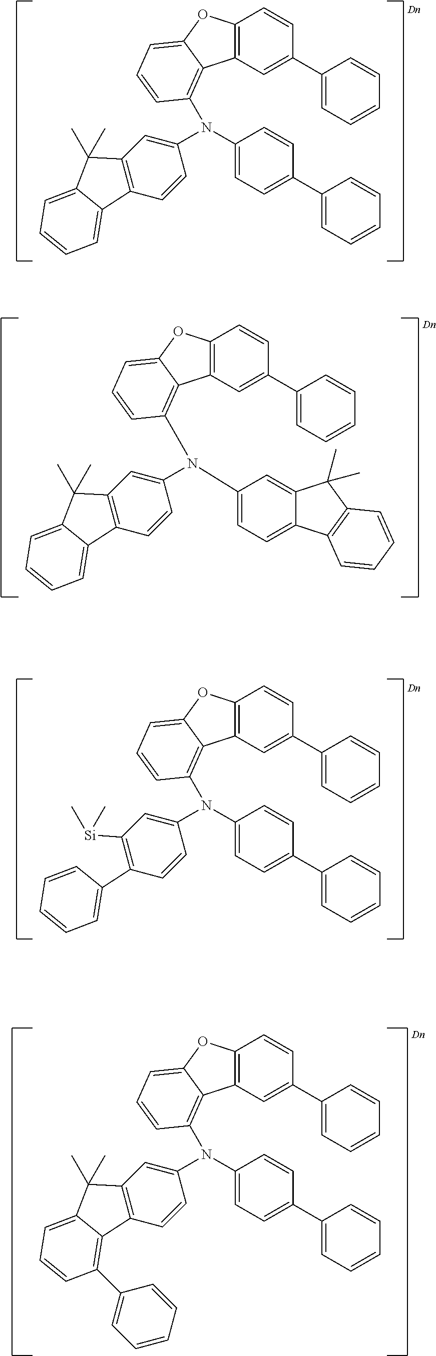

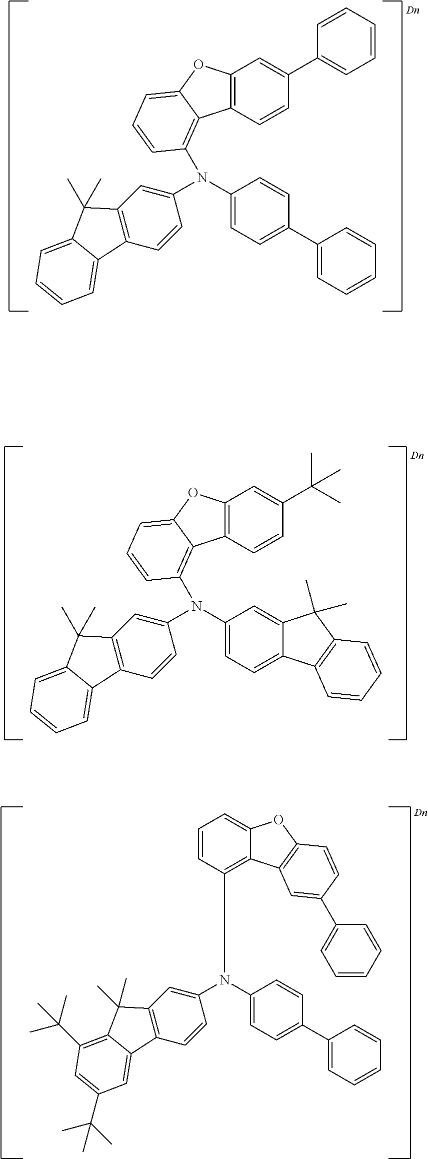

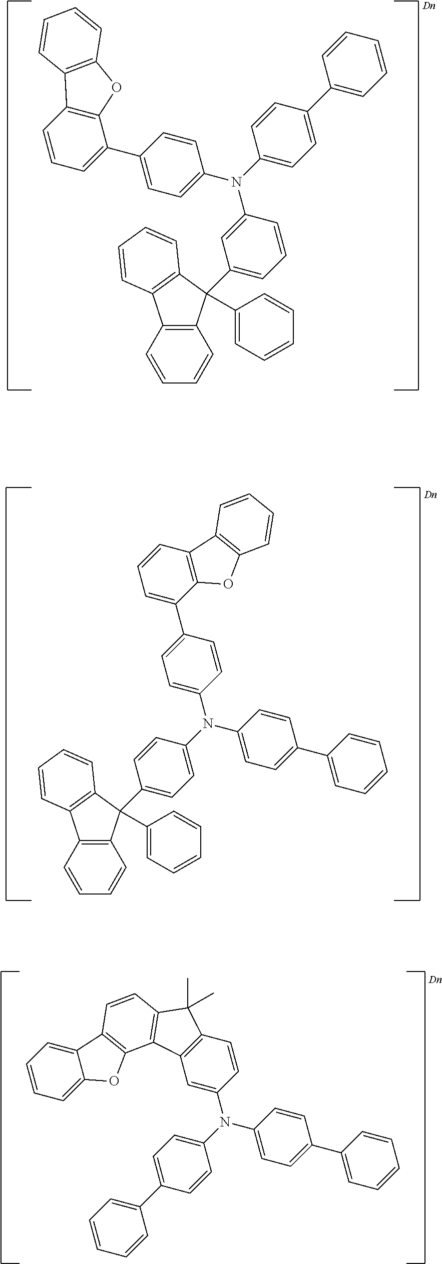

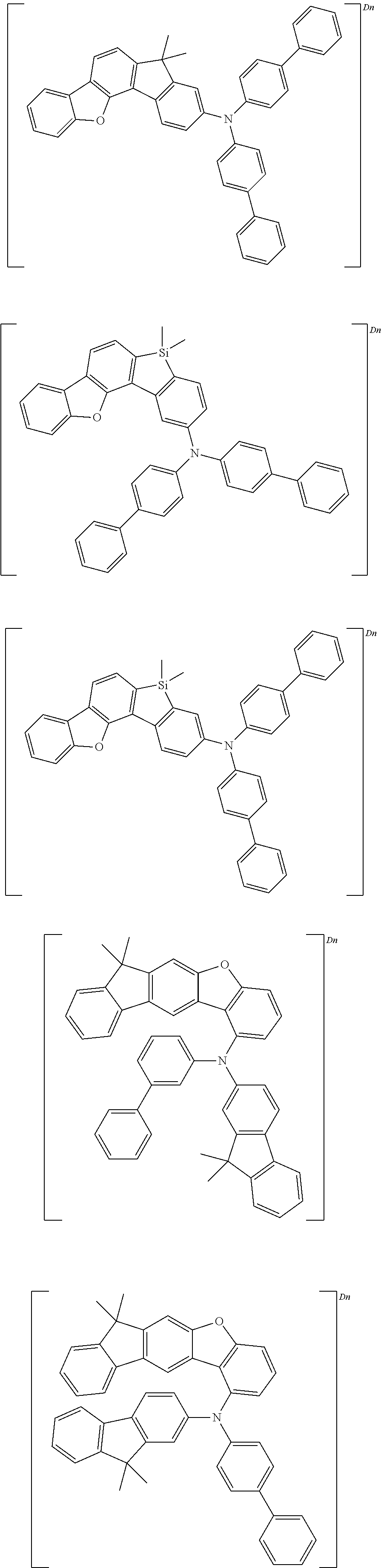

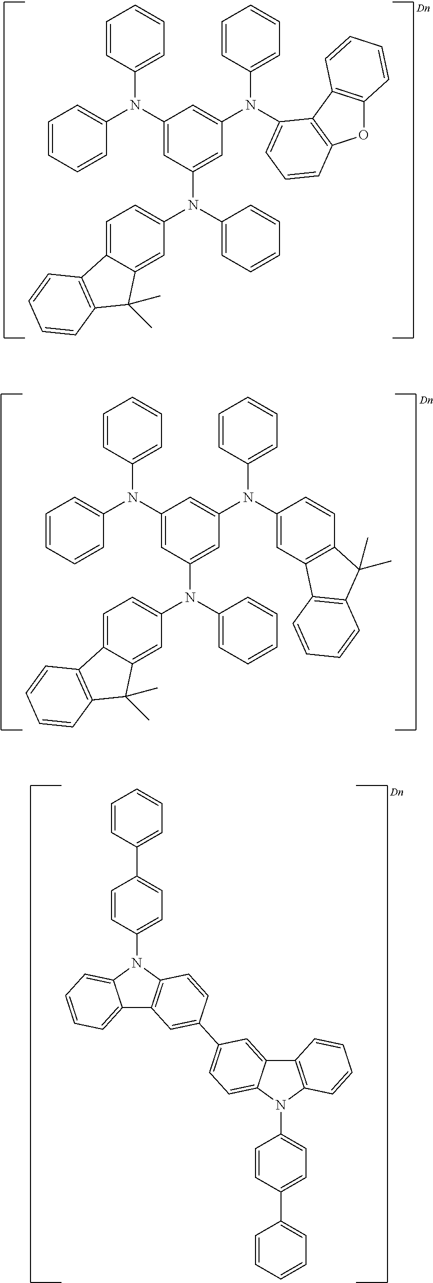

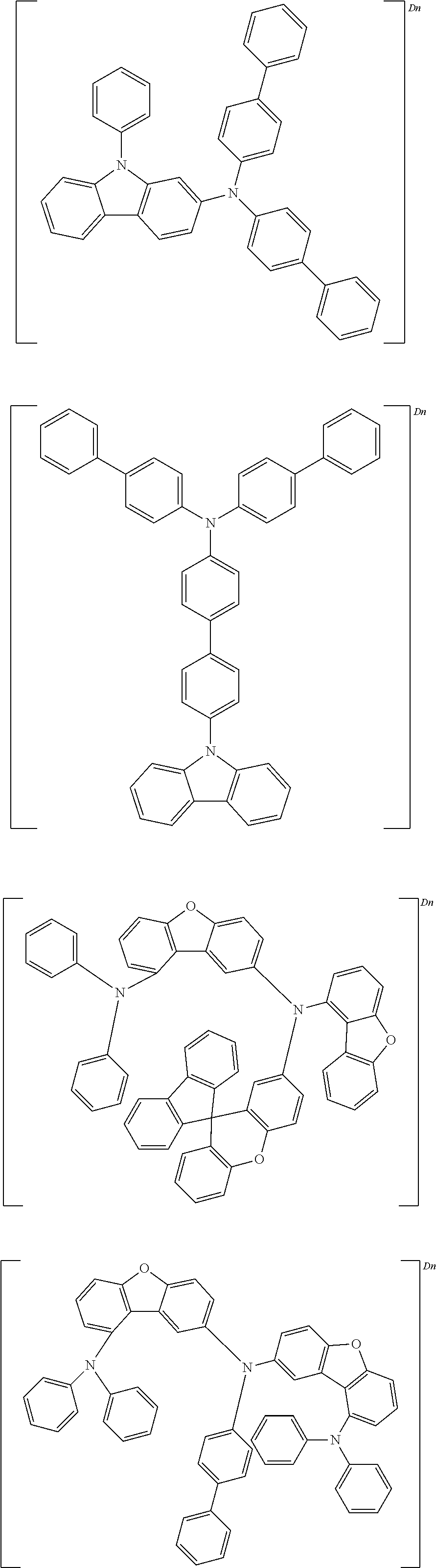

(Dn refers to the number of hydrogens replaced with deuterium and indicates a structure substituted with one or more deuterium atoms) However, as noted above, any hydrogen in any compound may be protium, deuterium, or tritium, based on natural or artificial substitution.

In the hole transport region, in addition to the compounds described above, other suitable compounds may also be used.

In an implementation, the charge transport region may be, e.g., the electron transport region 150.

The electron transport region 150 may help further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group D may be included in at least one of the electron transport layer and the electron transport auxiliary layer.

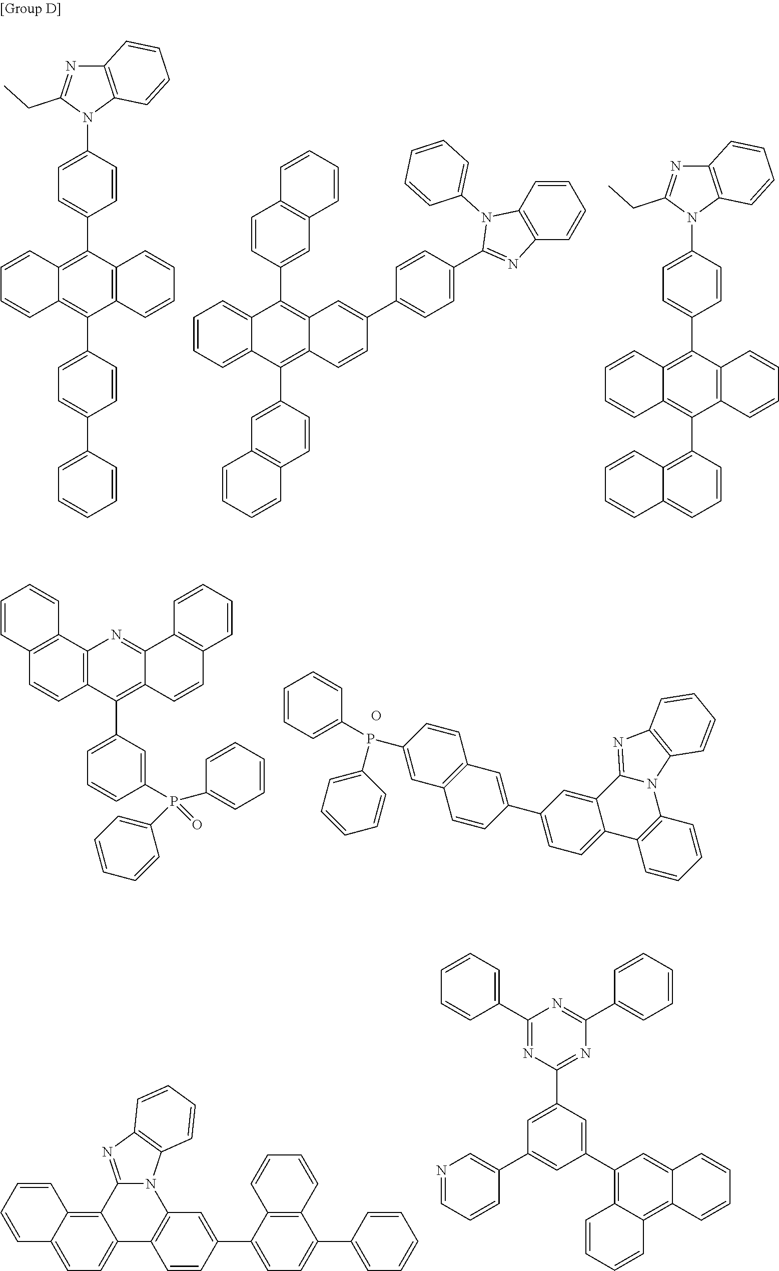

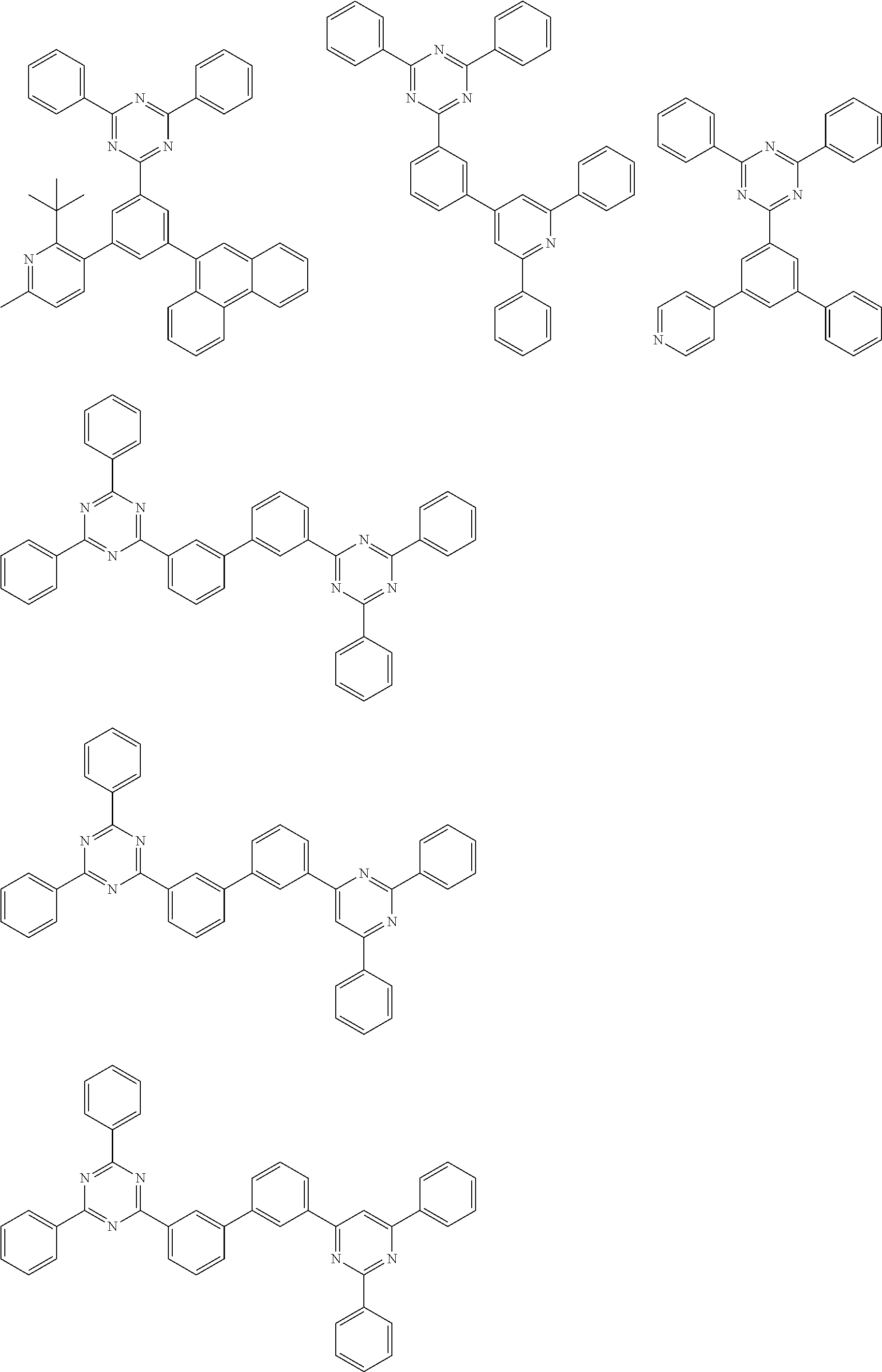

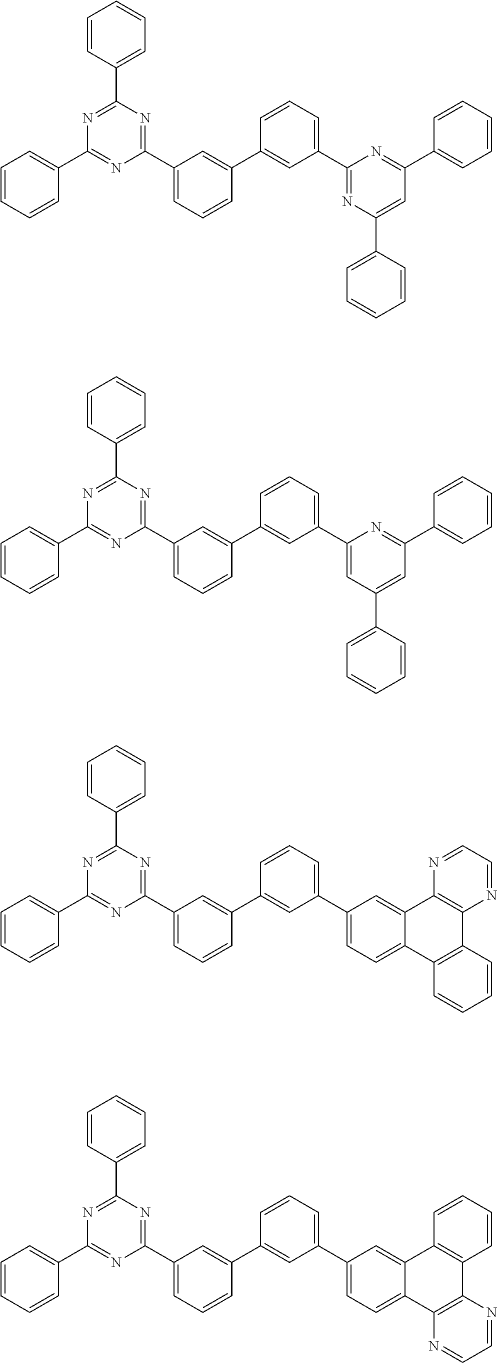 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

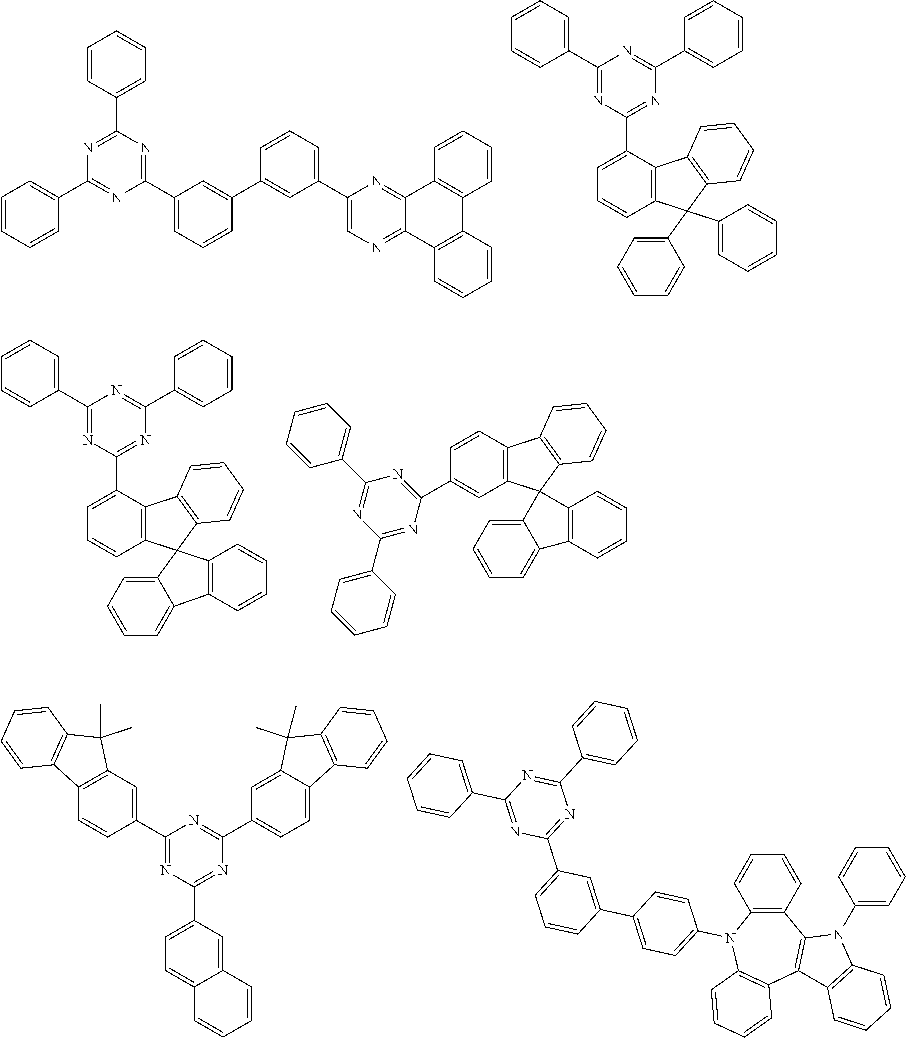

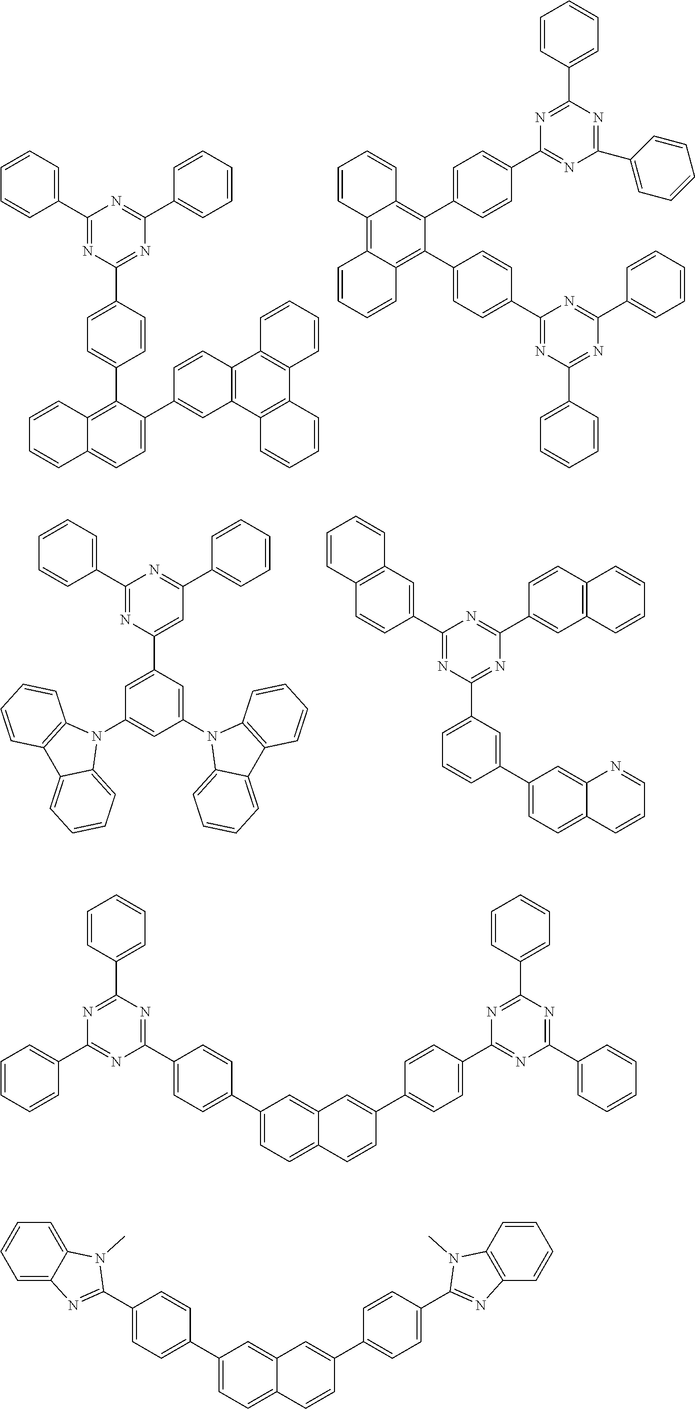

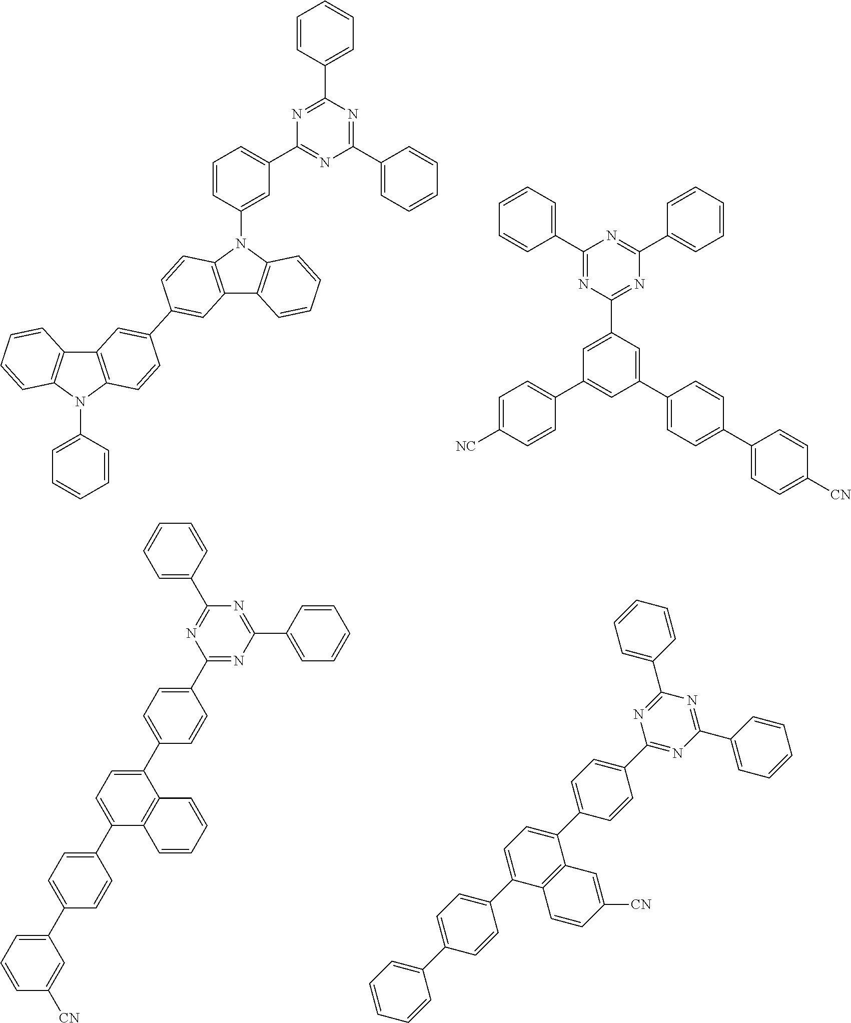 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

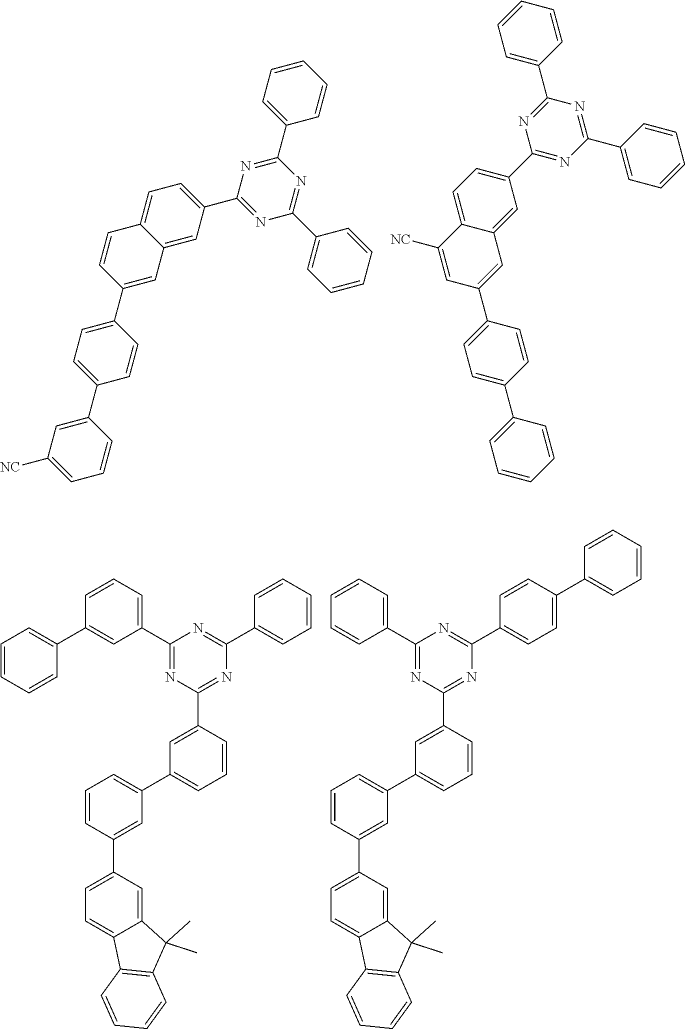

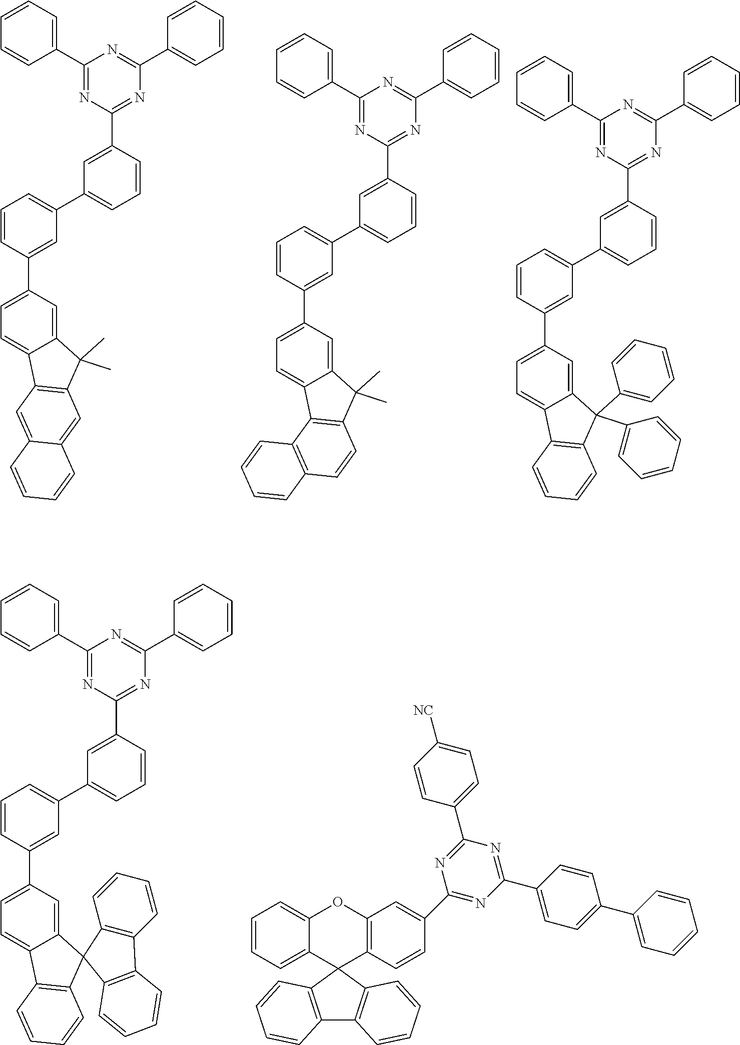

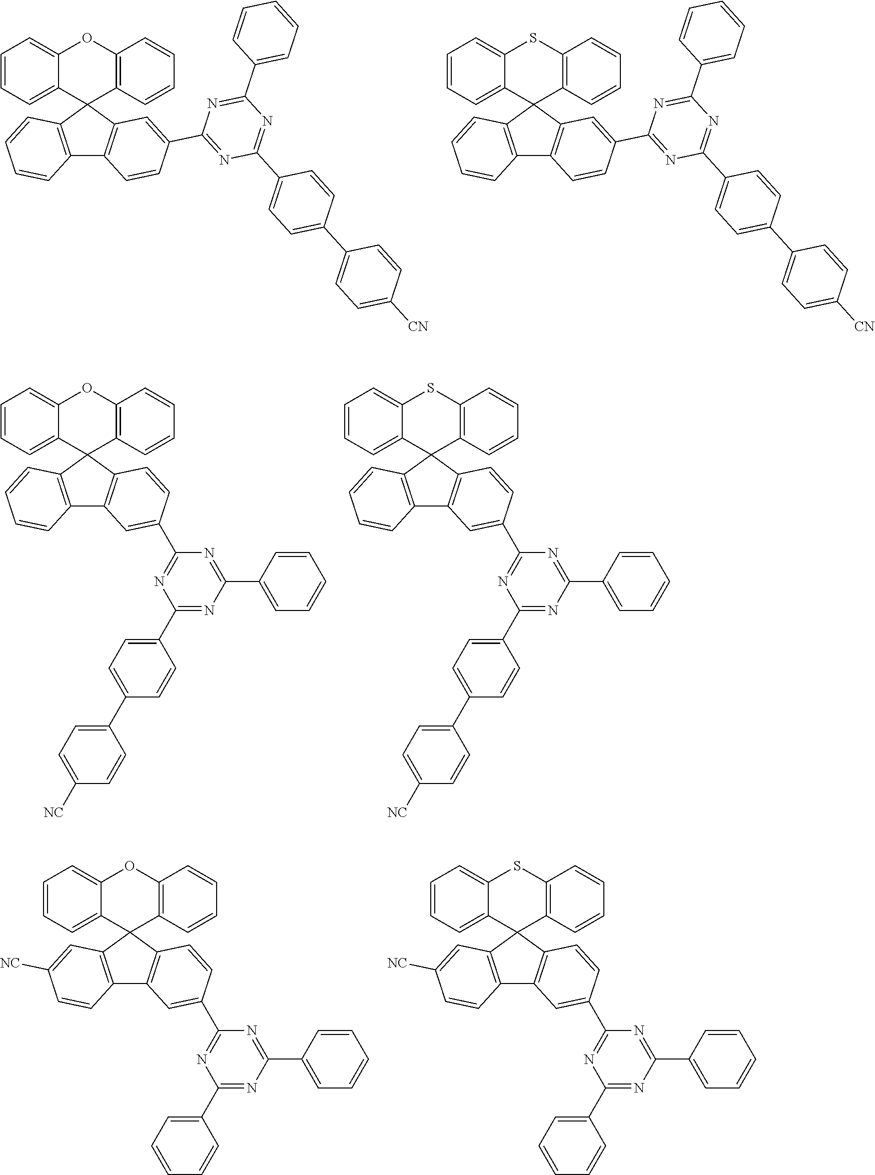

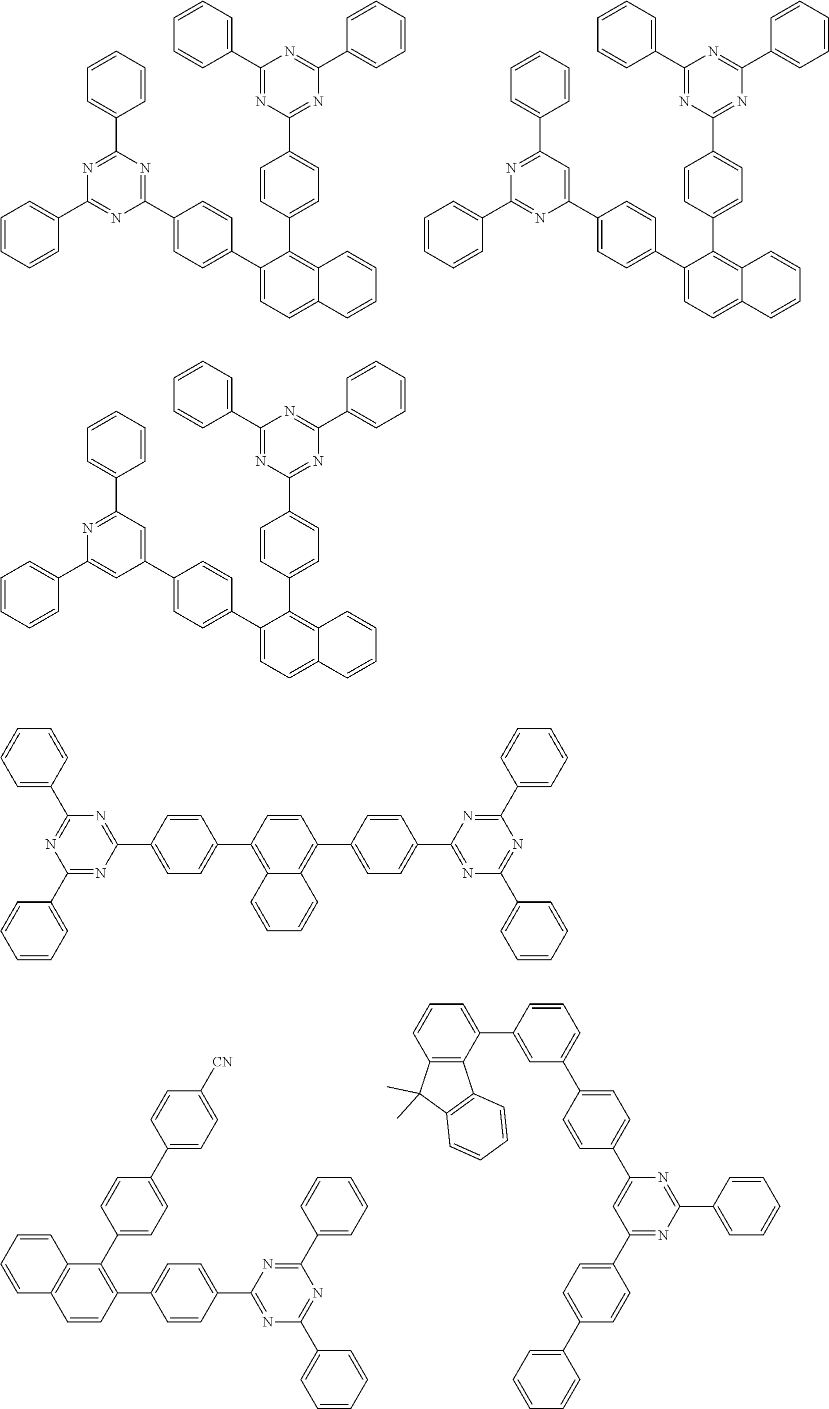

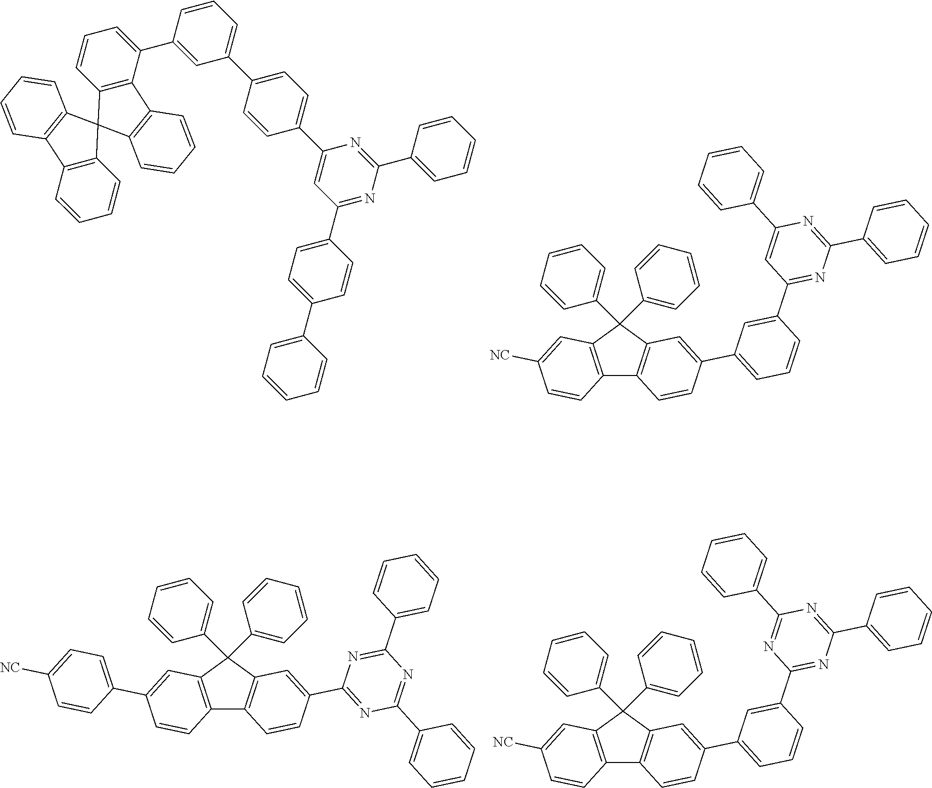

Some example embodiments may provide an organic light emitting diode including the light emitting layer as the organic layer.

Some example embodiments may provide an organic light emitting diode including a light emitting layer and a hole transport region as the organic layer.

Some example embodiments may provide an organic light emitting diode including a light emitting layer and an electron transport region as the organic layer.

Some example embodiments may provide an organic light emitting diode including a hole transport region 140 and an electron transport region 150 in addition to the light emitting layer 130 as the organic layer 105, as shown in the FIGURE.

In an implementation, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The aforementioned organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### (Preparation of Compound for Organic Optoelectronic Device)

### Synthesis Example 1: Synthesis of Compound 1-4

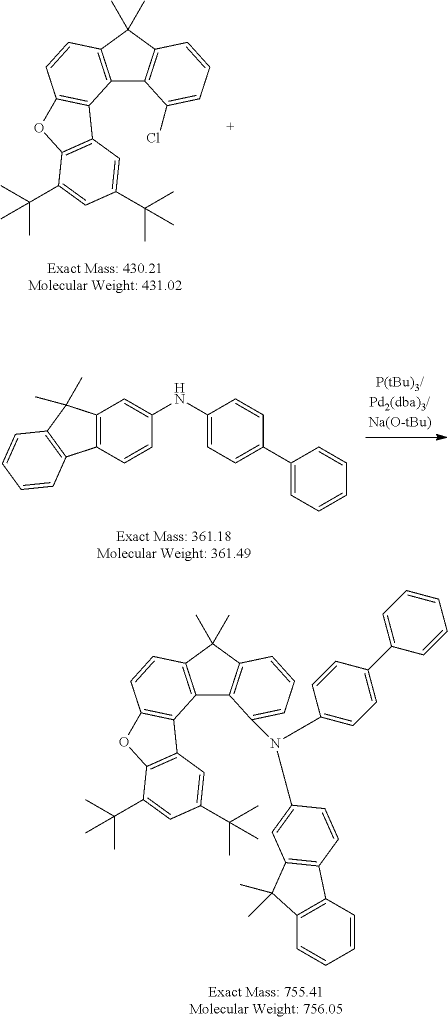 [SMILES: CC(C)(C)c1cc(C(C)(C)C)c2oc3ccc4c(c3c2c1)-c1c(Cl)cccc1C4(C)C ; CC1(C)c2ccccc2-c2ccc(Nc3ccc(-c4ccccc4)cc3)cc21 ; CC(C)(C)c1cc(C(C)(C)C)c2oc3ccc4c(c3c2c1)-c1c(N(c2ccc(-c3ccccc3)cc2)c2ccc3c(c2)C(C)(C)c2ccccc2-3)cccc1C4(C)C]

10.0 g (23.20 mmol) of 2,4-di-tert-butyl-12-chloro-8,8-dimethyl-8H-fluoreno[3,4-b]benzofuran, 10.06 g (27.84 mmol) of N-([1,1′-biphenyl]-4-yl)-9,9-dimethyl-9H-fluoren-2-amine, 4.46 g (46.40 mmol) of sodium t-butoxide, and 0.56 g (1.39 mmol) of tri-tert-butylphosphine were dissolved in 232 ml of toluene, and 0.64 g (0.696 mmol) of Pd₂(dba)₃ was added thereto and then, stirred under reflux under a nitrogen atmosphere for 12 hours. After a reaction was completed, an organic layer, was extracted with ethyl acetate and distilled water, was dried with anhydrous magnesium sulfate and filtered, and a filtrate therefrom was concentrated under a reduced pressure. A product therefrom was purified with n-hexane/dichloromethane (in a volume ratio of 3:1) through silica gel column chromatography to obtain 14.2 g (a yield: 81%) of Compound 1-4 as a white solid.
calculation value: C, 88.96; H, 7.07; N, 1.85; O, 2.12
analysis value: C, 88.96; H, 7.07; N, 1.85; O, 2.12

### Synthesis Example 2: Synthesis of Compound 1-89

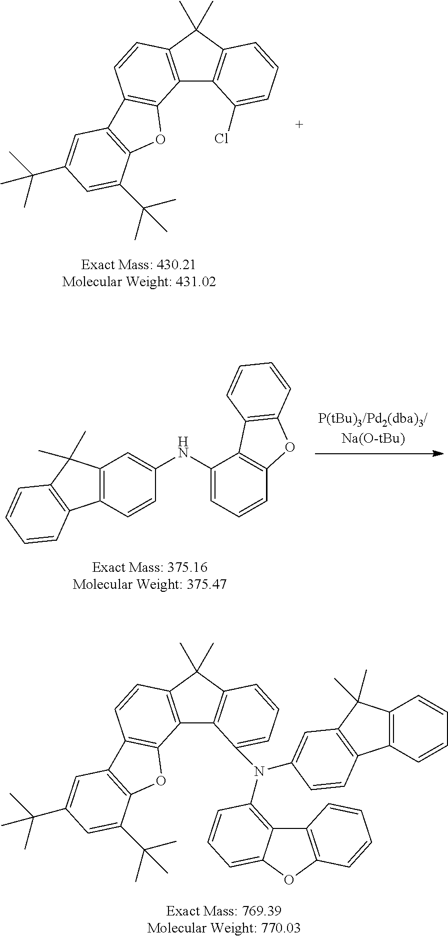 [SMILES: CC(C)(C)c1cc(C(C)(C)C)c2oc3c4c(ccc3c2c1)C(C)(C)c1cccc(Cl)c1-4 ; CC1(C)c2ccccc2-c2ccc(Nc3cccc4oc5ccccc5c34)cc21 ; CC(C)(C)c1cc(C(C)(C)C)c2oc3c4c(ccc3c2c1)C(C)(C)c1cccc(N(c2ccc3c(c2)C(C)(C)c2ccccc2-3)c2cccc3oc5ccccc5c23)c1-4]

Compound 1-89 (17.3 g, a yield of 81%) was synthesized in the same manner as in Synthesis Example 1, except that 12.0 g of 1,3-di-tert-butyl-11-chloro-7,7-dimethyl-7H-fluoreno[4,3-b]benzofuran and 12.54 g of N-(9,9-dimethyl-9H-fluoren-2-yl)dibenzo[b,d]furan-1-amine were used in an equivalent ratio of 1:1.2.
calculation value: C, 87.35; H, 6.68; N, 1.82; O, 4.16
analysis value: C, 87.35; H, 6.68; N, 1.82; 0, 4.16

### Synthesis Example 3: Synthesis of Compound 1-91

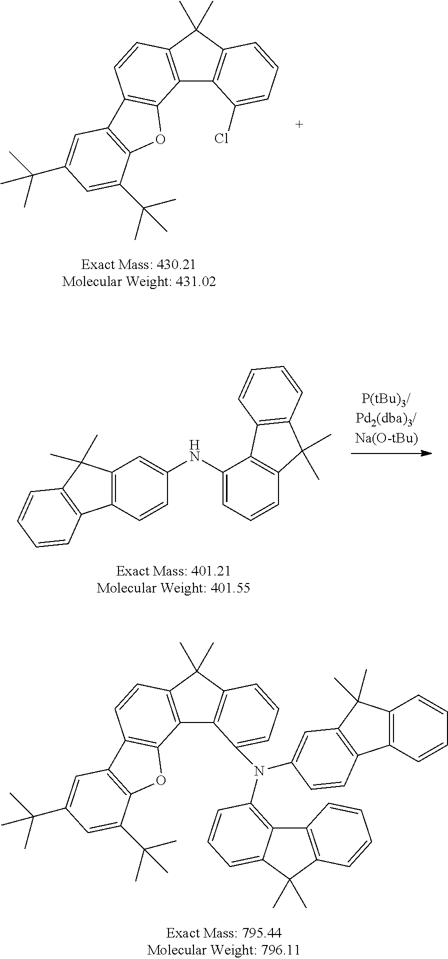

Compound 1-91 (14.0 g, a yield of 84%) was synthesized in the same manner as in Synthesis Example 1, except that 9.0 g of 1,3-di-tert-butyl-11-chloro-7,7-dimethyl-7H-fluoreno[4,3-b]benzofuran and 10.06 g of N-(9,9-dimethyl-9H-fluoren-4-yl)-9,9-dimethyl-9H-fluoren-2-amine were used in an equivalent ratio of 1:1.2.
calculation value: C, 89.01; H, 7.22; N, 1.76; O, 2.01
analysis value: C, 89.01; H, 7.22; N, 1.76; O, 2.01

### Synthesis Example 4: Synthesis of Compound 1-114

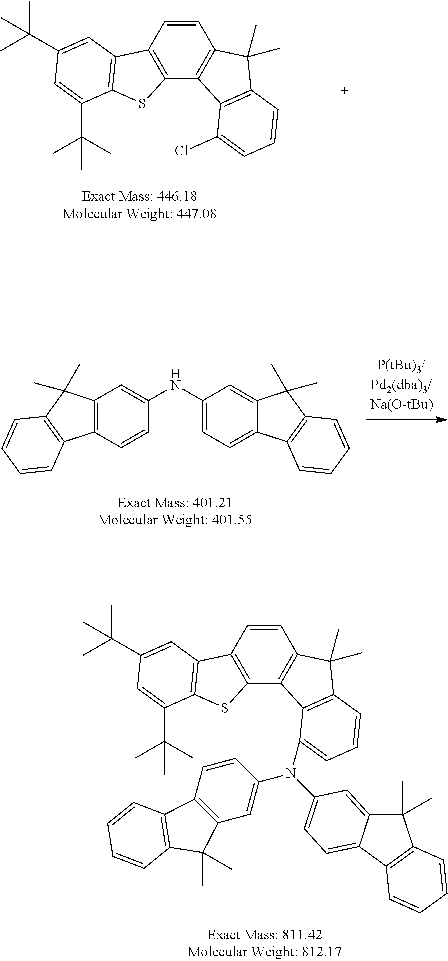

Compound 1-114 (14.9 g, a yield of 82%) was synthesized in the same manner as in Synthesis Example 1, except that 10.0 g of 1,3-di-tert-butyl-11-chloro-7,7-dimethyl-7H-benzo[b]fluoreno[3,4-d]thiophene and 10.78 g of bis(9,9-dimethyl-9H-fluoren-2-yl)amine were used in an equivalent ratio of 1:1.2.
calculation value: C, 87.25; H, 7.07; N, 1.72; S, 3.95
analysis value: C, 87.25; H, 7.07; N, 1.72; S, 3.95

### Synthesis Example 5: Synthesis of Compound 1-126

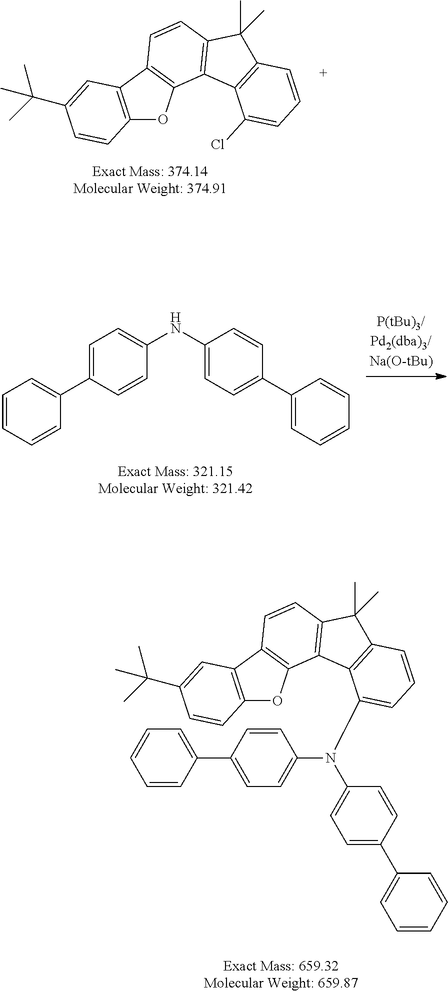 [SMILES: CC(C)(C)c1ccc2oc3c4c(ccc3c2c1)C(C)(C)c1cccc(Cl)c1-4 ; c1ccc(-c2ccc(Nc3ccc(-c4ccccc4)cc3)cc2)cc1 ; CC(C)(C)c1ccc2oc3c4c(ccc3c2c1)C(C)(C)c1cccc(N(c2ccc(-c3ccccc3)cc2)c2ccc(-c3ccccc3)cc2)c1-4]

Compound 1-126 (17.3 g, a yield of 82%) was synthesized in the same manner as in Synthesis Example 1, except that 12.0 g of 3-(tert-butyl)-11-chloro-7,7-dimethyl-7H-fluoreno[4,3-b]benzofuran and 12.35 g of di([1,1′-biphenyl]-4-yl)amine were used in an equivalent ratio of 1:1.2.
calculation value: C, 89.19; H, 6.26; N, 2.12; O, 2.42
analysis value: C, 89.19; H, 6.26; N, 2.12; O, 2.42

### Synthesis Example 6: Synthesis of Compound 1-156

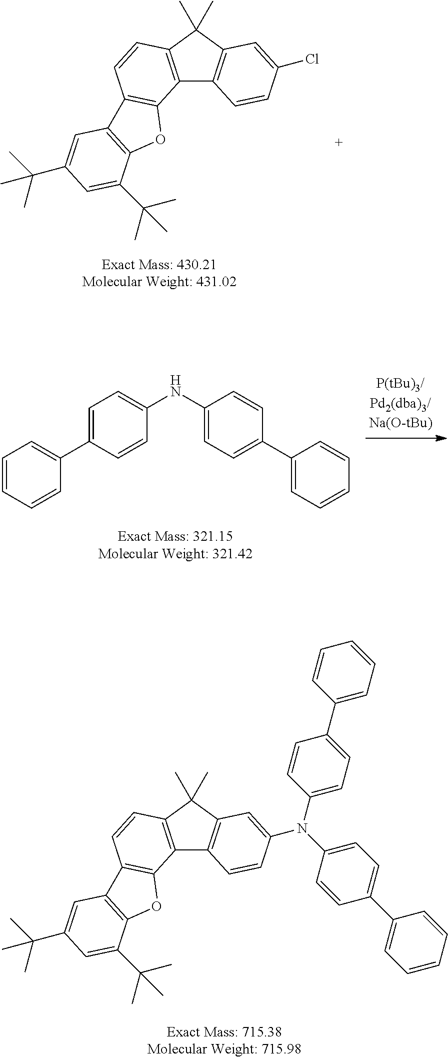 [SMILES: CC(C)(C)c1cc(C(C)(C)C)c2oc3c4c(ccc3c2c1)C(C)(C)c1cc(Cl)ccc1-4 ; c1ccc(-c2ccc(Nc3ccc(-c4ccccc4)cc3)cc2)cc1 ; CC(C)(C)c1cc(C(C)(C)C)c2oc3c4c(ccc3c2c1)C(C)(C)c1cc(N(c2ccc(-c3ccccc3)cc2)c2ccc(-c3ccccc3)cc2)ccc1-4]

Compound 1-156 (13.1 g, a yield of 79%) was synthesized in the same manner as in Synthesis Example 1, except that 10.0 g of 1,3-di-tert-butyl-9-chloro-7,7-dimethyl-7H-fluoreno[4,3-b]benzofuran and 8.95 g of di([1,1′-biphenyl]-4-yl)amine were used in an equivalent ratio of 1:1.2.
calculation value: C, 88.91; H, 6.90; N, 1.96; O, 2.23
analysis value: C, 88.91; H, 6.90; N, 1.96; O, 2.23

### Synthesis Example 7: Synthesis of Compound 1-186

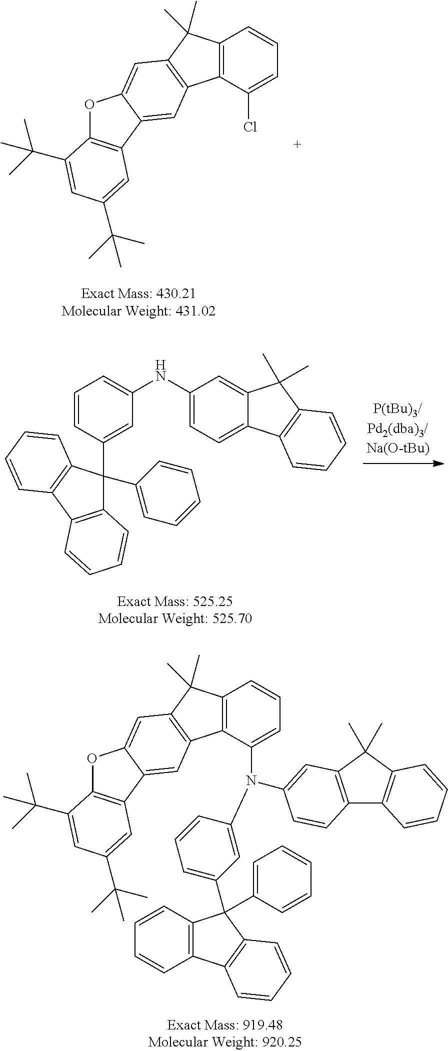 [SMILES: CC(C)(C)c1cc(C(C)(C)C)c2oc3cc4c(cc3c2c1)-c1c(Cl)cccc1C4(C)C ; CC1(C)c2ccccc2-c2ccc(Nc3cccc(C4(c5ccccc5)c5ccccc5-c5ccccc54)c3)cc21 ; CC(C)(C)c1cc(C(C)(C)C)c2oc3cc4c(cc3c2c1)-c1c(N(c2cccc(C3(c5ccccc5)c5ccccc5-c5ccccc53)c2)c2ccc3c(c2)C(C)(C)c2ccccc2-3)cccc1C4(C)C]

Compound 1-186 (17.8 g, a yield of 83%) was synthesized in the same manner as in Synthesis Example 1, except that 10.0 g of 2,4-di-tert-butyl-11-chloro-7,7-dimethyl-7H-fluoreno[2,3-b]benzofuran and 14.64 g of 9,9-dimethyl-N-(3-(9-phenyl-9H-fluoren-9-yl)phenyl)-9H-fluoren-2-amine were used in an equivalent ratio of 1:1.2.
calculation value: C, 90.06; H, 6.68; N, 1.52; O, 1.74
analysis value: C, 90.06; H, 6.68; N, 1.52; O, 1.74

### Synthesis Example 8: Synthesis of Compound 1-329

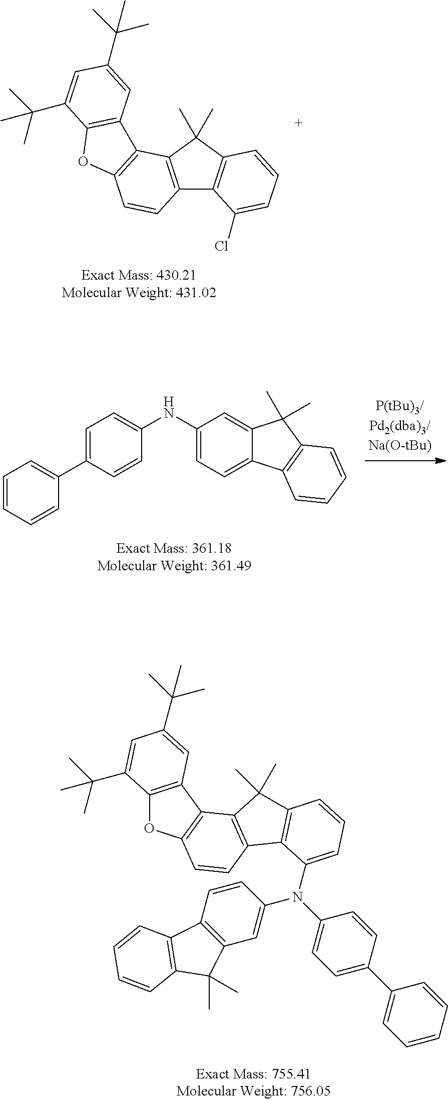 [SMILES: CC(C)(C)c1cc(C(C)(C)C)c2oc3ccc4c(c3c2c1)C(C)(C)c1cccc(Cl)c1-4 ; CC1(C)c2ccccc2-c2ccc(Nc3ccc(-c4ccccc4)cc3)cc21 ; CC(C)(C)c1cc(C(C)(C)C)c2oc3ccc4c(c3c2c1)C(C)(C)c1cccc(N(c2ccc(-c3ccccc3)cc2)c2ccc3c(c2)C(C)(C)c2ccccc2-3)c1-4]

Compound 1-329 (11.6 g, a yield of 83%) was synthesized in the same manner as in Synthesis Example 1, except that 8.0 g of 2,4-di-tert-butyl-8-chloro-12,12-dimethyl-12H-fluoreno[2,1-b]benzofuran and 8.05 g of N-([1,1′-biphenyl]-4-yl)-9,9-dimethyl-9H-fluoren-2-amine were used in an equivalent ratio of 1:1.2.
calculation value: C, 88.96; H, 7.07; N, 1.85; O, 2.12
analysis value: C, 88.96; H, 7.07; N, 1.85; O, 2.12

### Synthesis Example 9: Synthesis of Compound 1-336

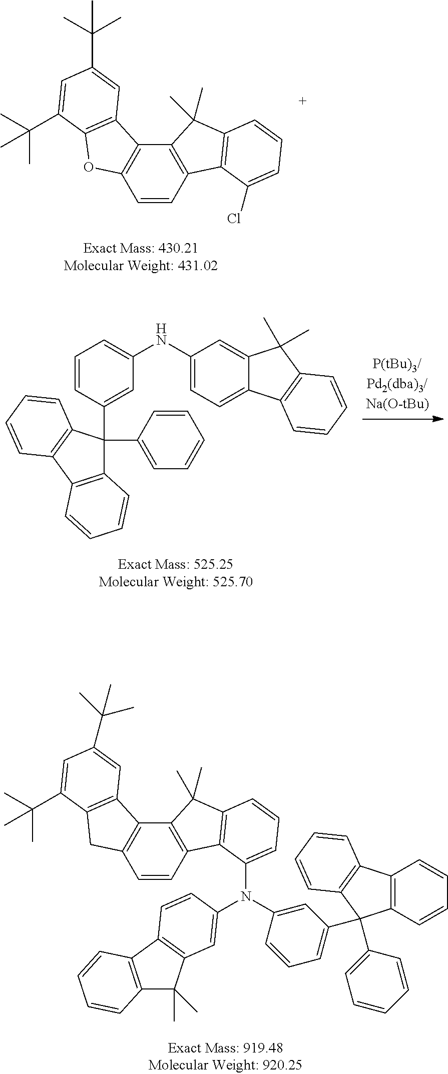 [SMILES: CC(C)(C)c1cc(C(C)(C)C)c2oc3ccc4c(c3c2c1)C(C)(C)c1cccc(Cl)c1-4 ; CC1(C)c2ccccc2-c2ccc(Nc3cccc(C4(c5ccccc5)c5ccccc5-c5ccccc54)c3)cc21 ; CC(C)(C)c1cc2c(c(C(C)(C)C)c1)Cc1ccc3c(c1-2)C(C)(C)c1cccc(N(c2cccc(C4(c5ccccc5)c5ccccc5-c5ccccc54)c2)c2ccc4c(c2)C(C)(C)c2ccccc2-4)c1-3]

Compound 1-336 (21.2 g, a yield of 83%) was synthesized in the same manner as in Synthesis Example 1, except that 12.0 g of 2,4-di-tert-butyl-8-chloro-12,12-dimethyl-12H-fluoreno[2,1-b]benzofuran and 17.56 g of 9,9-dimethyl-N-(3-(9-phenyl-9H-fluoren-9-yl)phenyl)-9H-fluoren-2-amine were used in an equivalent ratio of 1:1.2.
calculation value: C, 90.06; H, 6.68; N, 1.52; O, 1.74
analysis value: C, 90.06; H, 6.68; N, 1.52; O, 1.74

### Comparative Synthesis Example 1: Synthesis of Compound R1

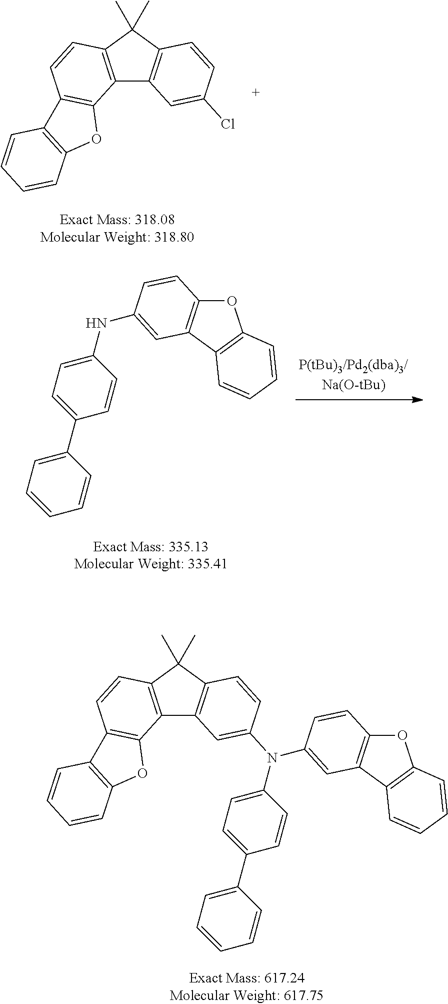 [SMILES: CC1(C)c2ccc(Cl)cc2-c2c1ccc1c2oc2ccccc21 ; c1ccc(-c2ccc(Nc3ccc4oc5ccccc5c4c3)cc2)cc1 ; CC1(C)c2ccc(N(c3ccc(-c4ccccc4)cc3)c3ccc4oc5ccccc5c4c3)cc2-c2c1ccc1c2oc2ccccc21]

Compound R1 (14.5 g, a yield of 75%) was synthesized in the same manner as in Synthesis Example 1, except that 10.0 g of 10-chloro-7,7-dimethyl-7H-fluoreno[4,3-b]benzofuran and 12.63 g of N-([1,1′-biphenyl]-4-yl)dibenzo[b,d]furan-2-amine were used in an equivalent ratio of 1:1.2.
calculation value: C, 87.49; H, 5.06; N, 2.27; 0, 5.18
analysis value: C, 87.49; H, 5.06; N, 2.27; 0, 5.18

### Comparative Synthesis Example 2: Synthesis of Compound R2

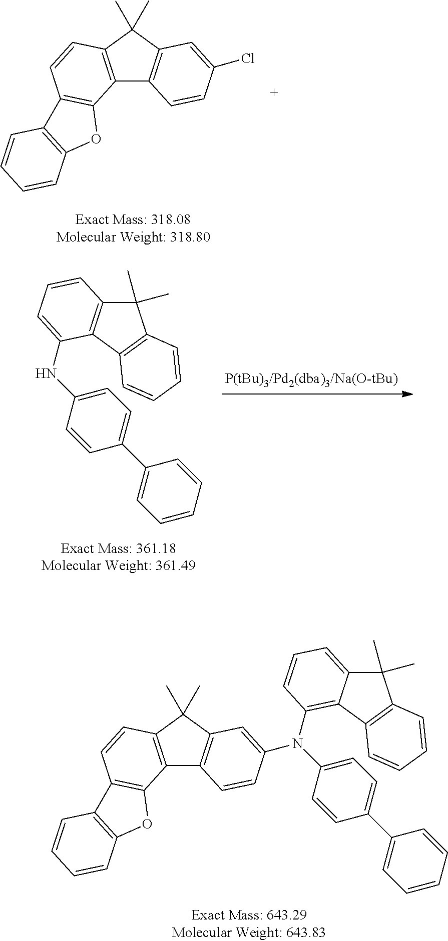 [SMILES: CC1(C)c2cc(Cl)ccc2-c2c1ccc1c2oc2ccccc21 ; CC1(C)c2ccccc2-c2c(Nc3ccc(-c4ccccc4)cc3)cccc21 ; CC1(C)c2ccccc2-c2c(N(c3ccc(-c4ccccc4)cc3)c3ccc4c(c3)C(C)(C)c3ccc5c(oc6ccccc65)c3-4)cccc21]

Compound R2 (15.1 g, a yield of 75%) was synthesized in the same manner as in Synthesis Example 1, except that 10.0 g of 9-chloro-7,7-dimethyl-7H-fluoreno[4,3-b]benzofuran and 13.61 g of N-([1,1′-biphenyl]-4-yl)-9,9-dimethyl-9H-fluoren-4-amine were used in an equivalent ratio of 1:1.2.
calculation value: C, 89.55; H, 5.79; N, 2.18; O, 2.48
analysis value: C, 89.55; H, 5.79; N, 2.18; O, 2.48

### Comparative Synthesis Example 3: Synthesis of Compound R3

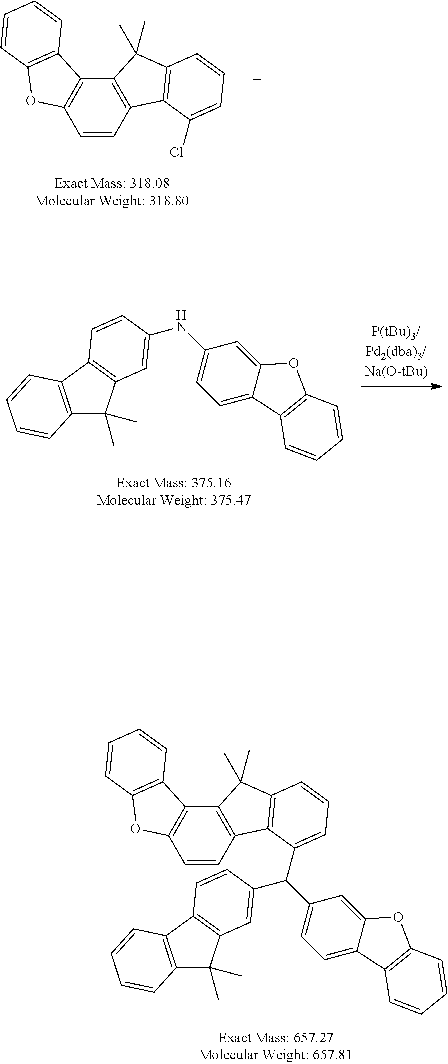 [SMILES: CC1(C)c2cccc(Cl)c2-c2ccc3oc4ccccc4c3c21 ; CC1(C)c2ccccc2-c2ccc(Nc3ccc4c(c3)oc3ccccc34)cc21 ; CC1(C)c2ccccc2-c2ccc(C(c3ccc4c(c3)oc3ccccc34)c3cccc4c3-c3ccc5oc6ccccc6c5c3C4(C)C)cc21]

Compound R3 (24.5 g, a yield of 79%) was synthesized in the same manner as in Synthesis Example 1, except that 15.0 g of 8-chloro-12,12-dimethyl-12H-fluoreno[2,1-b]benzofuran and 21.2 g of N-(9,9-dimethyl-9H-fluoren-2-yl)dibenzo[b,d]furan-3-amine were used in an equivalent ratio of 1:1.2.
calculation value: C, 87.64; H, 5.36; N, 2.13; O, 4.86
analysis value: C, 87.64; H, 5.36; N, 2.13; O, 4.86

### (Manufacturing of Organic Light Emitting Diode)

### Example 1

A glass substrate coated with a thin film of ITO/Ag/ITO was ultrasonically cleaned with distilled water. After washing with the distilled water, the glass substrate was ultrasonically washed with acetone or isopropyl alcohol, and dried and then, moved to a plasma cleaner, cleaned by using oxygen plasma for 10 minutes, and moved to a vacuum depositor. This prepared ITO/Ag/ITO (reflective electrode) was used as an anode, Compound A doped with 3% NDP-9 (Novaled GmbH) was vacuum-deposited on the ITO/Ag/ITO substrate to form a 100 Å-thick hole injection layer, and Compound A was deposited on the hole injection layer to a thickness of 1,240 Å to form a hole transport layer. Compound 1-4 obtained in Synthesis Example 1 was deposited on the hole transport layer to a thickness of 320 Å to form a hole transport auxiliary layer. On the hole transport auxiliary layer, 85 wt % of Host H1 (40 wt %) and Host H2 (60 wt %) were used as hosts, and 15 wt % of GD was doped as a dopant to form a 350 Å-thick light emitting layer by vacuum deposition. Subsequently, Compound C was deposited on the light emitting layer to a thickness of 50 Å to form an electron transport auxiliary layer, and Compound D and Liq were simultaneously vacuum-deposited in a ratio of 1:1 to form a 310 Å-thick electron transport layer. An organic light emitting diode was manufactured by sequentially vacuum-depositing Yb, AgMg on the electron transport layer to form a cathode.

ITO/Ag/ITO/Compound A (3% NDP-9 doping, 100 Å)/Compound A (1,240 Å)/hole transport auxiliary layer (320 Å)/light emitting layer [Host (Host H1, Host H2):GD=85 wt %:15 wt %] (350 Å)/Compound C (50 Å)/Compound D:Liq (310 Å)/Yb/AgMg.
Compound A: N-([1,1′-biphenyl]-2-yl)-N-(9,9-dimethyl-9H-fluoren-2-yl)-9,9′-spirobi[fluoren]-2-amine
Compound C: 2-(3′-(9,9-dimethyl-9H-fluoren-2-yl)-[1,1′-biphenyl]-3-yl)-4,6-diphenyl-1,3,5-triazine
Compound D: 6,6′-(naphthalene-1,2-diylbis(4,1-phenylene))bis(2,4-diphenyl-1,3,5-triazine)
Host H1: 2-([1,1′-biphenyl]-4-yl)-4-phenyl-6-(3-(triphenylen-2-yl)phenyl)-1,3,5-triazine
Host H2: 9,9″-diphenyl-9H,9″H-3,3′:9′,3″-tercarbazole

### GD:

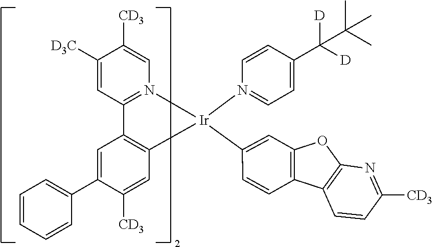

### Examples 2 to 9 and Comparative Examples 1 to 3

The diodes of Examples 2 to 9 and Comparative Examples 1 to 3 were manufactured in the same manner as Example 1, except that the compositions of the hole transport auxiliary layer were changed as shown in Table 1.

### Evaluation

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above.

Relative values based on the luminous efficiency of Comparative Example 1 as a reference are shown in Table 1.

### (4) Measurement of Life-Span

The results were obtained by maintaining the luminance (cd/m²) at 24000 cd/m² and measuring the time for the current efficiency (cd/A) to decrease to 97%.

Relative values based on the life-span measurements of Comparative Example 1 as a reference are shown in Table 1.

TABLE 1

| | Hole transport | Luminous efficiency | Life-span |
|---|---|---|---|
| | auxiliary layer | (%) | (%) |
| Example 1 | 1-4 | 108 | 145 |
| Example 2 | 1-89 | 109 | 149 |
| Example 3 | 1-91 | 108 | 144 |
| Example 4 | 1-114 | 108 | 141 |
| Example 5 | 1-126 | 109 | 148 |
| Example 6 | 1-156 | 107 | 139 |
| Example 7 | 1-186 | 109 | 145 |
| Example 8 | 1-329 | 108 | 147 |
| Example 9 | 1-336 | 107 | 142 |
| Comparative | R1 | 100 | 100 |
| Example 1 | | | |
| Comparative | R2 | 97 | 86 |
| Example 2 | | | |
| Comparative | R3 | 95 | 91 |
| Example 3 | | | |

Referring to Table 1, the organic light emitting diodes to which the compounds according to Examples were applied had significantly improved efficiency characteristics and life-span characteristics, compared to the organic light emitting diodes according to Comparative Examples.

One or more embodiments may provide a compound for an organic optoelectronic device capable of implementing an organic optoelectronic device having low driving, high efficiency, and a long life-span.

An organic optoelectronic device having high efficiency and a long life-span may be realized.

Example embodiments have been disclosed herein, and although specific terms are employed, they are used and are to be interpreted in a generic and descriptive sense only and not for purposes of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by a combination of Chemical Formula 1 and Chemical Formula 2: 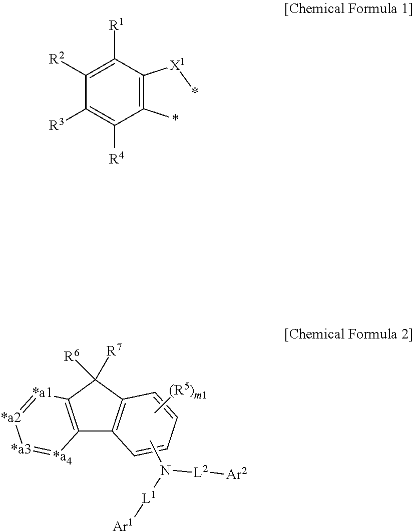
wherein, in Chemical Formula 1 and Chemical Formula 2,
two adjacent ones of a1* to a4* of Chemical Formula 2 are linking carbons linked at * of Chemical Formula 1, the remaining two of a1* to a4* of Chemical Formula 2, not linked at * of Chemical Formula 1, are each independently CR^{a},
X¹ is O or S,
R^{a} and R¹ to R⁵ are each independently hydrogen, deuterium, a cyano group, a substituted or unsubstituted C1 to C20 alkyl group, a substituted or unsubstituted C6 to C20 aryl group, or a combination thereof,
R⁶ and R⁷ are each independently a substituted or unsubstituted C1 to C20 alkyl group or a substituted or unsubstituted C6 to C20 aryl group,
L¹ and L² are each independently a single bond, a substituted or unsubstituted C6 to C20 arylene group, a substituted or unsubstituted C2 to C30 heterocyclic group, or a combination thereof,
Ar¹ and Ar² are each independently a substituted or unsubstituted C6 to C30 aryl group or a substituted or unsubstituted C2 to C30 heterocyclic group,
m1 is an integer of 1 to 3, provided that when m1 is 2 or 3, each R⁵ is the same or different from each other, and
at least one of R¹ to R⁴ is a substituted or unsubstituted tert-butyl group.

2. The compound for an organic optoelectronic device as claimed in claim 1, wherein:
the combination of Chemical Formula 1 and Chemical Formula 2 is represented by one of Chemical Formula 1A to Chemical Formula 1F: 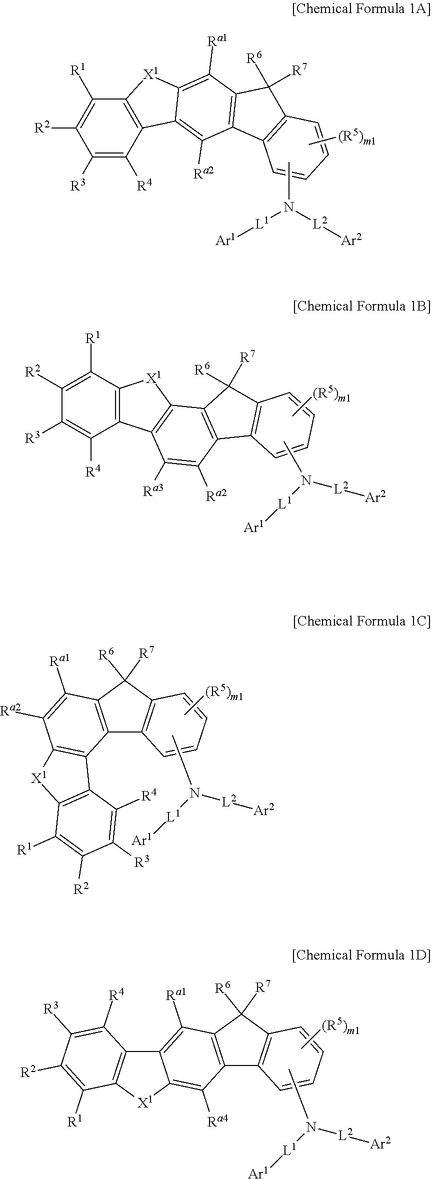 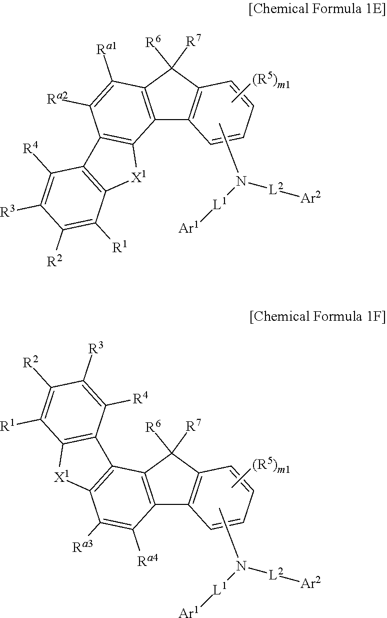
in Chemical Formula 1A to Chemical Formula 1F,
X¹, R¹ to R⁷, L¹, L², Ar¹, Ar², and m1 are defined the same as those of Chemical Formula 1 and Chemical Formula 2, and
R^{a1} to R^{a4} are each independently defined the same as R^{a} of Chemical Formula 2.

3. The compound for an organic optoelectronic device as claimed in claim 1, wherein:
the combination of Chemical Formula 1 and Chemical Formula 2 is represented by Chemical Formula 1A-4, Chemical Formula 1C-4, Chemical Formula 1E-2, Chemical Formula 1E-4, or Chemical Formula 1F-4: 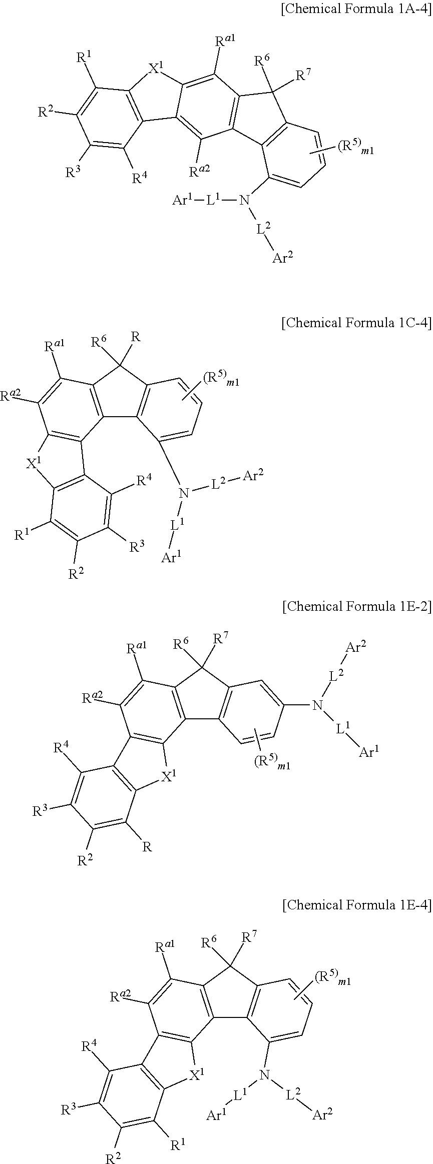 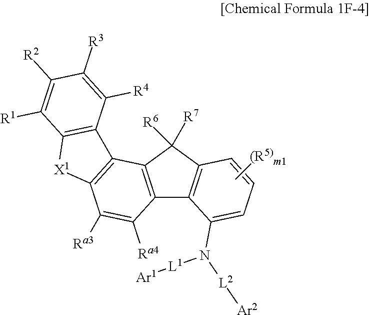
in Chemical Formula 1A-4, Chemical Formula 1C-4, Chemical Formula 1E-2, Chemical Formula 1E-4, and Chemical Formula 1F-4,
X¹, R¹ to R⁷, L¹, L², Ar¹, Ar², and m1 are defined the same as those of Chemical Formula 1 and Chemical Formula 2, and
R^{a1} to R^{a4} are each independently defined the same as R^{a} of Chemical Formula 2.

4. The compound for an organic optoelectronic device as claimed in claim 1, wherein at least two of R¹ to R⁴ are a substituted or unsubstituted tert-butyl group.

5. The compound for an organic optoelectronic device as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

6. The compound for an organic optoelectronic device as claimed in claim 1, wherein:
moieties *-L¹-Ar¹ and *-L²-Ar² are each independently a moiety of Group I: 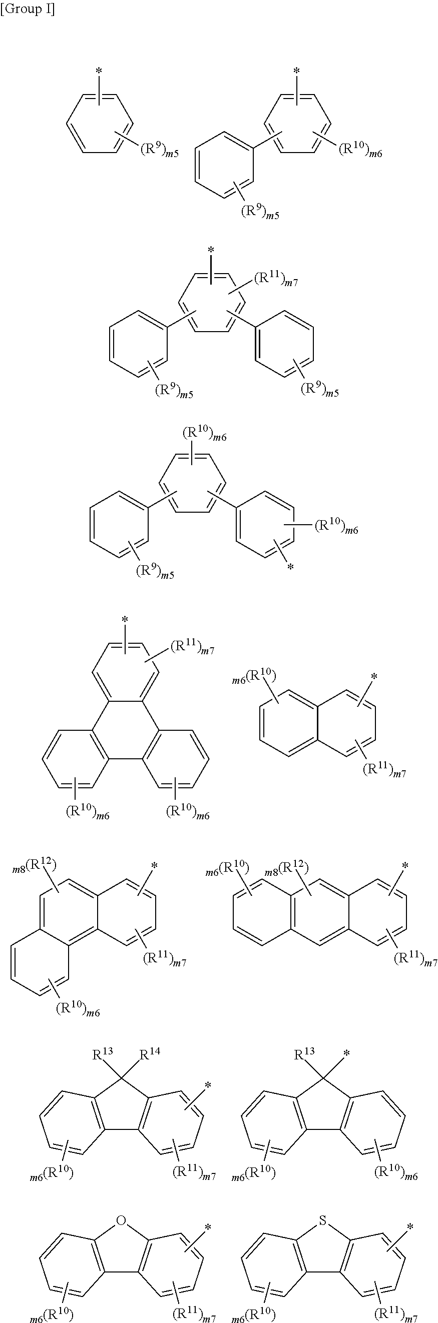 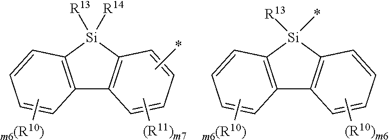
in Group I,
R⁹ to R¹² are each independently hydrogen, deuterium, a cyano group, a substituted or unsubstituted C1 to C10 alkyl group, or a substituted or unsubstituted C6 to C12 aryl group,
R¹³ and R¹⁴ are each independently a substituted or unsubstituted C1 to C10 alkyl group or a substituted or unsubstituted C6 to C12 aryl group,
m5 is an integer of 1 to 5,
m6 is an integer of 1 to 4,
m7 is an integer of 1 to 3,
m8 is 1 or 2, and
* is a linking point.

7. A compound for an organic optoelectronic device, the compound being a compound of Group 1: 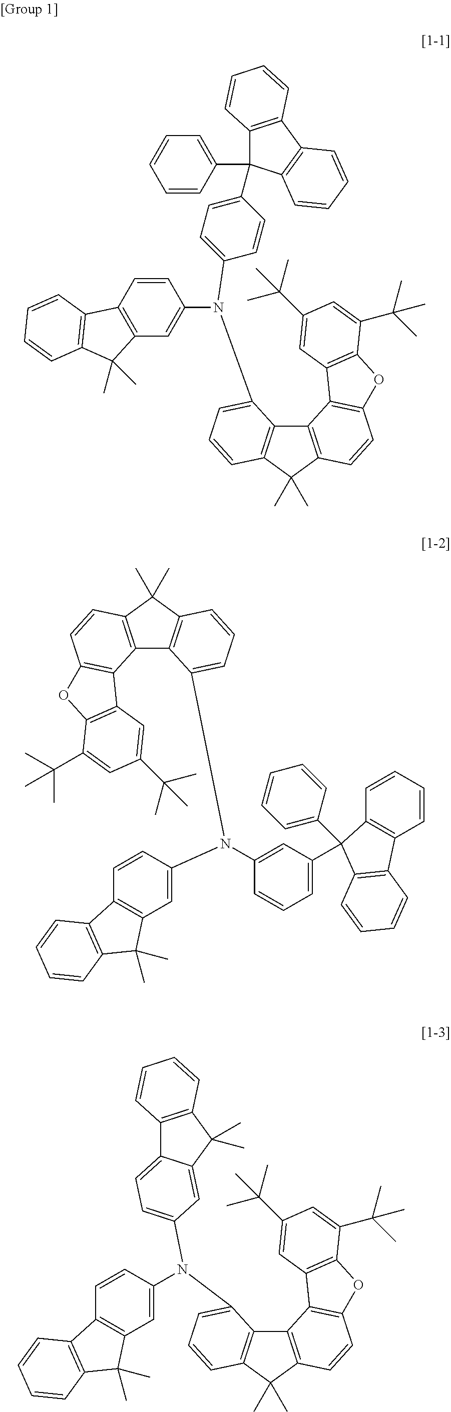 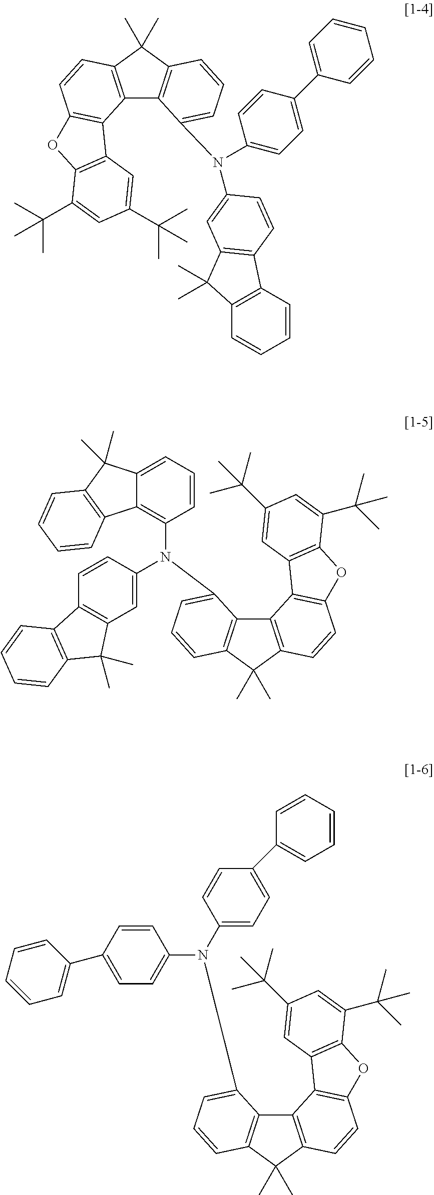 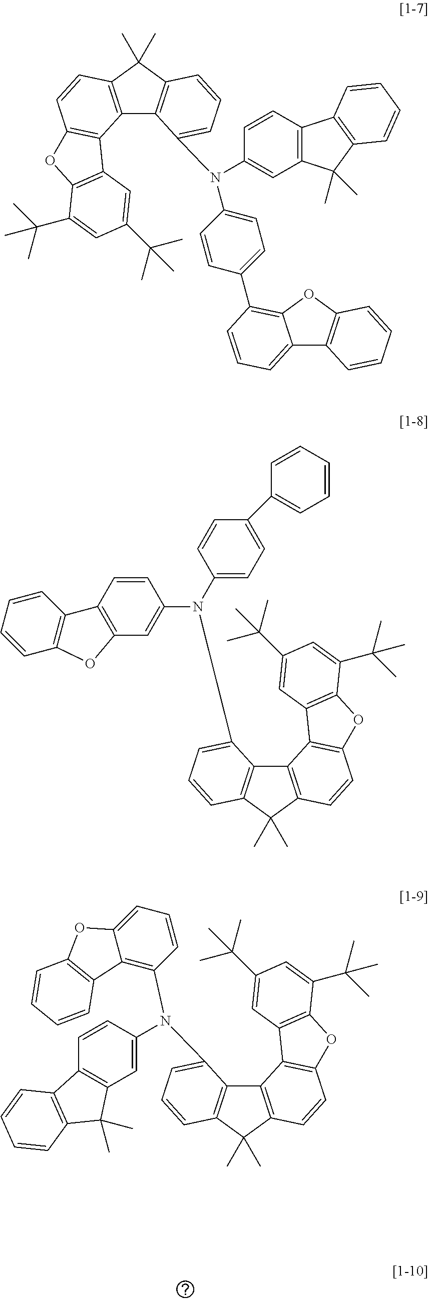 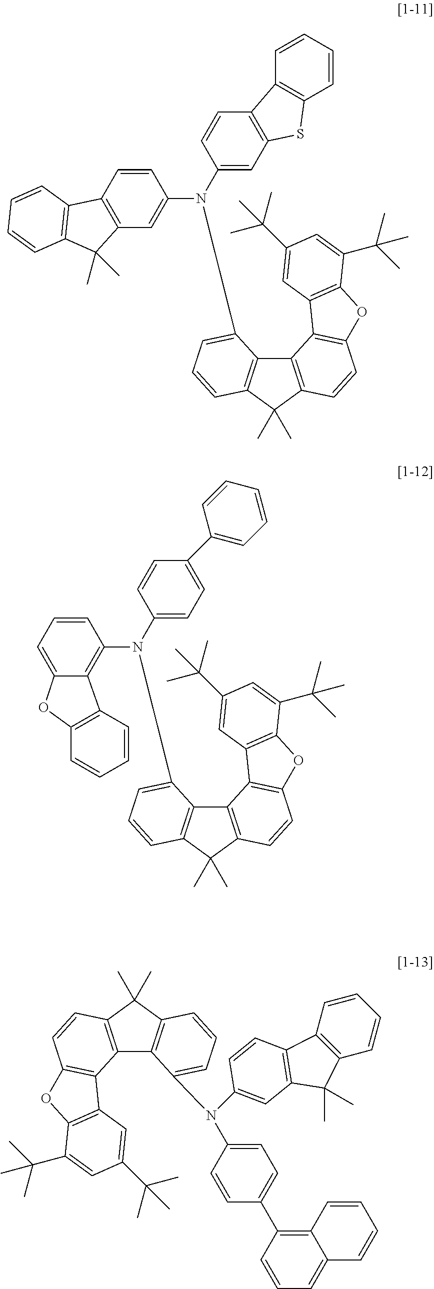 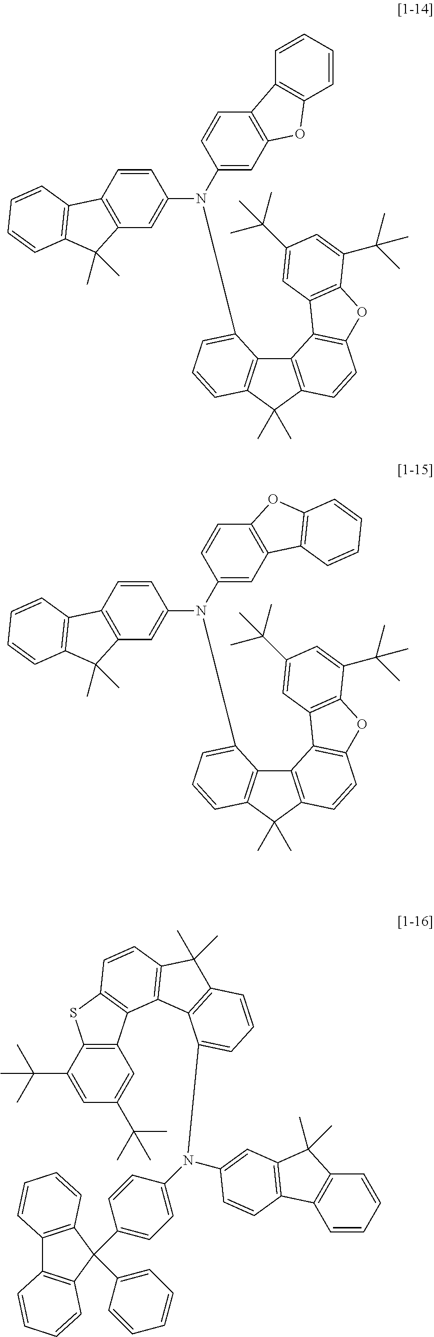 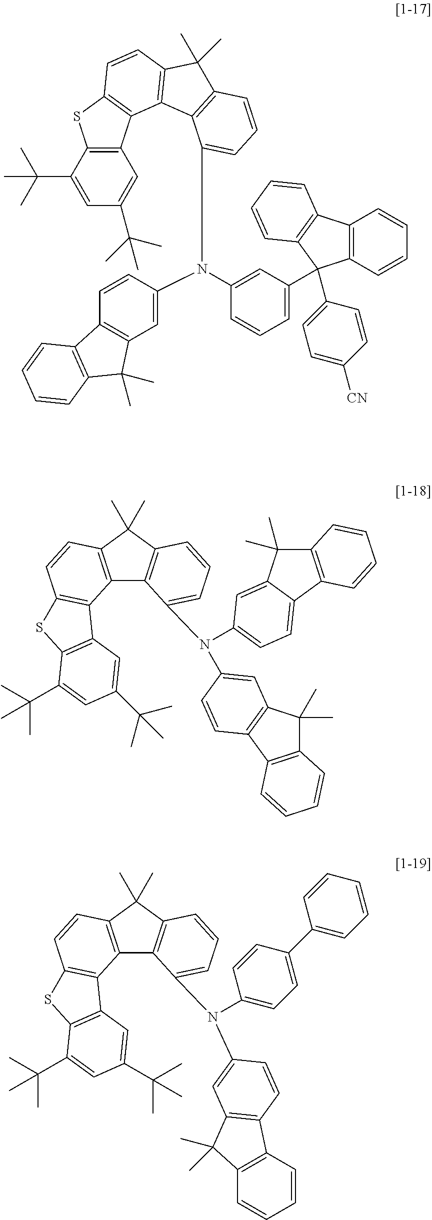 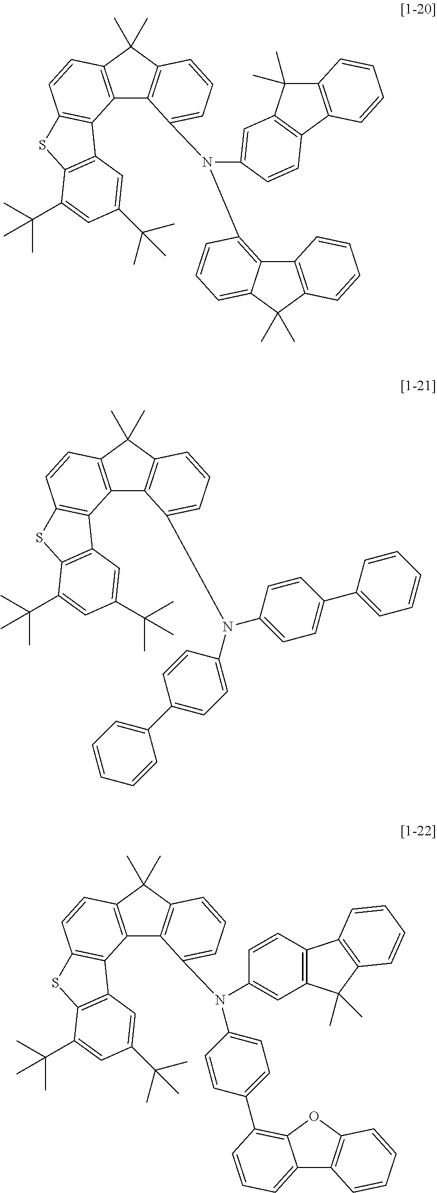 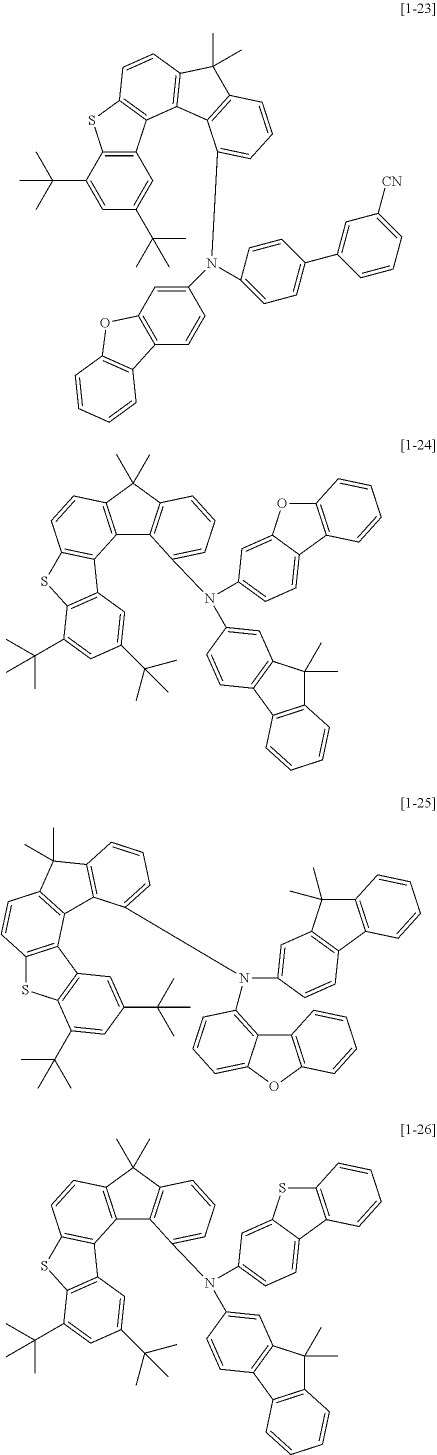 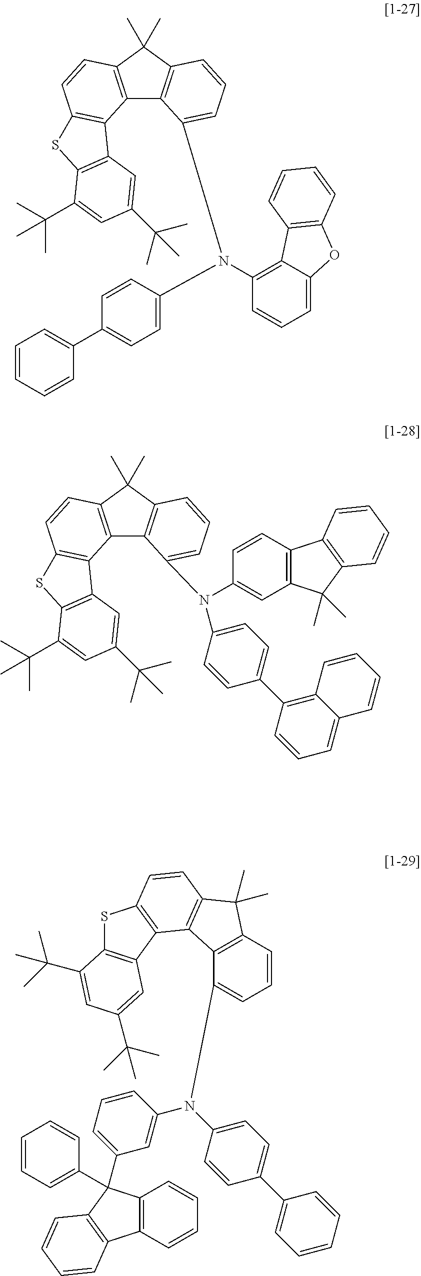 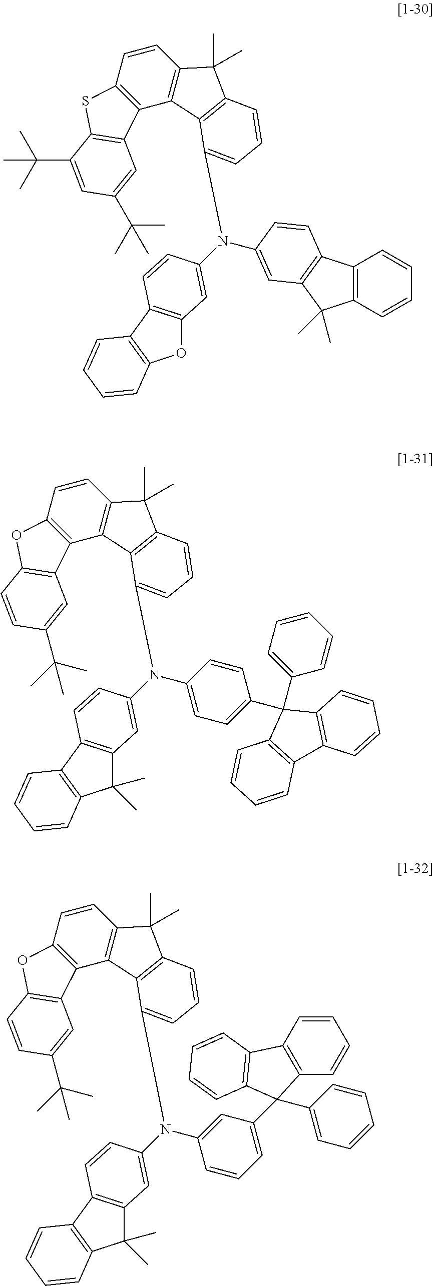 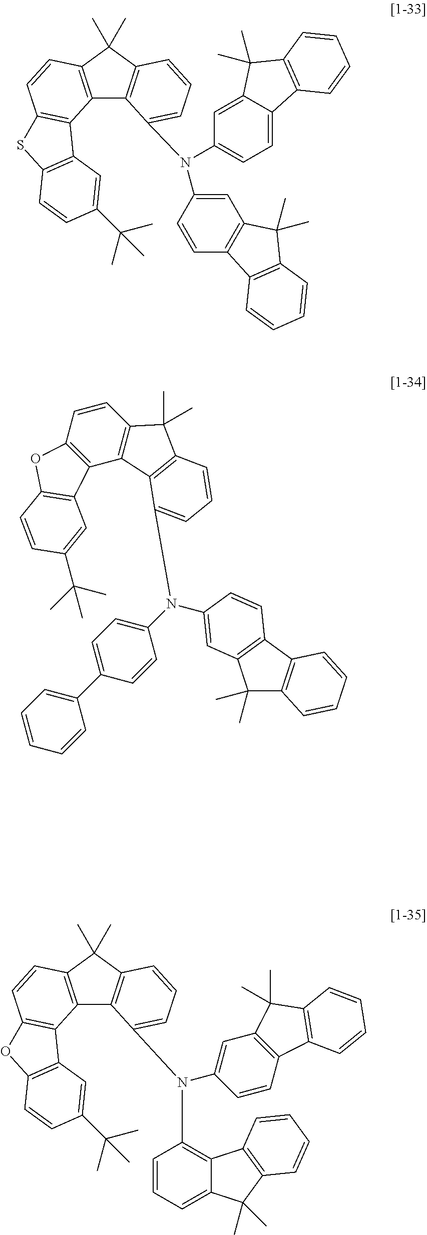 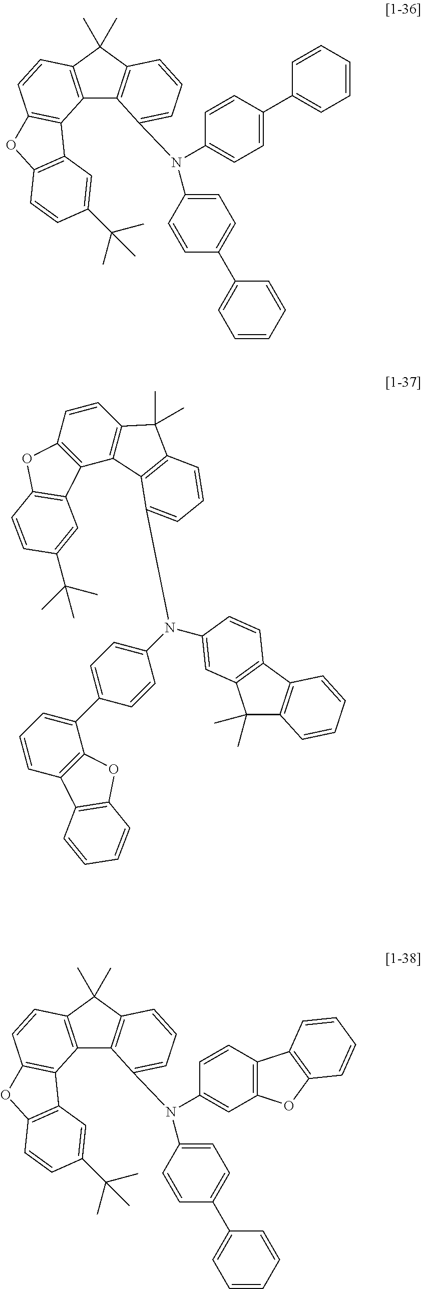 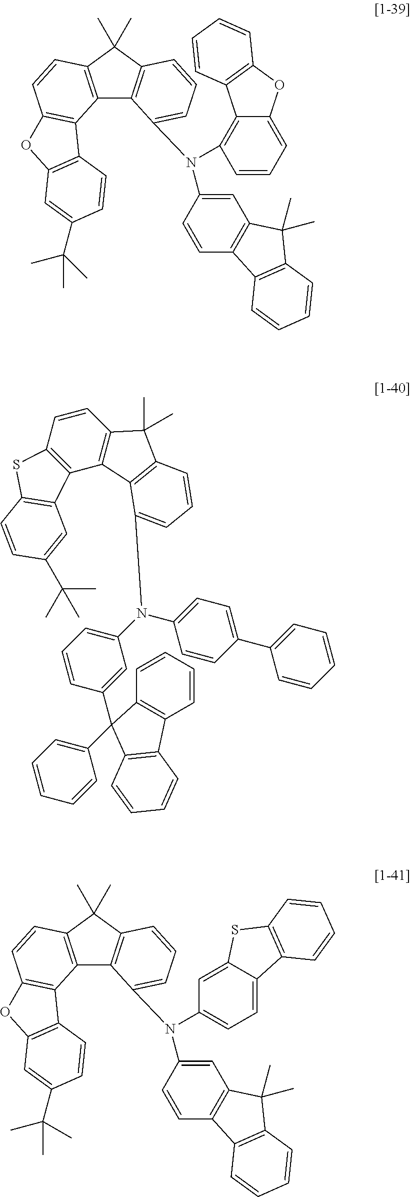 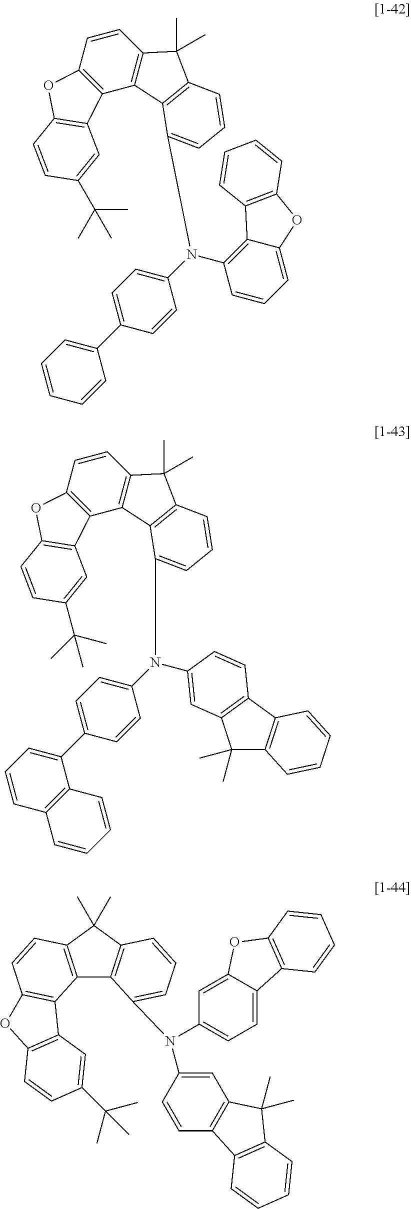 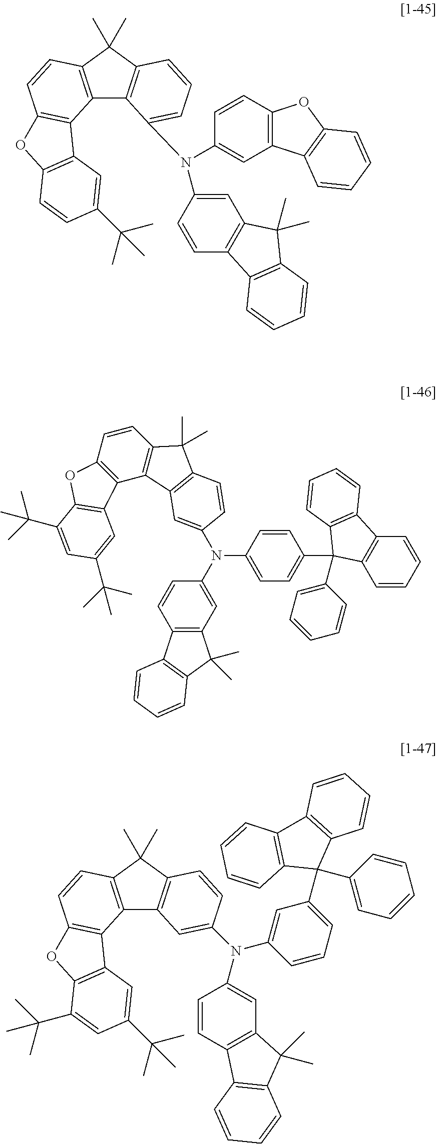 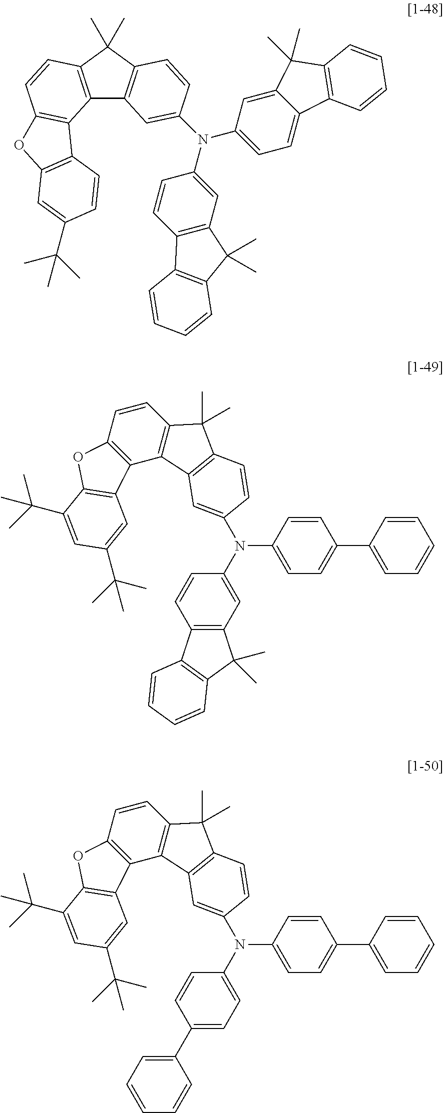 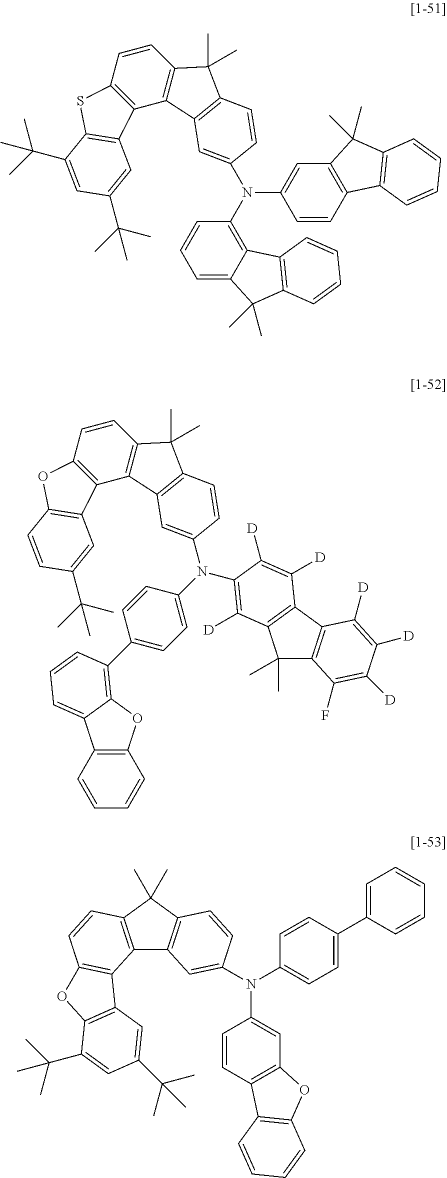 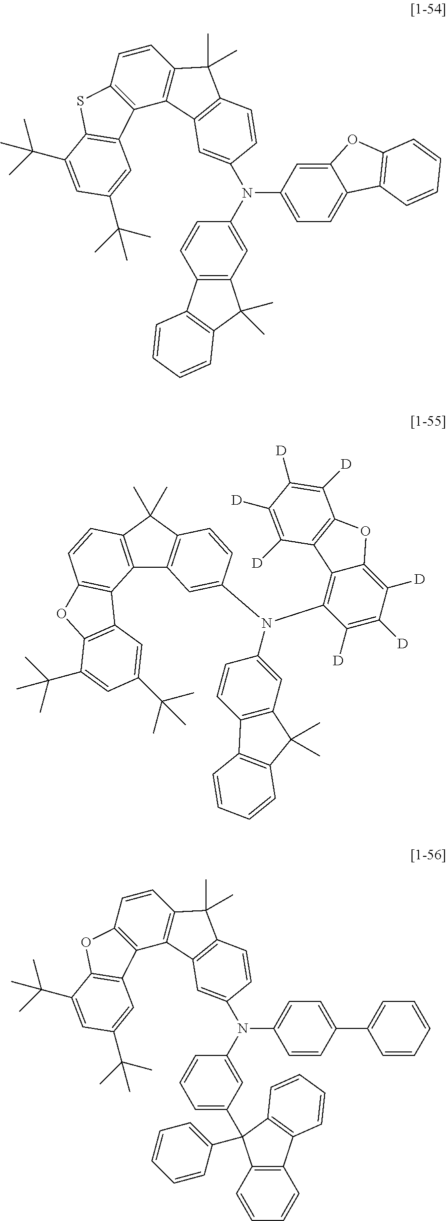 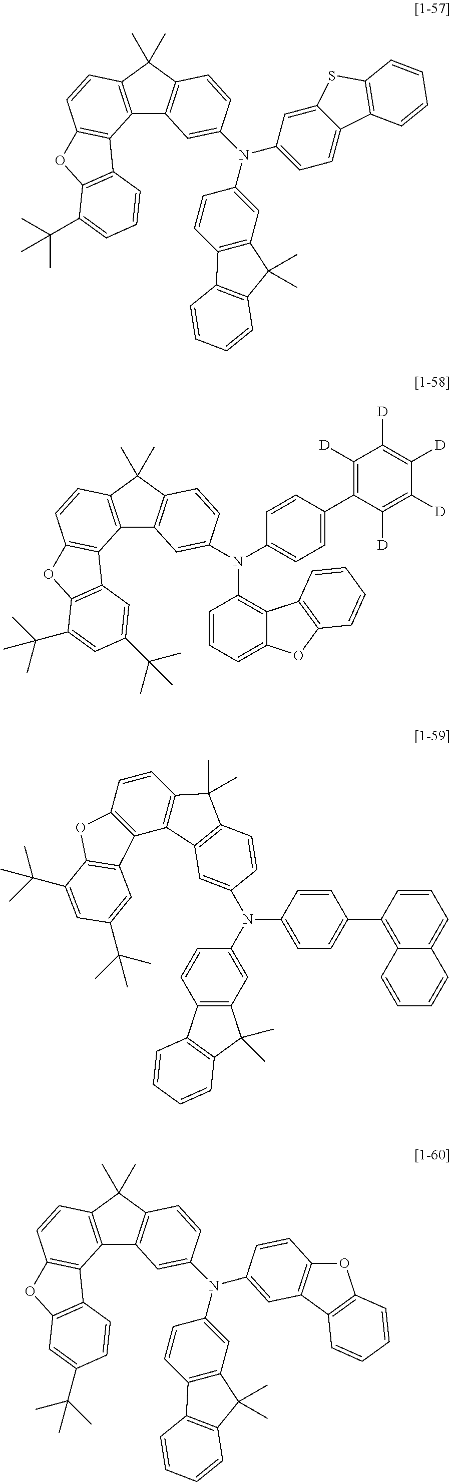 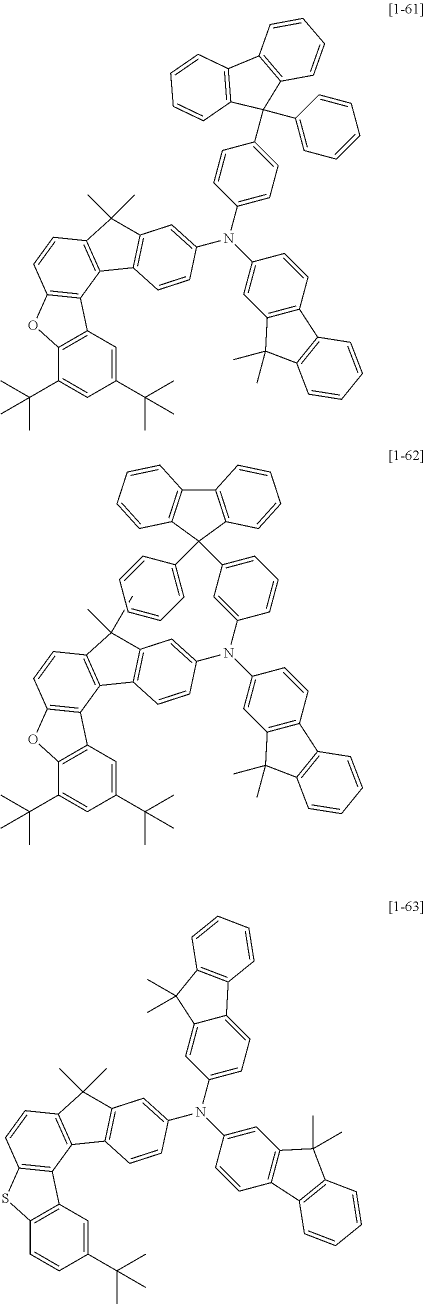 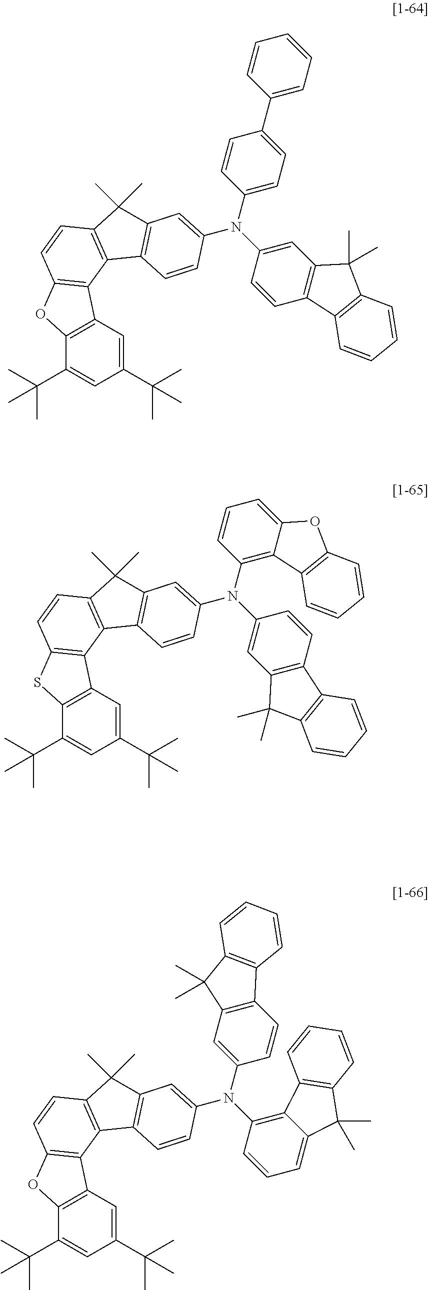 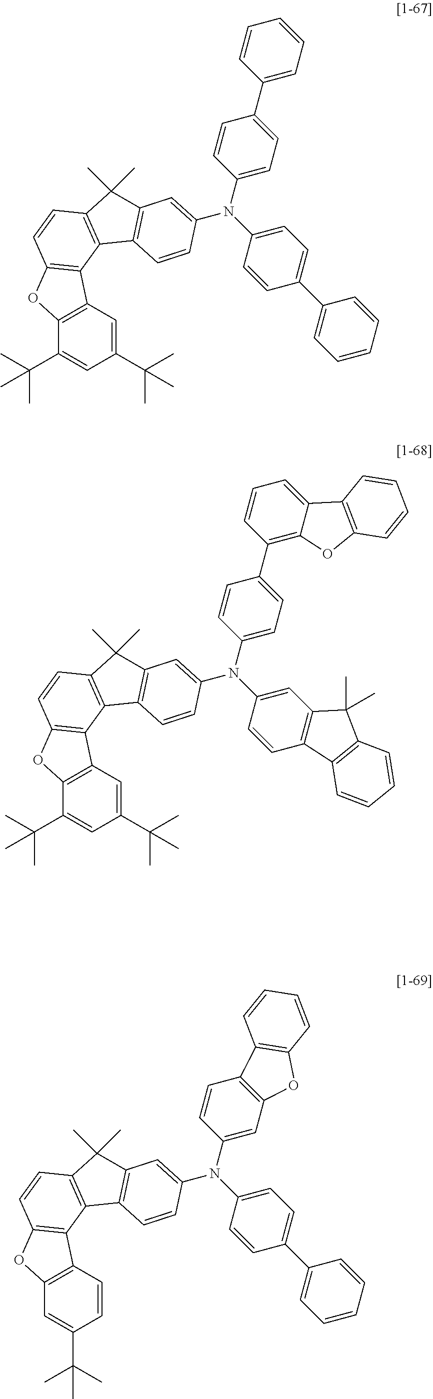 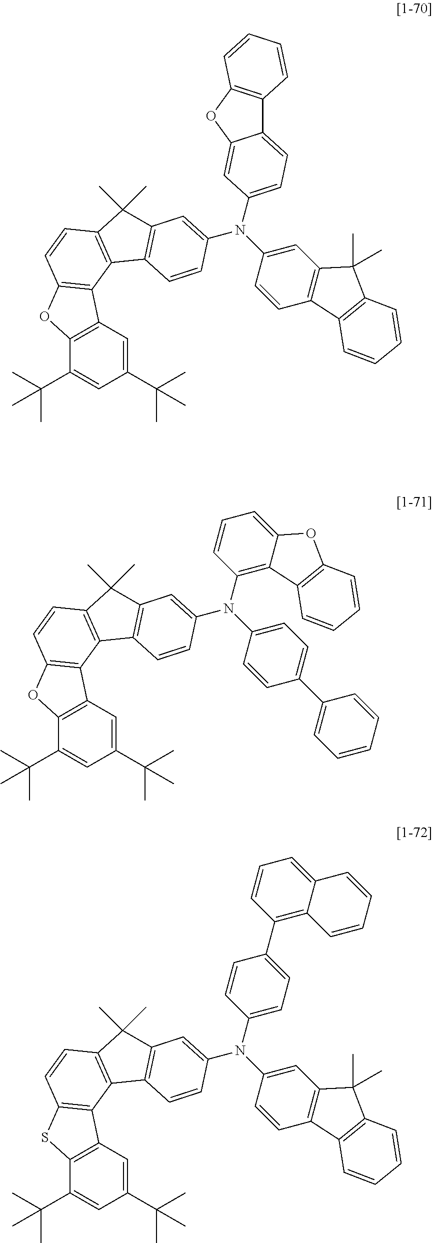 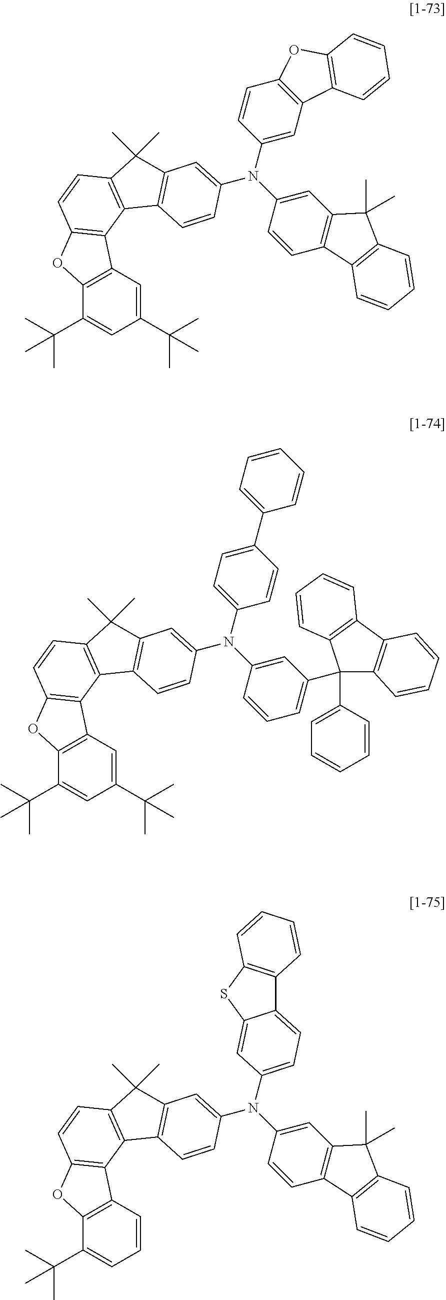 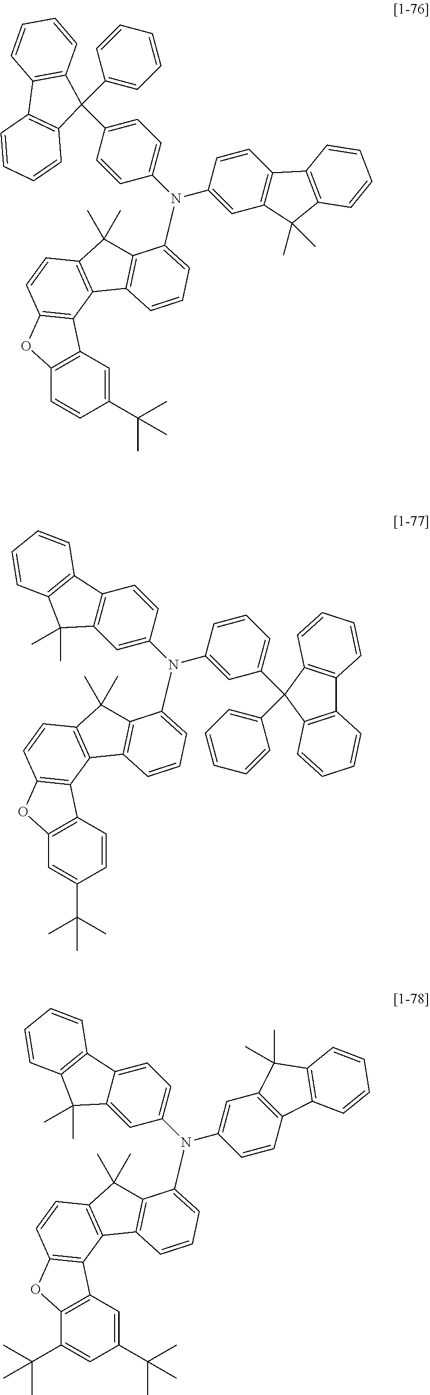 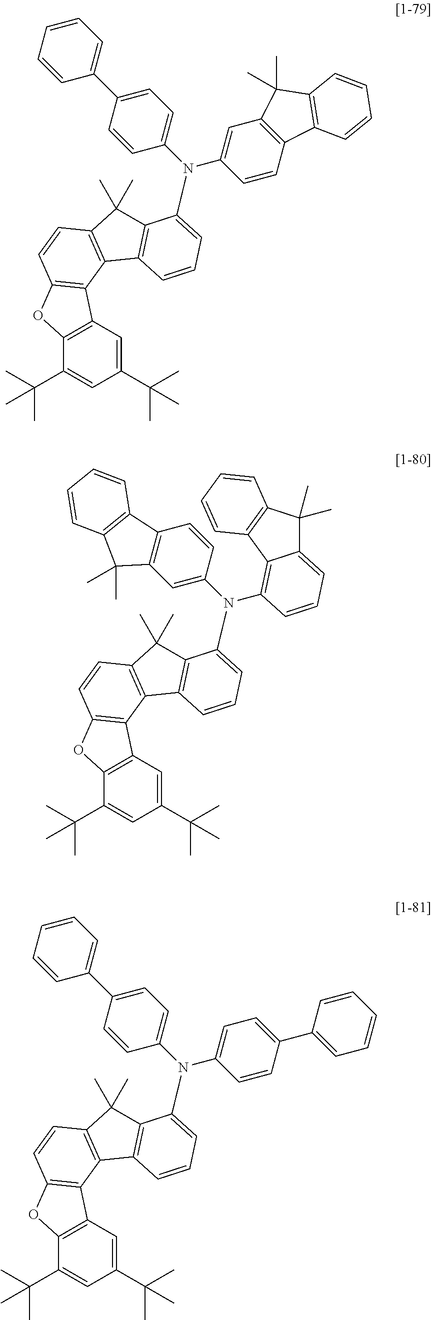 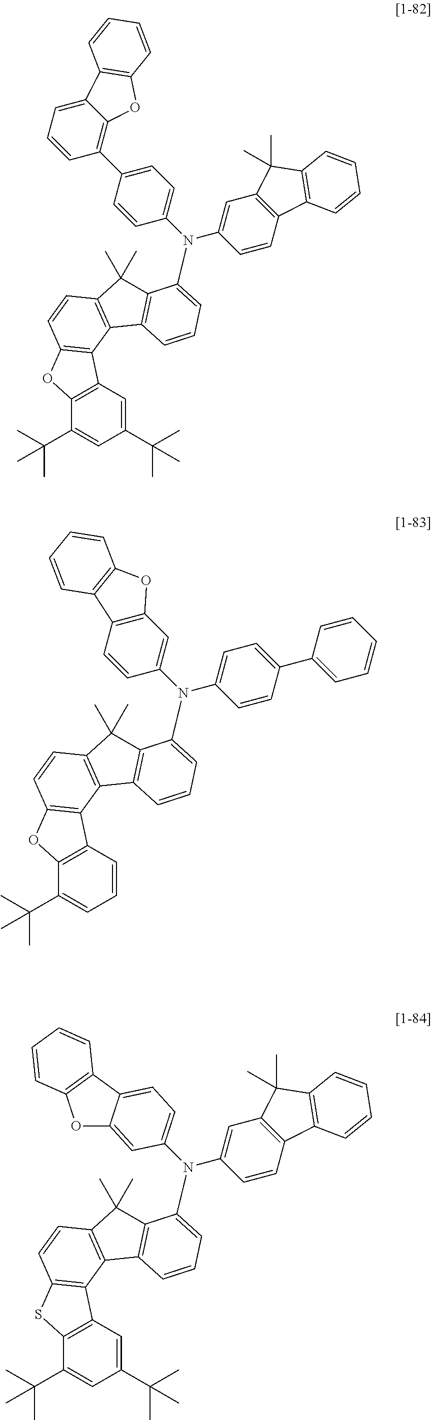 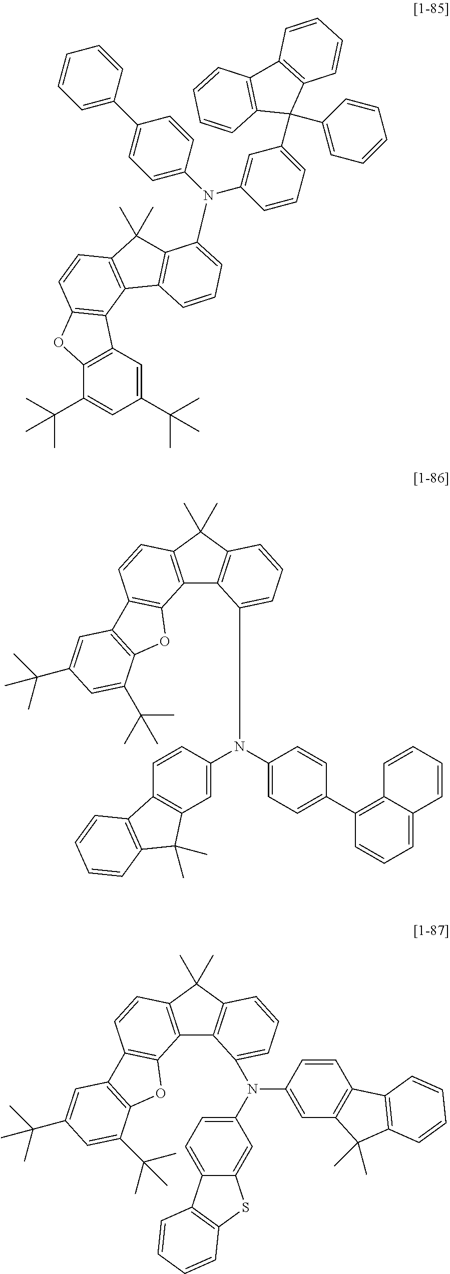 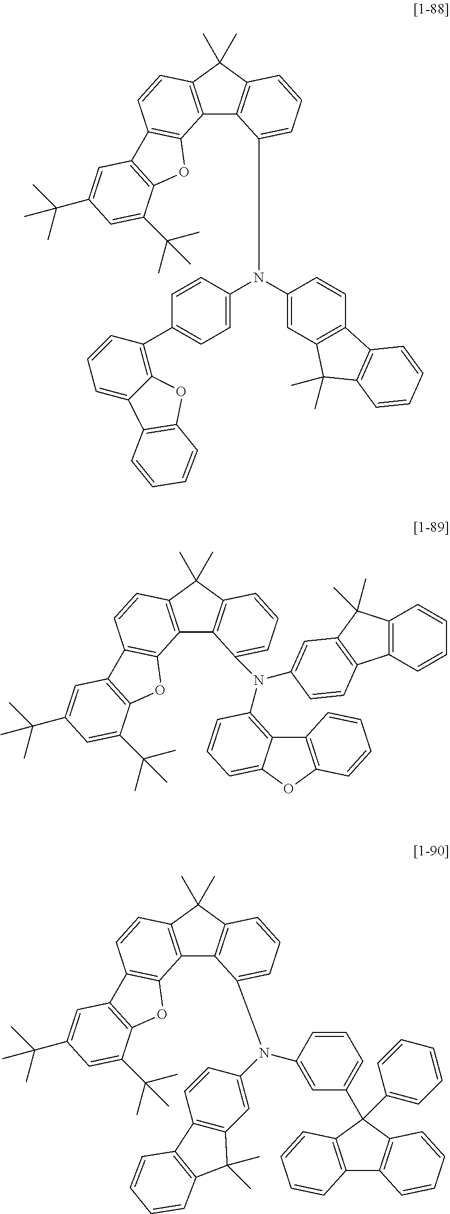 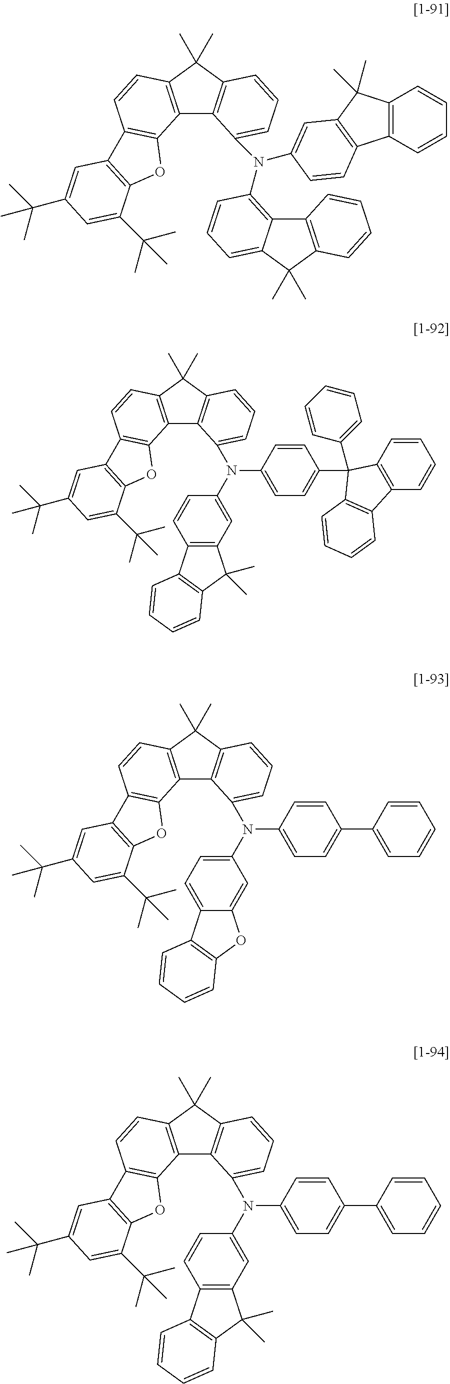 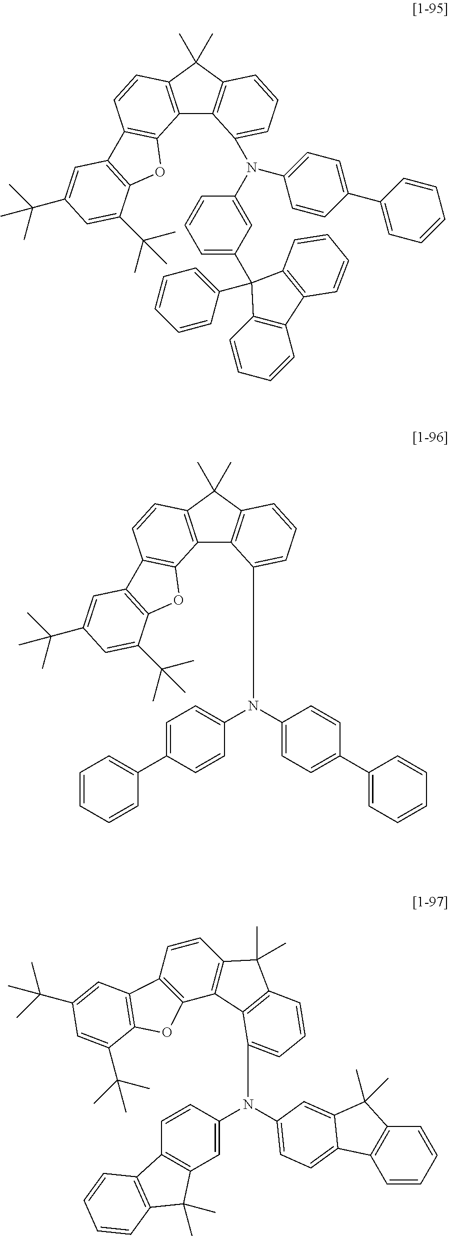 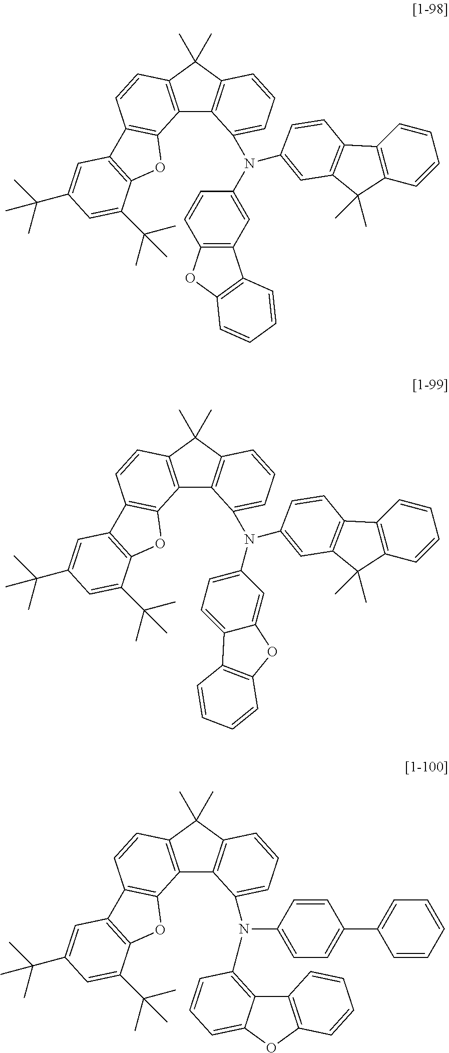 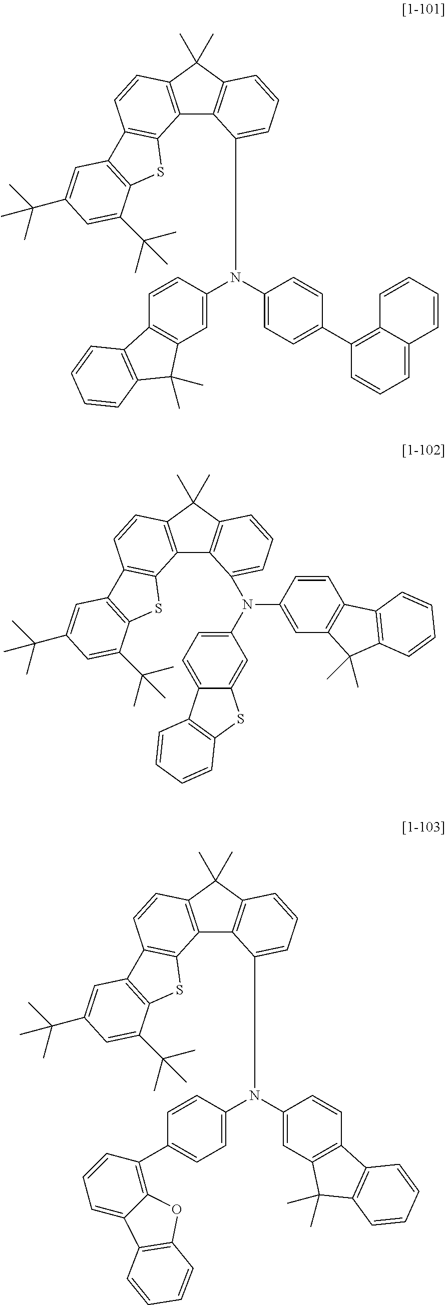 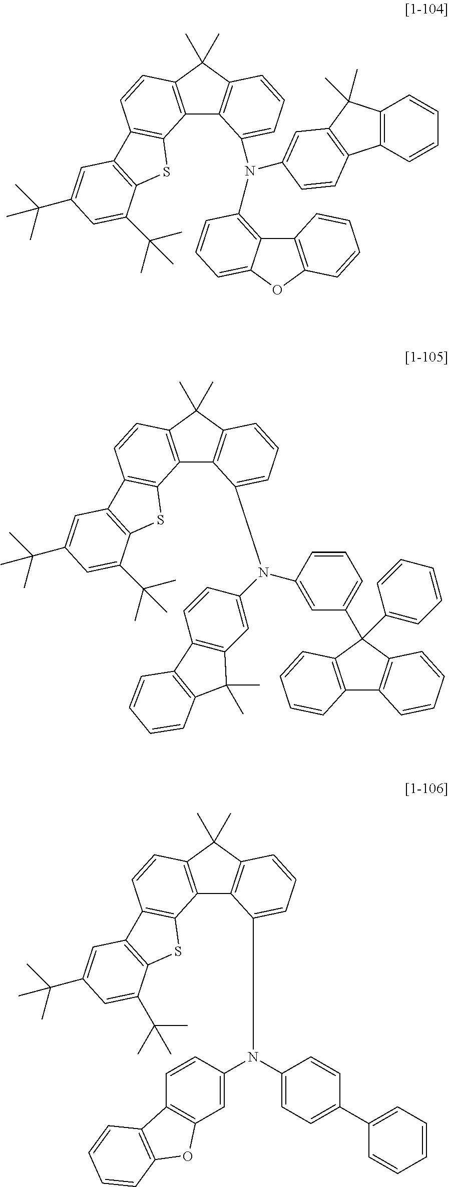 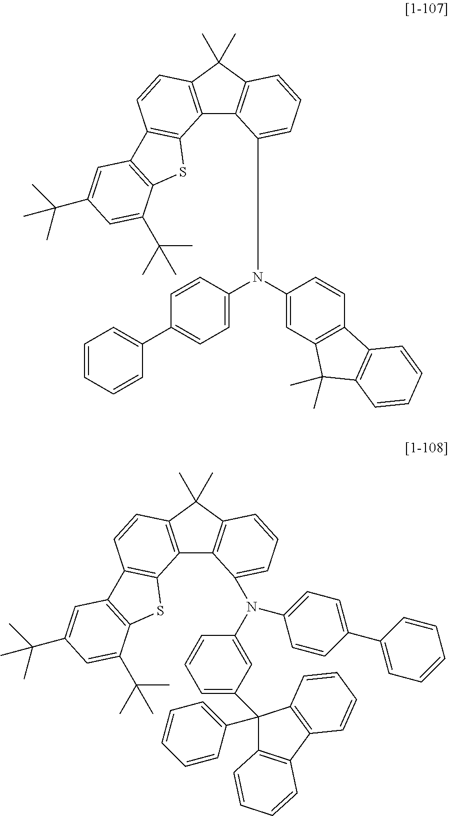 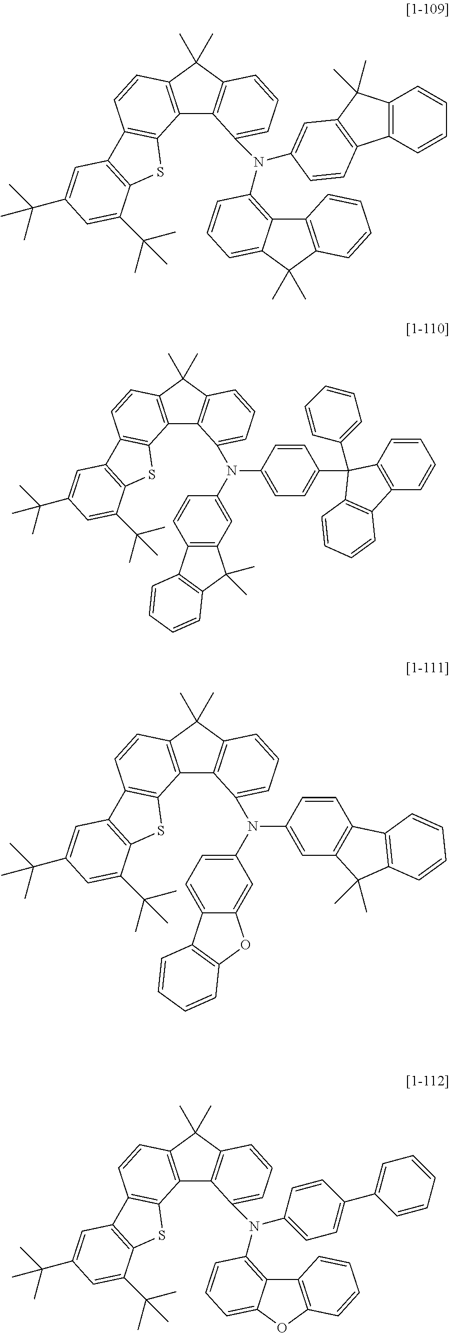 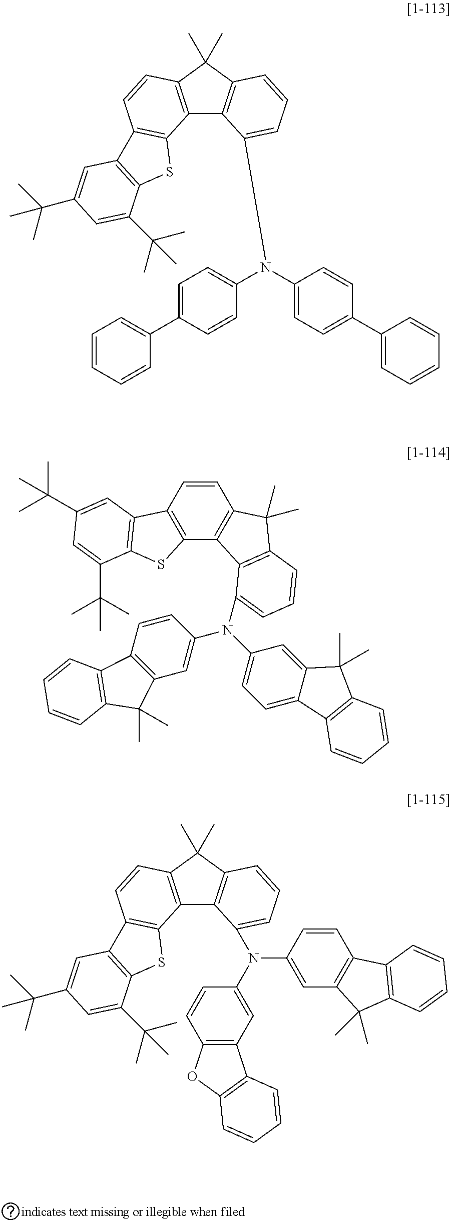 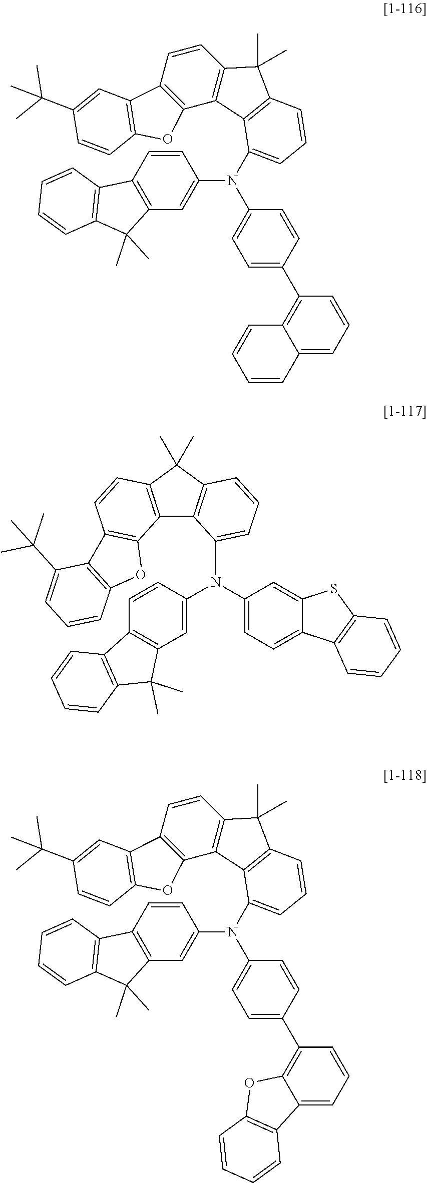 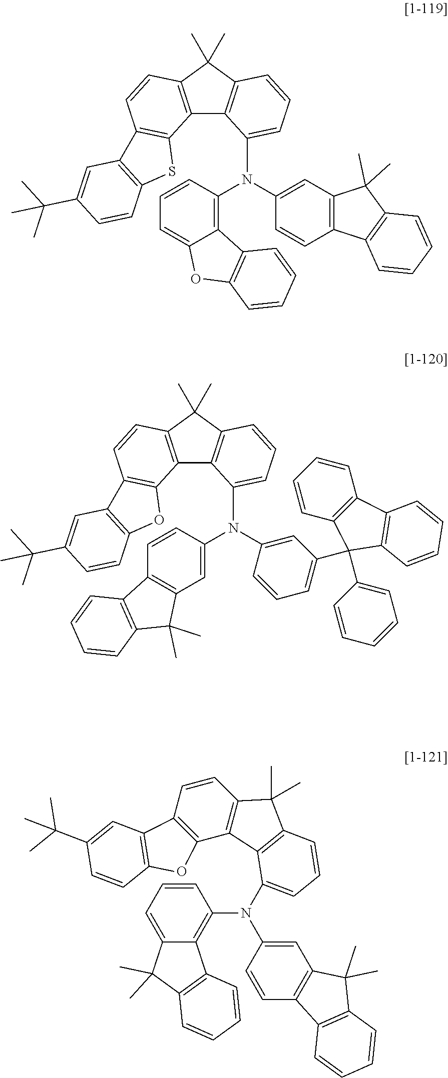 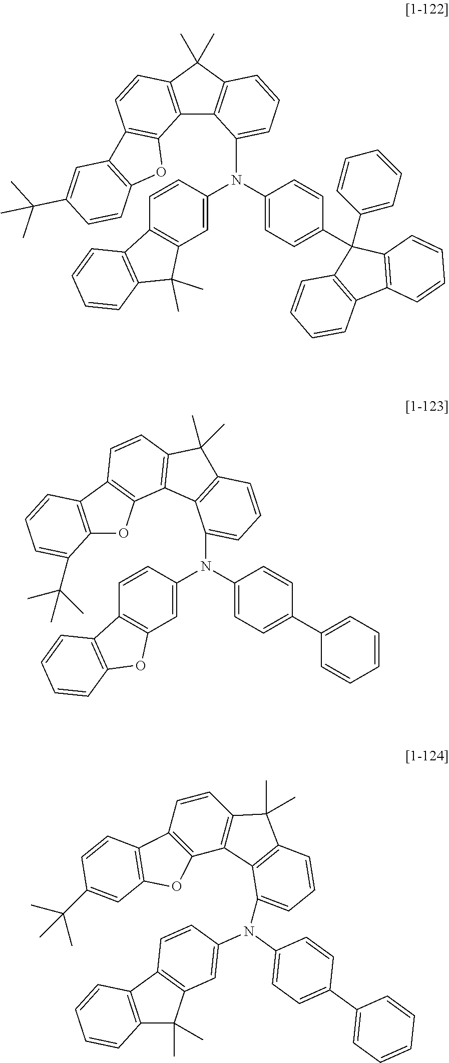 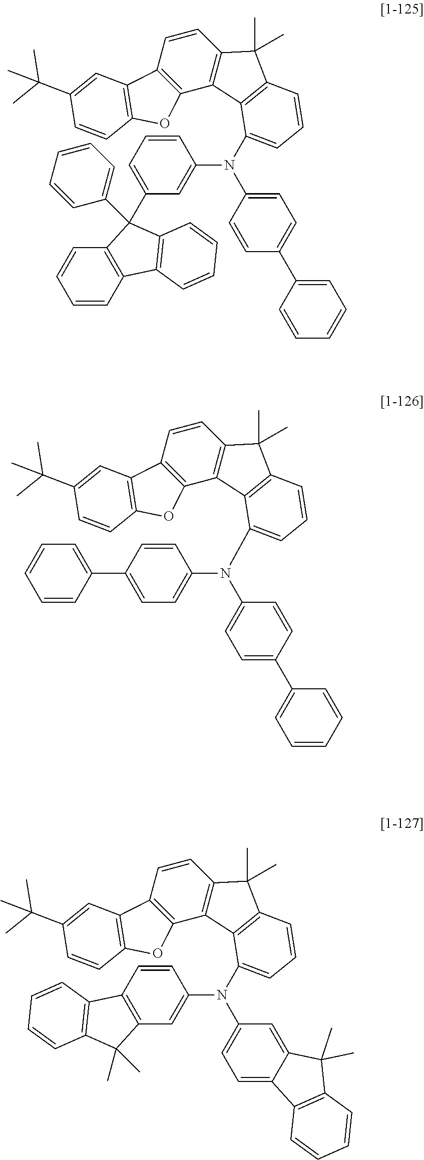 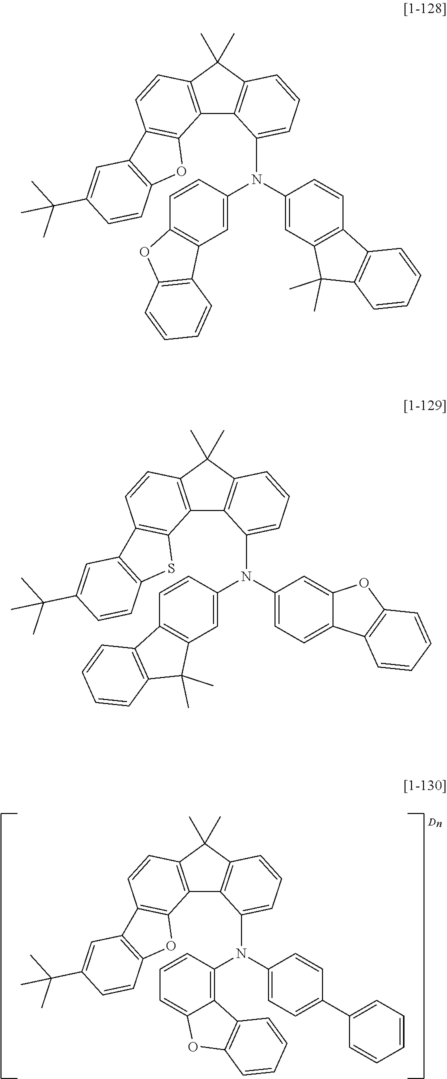 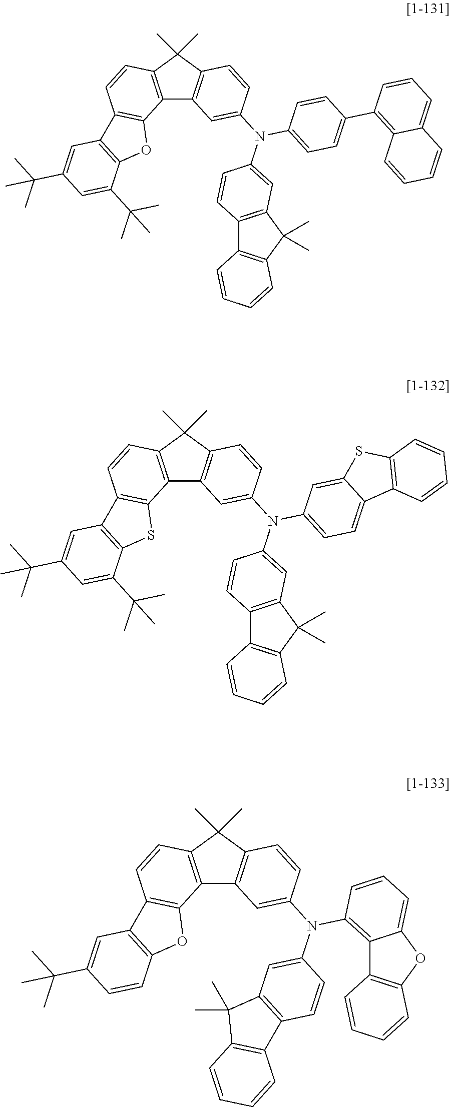 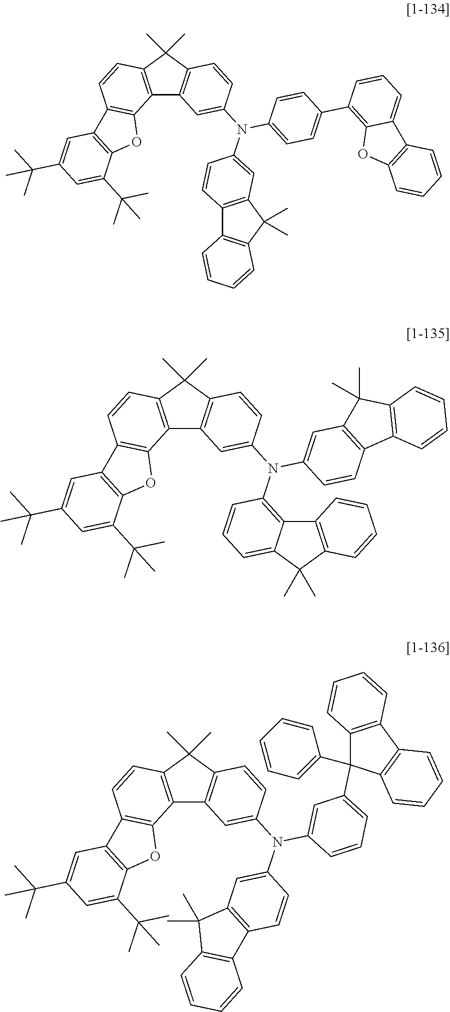 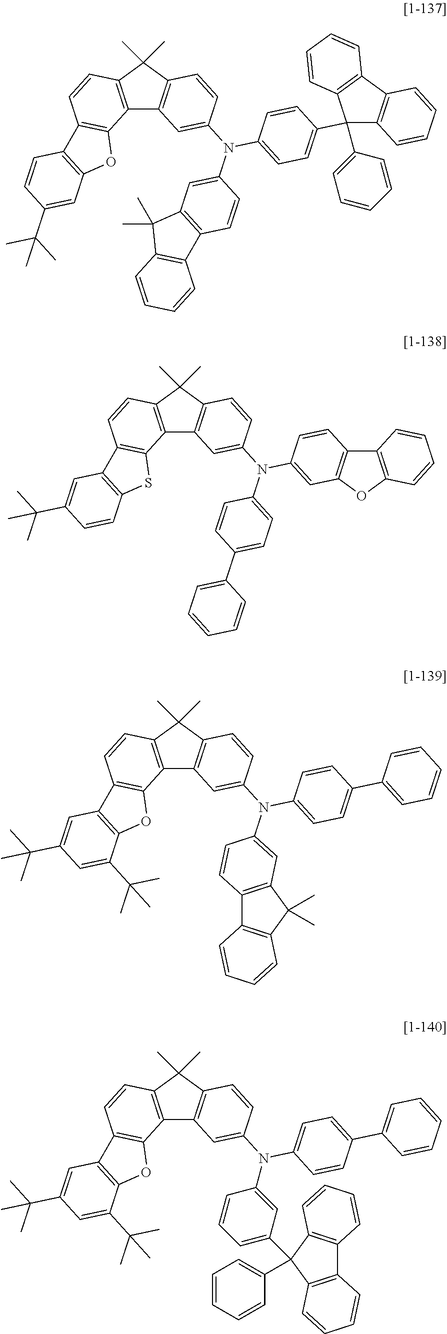 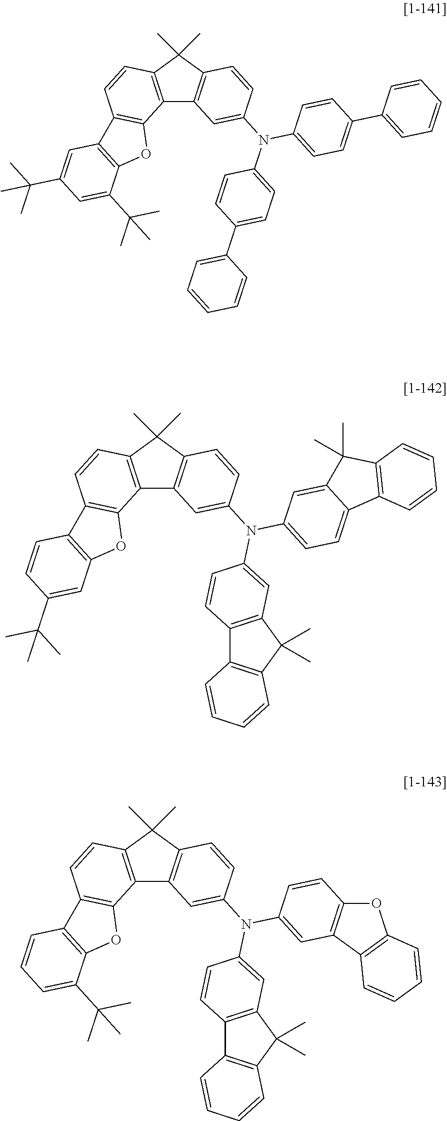 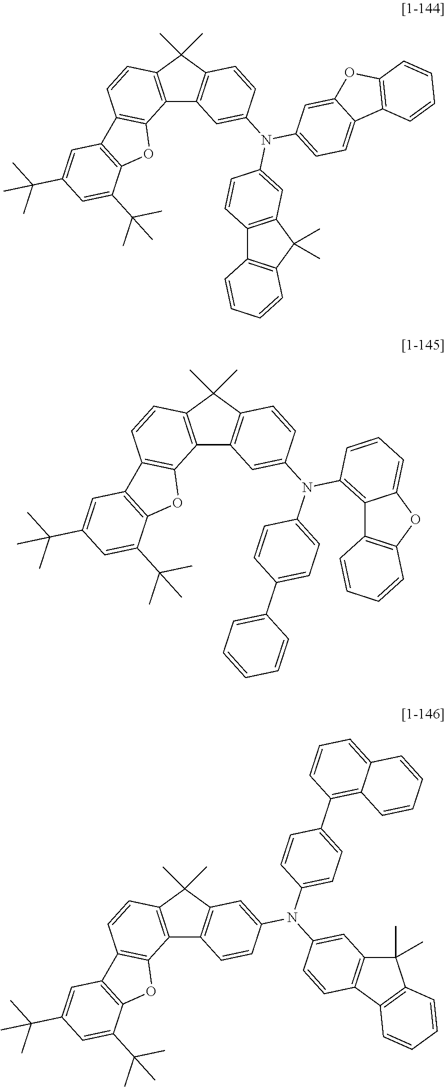 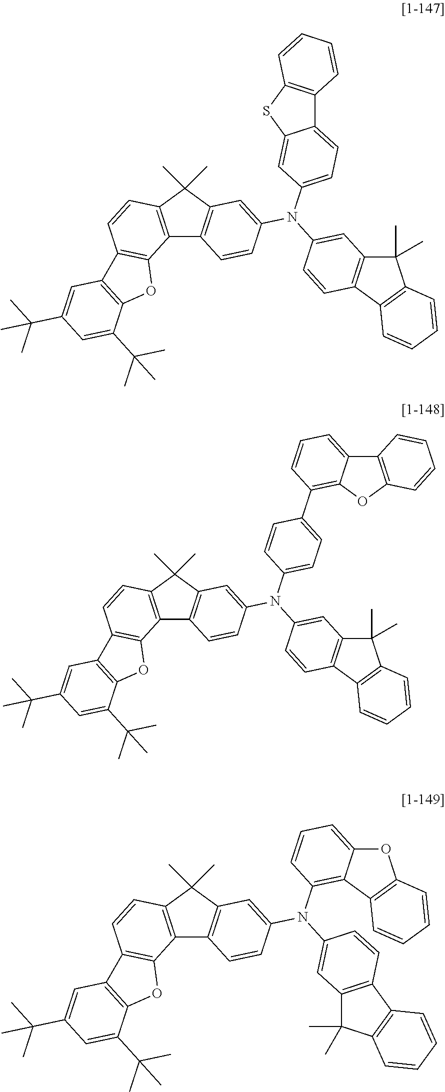 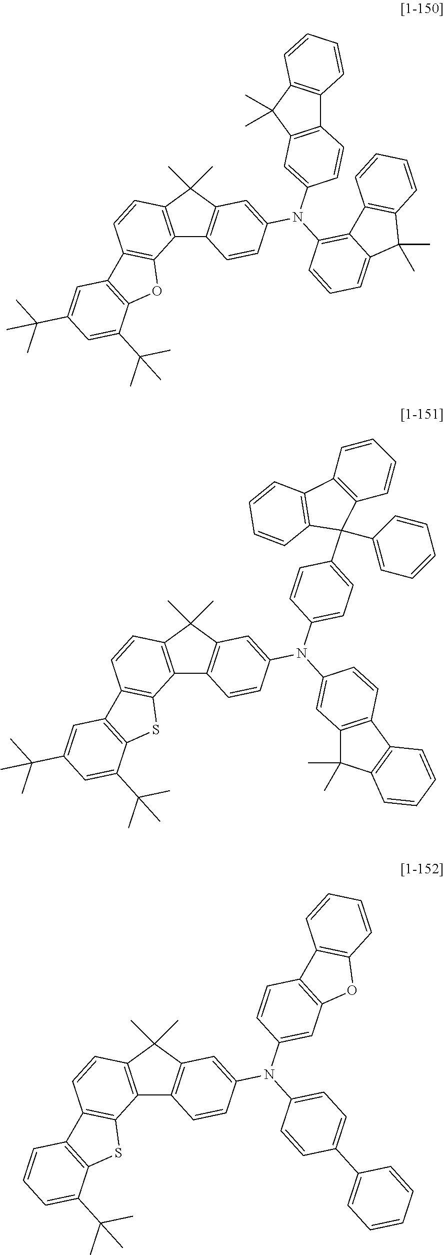 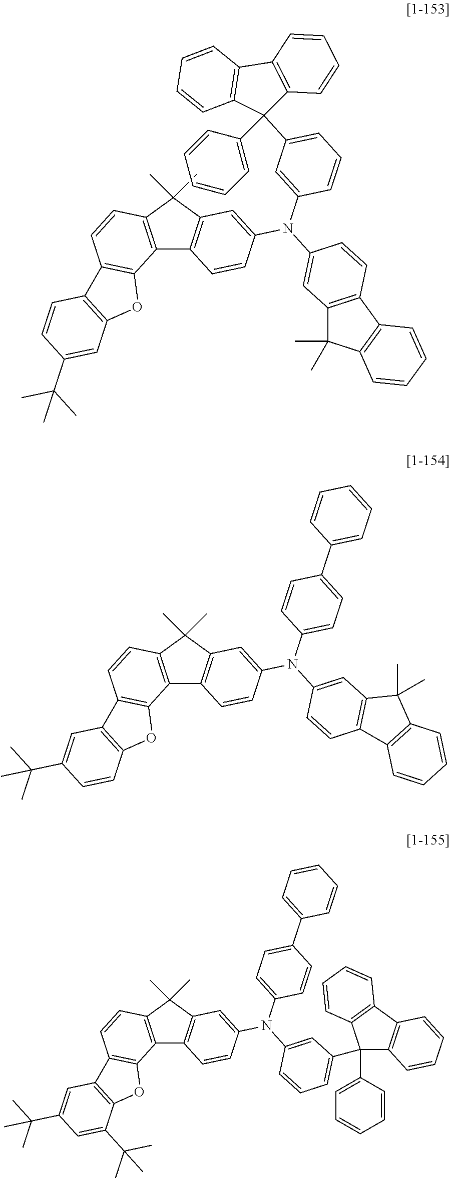 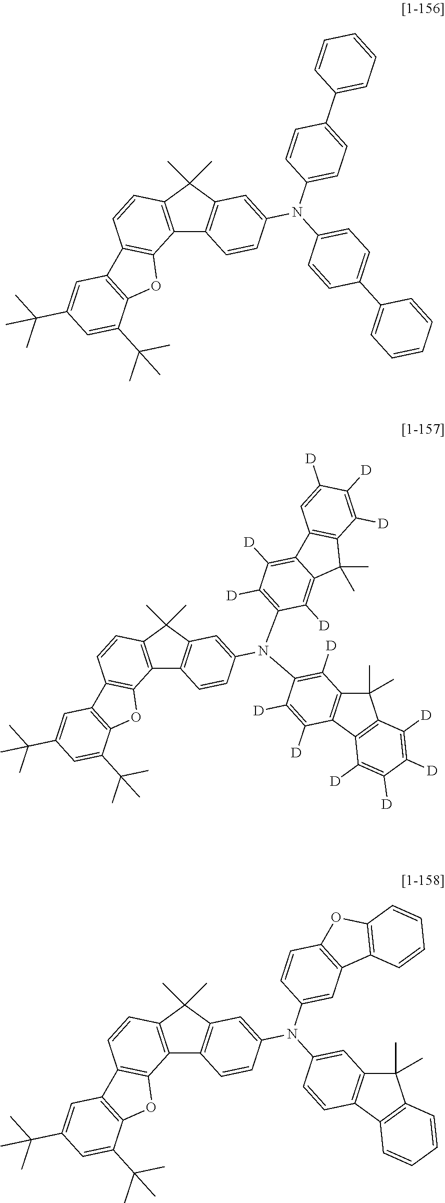 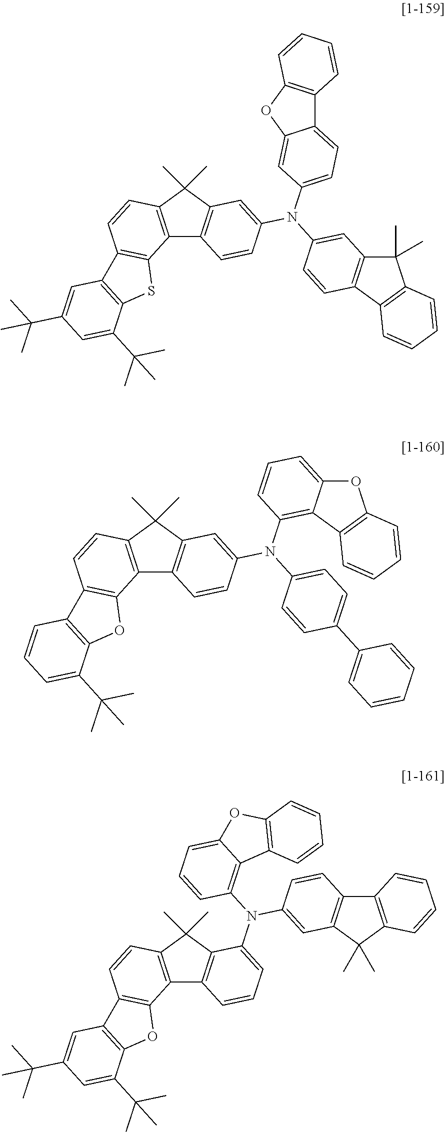 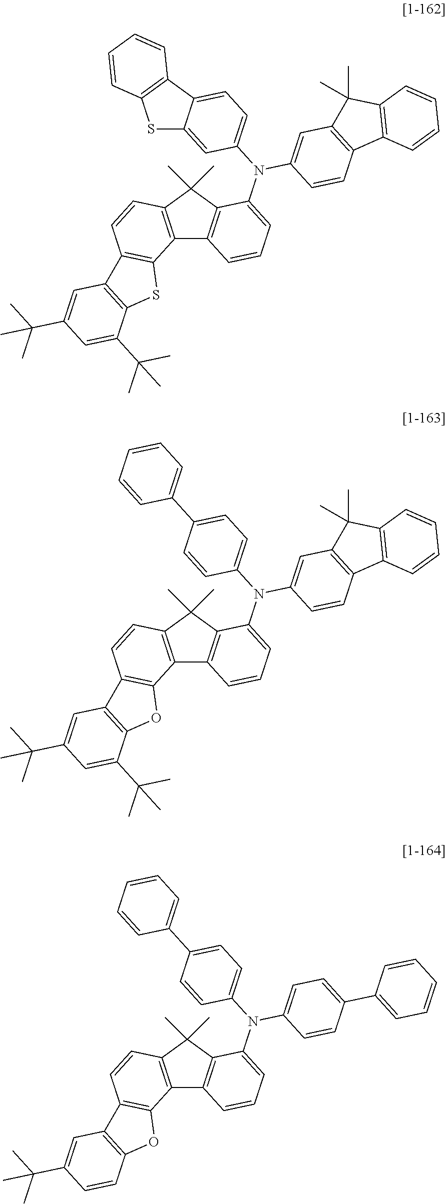 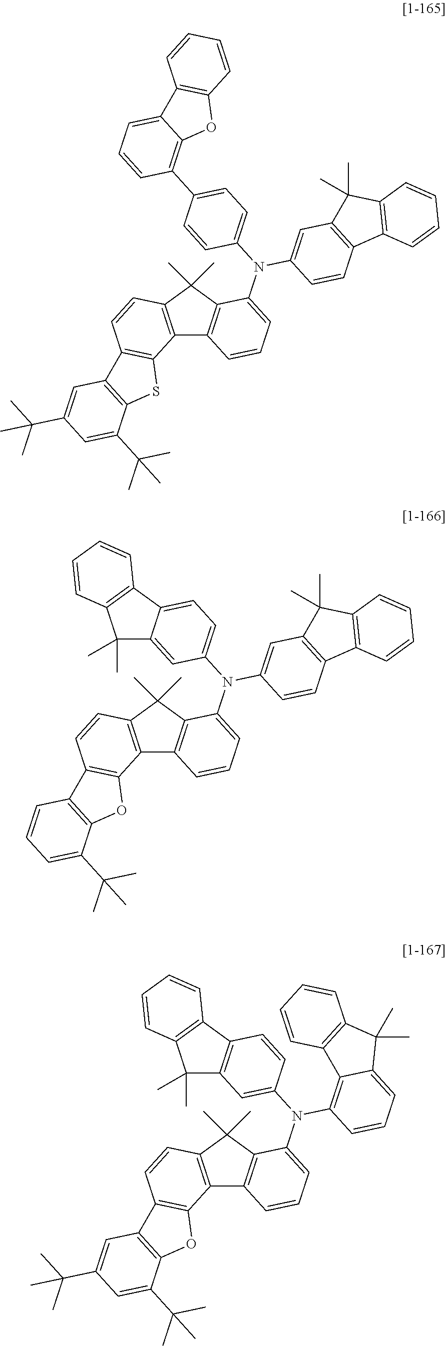 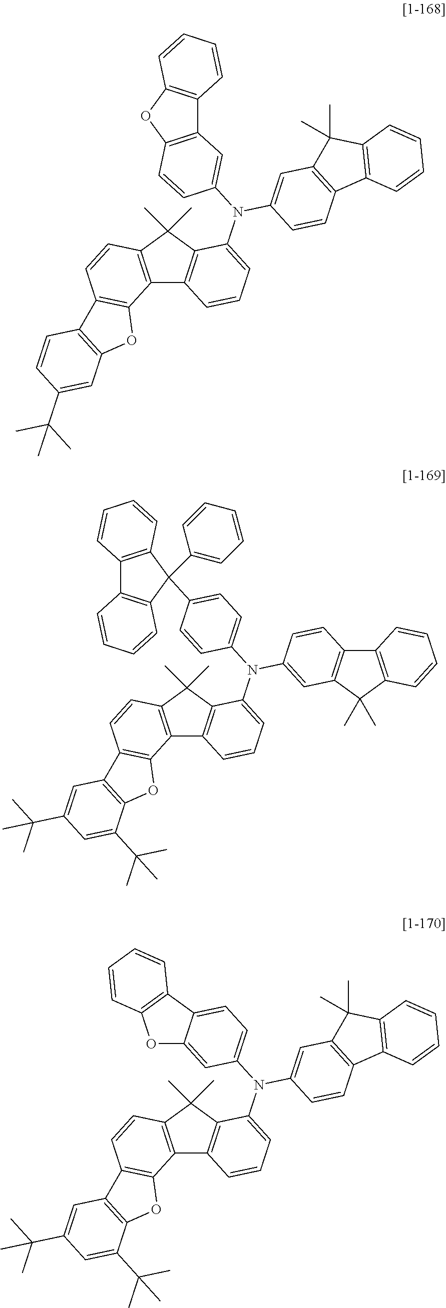 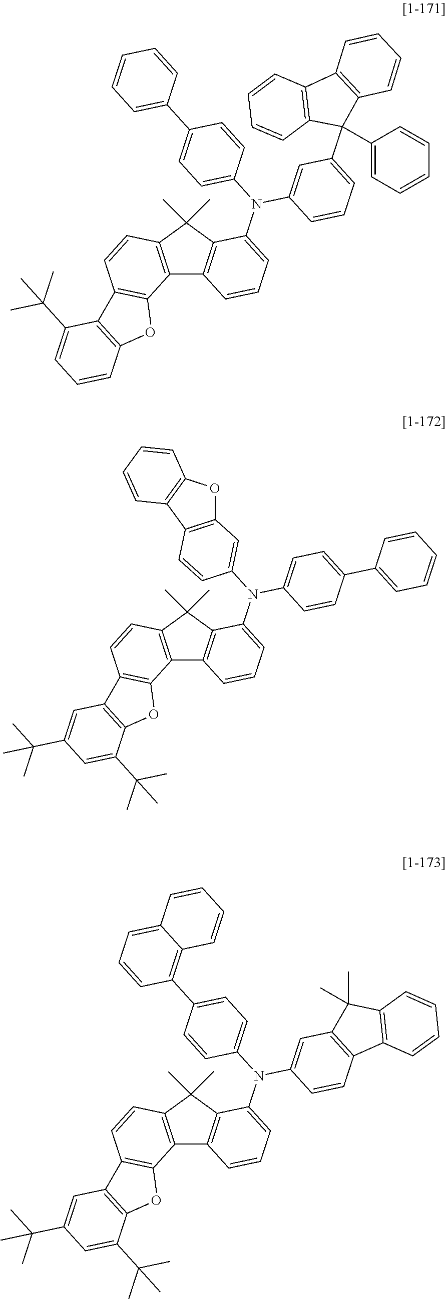 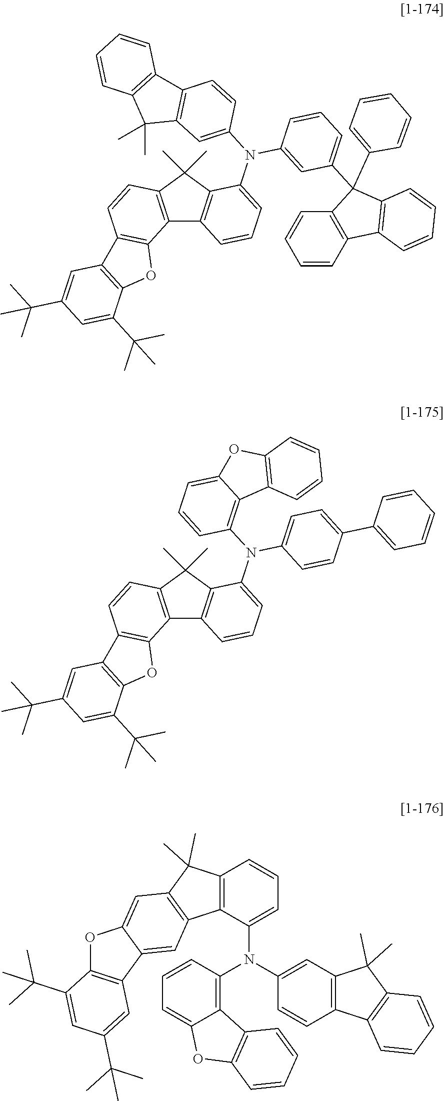 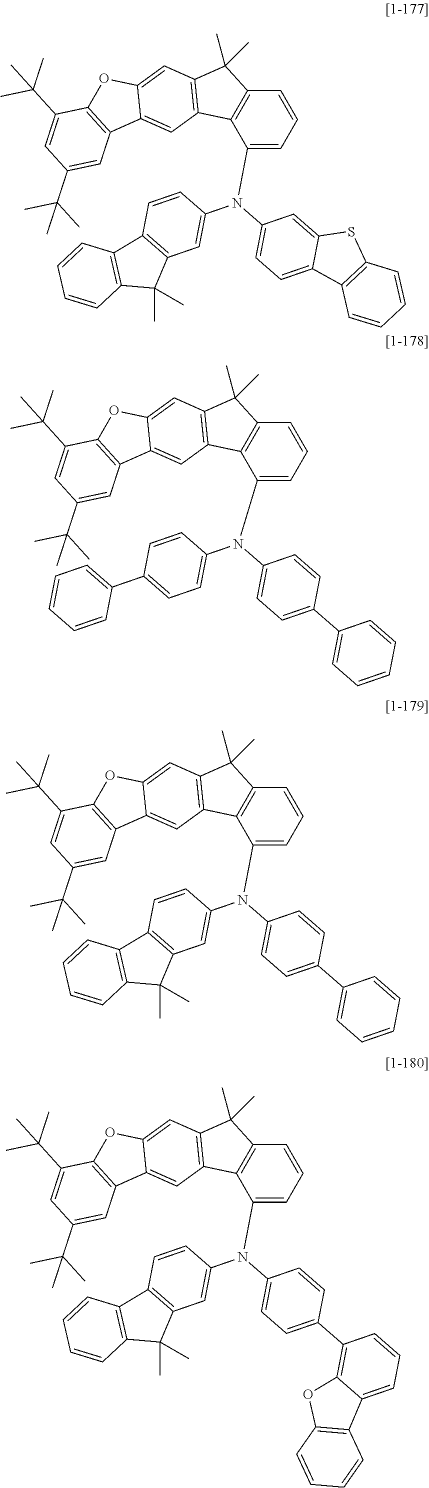 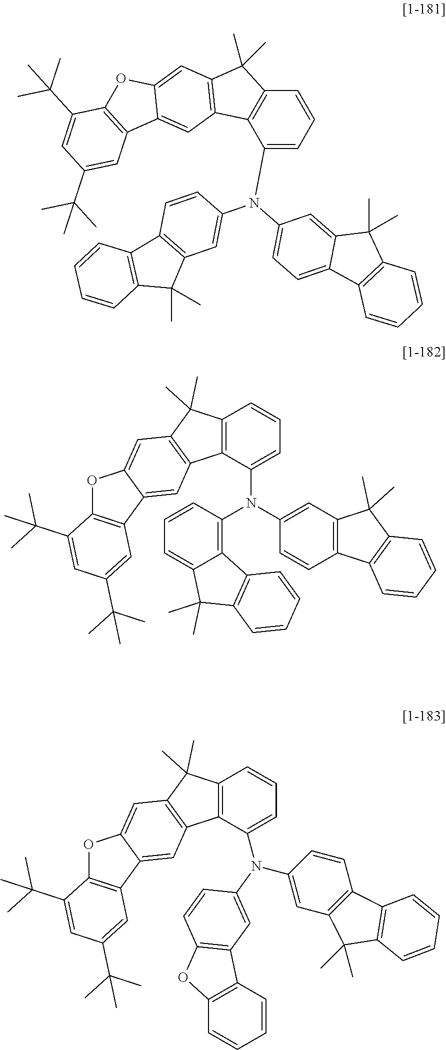 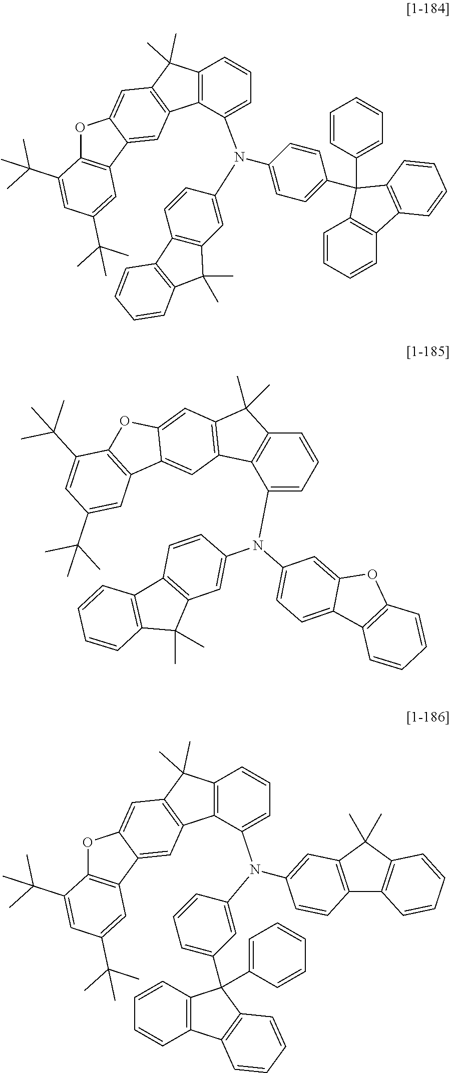 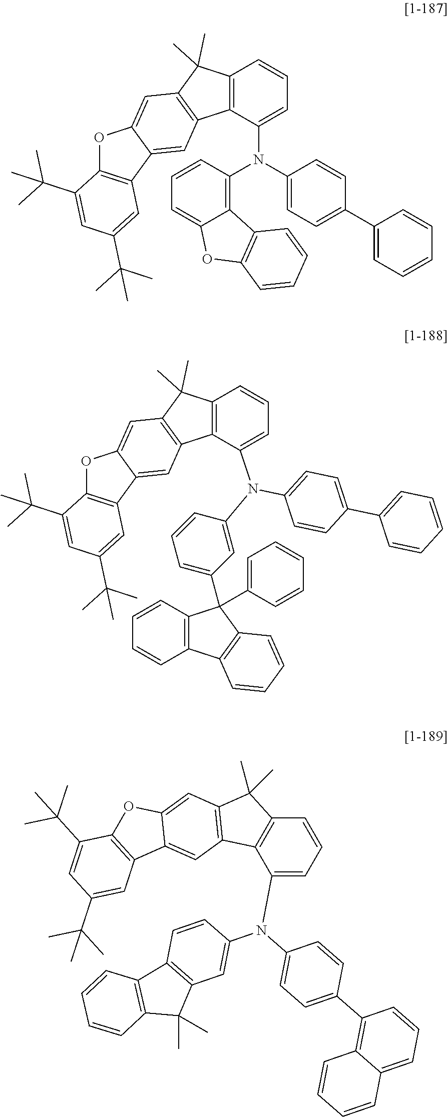 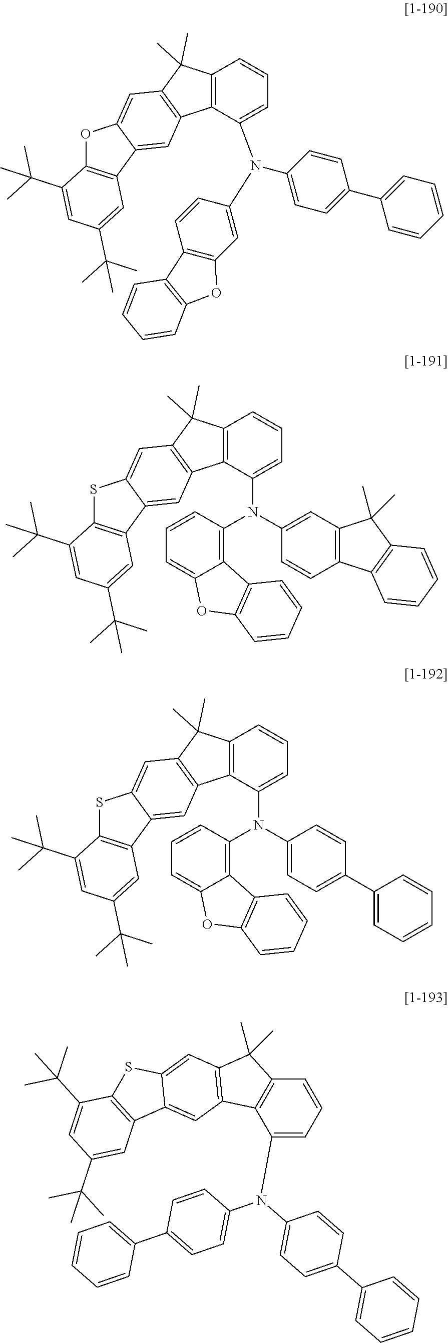 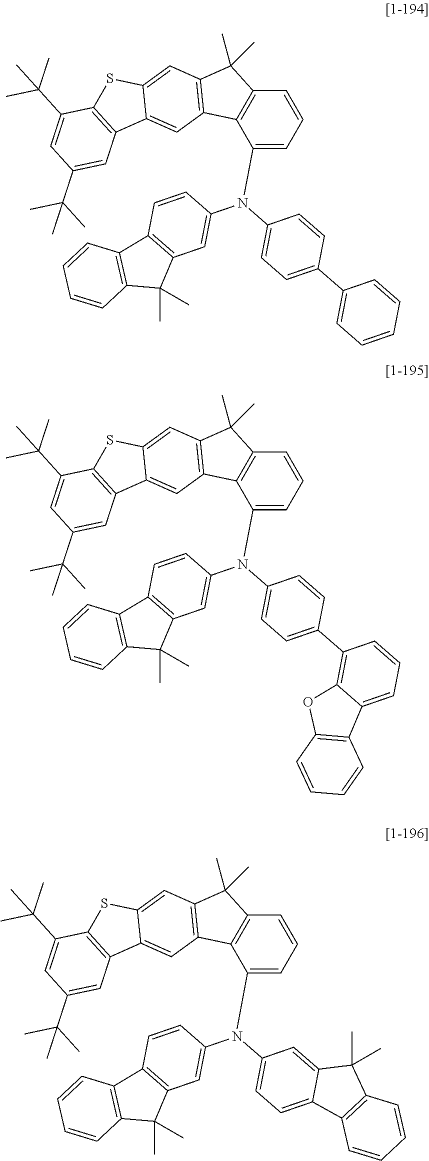 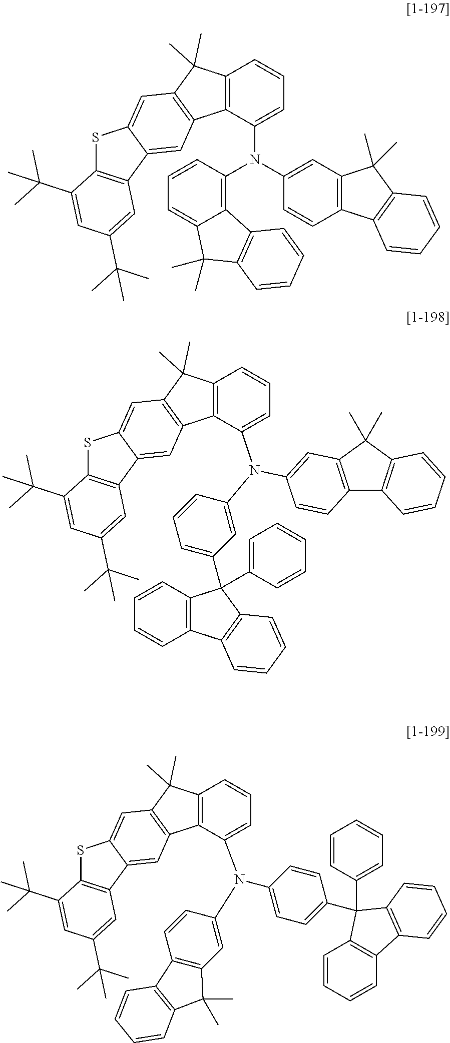 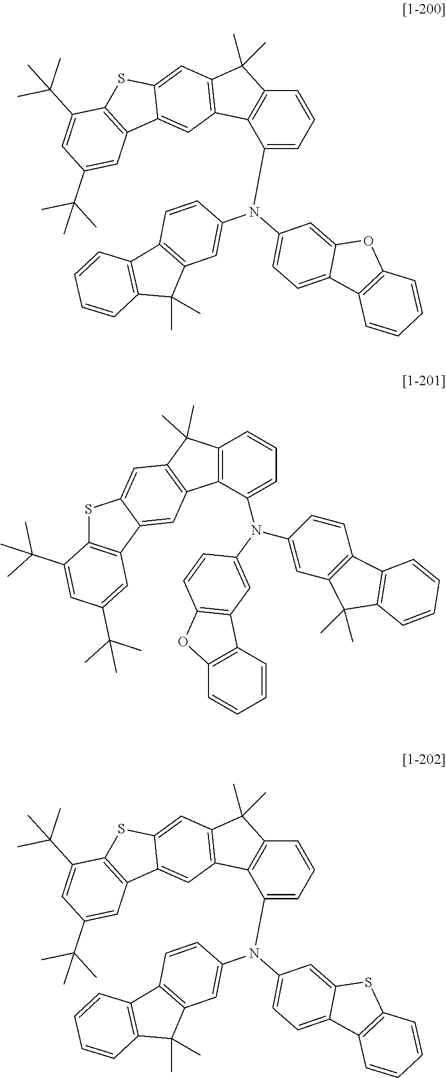 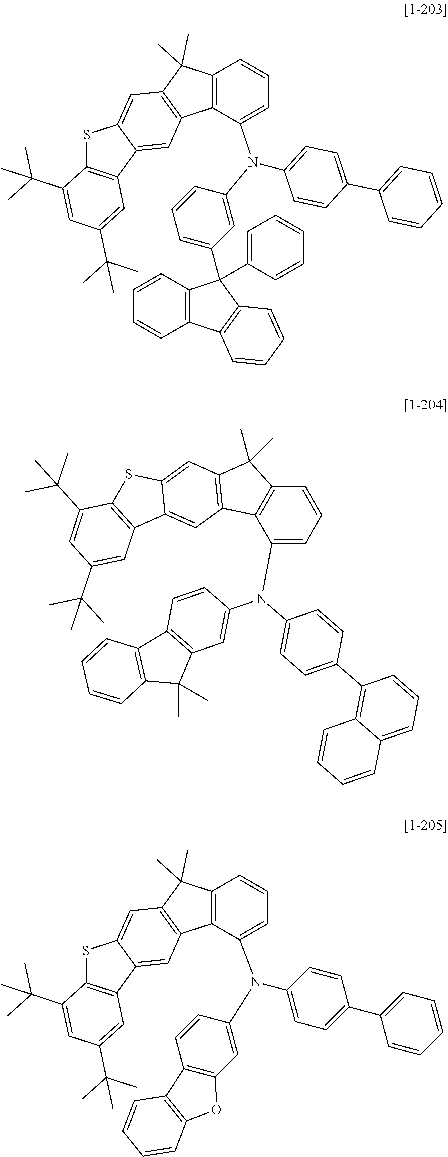 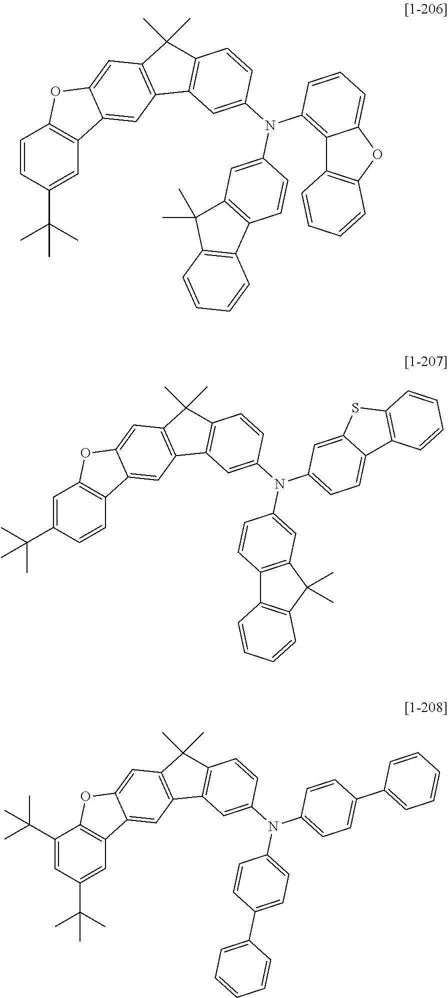 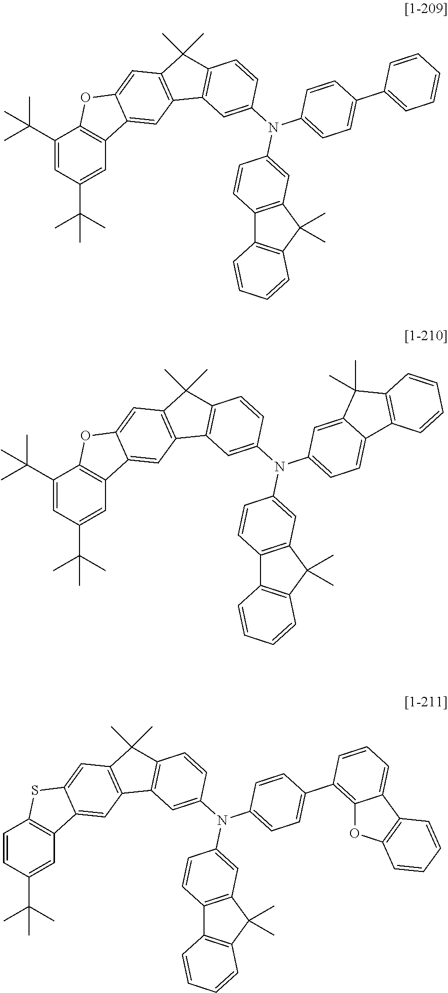 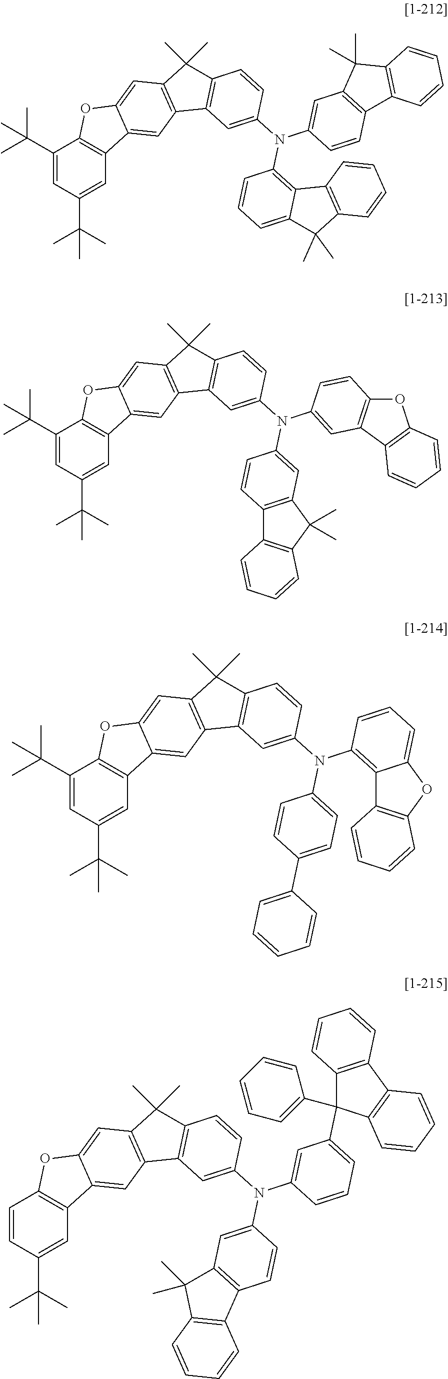 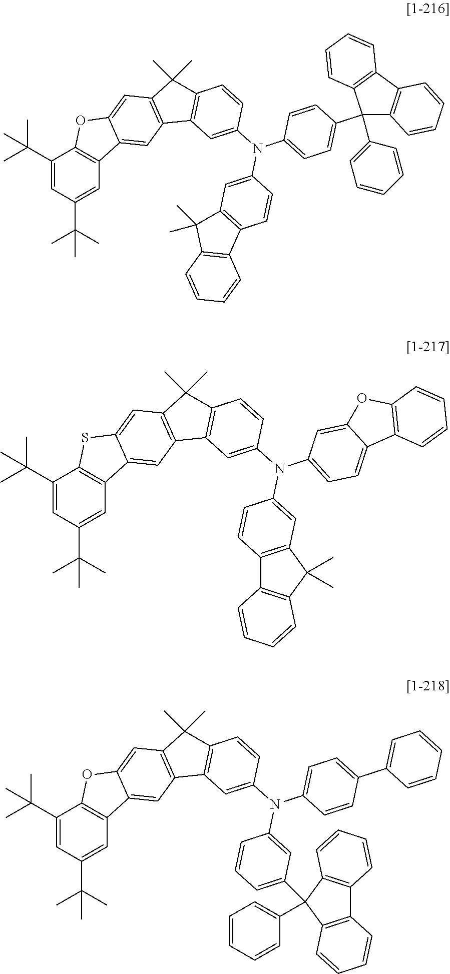 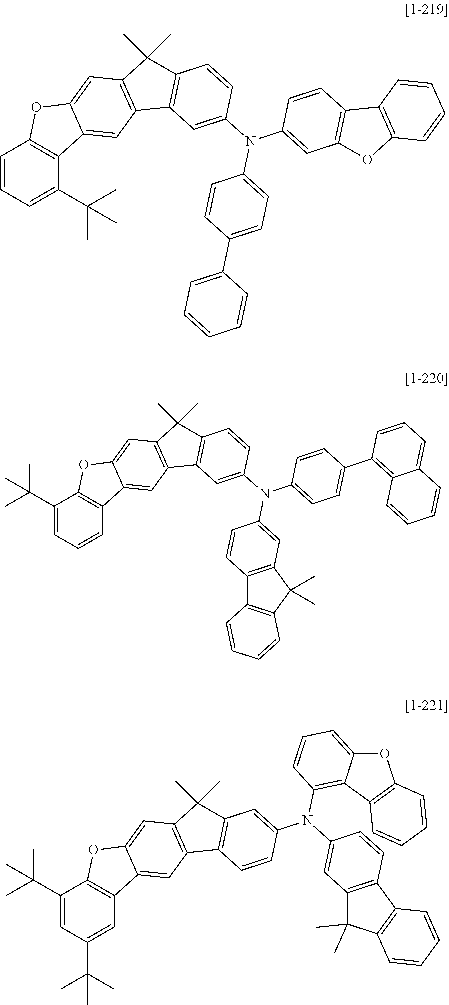 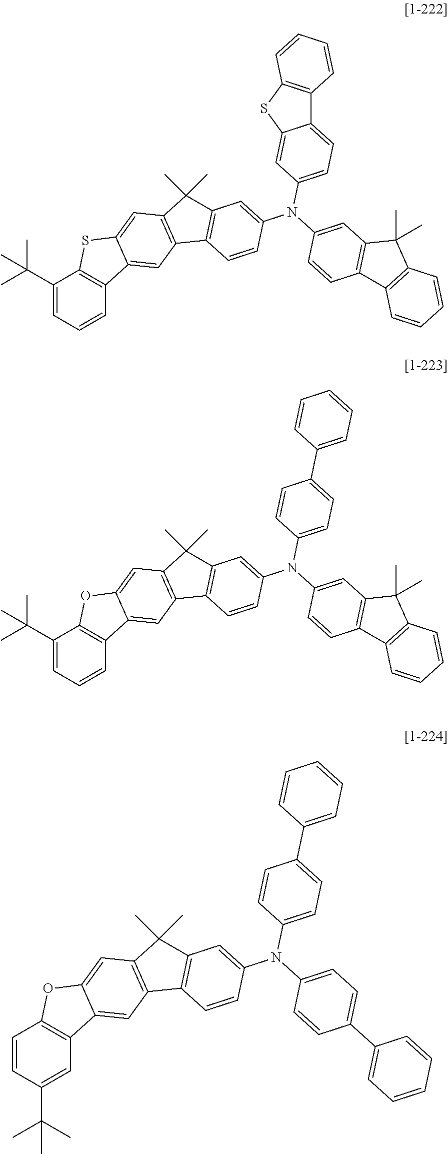 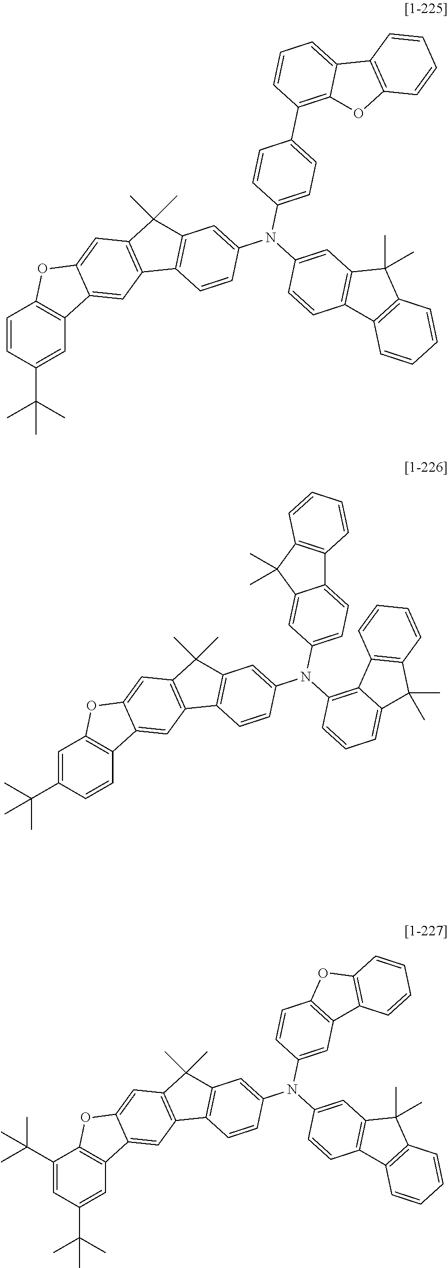 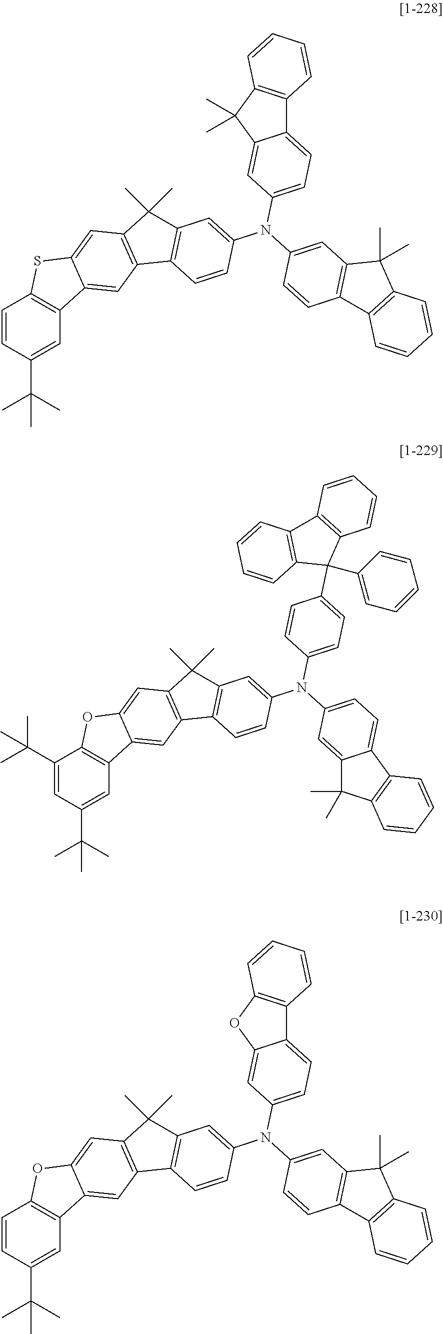 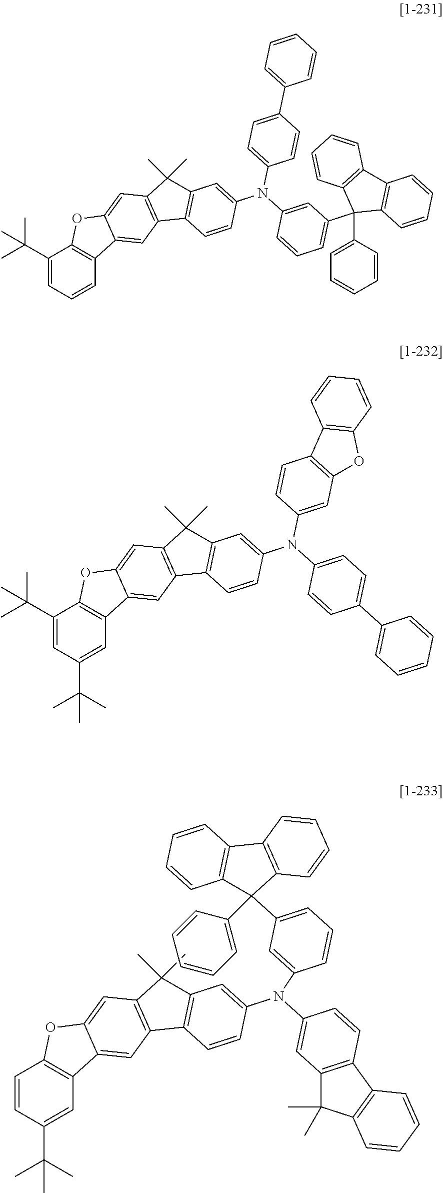 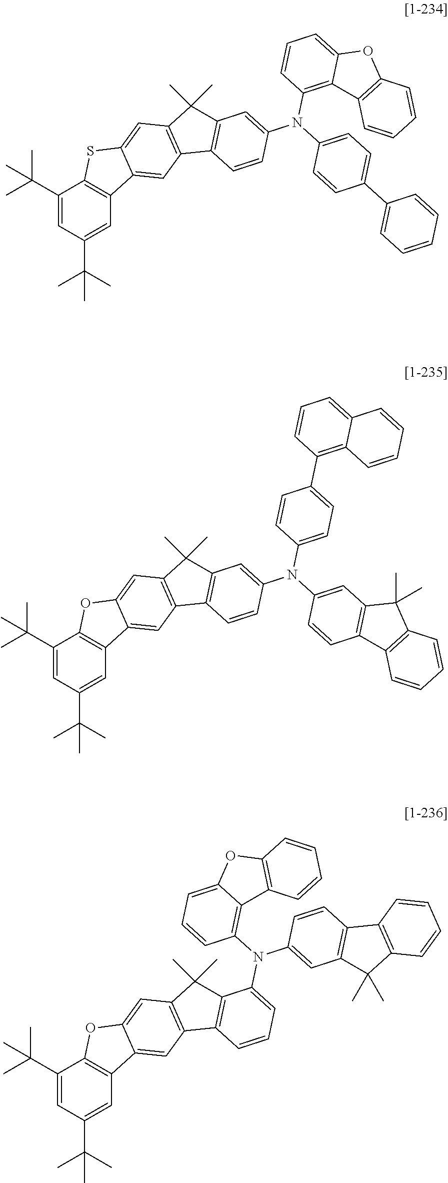 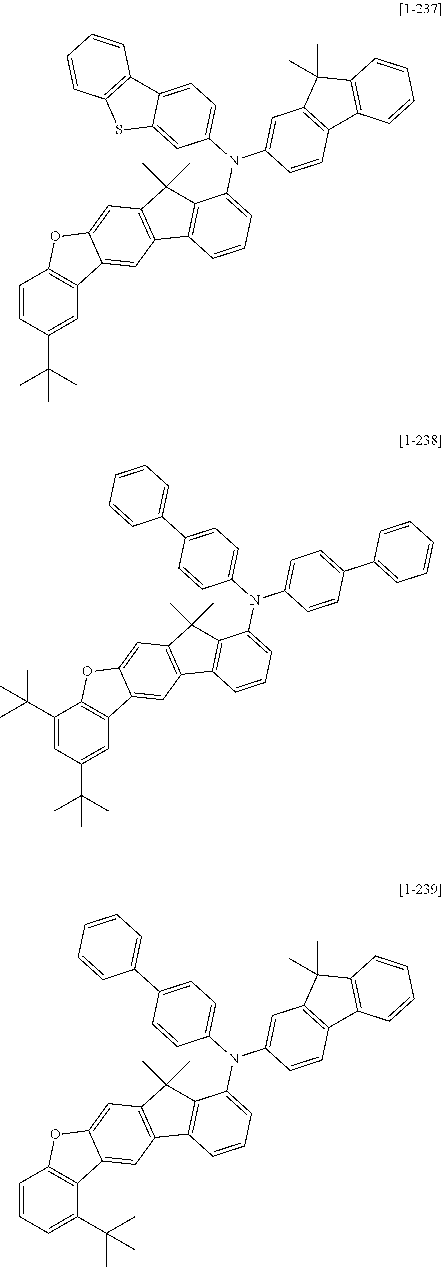 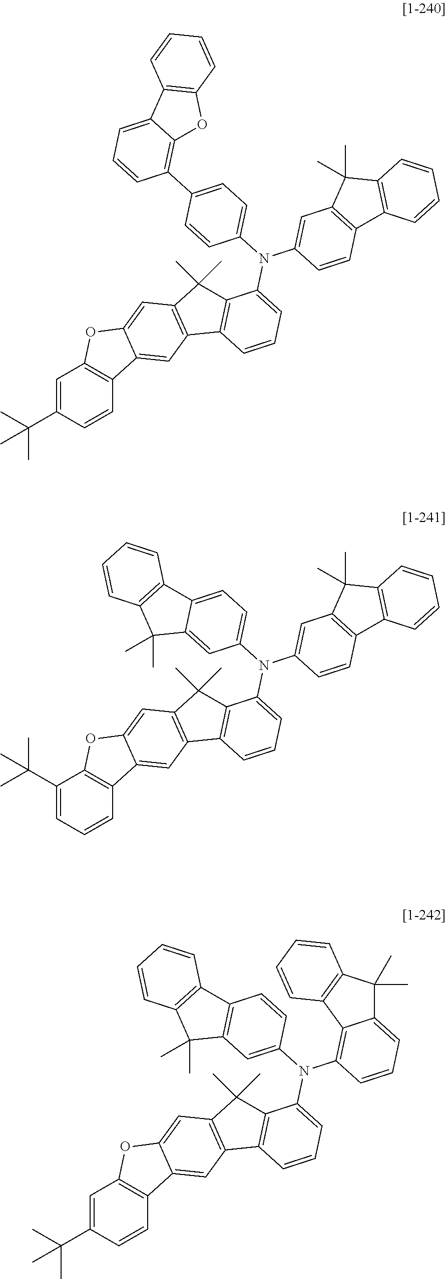 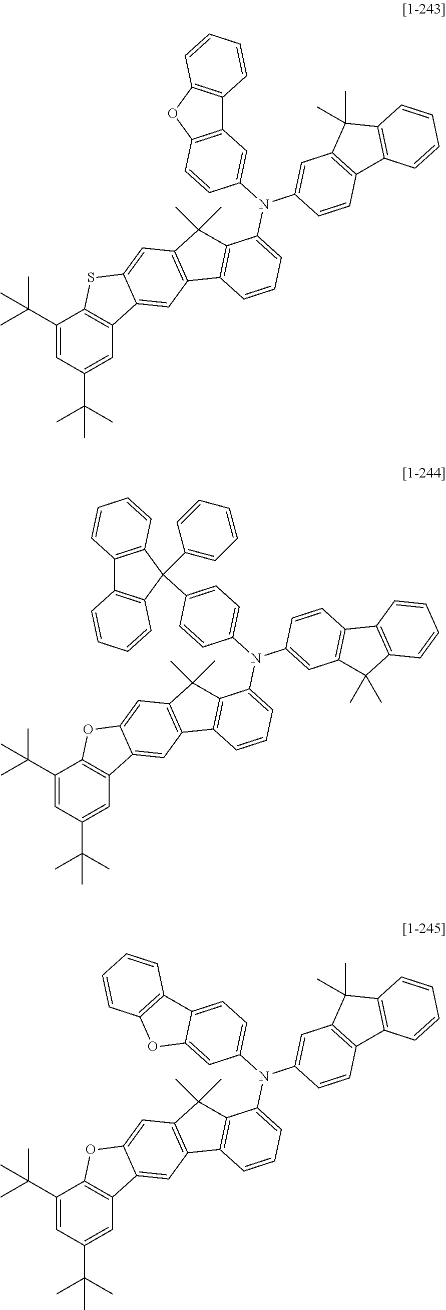 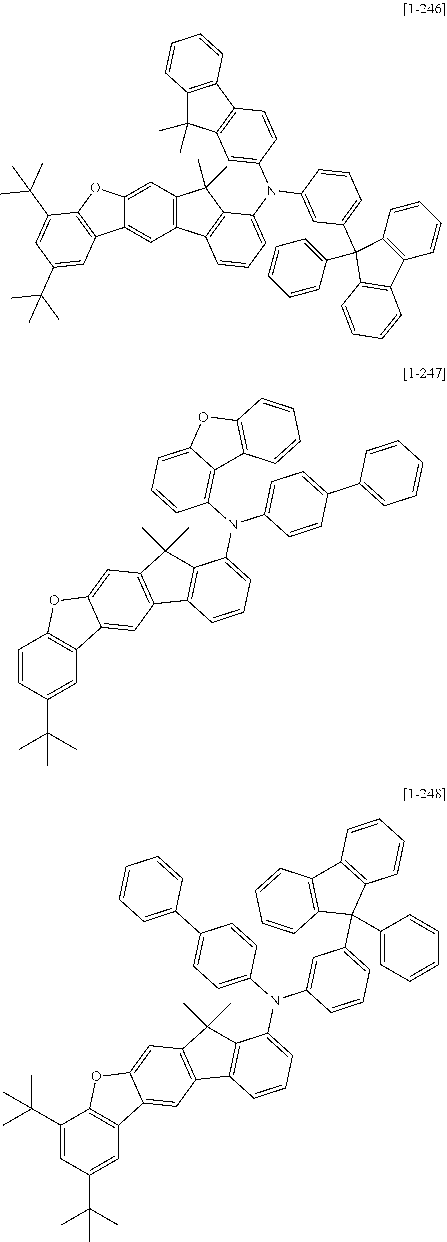 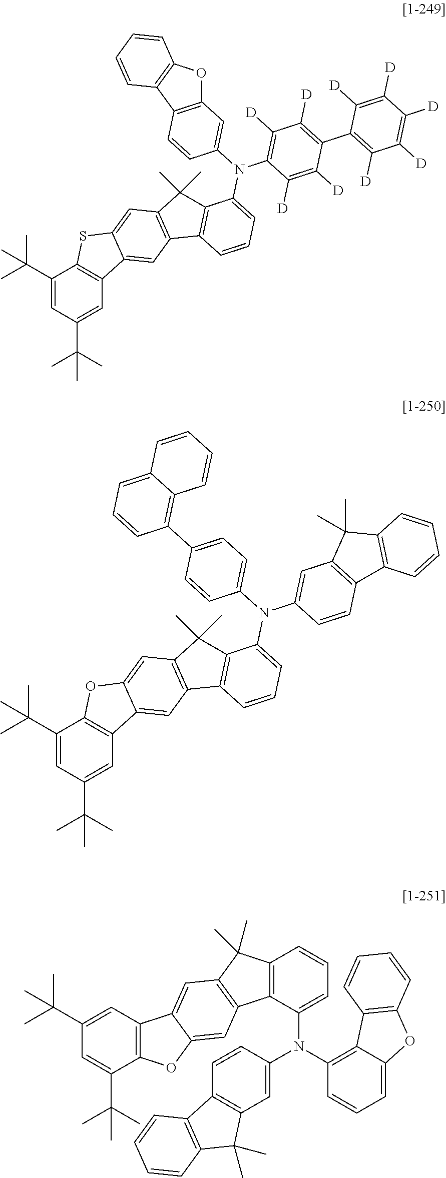 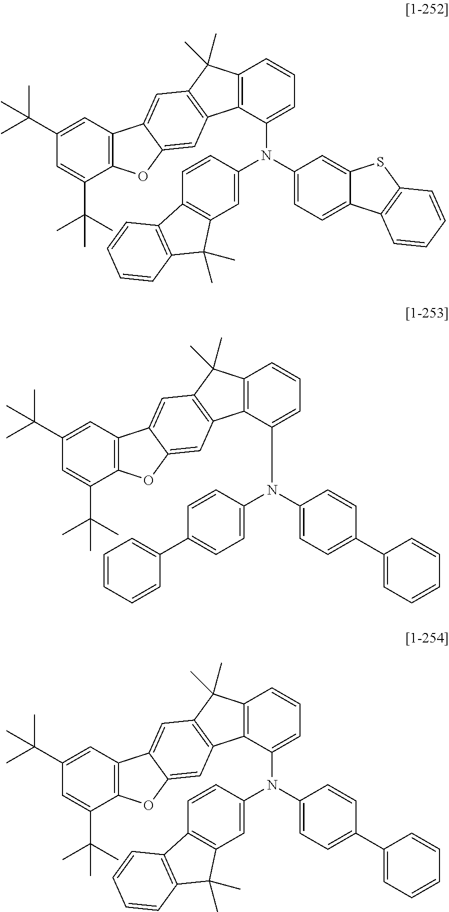 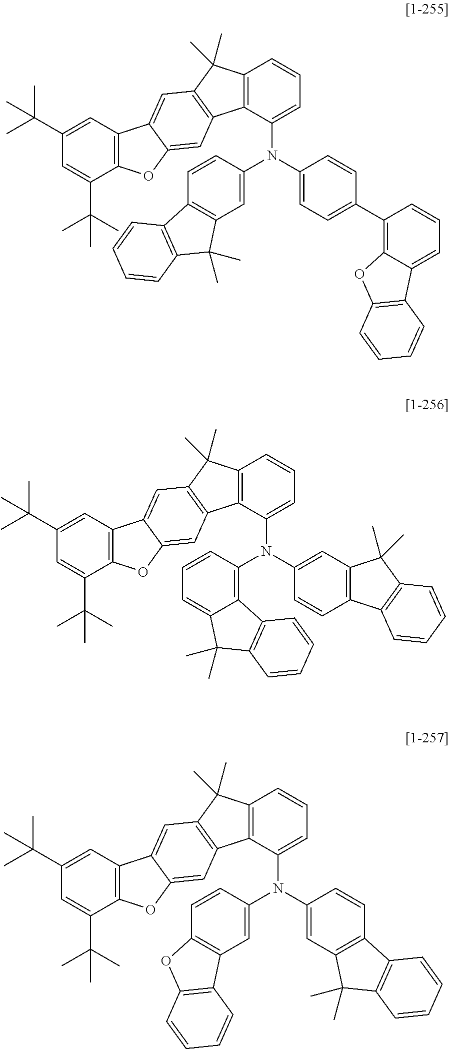 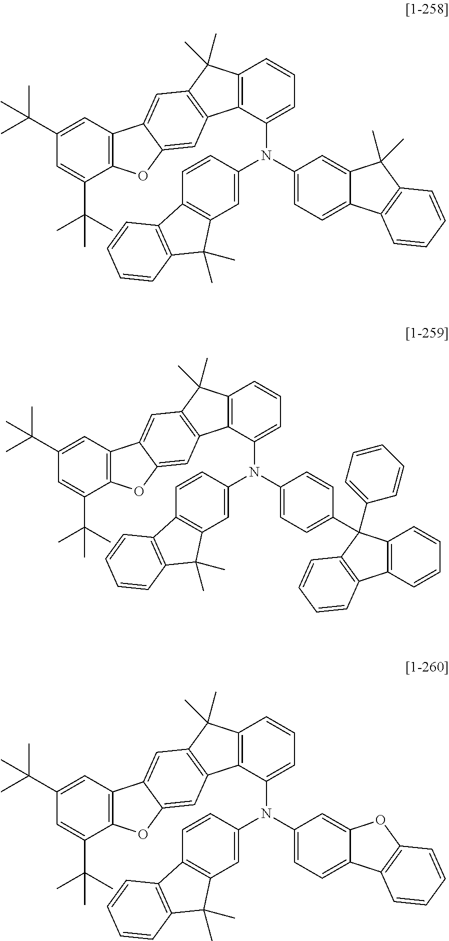 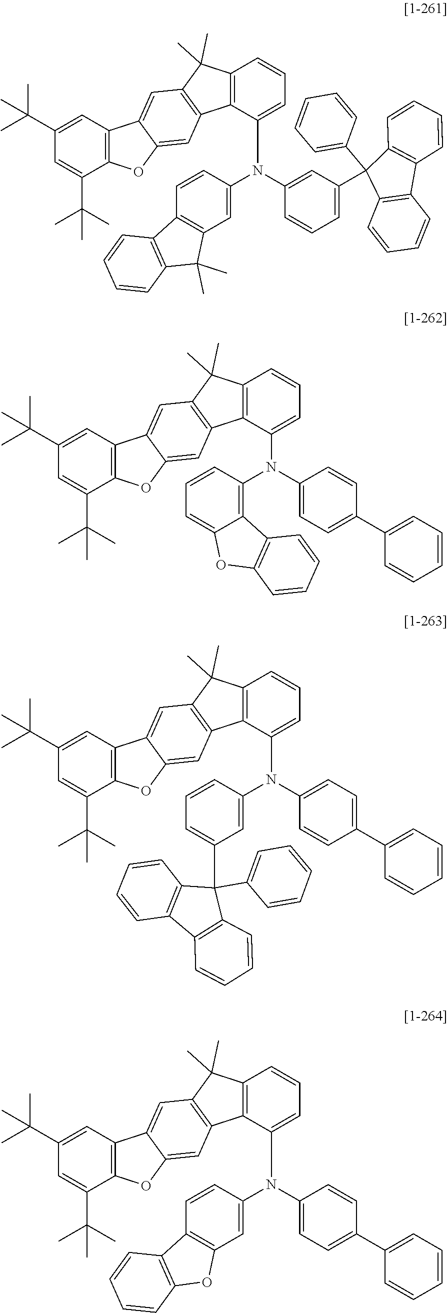 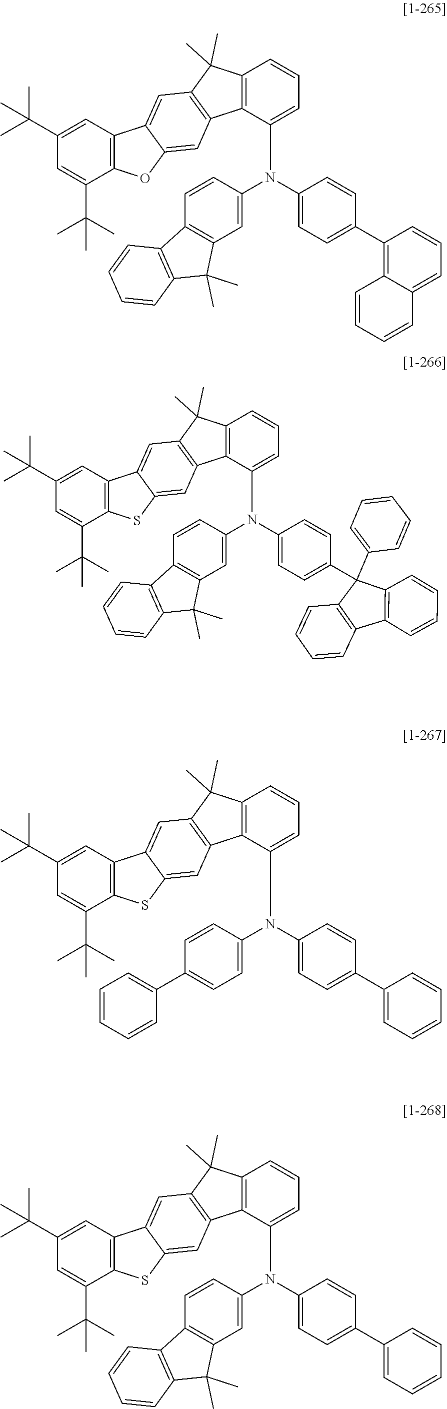 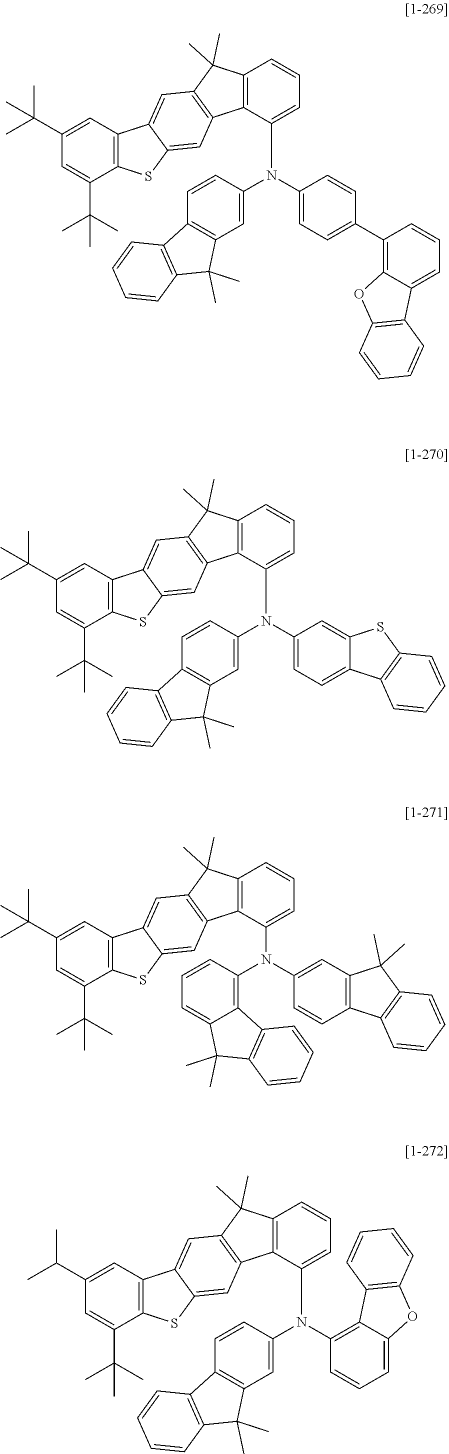 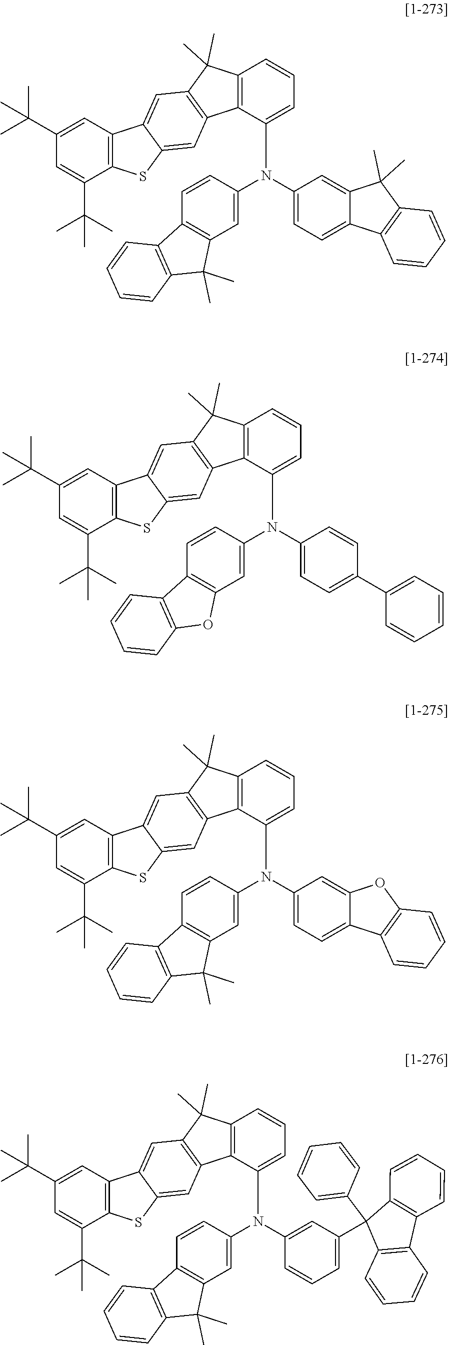 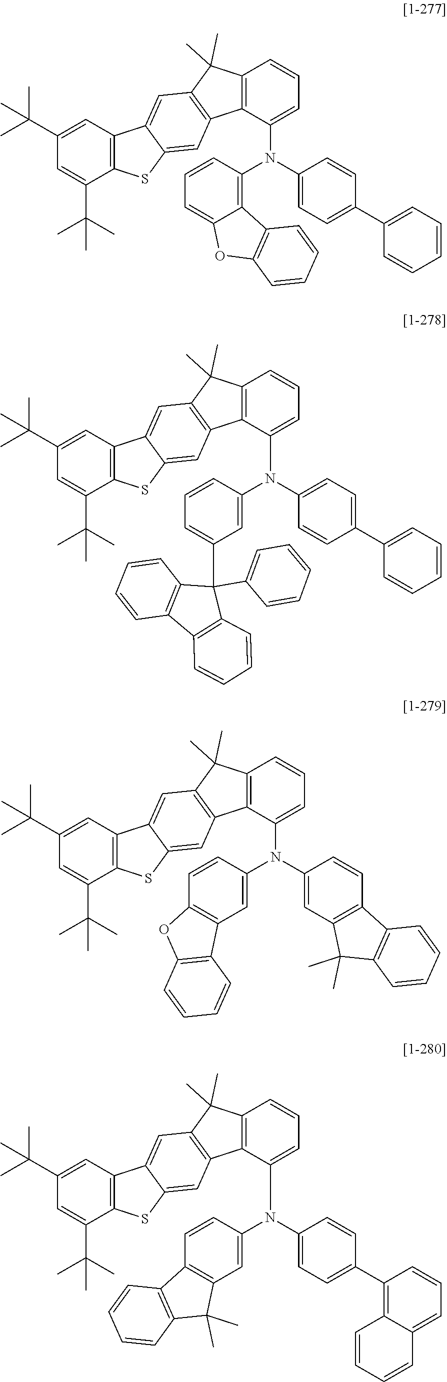 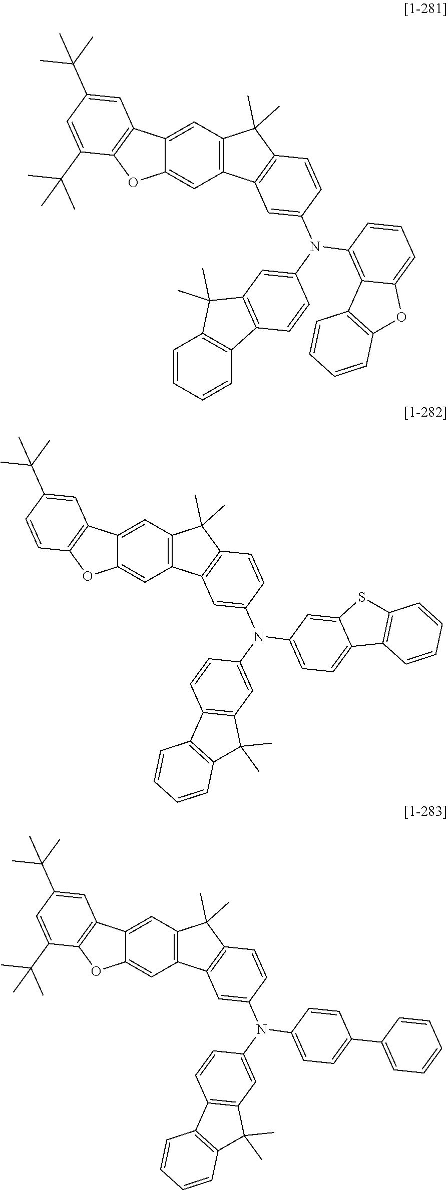 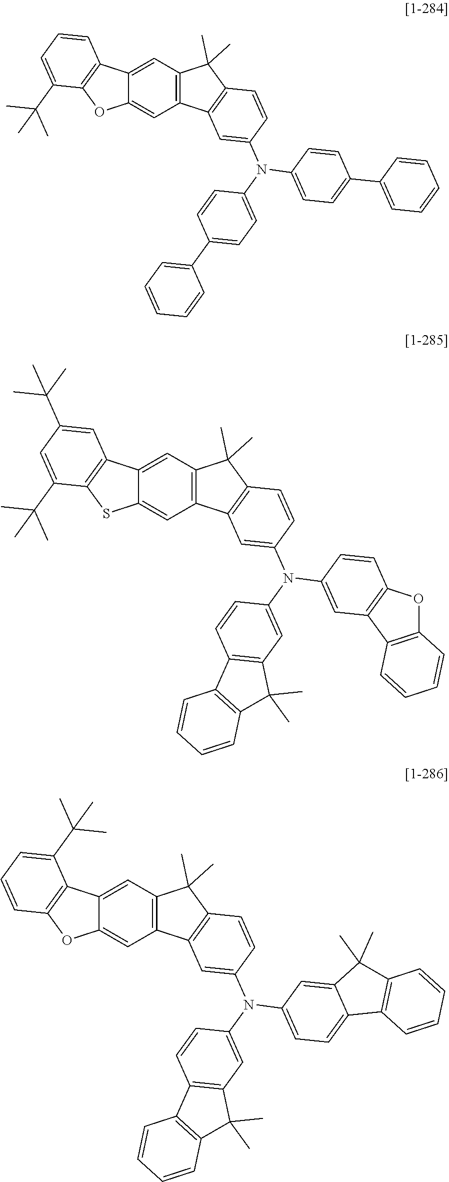 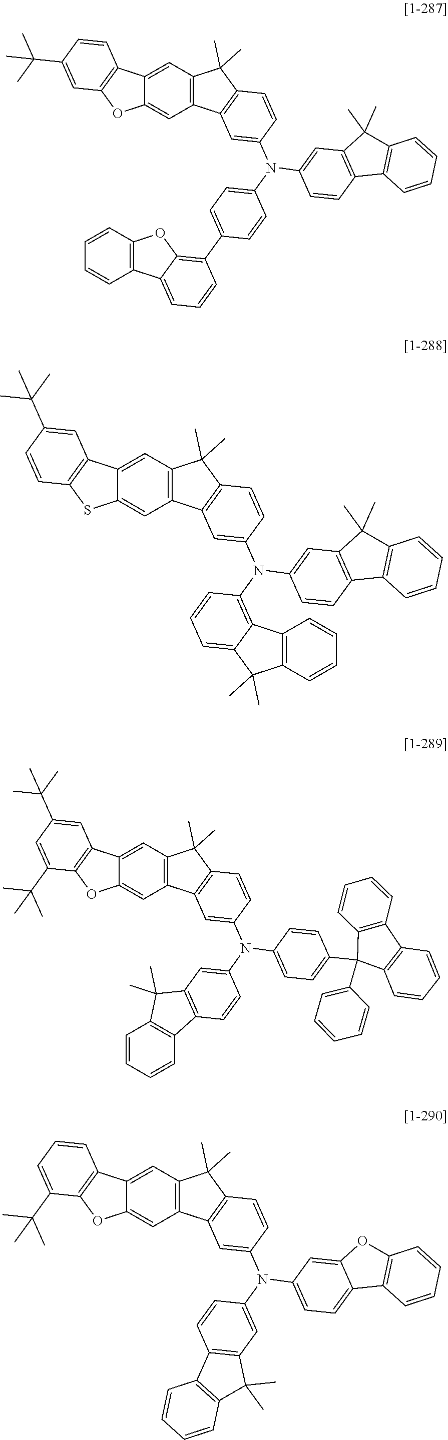 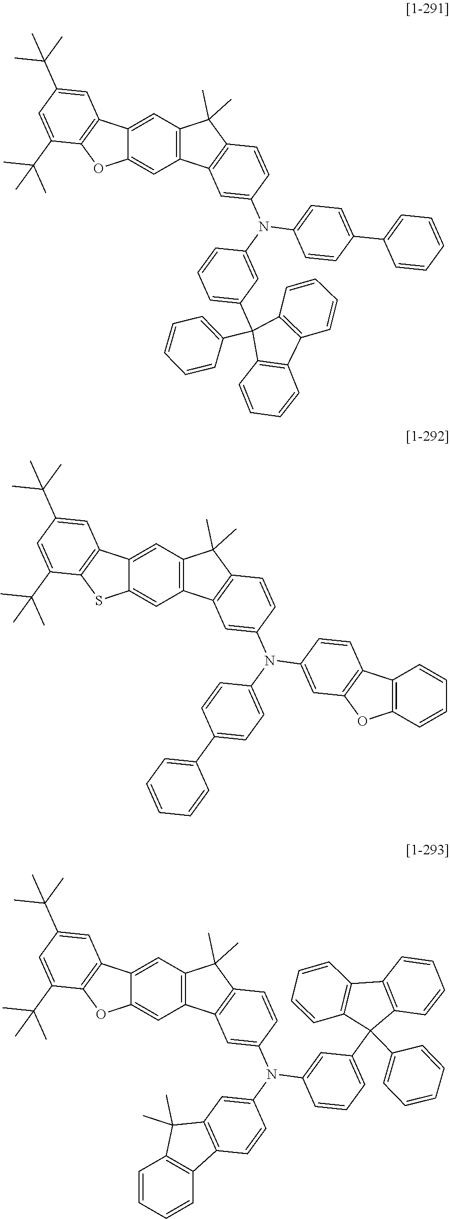 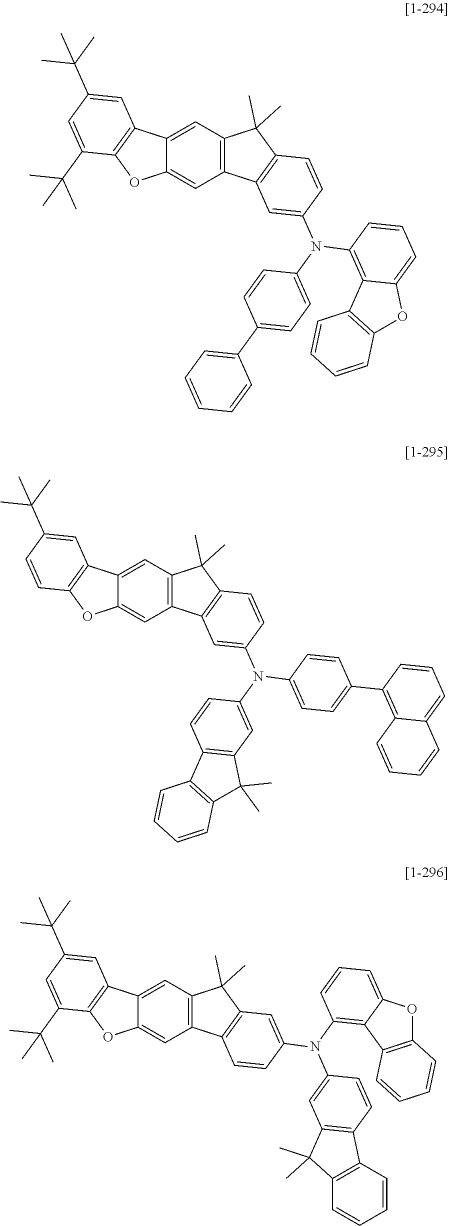 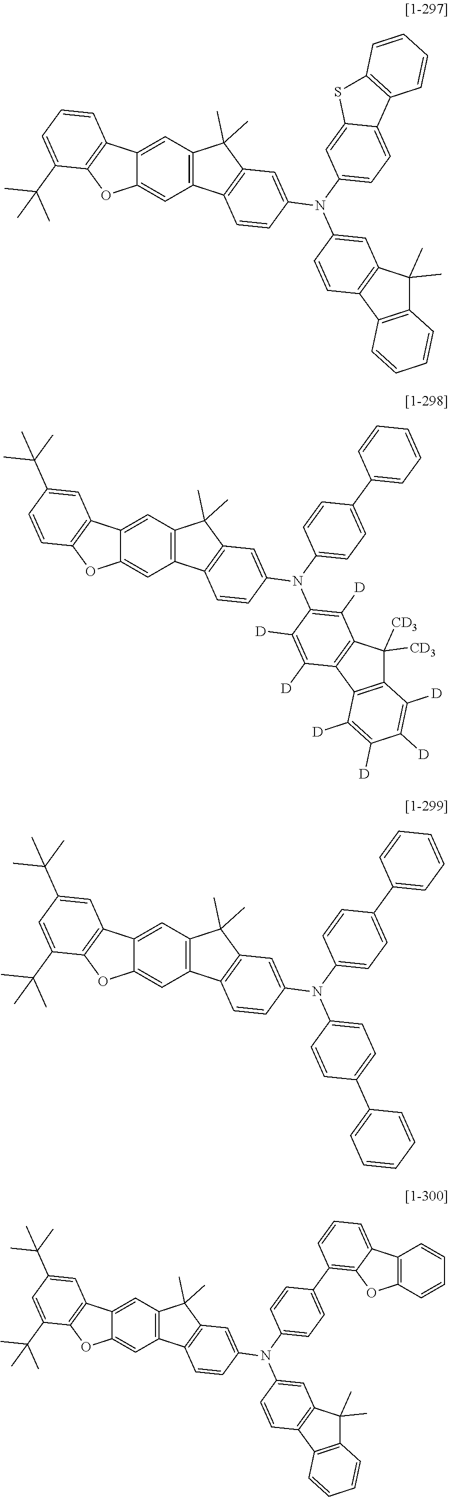 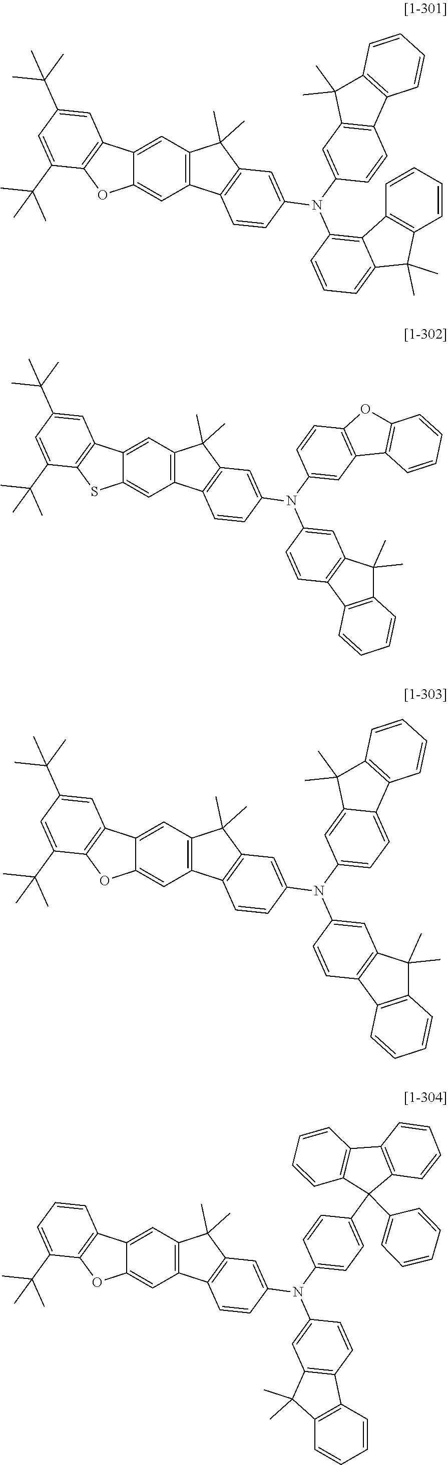 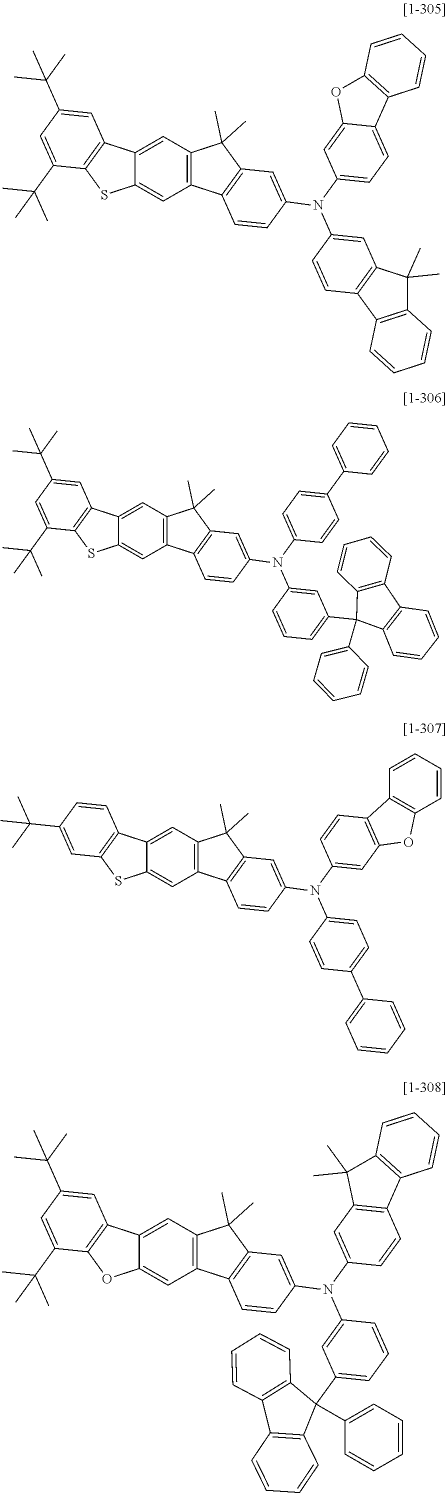 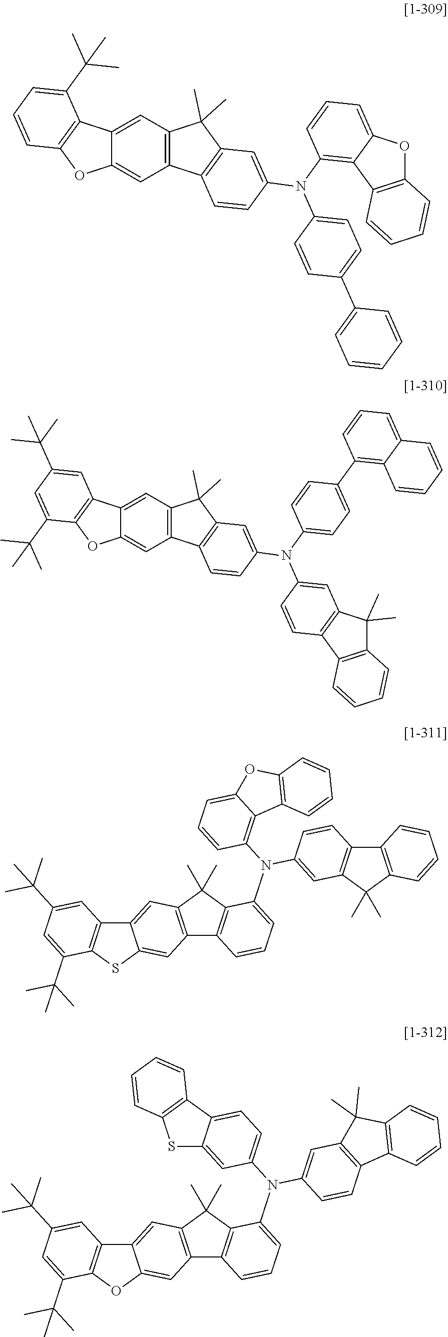 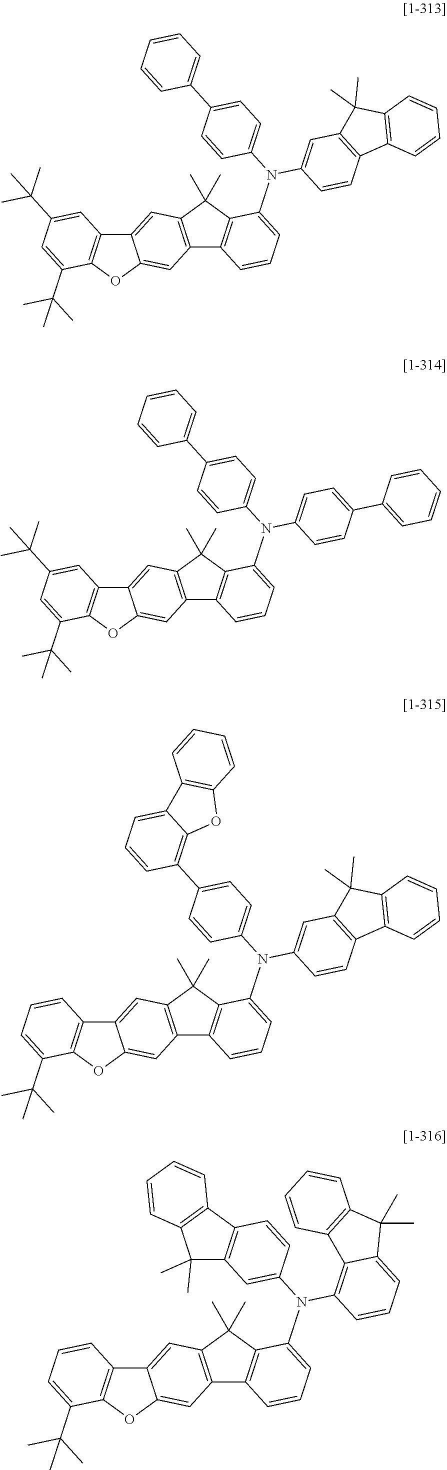 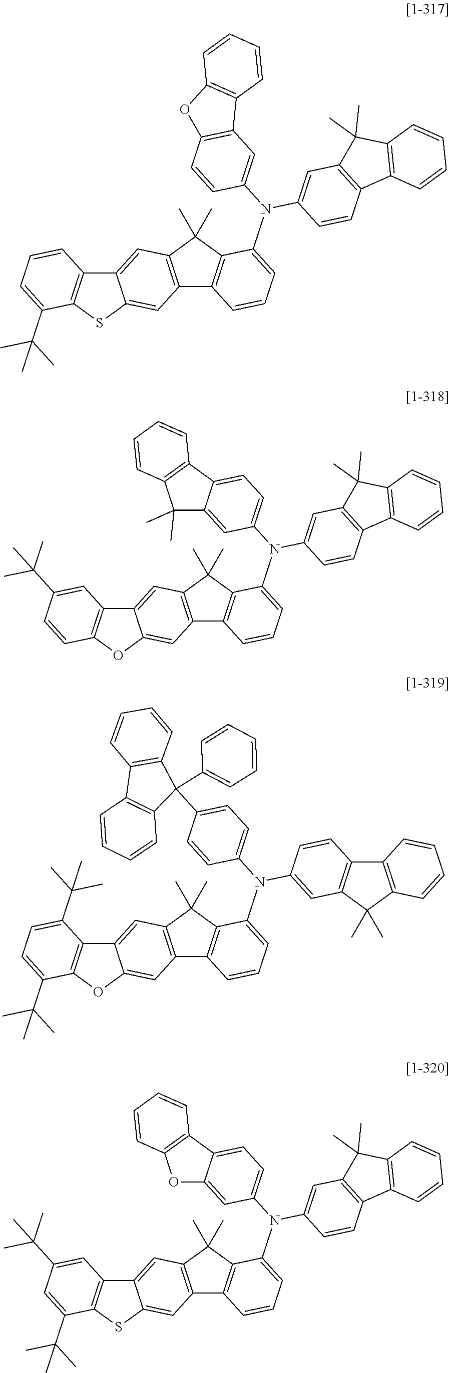 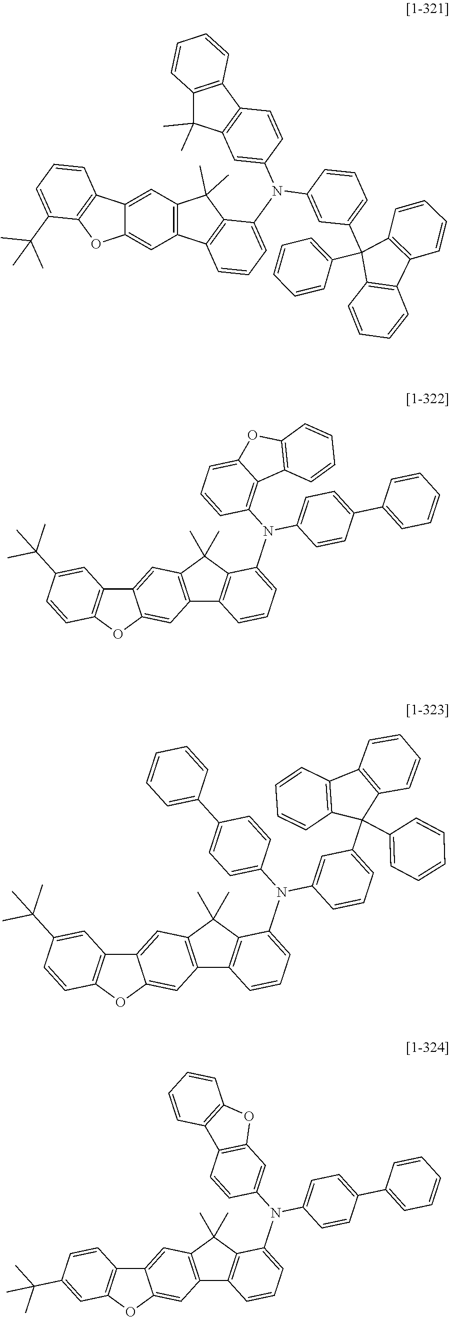 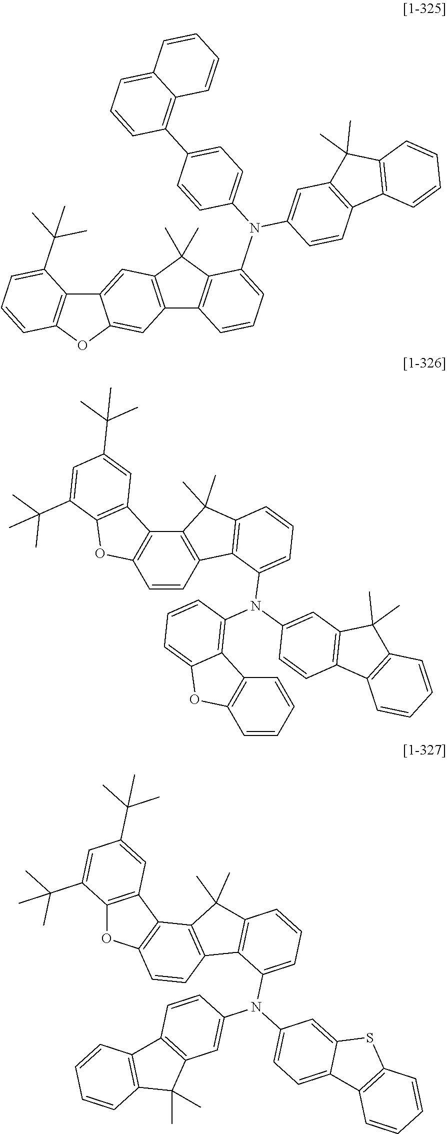 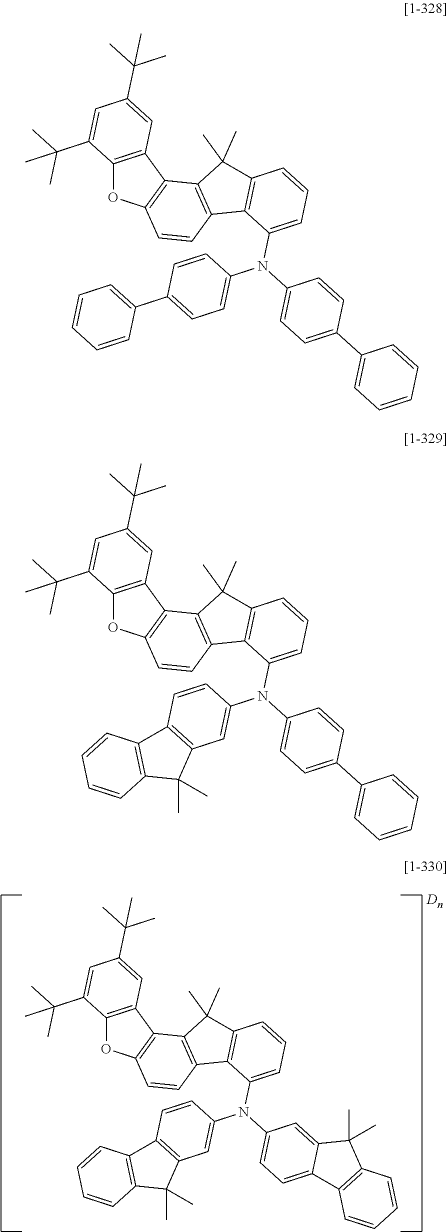 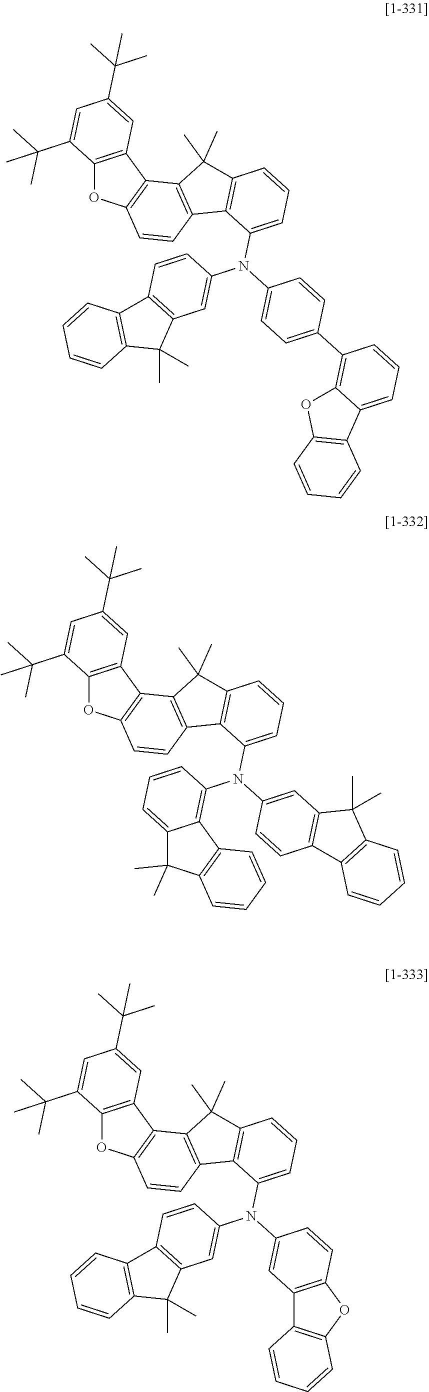 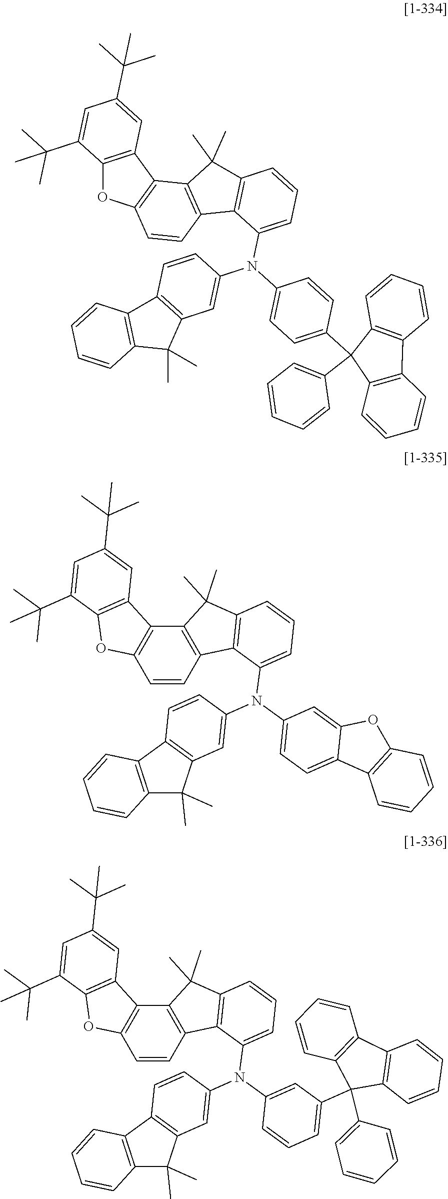 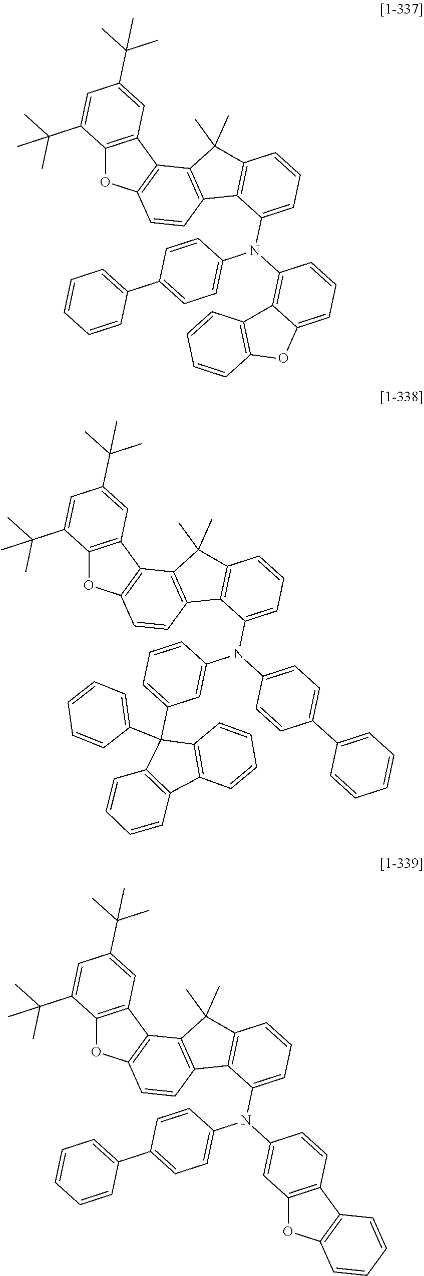 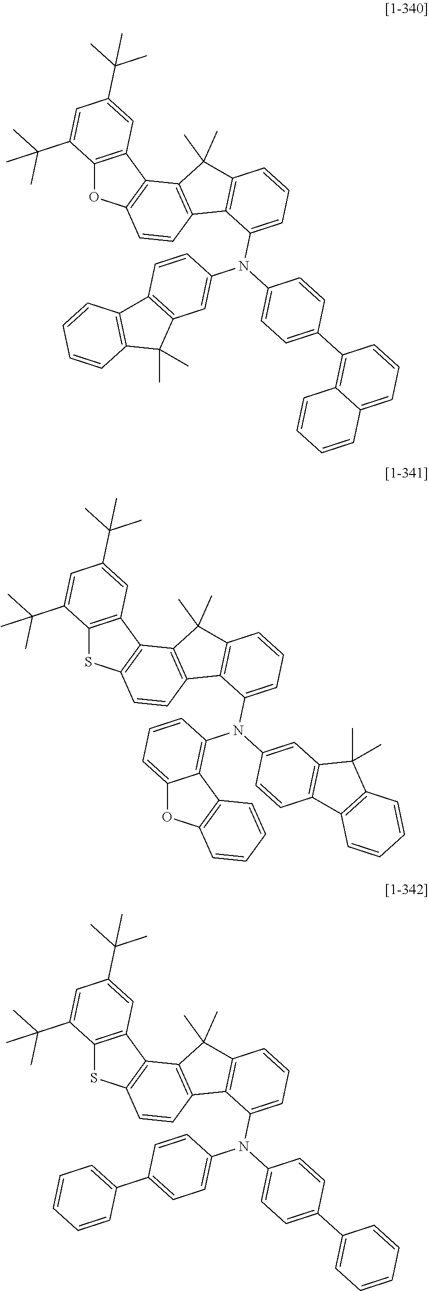 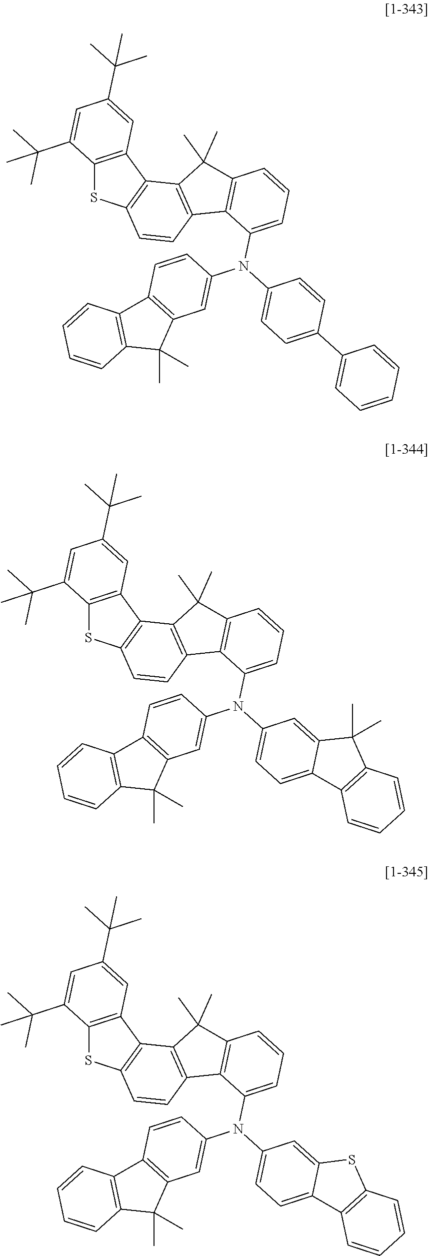 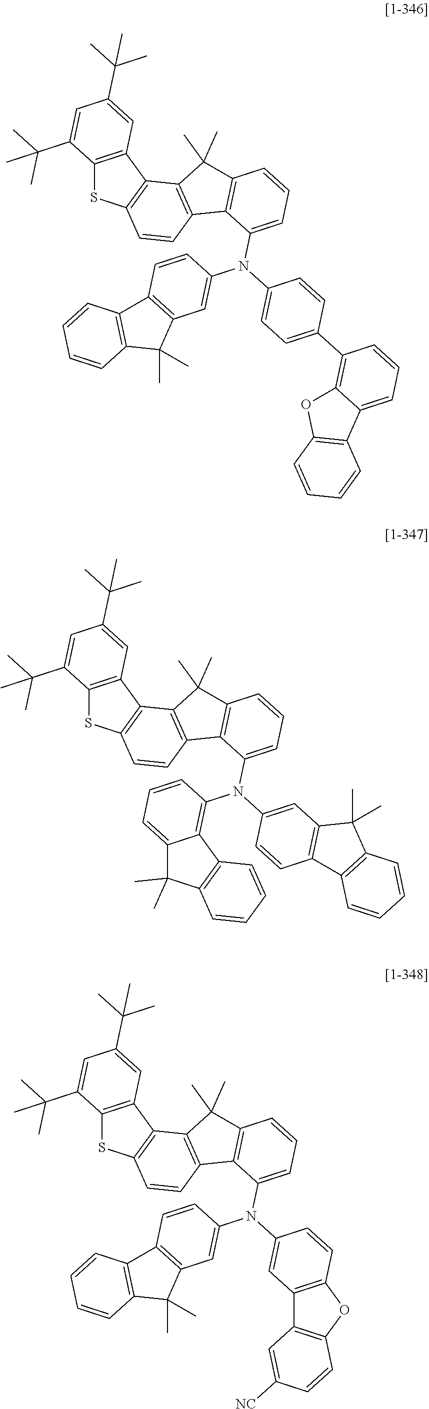 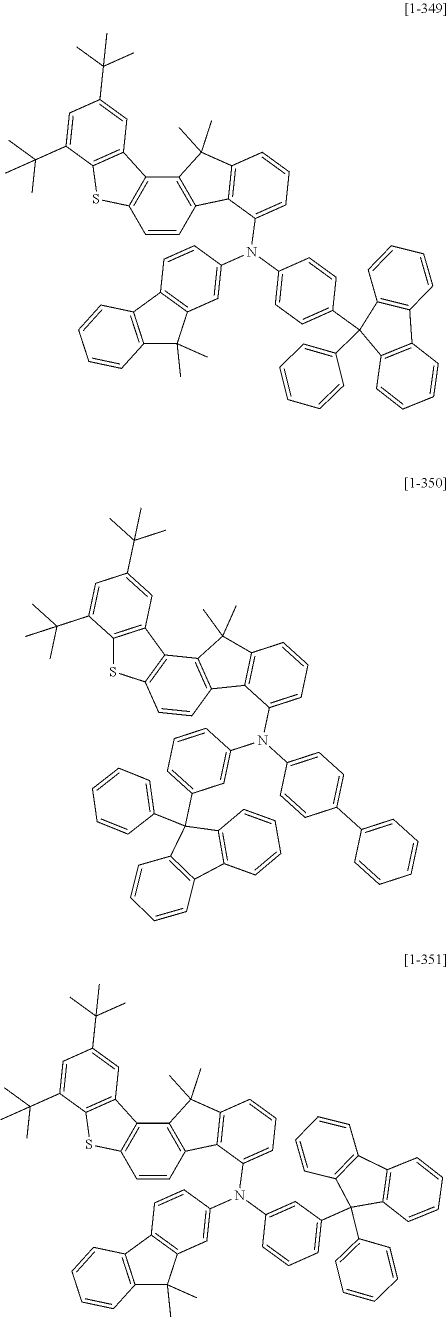 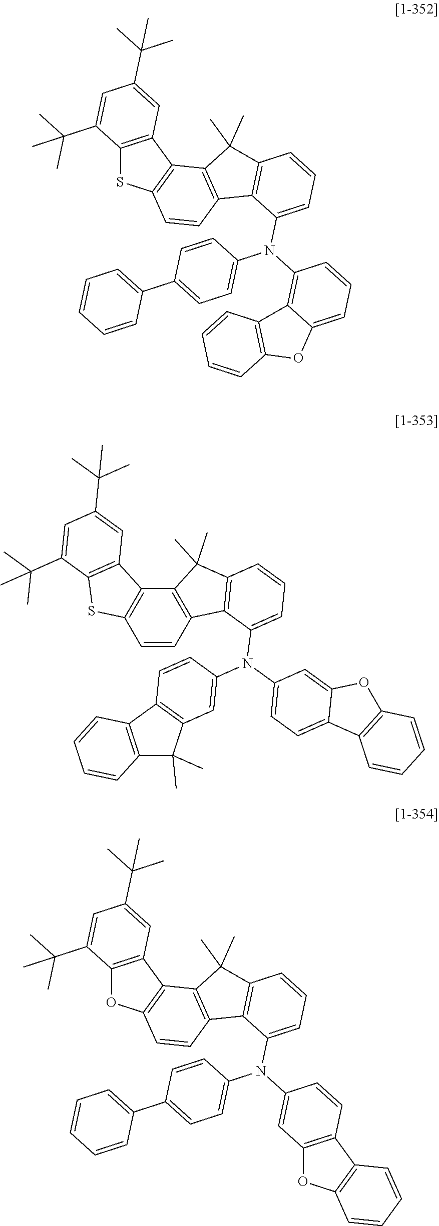 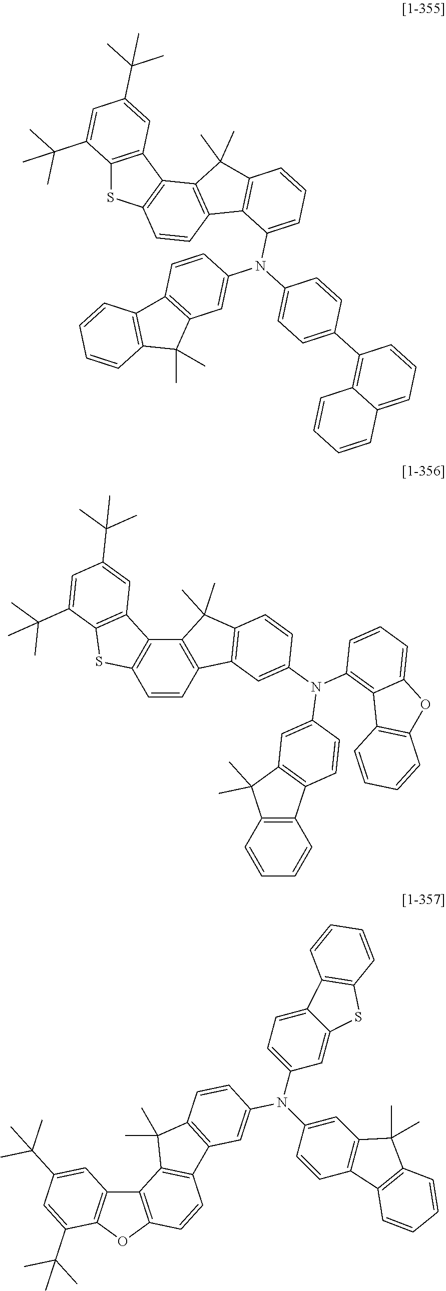 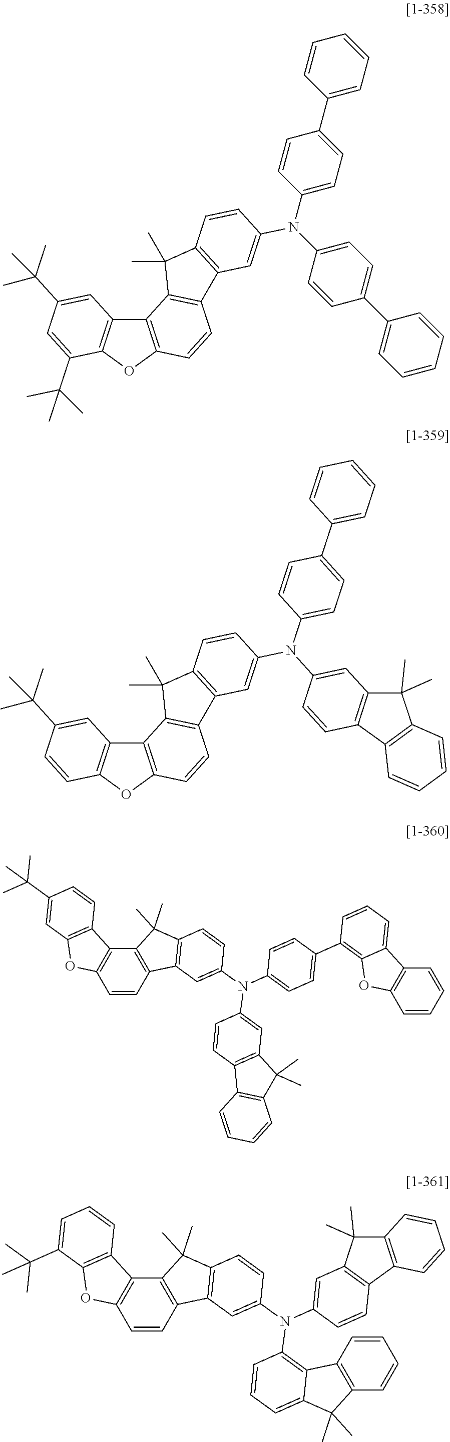 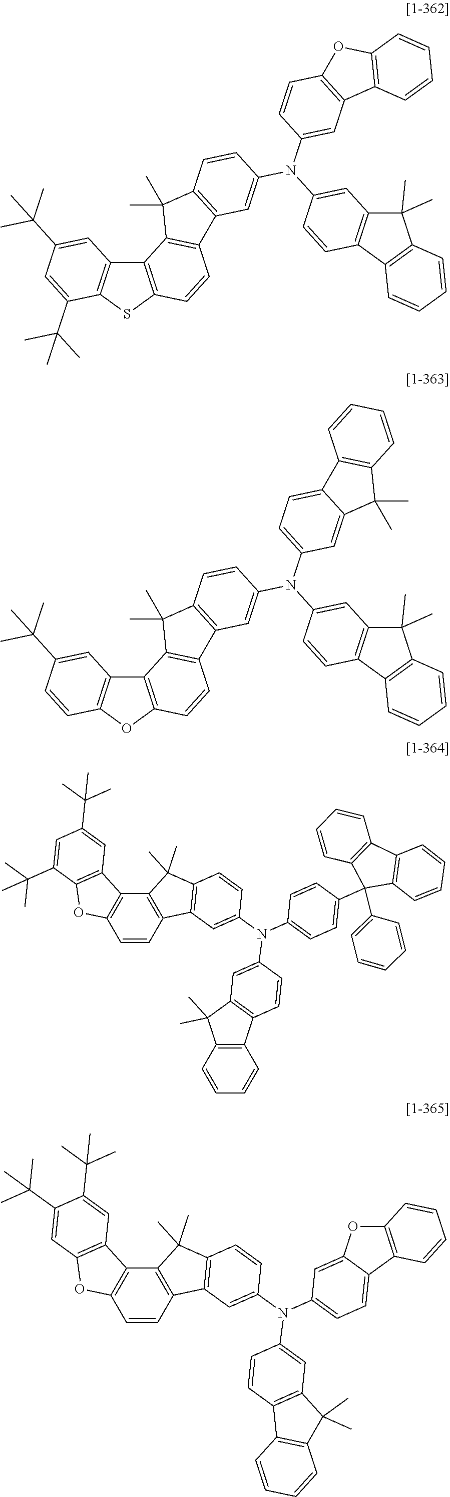 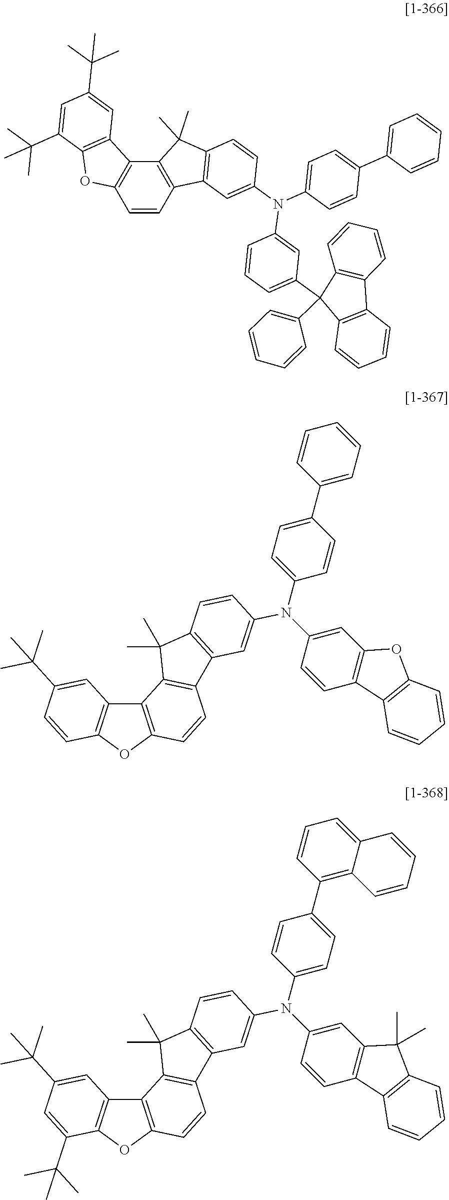 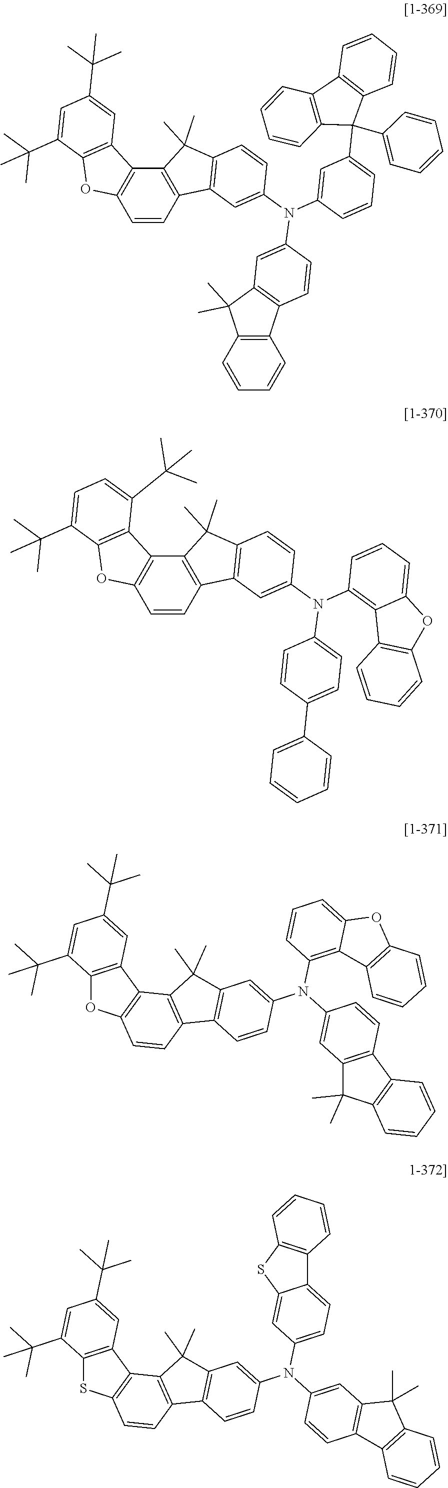 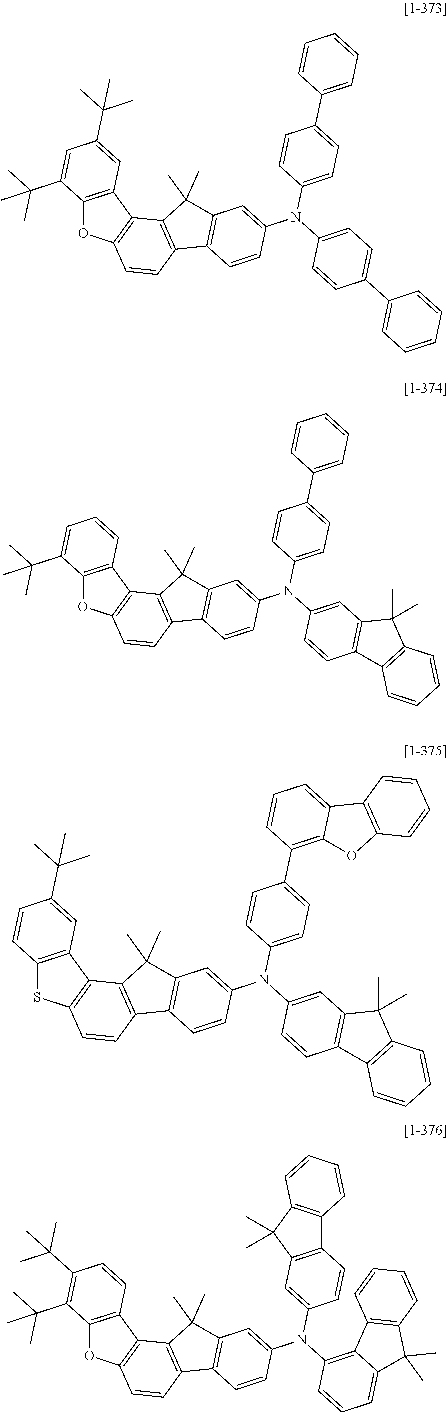 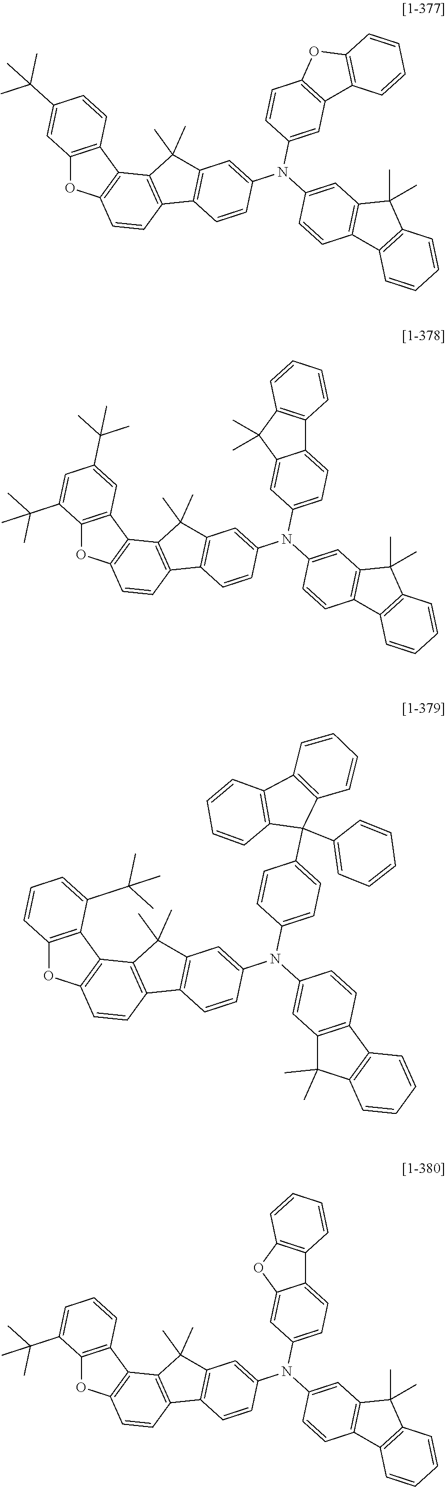 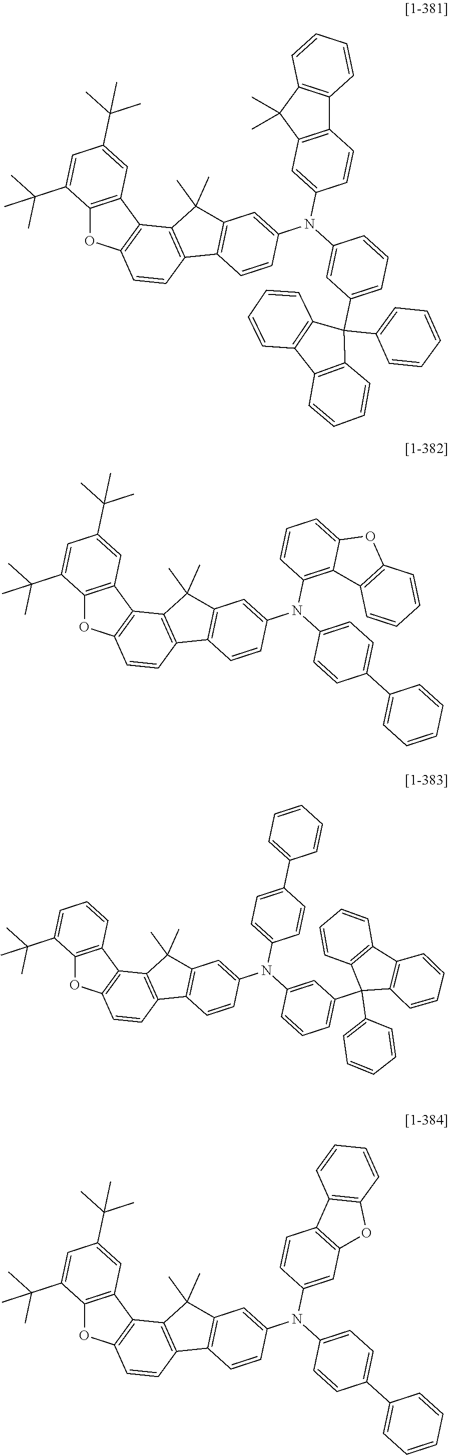 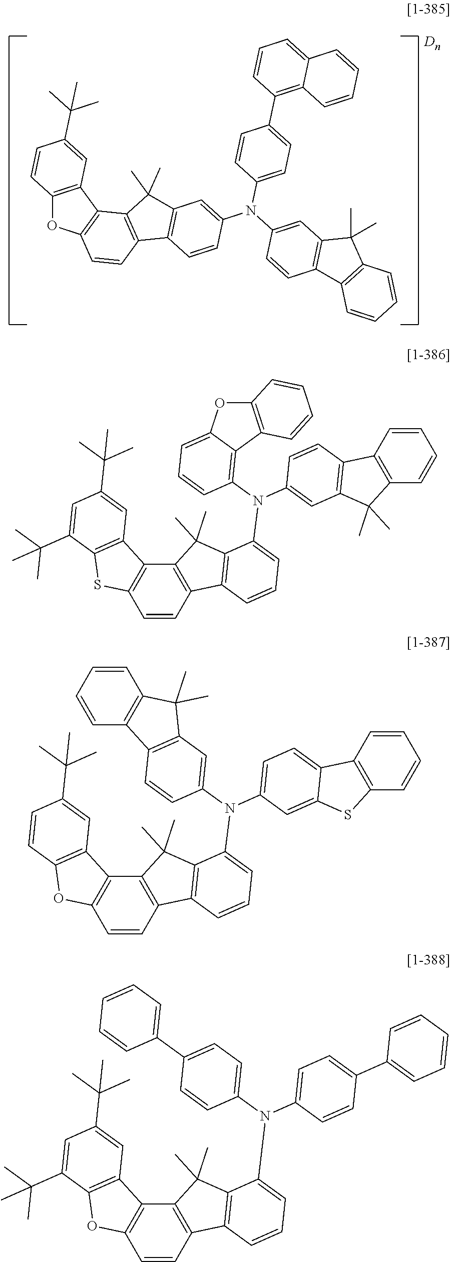 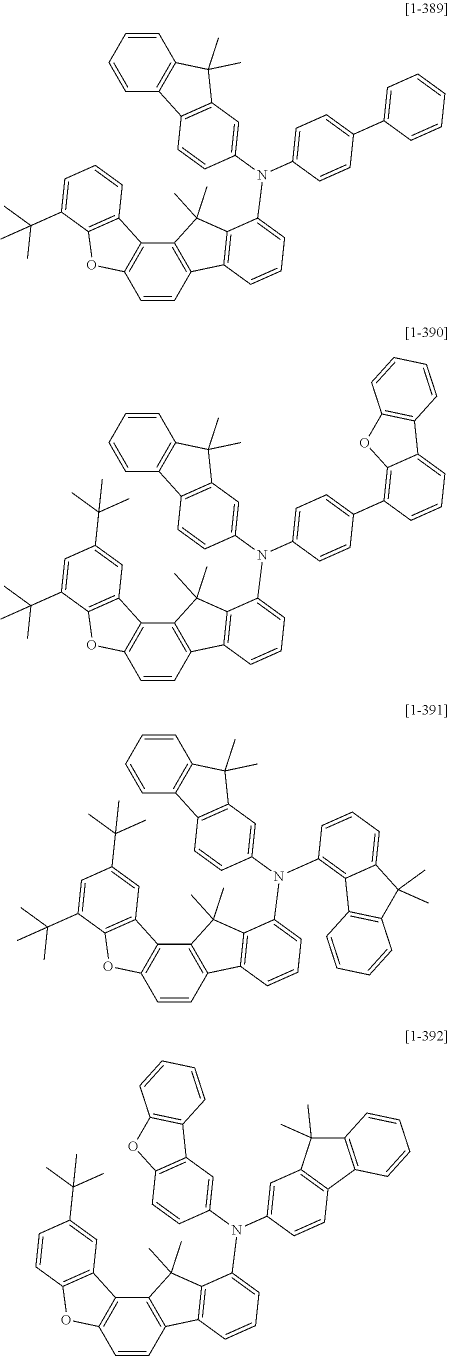 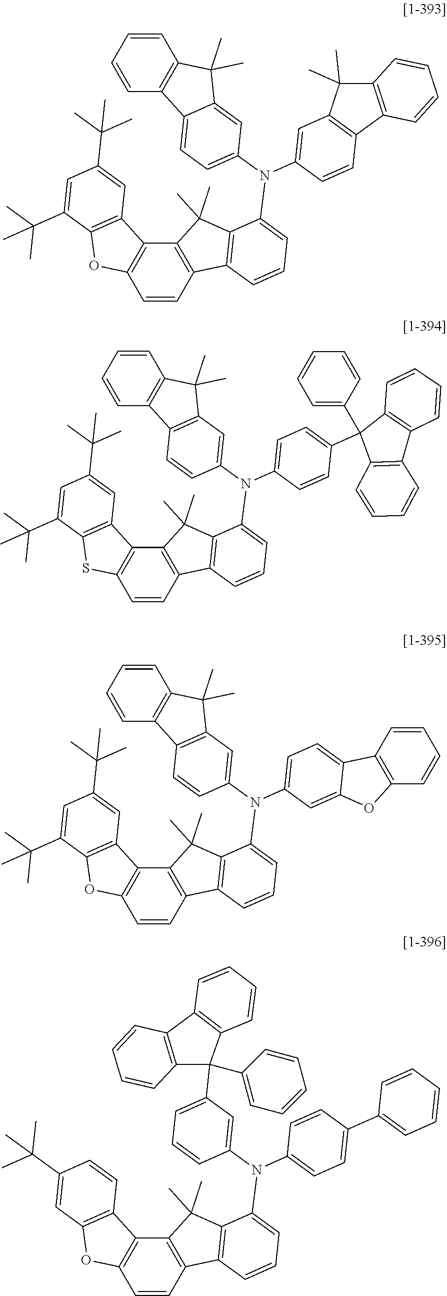 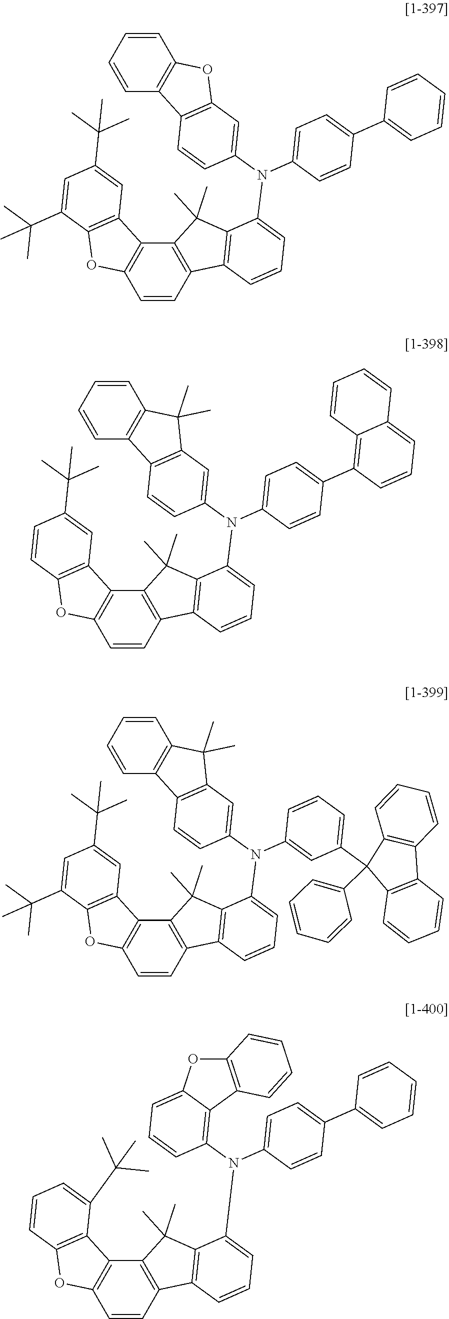 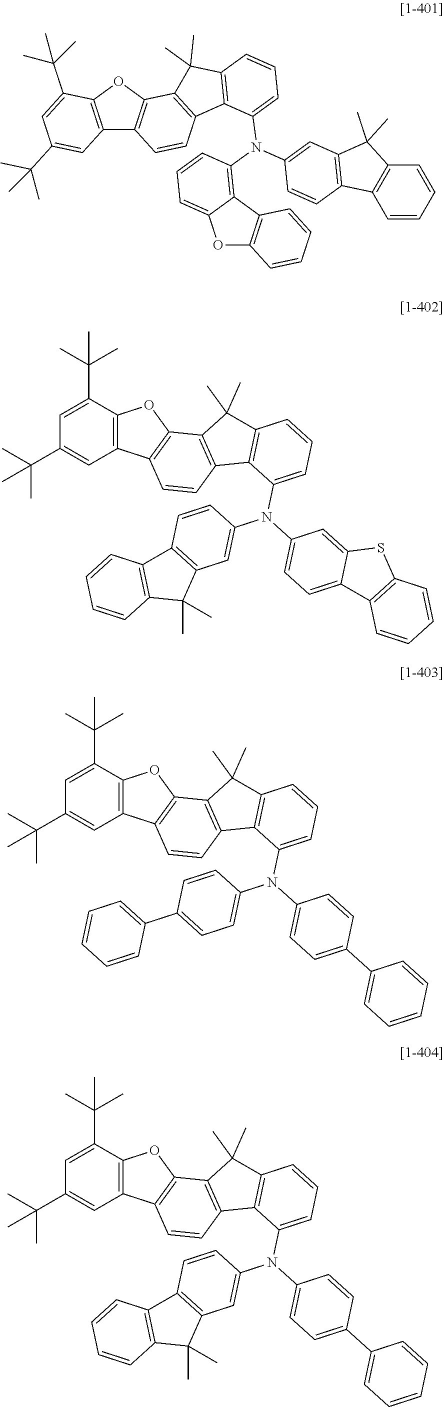 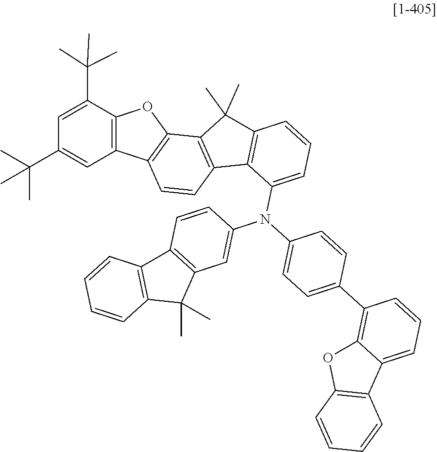 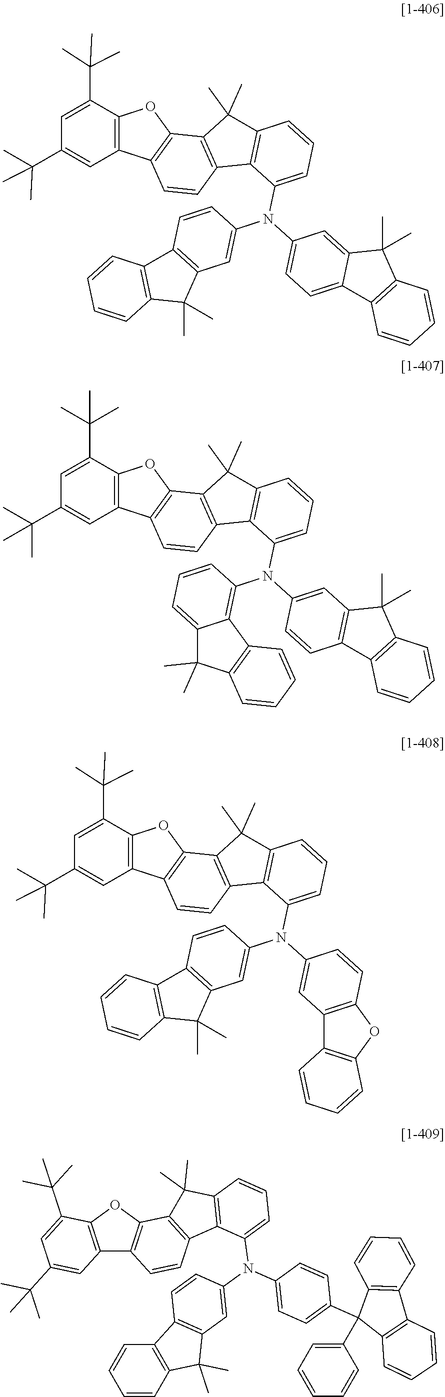 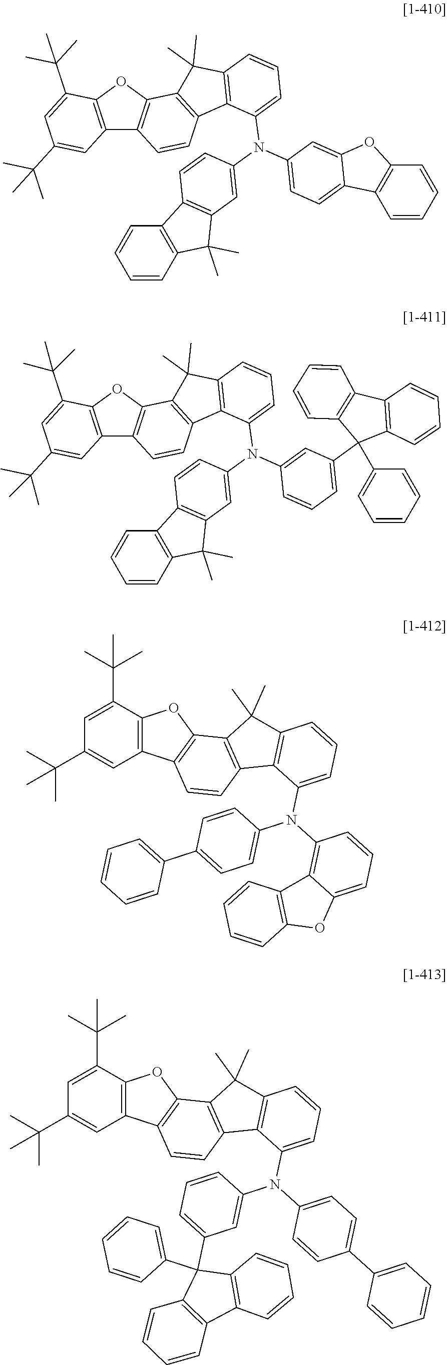 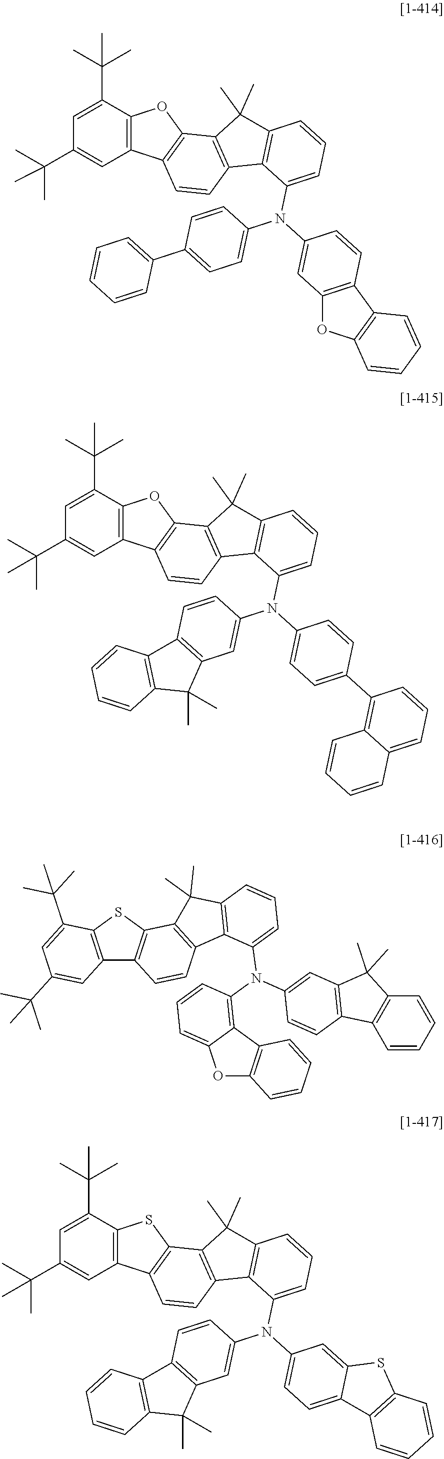 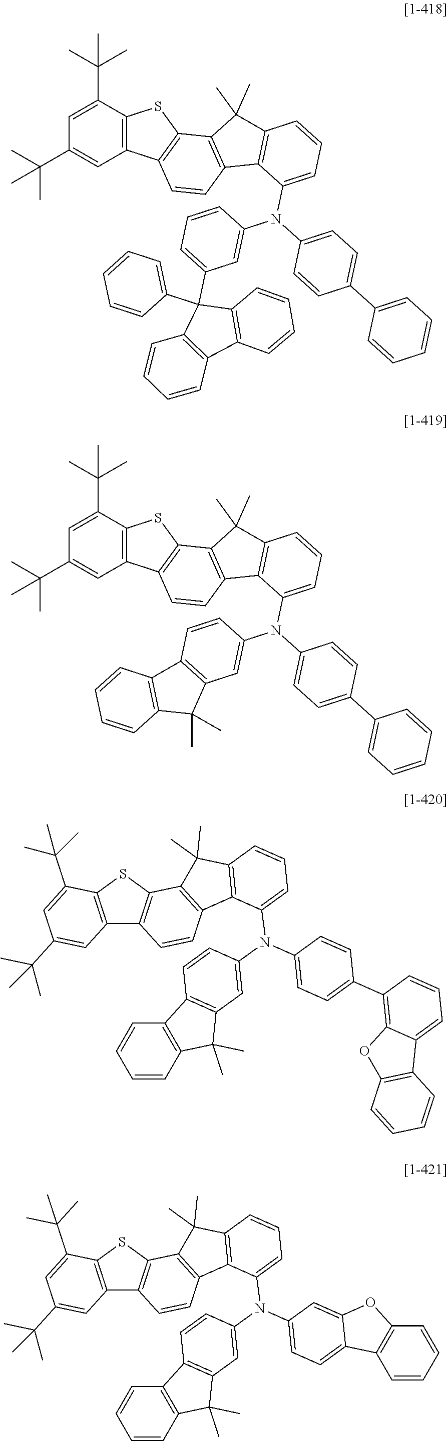 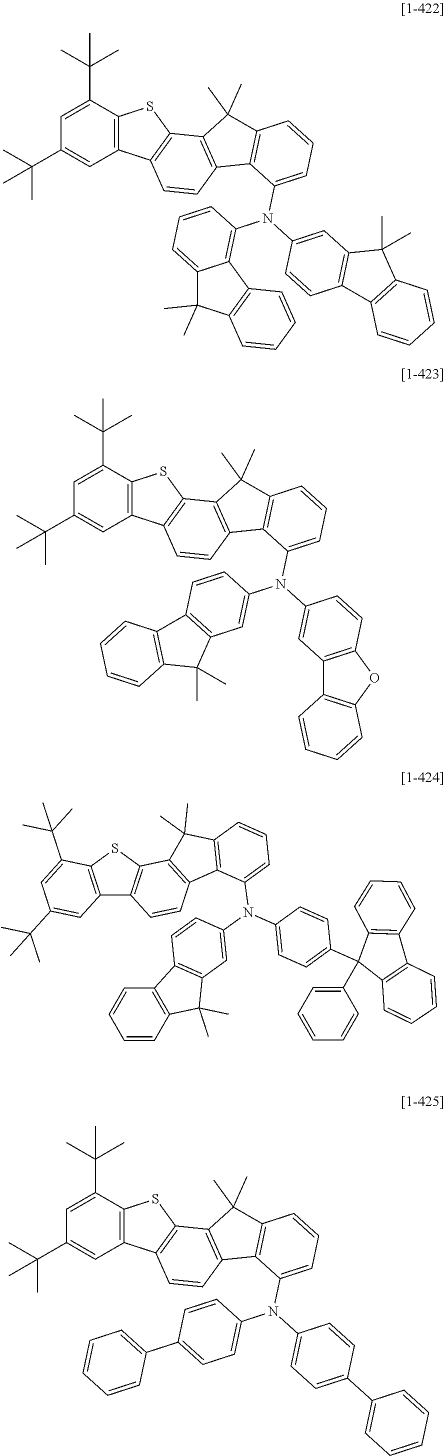 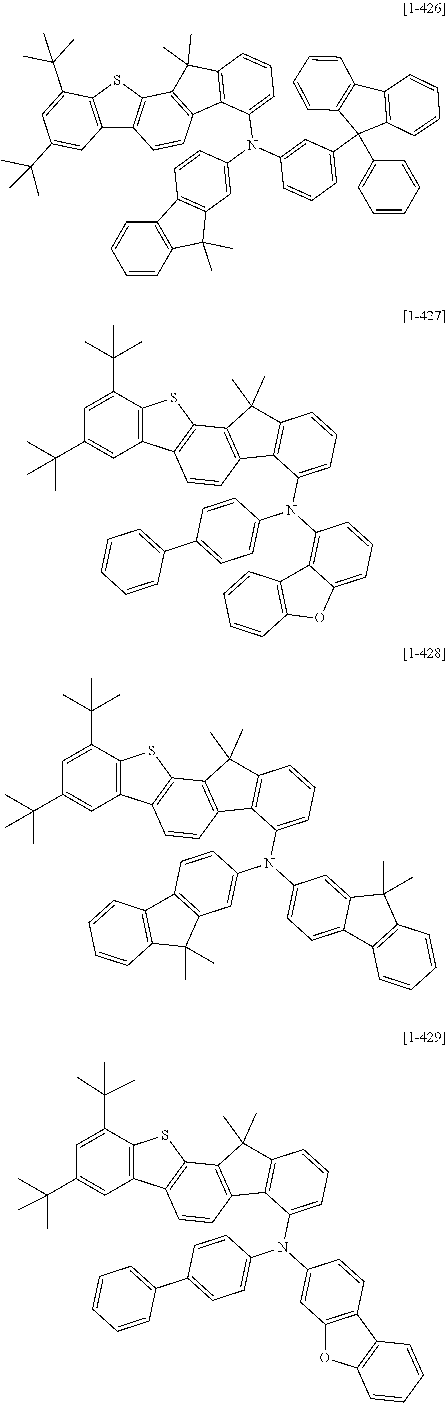 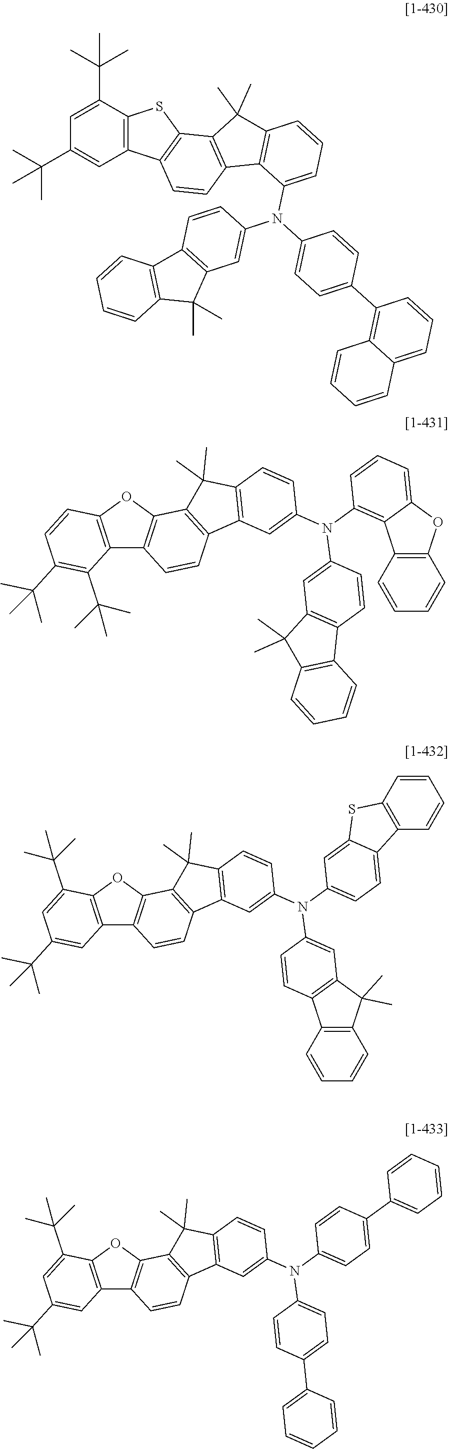 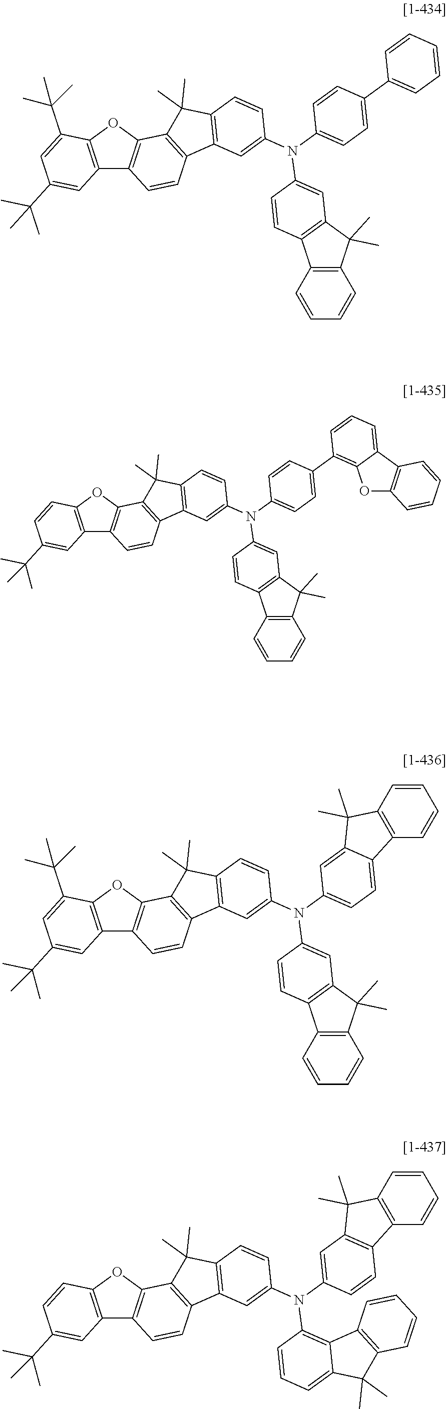 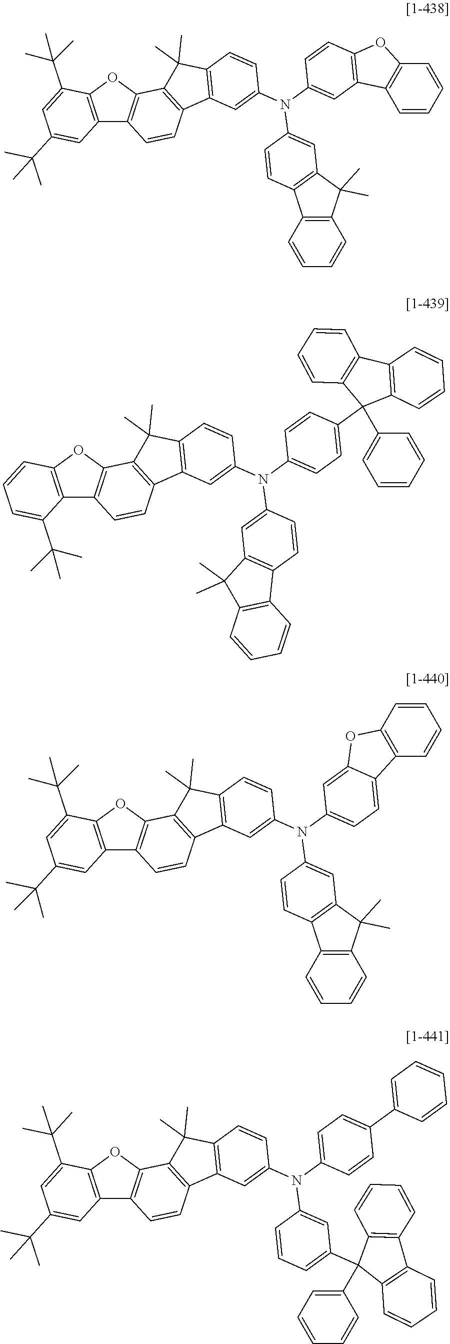 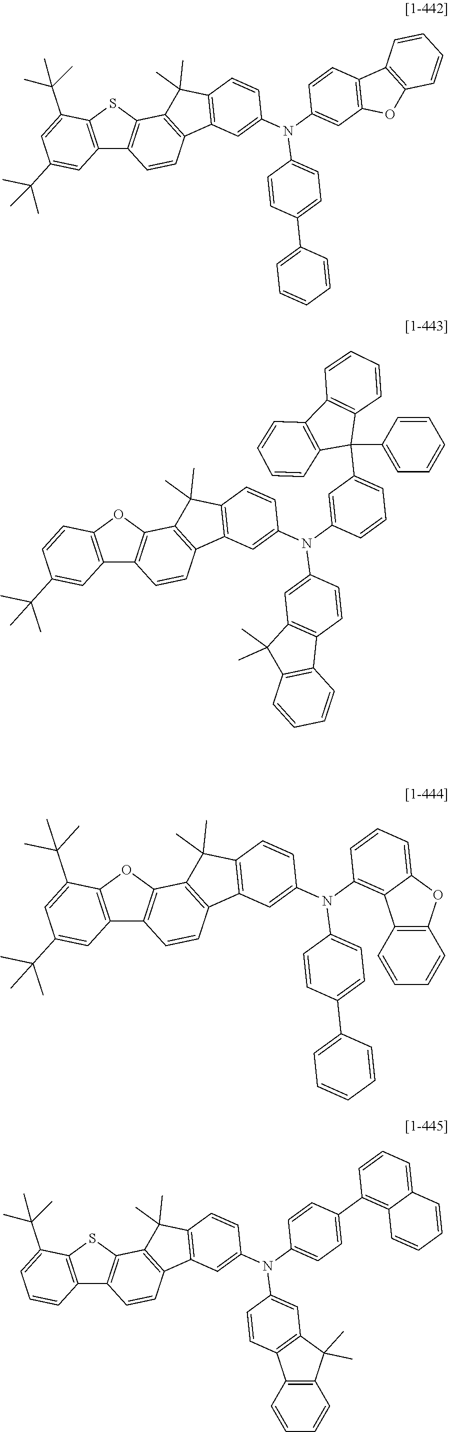 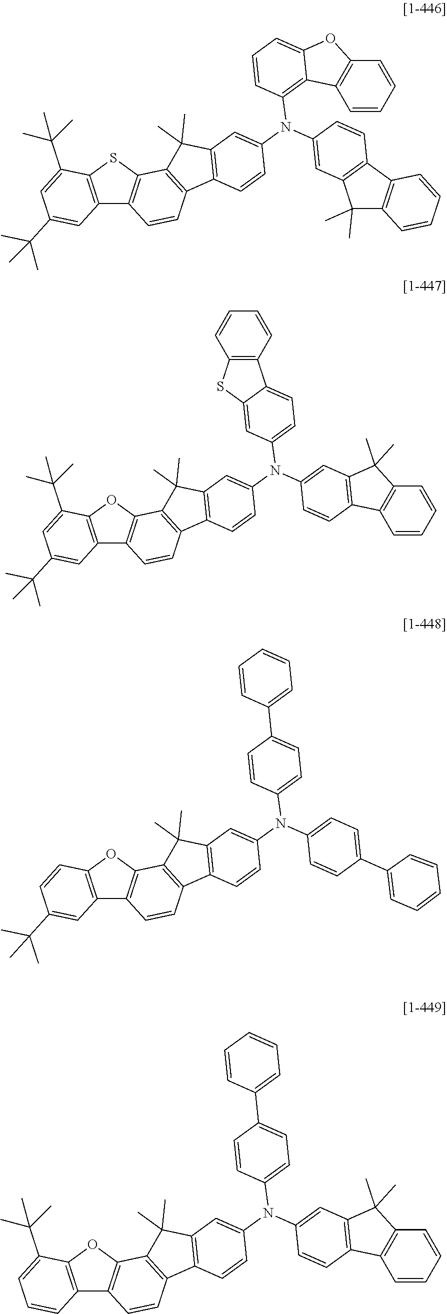 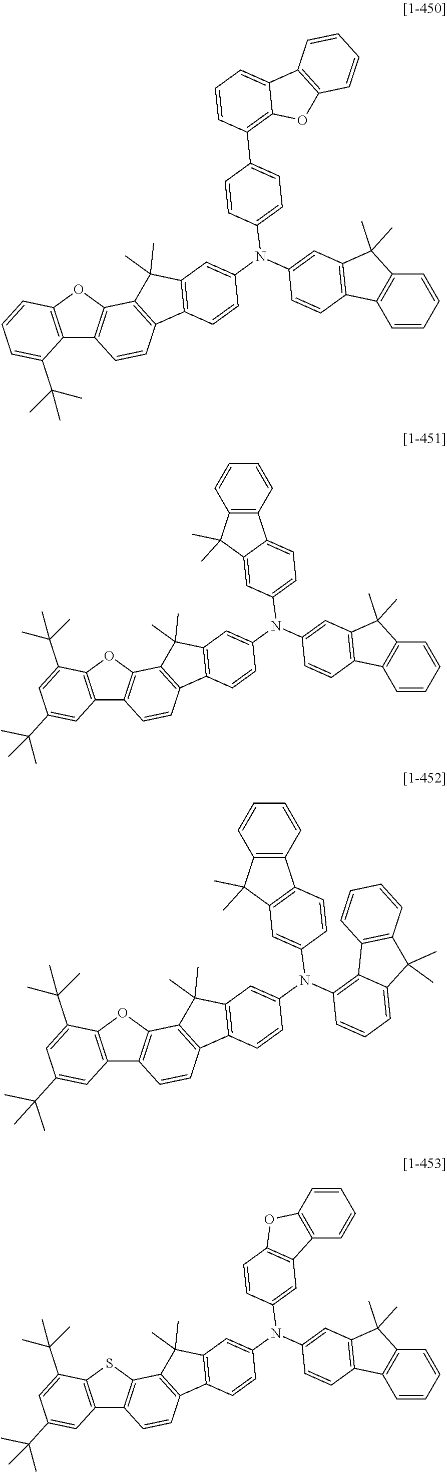 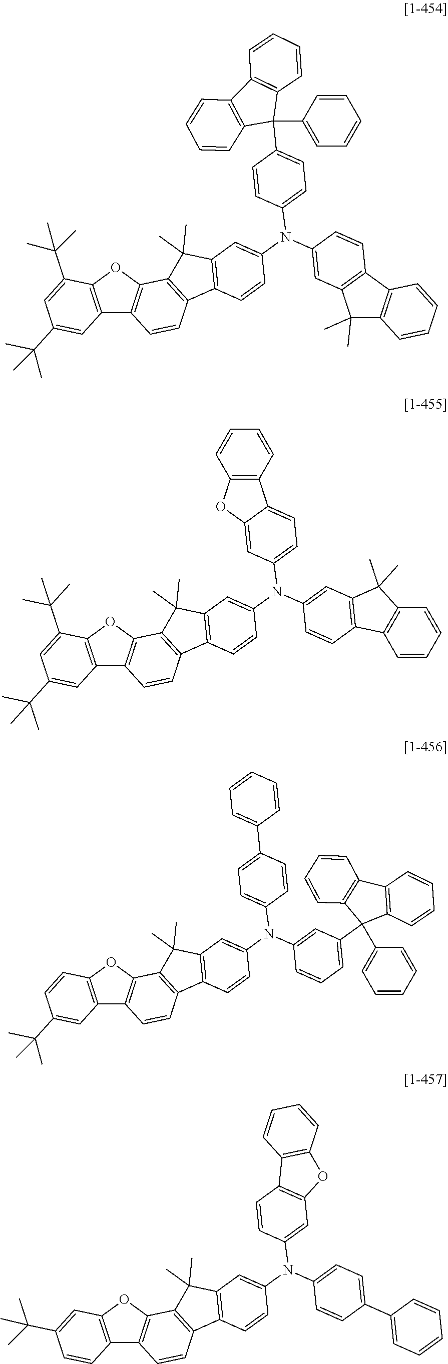 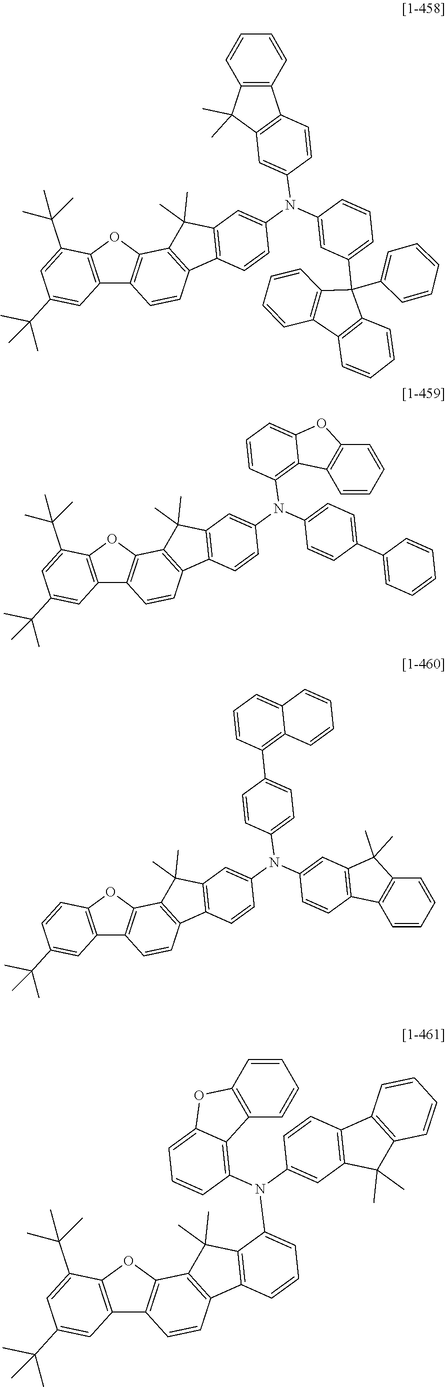 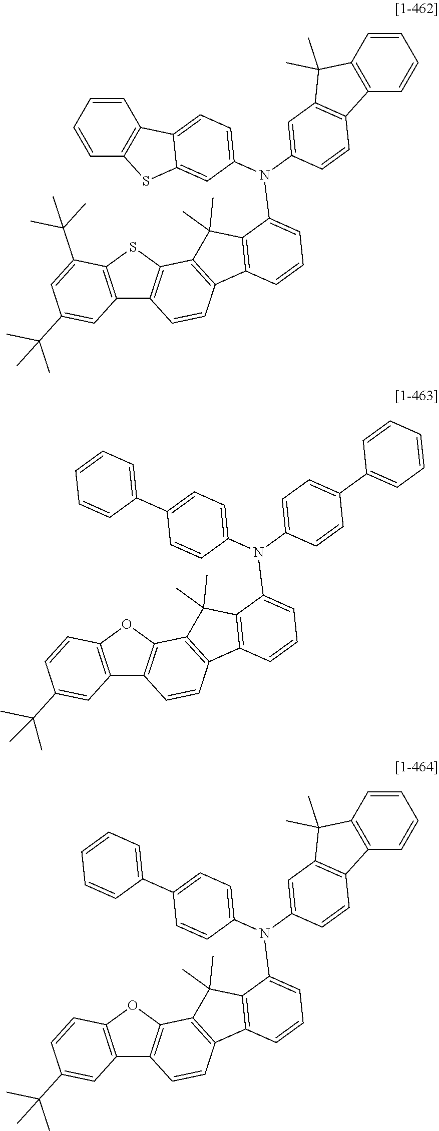 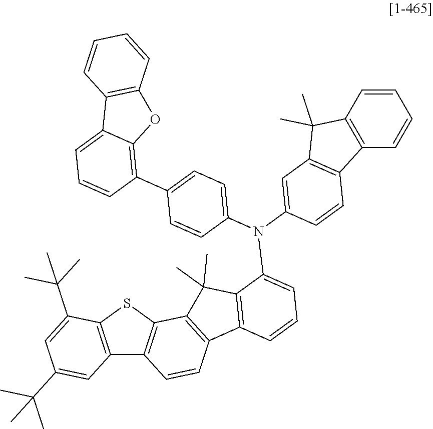
wherein, in Group 1, Dn refers to a number of hydrogens replaced with deuterium, and the number of n of Dn is 0 or more and is the maximum number that can be replaced.

8. An organic optoelectronic device, comprising:
an anode and a cathode facing each other, and
at least one organic layer between the anode and the cathode,
wherein the at least one organic layer includes the compound for an organic optoelectronic device as claimed in claim 1.

9. The organic optoelectronic device as claimed in claim 8, wherein:
the at least one organic layer includes a light emitting layer, and
the light emitting layer includes the compound for an organic optoelectronic device.

10. The organic optoelectronic device as claimed in claim 8, wherein:
the at least one organic layer includes:
a light emitting layer,
a hole transport layer between the anode and the light emitting layer, and
a hole transport auxiliary layer between the light emitting layer and the hole transport layer, and
the hole transport auxiliary layer includes the compound for an organic optoelectronic device.

11. A display device comprising the organic optoelectronic device as claimed in claim 8.
